(19) United States
(12) Patent Application Publication
(10) Pub. No.: US 2007/0072904 A1
(43) Pub. Date: Mar. 29, 2007

(54) PPAR active compounds

(76) Inventors: Jack Lin, Hercules, CA (US); Patrick Womack, Berkeley, CA (US); Byunghun Lee, Marina, CA (US); Shenghua Shi, San Diego, CA (US); Chao Zhang, Moraga, CA (US); Rebecca Zuckerman, Alameda, CA (US); Dean R. Artis, Kensington, CA (US); Prabha N. Ibrahim, Mountain View, CA (US); Weiru Wang, Lafayette, CA (US)

Correspondence Address: FOLEY & LARDNER LLP, P.O. BOX 80278, SAN DIEGO, CA, 92138-0278, US

(21) Appl. No.: 11/517,573

(22) Filed: Sep. 6, 2006

Related U.S. Application Data
(60) Provisional application No. 60/715,327, filed on Sep. 7, 2005.

(51) Int. Cl. A61K 31/4709; A61K 31/404; C07D 409/02

(52) U.S. Cl. 514/314; 514/418; 514/337; 514/414; 548/454; 546/113; 546/159

(57) ABSTRACT

Compounds are described that are active on PPARs, including pan-active compounds and compounds selective for any one or any two of PPARα, PPARα and PPARδ. Also described are methods of use of the compounds in treating various diseases.

### RELATED PATENT APPLICATIONS

This application claims the benefit of U.S. Prov. App. No. 60/715,327, filed Sep. 7, 2005, which is incorporated herein by reference in its entirety and for all purposes.

### FIELD OF THE INVENTION

The present invention relates to the field of modulators for members of the family of nuclear receptors identified as peroxisome proliferator-activated receptors.

### BACKGROUND OF THE INVENTION

The following description is provided solely to assist the understanding of the reader. None of the references cited or information provided is admitted to be prior art to the present invention. Each of the references cited herein is incorporated by reference in its entirety, to the same extent as if each reference were individually indicated to be incorporated by reference herein in its entirety.

The peroxisome proliferator-activated receptors (PPARs) form a subfamily in the nuclear receptor superfamily. Three isoforms, encoded by separate genes, have been identified thus far: PPARγ, PPARα, and PPARδ.

There are two PPARγ isoforms expressed at the protein level in mouse and human, γ1 and γ2. They differ only in that the latter has 30 additional amino acids at its N terminus due to differential promoter usage within the same gene, and subsequent alternative RNA processing. PPARγ2 is expressed primarily in adipose tissue, while PPARγ1 is expressed in a broad range of tissues.

Murine PPARα was the first member of this nuclear receptor subclass to be cloned; it has since been cloned from humans. PPARα is expressed in numerous metabolically active tissues, including liver, kidney, heart, skeletal muscle, and brown fat. It is also present in monocytes, vascular endothelium, and vascular smooth muscle cells. Activation of PPARα induces hepatic peroxisome proliferation, hepatomegaly, and hepatocarcinogenesis in rodents. These toxic effects are not observed in humans, although the same compounds activate PPARα across species.

Human PPARδ was cloned in the early 1990s and subsequently cloned from rodents. PPARδ is expressed in a wide range of tissues and cells with the highest levels of expression found in the digestive tract, heart, kidney, liver, adipose, and brain.

The PPARs are ligand-dependent transcription factors that regulate target gene expression by binding to specific peroxisome proliferator response elements (PPREs) in enhancer sites of regulated genes. PPARs possess a modular structure composed of functional domains that include a DNA binding domain (DBD) and a ligand binding domain (LBD). The DBD specifically binds PPREs in the regulatory region of PPAR-responsive genes. The DBD, located in the C-terminal half of the receptor contains the ligand-dependent activation domain, AF-2. Each receptor binds to its PPRE as a heterodimer with a retinoid X receptor (RXR). Upon binding an agonist, the conformation of a PPAR is altered and stabilized such that a binding cleft, made up in part of the AF-2 domain, is created and recruitment of transcriptional coactivators occurs. Coactivators augment the ability of nuclear receptors to initiate the transcription process. The result of the agonist-induced PPAR-coactivator interaction at the PPRE is an increase in gene transcription. Downregulation of gene expression by PPARs appears to occur through indirect mechanisms. (Bergen & Wagner, 2002, Diabetes Tech. & Ther., 4:163-174).

The first cloning of a PPAR (PPARα) occurred in the course of the search for the molecular target of rodent hepatic peroxisome proliferating agents. Since then, numerous fatty acids and their derivatives, including a variety of eicosanoids and prostaglandins, have been shown to serve as ligands of the PPARs. Thus, these receptors may play a central role in the sensing of nutrient levels and in the modulation of their metabolism. In addition, PPARs are the primary targets of selected classes of synthetic compounds that have been used in the successful treatment of diabetes and dyslipidemia. As such, an understanding of the molecular and physiological characteristics of these receptors has become extremely important to the development and utilization of drugs used to treat metabolic disorders. [00101 Kota et al., 2005, Pharmacological Research 51: 85-94, provides a review of biological mechanisms involving PPARs that includes a discussion of the possibility of using PPAR modulators for treating a variety of conditions, including chronic inflammatory disorders such as atherosclerosis, arthritis and inflammatory bowel syndrome, retinal disorders associated with angiogenesis, increased fertility, and neurodegenerative diseases.

Yousef et al., 2004, Journal of Biomedicine and Biotechnology 2004(3):156-166, discusses the anti-inflammatory effects of PPARα, PPARγ and PPARδ agonists, suggesting that PPAR agonists may have a role in treating neuronal diseases such as Alzheimer's disease, and autoimmune diseases such as inflammatory bowel disease and multiple sclerosis. A potential role for PPAR agonists in the treatment of Alzheimer's disease has been described in Combs et al., 2000, Journal of Neuroscience 20(2): 558, and such a role for PPAR agonists in Parkinson's disease is discussed in Breidert et al. 2002, Journal of Neurochemistry, 82: 615. A potential related function of PPAR agonists in treatment of Alzheimer's disease, that of regulation of the APP-processing enzyme BACE, has been discussed in Sastre et al. 2003, Journal of Neuroscience 23(30):9796. These studies collectively indicate PPAR agonists may provide advantages in treating a variety of neurodegenerative diseases by acting through complementary mechanisms.

Discussion of the anti-inflammatory effects of PPAR agonists is also available in Feinstein, 2004, Drug Discovery Today: Therapeutic Strategies 1(1):29-34 in relation to multiple sclerosis and Alzheimer's disease; Patel et al., 2003, The Journal of Immunology, 170:2663-2669 in relation to chronic obstructive pulmonary disease (COPD) and asthma; Lovett-Racke et al., 2004, The Journal of Immunology, 172:5790-5798 in relation to autoimmune disease; Malhotra et al., 2005, Expert Opinions in Pharmacotherapy, 6(9):1455-1461 in relation to psoriasis; and Storer et al., 2005, Journal of Neuroimmunology, 161:113-122 in relation to multiple sclerosis.

This wide range of roles for the PPARs that have been discovered suggest that PPARα, PPARγ and PPARδ may play a role in a wide range of events involving the vasculature, including atherosclerotic plaque formation and stability, thrombosis, vascular tone, angiogenesis, cancer, pregnancy, pulmonary disease, autoimmune disease, and neurological disorders.

Among the synthetic ligands identified for PPARs are thiazolidinediones (TZDs). These compounds were originally developed on the basis of their insulin-sensitizing effects in animal pharmacology studies. Subsequently, it was found that TZDs induced adipocyte differentiation and increased expression of adipocyte genes, including the adipocyte fatty acid-binding protein aP2. Independently, it was discovered that PPARδ interacted with a regulatory element of the aP2 gene that controlled its adipocyte-specific expression. On the basis of these seminal observations, experiments were performed that determined that TZDs were PPARδ ligands and agonists and demonstrated a definite correlation between their in vitro PPARγ activities and their in vivo insulin-sensitizing actions. (Bergen & Wagner, supra).

Several TZDs, including troglitazone, rosiglitazone, and pioglitazone, have insulin-sensitizing and anti-diabetic activity in humans with type 2 diabetes and impaired glucose tolerance. Farglitazar is a very potent non-TZD PPARδ-selective agonist that was recently shown to have antidiabetic as well as lipid-altering efficacy in humans. In addition to these potent PPARδ ligands, a subset of the non-steroidal antiinflammatory drugs (NSAIDs), including indomethacin, fenoprofen, and ibuprofen, have displayed weak PPARδ and PPARα activities. (Bergen & Wagner, supra).

The fibrates, amphipathic carboxylic acids that have been proven useful in the treatment of hypertriglyceridemia, are PPARα ligands. The prototypical member of this compound class, clofibrate, was developed prior to the identification of PPARs, using in vivo assays in rodents to assess lipid-lowering efficacy. (Bergen & Wagner, supra).

Fu et al., Nature, 2003, 425:9093, demonstrated that the PPARα binding compound, oleylethanolamide, produces satiety and reduces body weight gain in mice.

Clofibrate and fenofibrate have been shown to activate PPARα with a 10-fold selectivity over PPARγ. Bezafibrate acts as a pan-agonist that shows similar potency on all three PPAR isoforms. Wy-14643, the 2-arylthioacetic acid analogue of clofibrate, is a potent murine PPARα agonist as well as a weak PPARγ agonist. In humans, all of the fibrates must be used at high doses (200-1,200 mg/day) to achieve efficacious lipid-lowering activity.

TZDs and non-TZDs have also been identified that are dual PPARγ/α agonists. By virtue of the additional PPARα agonist activity, this class of compounds has potent lipid-altering efficacy in addition to antihyperglycemic activity in animal models of diabetes and lipid disorders. KRP-297 is an example of a TZD dual PPARγ/α agonist (Fajas, 1997, J. Biol. Chem., 272:18779-18789); furthermore DRF-2725 and AZ-242 are non-TZD dual PPARγ/α a agonists. (Lohray, et al., 2001, J. Med. Chem., 44:2675-2678; Cronet, et al., 2001, Structure (Camb.) 9:699-706).

In order to define the physiological role of PPARδ, efforts have been made to develop novel compounds that activate this receptor in a selective manner. Amongst the α-substituted carboxylic acids previously described, the potent PPARδ ligand L-165041 demonstrated approximately 30-fold agonist selectivity for this receptor over PPARγ, and it was inactive on murine PPARα (Liebowitz, et al., 2000, FEBS Lett., 473:333-336). This compound was found to increase high-density lipoprotein levels in rodents. It was also reported that GW501516 was a potent, highly-selective PPARδ agonist that produced beneficial changes in serum lipid parameters in obese, insulin-resistant rhesus monkeys. (Oliver et al., 2001, Proc. Natl. Acad. Sci., 98:5306-5311).

In addition to the compounds discussed above, certain thiazole derivatives active on PPARs have been described. (Cadilla et al., Intemat. Appl. PCT/US01/149320, Intemat. Publ. WO 02/062774, incorporated herein by reference in its entirety.)

Some tricyclic-α-alkyloxyphenylpropionic acids have been described as dual PPARα/γ agonists in Sauerberg et al., 2002, J. Med. Chem. 45:789-804.

A group of compounds that are stated to have equal activity on PPARα/γ/δ is described in Morgensen et al., 2002, Bioorg. & Med. Chem. Lett. 13:257-260.

Oliver et al., describes a selective PPARδ agonist that promotes reverse cholesterol transport. (Oliver et al., supra).

Yamamoto et al., U.S. Pat. No.3,489,767 describes “1-(phenylsulfonyl)-indolyl aliphatic acid derivatives” that are stated to have “antiphlogistic, analgesic and antipyretic actions.” (Col. 1, lines 16-19.)

Kato et al., European Patent Application 94101551.3, Publication No. 0 610 793 A1, describes the use of 3 -(5-methoxy-1-p-toluenesulfonylindol-3-yl)propionic acid (page 6) and 1-(2,3,6-triisopropylphenylsulfonyl)-indole-3-propionic acid (page 9) as intermediates in the synthesis of particular tetracyclic morpholine derivatives useful as analgesics.

This application is related to the following published patent applications: WO 2005009958, US 20050038246, and US 20050288354, each of which are hereby incorporated by reference herein in their entireties including all specifications, figures, and tables, and for all purposes.

### SUMMARY OF THE INVENTION

The present invention relates to compounds active on PPARs, which are useful for a variety of applications, e.g., therapeutic and/or prophylactic methods involving modulation of at least one of PPARα, PPARδ, and PPARγ. Included are compounds that have significant pan-activity across the PPAR family (PPARα, PPARδ, and PPARγ), as well as compounds that have significant specificity (at least 5-, 10-, 20-, 50-, or 100-fold greater activity) on a single PPAR, or on two of the three PPARs.

In one embodiment, the invention involves the use of compounds of Formula I as modulators of one or more of the PPARs, PPARα, PPARδ, and PPARγ, where Formula I is: 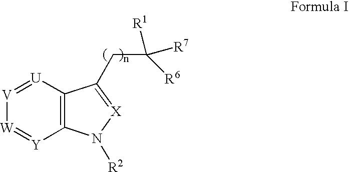
all salts, prodrugs, tautomers and isomers thereof,
wherein:
U, V, W, X, and Y are independently N or CR⁸, wherein at most two of U, V, W, and Y are N;
R¹ is selected from the group consisting of C(O)OR¹⁶ and a carboxylic acid isostere;
R² is selected from the group consisting of hydrogen, optionally substituted lower alkyl, —CH₂—CR¹²═CR¹³R¹⁴, —CH₂—C≡CR¹⁵, optionally substituted cycloalkyl, optionally substituted heterocycloalkyl, optionally substituted aryl, optionally substituted heteroaryl, —C(Z)NR¹⁰R¹¹, —C(Z)R²⁰, —S(O)₂NR¹⁰R¹¹ and —S(O)₂R²¹;
R⁶ and R⁷ are independently selected from the group consisting of hydrogen, optionally substituted lower alkyl, optionally substituted cycloalkyl, optionally substituted heterocycloalkyl, optionally substituted aryl, and optionally substituted heteroaryl; or
R⁶ and R⁷ combine to form a 3-7 membered monocyclic cycloalkyl or 5-7 membered monocyclic heterocycloalkyl;
R⁸ is selected from the group consisting of hydrogen, halogen, optionally substituted lower alkyl, —CH₂—CR¹²═CR¹³R¹⁴, —CH₂—C≡CR¹⁵, optionally substituted cycloalkyl, optionally substituted heterocycloalkyl, optionally substituted aryl, optionally substituted heteroaryl, —OR⁹, —SR⁹, —NR¹⁰R¹¹, —C(Z)NR¹⁰R¹¹, —C(Z)R²⁰, —S(O)₂NR¹⁰R¹¹, and —S(O)₂R²¹;
R⁹ is selected from the group consisting of optionally substituted lower alkyl, optionally substituted cycloalkyl, optionally substituted heterocycloalkyl, optionally substituted aryl, and optionally substituted heteroaryl;
R¹⁰ and R¹¹ are independently selected from the group consisting of hydrogen, optionally substituted lower alkyl, optionally substituted cycloalkyl, optionally substituted heterocycloalkyl, optionally substituted aryl, and optionally substituted heteroaryl; or
R¹⁰ and R¹¹ together with the nitrogen to which they are attached form a 5-7 membered monocyclic heterocycloalkyl or a 5 or 7 membered monocyclic nitrogen containing heteroaryl;
R¹⁶ is selected from the group consisting of hydrogen, optionally substituted lower alkyl, optionally substituted cycloalkyl, optionally substituted heterocyclyoalkyl, optionally substituted aryl, and optionally substituted heteroaryl;
R²⁰ is selected from the group consisting of —CH₂—CR¹²═CR¹³R¹⁴, —CH₂—C≡CR¹⁵, optionally substituted lower alkyl, optionally substituted cycloalkyl, optionally substituted heterocycloalkyl, optionally substituted aryl, and optionally substituted heteroaryl;
R²¹ is selected from the group consisting of —OR¹⁷, —CH₂—CR¹²═CR¹³R¹⁴, —CH₂—C≡CR¹⁵, optionally substituted cycloalkyl, optionally substituted heterocycloalkyl, optionally substituted aryl, and optionally substituted heteroaryl; R¹², R¹³, R¹⁴, and R¹⁵ are independently selected from the group consisting of optionally substituted lower alkyl, optionally substituted cycloalkyl, optionally substituted heterocycloalkyl, optionally substituted aryl, and optionally substituted heteroaryl;
R¹⁷ is selected from the group consisting of optionally substituted lower alkyl, optionally substituted cycloalkyl, optionally substituted heterocycloalkyl, optionally substituted aryl, optionally substituted heteroaryl, and -C(O)R¹⁸;
R¹⁸ is selected from the group consisting of hydrogen, optionally substituted lower alkyl, optionally substituted cycloalkyl, optionally substituted heterocyclyoalkyl, optionally substituted aryl, and optionally substituted heteroaryl;
Z is O or S; and
n=0, 1, or 2.

In some embodiments involving compounds of Formula I, the bicyclic core shown for Formula I has one of the following structures: 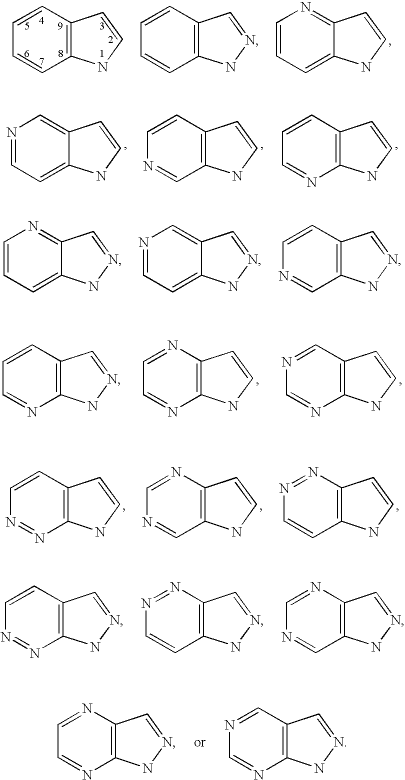
Unless indicated to the contrary, reference to positional numbering of bicyclic structures provided herein is based on the numbering of indole as shown above.

In some embodiments involving compounds of Formula I including a bicyclic core as shown above, such compounds can include substituents as described for Formula I, with the understanding that ring nitrogens other than the nitrogen corresponding to position 1 of the indole structure are unsubstituted. In some embodiments, the compounds have one of the bicyclic cores shown above and substitution selections as shown herein for compounds having an indolyl core; the compounds have one of the bicyclic cores above, and the substituents shown at the 5-position are instead attached at the 6-position.

In some embodiments involving compounds of Formula I, the compounds have a structure of Formula Ia, namely 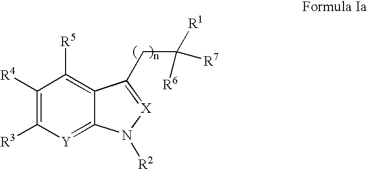
all salts, prodrugs, tautomers and isomers thereof,
wherein:
U is CR⁸, wherein R⁸ is R⁵;
V is CR⁸, wherein R⁸ is R⁴;
W is CR⁸, wherein R⁸ is R³;
R³, R⁴, and R⁵ are independently selected from the group consisting of hydrogen, halogen, optionally substituted lower alkyl, —CH₂—CR¹²═CR¹³R¹⁴, —CH₂—C≡CR¹⁵ , optionally substituted cycloalkyl, optionally substituted heterocycloalkyl, optionally substituted aryl, optionally substituted heteroaryl, —OR⁹, —SR⁹, —NR¹⁰R¹¹, —C(Z)NR¹⁰R¹¹, —C(Z)R²⁰, —S(O)₂NR¹⁰R¹¹, and —S(O)₂R²¹; and
   n, X, Y, R¹, R², R⁶, R⁷, R⁹, R¹⁰, R¹¹, R¹², R¹³, R¹⁴, R¹⁵, R²⁰ and R²¹ are as defined in Formula I above.

In some embodiments, such compounds are compounds of Formula I with Y=N; with Y=CR⁸; with Y=CH; with all R substituents other than R¹, R², and R⁴ as H (for each of X as N, X as CH, and X as CR⁸); and with R⁶ and R⁷ as H (for each of X as N, X as CH, and X as CR⁸).

In some embodiments, n=1; n=1 and X and/or Y is CH; n=1, X and/or Y is CH, and R⁶ and R⁷ are H; n=1 and X and/or Y=CR^{8.}

In some embodiments, n=1, R² is —S(O)₂R²¹, with R²¹ being optionally substituted aryl or optionally substituted heteroaryl. In some embodiments, in which n=1, and R² is —S(O)₂R²¹, with R²¹ being optionally substituted aryl or optionally substituted heteroaryl, the aryl group is a 5- or 6-membered ring; the aryl group is a 6-membered ring; in further embodiments in which the aryl group is a 6-membered ring, the ring is substituted with one or two groups independently selected from the group consisting of halogen, aryl substituted lower alkyl, heteroaryl substituted lower alkyl, lower alkoxy, aryl substituted lower alkoxy, heteroaryl substituted lower alkoxy, cycloalkyl, aryl, aryloxy, heteroaryl, and heteroaryloxy; in further embodiments in which a 6-membered ring is substituted with halogen or lower alkoxy, the ring is substituted at the 3-position (meta), 4-position (para), or 3- and 4-positions (meta and para); in further embodiments in which a 6-membered ring is substituted at the 4-position, or 3- and 4-positions, or the 4-position substitutent is lower alkyl, or the 4-position substituent is not lower alkyl, or the 4-position substituent is halogen (e.g., fluoro or chloro), or the 3- and 4-position substituents are fluoro, or the 3- and 4-position substitutents are chloro, or one of the 3- and 4-position substituents is fluoro and the other is chloro, or the 3-position is halogen (e.g., fluoro or chloro) and the 4-position is lower alkoxy (e.g., methoxy or ethoxy), or the 3-position is lower alkoxy (e.g., methoxy or ethoxy) and the 4-position is halogen (e.g., fluoro or chloro), or the 3-position is chloro and the 4-position is lower alkoxy, or the 3-position is lower alkoxy and the 4-position is chloro; or the 6-membered ring is fused with a second 5- or 6-membered aromatic or non-aromatic carbocyclic or heterocyclic ring. In further embodiments in which the aryl group is a 5-membered ring, the ring is substituted with one or two groups located at ring positions not adjacent to the ring atom linked to the —S(O)₂— group; or the 5-membered ring is substituted with one or two ring substituents selected from the group consisting of halogen, aryl substituted lower alkyl, heteroaryl substituted lower alkyl, lower alkoxy, aryl substituted lower alkoxy, heteroaryl substituted lower alkoxy, cycloalkyl, aryl, aryloxy, heteroaryl, and heteroaryloxy; the ring is substituted with chloro; the ring is substituted with lower alkoxy; or the ring is substituted with lower alkyl; or the ring is substituted with optionally substituted aryl or optionally substituted heteroaryl; or the ring is substituted with optionally substituted aryloxy or optionally substituted heteroaryloxy; or the 5-membered ring is fused with a second 5- or 6-membered aromatic or non-aromatic carbocyclic or heterocyclic ring.

In some embodiments in which n=1, and R² is —S(O)₂R²¹, with R²¹ being optionally substituted aryl or optionally substituted heteroaryl, R⁴ is not H or lower alkoxy, or R⁴ is not H or OR⁹.

In some embodiments, n=2; or n=2 and X and/or Y is CH; or n=2, X and/or Y is CH, and R⁶ and R⁷ are H; or n=2 and X and/or Y is CR⁸; or n=2 and X and/or Y are N.

In some embodiments in which n=2, R⁴ is not H, halogen, lower alkyl, lower alkoxy, or lower alkylthio; or R⁴ is not H, halogen, C₁₋₃ alkyl, C₁₋₃ alkoxy, or C₁₋₃ alkylthio; R⁴ is not C₁₋₃ alkoxy; or R⁴ is not methoxy.

In some embodiments, n=2, R² is —S(O)₂R²¹, with R²¹ being optionally substituted aryl or optionally substituted heteroaryl. In some embodiments, in which n=2, and R² is —S(O)₂R²¹, with R²¹ being optionally substituted aryl or optionally substituted heteroaryl, the aryl group is a 5- or 6-membered ring; or the aryl group is a 6-membered ring; in further embodiments in which the aryl group is a 6-membered ring, the ring is substituted with one or two groups independently selected from the group consisting of halogen, lower alkyl, aryl substituted lower alkyl, heteroaryl substituted lower alkyl, aryl substituted lower alkoxy, heteroaryl substituted lower alkoxy, cycloalkyl, aryl, aryloxy, heteroaryl, and heteroaryloxy; in further embodiments in which a 6-membered ring is substituted with halogen or lower alkoxy, the ring is substituted at the 3-position (meta), 4-position (para), or 3- and 4-positions (meta and para); in further embodiments in which a 6-membered ring is substituted at the 4-position, or 3- and 4-positions, the 4-position substitutent is lower lower alkyl, or the 4-position substituent is not lower alkyl, or the 4-position position substituent is halogen (e.g., fluoro or chloro), or the 3- and 4-position substituents are fluoro, or the 3- and 4-position substitutents are chloro, or one of the 3- and 4-position substituents is fluoro and the other is chloro, or the 3-position is halogen (e.g., fluoro or chloro) and the 4-position is lower alkoxy (e.g., methoxy or ethoxy), or the 3-position is lower alkoxy (e.g., methoxy or ethoxy) and the 4-position is halogen (e.g., fluoro or chloro), or the 3-position is chloro and the 4-position is lower alkoxy, or the 3-position is lower alkoxy and the 4-position is chloro; or the 6-membered ring is fused with a second 5- or 6-membered aromatic or non-aromatic carbocyclic or heterocyclic ring. In further embodiments in which the aryl group is a 5-membered ring, the ring is substituted with one or two groups located at ring positions not adjacent to the ring atom linked to the —S(O)₂— group; or the 5-membered ring is substituted with one or two ring substituents selected from the group consisting of halogen, aryl substituted lower alkyl, heteroaryl substituted lower alkyl, lower alkoxy, aryl substituted lower alkoxy, heteroaryl substituted lower alkoxy, cycloalkyl, aryl, aryloxy, heteroaryl, and heteroaryloxy; or the ring is substituted with chloro; or the ring is substituted with lower alkoxy; or the ring is substituted with lower alkyl; or the ring is substituted with optionally substituted aryl or optionally substituted heteroaryl; or the ring is substituted with optionally substituted aryloxy or optionally substituted heteroaryloxy; or the 5-membered ring is fused with a second 5- or 6-membered aromatic or non-aromatic carbocyclic or heterocyclic ring.

In some embodiments, in which n=2, and R² is —S(O)₂R²¹, with R²¹ being a substituted 6-membered aryl group, the substitution on the aryl group is not methoxy, or the substitution on the aryl group is not lower alkoxy; or R⁴ and the substitution on the aryl group are not both lower alkoxy; or R⁴ and the substitution on the aryl group are not both methoxy; or R⁴ is not lower alkoxy; or R⁴ is not methoxy.

Certain further embodiments include compounds described for corresponding embodiments as described above for both n=1 and n=2.

In some embodiments, compounds of Formula I have a structure of Formula lb as shown below: 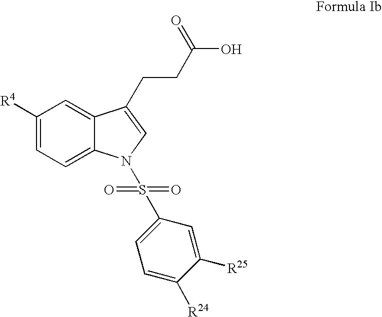
all salts, prodrugs, tautomers and isomers thereof,
wherein:
U is CR⁸, wherein R⁸ is H;
V is CR⁸, wherein R⁸ is R⁴;
W is CR⁸, wherein R⁸ is H;
X is CR⁸, wherein R⁸ is H;
Y is CR⁸, wherein R⁸ is H;
n is 1;
R¹ is —COOH;
R⁶ and R⁷ are hydrogen;
R² is —S(O)₂R²¹, wherein R²¹ is 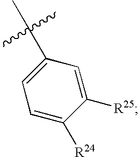
R⁴ is selected from the group consisting of hydrogen, halogen, optionally substituted lower alkyl, optionally substituted cycloalkyl, optionally substituted heterocycloalkyl, optionally substituted aryl, optionally substituted heteroaryl, —OR⁹, —SR⁹, —NR¹⁰R¹¹, —C(Z)NR¹⁰R¹¹, —C(Z)R²⁰, —S(O)₂NR¹⁰R¹¹, and —S(O)₂R²¹;
R²⁴ is selected from the group consisting of hydrogen, halogen, optionally substituted lower alkyl, —OR¹⁹, and —O(CH₂)ₚO-aryl;
p is 1,2,3,or4;
R²⁵ is selected from the group consisting of hydrogen, halogen, optionally substituted lower alkyl, and —OR¹⁹; or
R²⁴ and R²⁵ combine to form cycloalkyl, heterocycloalkyl, aryl or heteroaryl fused with the phenyl ring;
R¹⁹ is selected from the group consisting of optionally substituted lower alkyl and optionally substituted aryl; and
R⁹, R¹⁰, R¹¹, R²⁰ and R²¹ are as defined in Formula I above.

In some embodiments, R⁴ is optionally substituted lower alkoxy (e.g., methoxy, ethoxy, propoxy, isopropoxy), optionally substituted aryloxy, optionally substituted heteroaryloxy, optionally substituted lower alkyl (e.g., methyl or ethyl), optionally substituted cycloalkyl, optionally substituted heterocycloalkyl, optionally substituted aryl, optionally substituted heteroaryl, or halogen.

In some embodiments, R⁴ is optionally substituted lower alkoxy (e.g., methoxy, ethoxy, propoxy, isopropoxy), optionally substituted lower alkyl (e.g., methyl or ethyl), optionally substituted aryl, optionally substituted heteroaryl, or halogen.

In some embodiments, compounds of Formula I can be as specified for Formula lb, but with the phenyl ring to which R²⁴ and R²⁵ are attached as a heteroaryl ring, wherein when the heteroaryl ring is a 5-membered ring, R²⁴ and R²⁵ are not attached to the 5-membered ring atoms that are adjacent to the 5-membered ring atom attached to the sulfonyl group shown in Formula lb.

In some embodiments of compounds of Formula lb, R⁴ is lower alkoxy and R²⁴ and R²⁵ are chloro; or R⁴ is lower alkoxy and R²⁴ and R²⁵ are fluoro; or R⁴ is lower alkoxy and R²⁴ is lower alkoxy; or R⁴ is lower alkoxy and R²⁴ is lower alkyl; or R⁴ is methoxy or ethoxy and R²⁴ and R²⁵ are chloro; or R⁴ is methoxy or ethoxy and R²⁴ is lower alkoxy; or R⁴ is methoxy or ethoxy and R²⁴ is lower alkyl.

In some embodiments of compounds of Formula lb, R²⁴ and R²⁵ are not lower alkyl; or R²⁴ is H and R²⁵ is not lower alkyl; or R²⁵ is H and R²⁴ is not lower alkyl.

In some embodiments, the invention involves compounds of Formula II as follows: 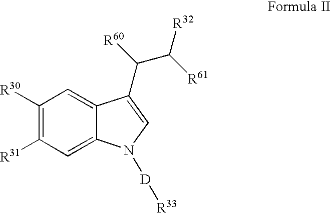
all salts, prodrugs, tautomers and isomers thereof,
wherein:
R³⁰ and R³¹ are independently selected from the group consisting of hydrogen, halogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted heteroaryl, —OH, —OR³⁴, —SR³⁵, —NR³⁶R³⁷, —C(Z)NR³⁸R³⁹, —C(Z)R⁴⁰, —S(O)₂NR³⁸ R³⁹ and —S(O)ₙR⁴¹; or
R³⁰ and R³¹ combine to form a fused ring, wherein the combined R³⁰ and R³¹ are of the formula 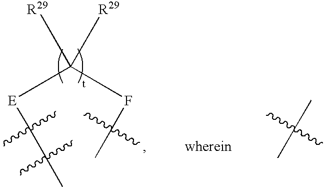
   indicates the point of attachment of R³⁰ to the indole ring and 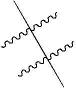
   indicates the point of attachment of R³¹ to the indole ring;
E and F are independently selected from the group consisting of CR²⁹R²⁹, O, S(O)₂ and NR44;
R²⁹ at each occurrence is independently selected from the group consisting of hydrogen, fluoro, optionally fluoro substituted lower alkyl, optionally fluoro substituted lower alkoxy, and optionally fluoro substituted lower alkylthio;
R⁴⁴ is hydrogen or lower alkyl; t is 1 or 2;
R³² is selected from the group consisting of —C(O)OR²⁶, —C(O)NR²⁷R²⁸, and a carboxylic acid isostere;
R³³ is L—R⁴² or heteroaryl optionally substituted with one or more substituents selected from the group consisting of halogen, optionally substituted lower alkyl, optionally substituted lower alkenyl, optionally substituted lower alkynyl, optionally substituted cycloalkyl, optionally substituted aryl, optionally substituted heterocycloalkyl, optionally substituted heteroaryl, —OH, —NO₂, —CN, —OR³⁴, —SR³⁵, —NR³⁶R³⁷, —C(Z)NR³⁸R³⁹, —C(Z)R⁴⁰, —S(O)₂NR³⁸R³⁹, and —S(O)ₙR⁴¹;
L is —(CR⁵¹R⁵²)ₘ— or —CR⁵⁵═CR⁵⁶—;
D is —CR⁵¹R⁵²— or —S(0)₂—;
R³⁴ is selected from the group consisting of optionally substituted lower alkyl, optionally substituted C₃₋₆ alkenyl, provided, however, that when R³⁴ is optionally substituted C₃₋₆ alkenyl, no alkene carbon thereof is bound to the O of —OR³⁴, optionally substituted C₃₋₆ alkynyl, provided, however, that when R³⁴ is optionally substituted C₃₋₆ alkynyl, no alkyne carbon thereof is bound to the O of —OR³⁴, optionally substituted cycloalkyl, optionally substituted heterocycloalkyl, optionally substituted aryl, optionally substituted heteroaryl, —C(Z)R⁴⁰, and —C(Z)NR³⁸R³⁹;
R³⁵ is selected from the group consisting of optionally substituted lower alkyl, optionally substituted C₃₋₆ alkenyl, provided, however, that when R³⁵ is optionally substituted C₃₋₆ alkenyl, no alkene carbon thereof is bound to the S of —SR³⁵ or the O of —OR³⁵, optionally substituted C₃₋₆ alkynyl, provided, however, that when R³⁵ is optionally substituted C₃₋₆ alkynyl, no alkyne carbon thereof is bound to the S of —SR³⁵ or the O of —OR³⁵, optionally substituted cycloalkyl, optionally substituted heterocycloalkyl, optionally substituted aryl, and optionally substituted heteroaryl;
R³⁶ and R³⁷ are independently selected from the group consisting of hydrogen, optionally substituted lower alkyl, optionally substituted C₃₋₆ alkenyl, provided, however, that when R³⁶ and/or R³⁷ are optionally substituted C₃₋₆ alkenyl, no alkene carbon thereof is bound to the N of —NR³⁶R³⁷, optionally substituted C₃₋₆ alkynyl, provided, however, that when R³⁶ and/or R³⁷ are optionally substituted C₃₋₆ alkynyl, no alkyne carbon thereof is bound to the N of —NR³⁶R³⁷, optionally substituted cycloalkyl, optionally substituted heterocycloalkyl, optionally substituted aryl, optionally substituted heteroaryl, —C(Z)R⁴⁰, —C(Z)NR³⁸R³⁹, —S(O)₂R⁴¹, and —S(O)₂NR³⁸R³⁹;
R³⁸ and R³⁹ are independently selected from the group consisting of hydrogen, optionally substituted lower alkyl, optionally substituted C₃₋₆ alkenyl, provided, however, that when R³⁸ and/or R³⁹ are optionally substituted C₃₋₆ alkenyl, no alkene carbon thereof is bound to the N of NR³⁸R³⁹, optionally substituted C₃₋₆ alkynyl, provided, however, that when R³⁸ and/or R³⁹ are optionally substituted C₃₋₆ alkynyl, no alkyne carbon thereof is bound to the N of NR³⁸R³⁹, optionally substituted cycloalkyl, optionally substituted heterocycloalkyl, optionally substituted aryl, and optionally substituted heteroaryl;
R⁴⁰ is selected from the group consisting of optionally substituted lower alkyl, optionally substituted C₃₋₆ alkenyl, provided, however, that when R⁴⁰ is optionally substituted C₃₋₆ alkenyl, no alkene carbon thereof is bound to —C(Z)—, optionally substituted C₃₋₆ alkynyl, provided, however, that when R⁴⁰ is optionally substituted C₃₋₆ alkynyl, no alkyne carbon thereof is bound to —C(Z)—, optionally substituted cycloalkyl, optionally substituted heterocycloalkyl, optionally substituted aryl, optionally substituted heteroaryl, —OH, and —OR³⁵;
R⁴¹ is selected from the group consisting of optionally substituted lower alkyl, optionally substituted C₃₋₆ alkenyl, provided, however, that when R⁴¹ is optionally substituted C₃₋₆ alkenyl, no alkene carbon thereof is bound to —S(O)ₙ—, optionally substituted C₃₋₆ alkynyl, provided, however, that when R⁴¹ is optionally substituted C₃₋₆ alkynyl, no alkyne carbon thereof is bound to —S(O)ₙ—, optionally substituted cycloalkyl, optionally substituted heterocycloalkyl, optionally optionally substituted aryl, and optionally substituted heteroaryl;
R⁴² is aryl or heteroaryl, wherein aryl or heteroaryl are optionally substituted with one or more substituents selected from the group consisting of halogen, optionally substituted lower alkyl, optionally substituted lower alkenyl, optionally substituted lower alkynyl, optionally substituted cycloalkyl, optionally substituted aryl, optionally substituted heterocycloalkyl, optionally substituted heteroaryl, —OH, —NO₂, —CN, —OR³⁴ , —SR³, —NR³⁶R³⁷, —C(Z)NR³⁸R³⁹, —C(Z)R⁴⁰, —S(O)₂NR³⁹, and —S(O)ₙR⁴¹;
R⁵¹ and R⁵² are independently selected from the group consisting of hydrogen, fluoro, optionally substituted lower alkyl, optionally substituted cycloalkyl, optionally substituted heterocycloalkyl, optionally substituted aryl, and optionally substituted heteroaryl; or any two of R⁵¹ and R⁵² on the same carbon or on adjacent carbons may be combined to form an optionally substituted 3-7 membered monocyclic cycloalkyl or optionally substituted 5-7 membered monocyclic heterocycloalkyl;
R⁵⁵ and R⁵⁶ are independently selected from the group consisting of hydrogen, optionally substituted lower alkyl, optionally substituted cycloalkyl, optionally substituted heterocycloalkyl, optionally substituted aryl, and optionally substituted heteroaryl; or
R⁵⁵ and R⁵⁶ combine to form an optionally substituted 5-7 membered monocyclic cycloalkyl or optionally substituted 5-7 membered monocyclic heterocycloalkyl;
R⁶⁰ and R⁶¹ are each hydrogen, or R⁶⁰ and R⁶¹ combine to form optionally substituted 3-7 membered monocyclic cycloalkyl;
R²⁶ is selected from the group consisting of hydrogen, lower alkyl, phenyl, 5-7 membered monocyclic heteroaryl, 3-7 membered monocyclic cycloalkyl, and 5-7 membered monocylic heterocycloalkyl, wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, and wherein lower alkyl is optionally substituted with one or more substituents selected from the group consisting of fluoro, —OH, —NH₂, lower alkoxy, fluoro substituted lower alkoxy, lower alkylthio and fluoro substituted lower alkylthio, provided, however, that when R²⁶ is lower alkyl, any substitution on the lower alkyl carbon bound to the O of OR²⁶ is fluoro;
R²⁷ and R²⁸ are independently selected from the group consisting of hydrogen, lower alkyl, phenyl, 5-7 membered monocyclic heteroaryl, 3-7 membered monocyclic cycloalkyl, and 5-7 membered monocylic heterocycloalkyl, wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, and wherein lower alkyl is optionally substituted with one or more substituents selected from the group consisting of fluoro, —OH, —NH₂, lower alkoxy, fluoro substituted lower alkoxy, lower alkylthio and fluoro substituted lower alkylthio, provided, however, that when R²⁷ and/or R²⁸ is lower alkyl, any substitution on the lower alkyl carbon bound to the N of NR²⁷R²⁸ is fluoro; or
R²⁷ and R²⁸ together with the nitrogen to which they are attached form a 5-7 membered monocyclic heterocycloalkyl or a 5 or 7 membered nitrogen containing monocyclic heteroaryl, wherein the monocyclic heterocycloalkyl or monocyclic nitrogen containing heteroaryl is optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
n is 1, or 2;
m is 1,2, or3; and
Z is O or S, provided, however, that when D is —S(O)₂—, R³⁰ is —OCH₃, R³¹ is H, and R³² is —COOH or —COOCH₃, R³³ is not unsubstituted thiophenyl

In one embodiment of compounds of Formula II, R³³ is not unsubstituted thiophenyl. In another embodiment, R³³ is substituted heteroaryl. In another embodiment, R³³ is heteroaryl substituted with one or more substituents selected from the group consisting of lower alkyl, wherein lower alkyl is substituted with optionally substituted cycloalkyl, optionally substituted heterocycloalkyl, optionally substituted aryl or optionally substituted heteroaryl, optionally substituted cycloalkyl, optionally substituted aryl, optionally substituted heterocycloalkyl, optionally substituted heteroaryl, —OR —SR³⁵, —NR³⁶R³⁷, —C(Z)NR³⁸R³⁹, —C(Z)R⁴⁰, —S(O)₂NR³⁸R³⁹, and —S(O)ₙR⁴¹, wherein one of R³⁶ and R³⁷ is selected from the group consisting of lower alkyl, wherein lower alkyl is substituted with optionally substituted aryl or optionally substituted heteroaryl, optionally substituted cycloalkyl, optionally substituted heterocycloalkyl, optionally substituted aryl, optionally substituted heteroaryl, —C(Z)R⁴⁰, —C(Z)NR³⁸R³⁹, —S(O)₂R⁴¹, and —S(O)₂NR³⁸R³⁹, and the other of R³⁶ and R³⁷ is hydrogen or lower alkyl, one of R³⁸ and R³⁹ is selected from the group consisting of lower alkyl, wherein lower alkyl is substituted with optionally substituted aryl or optionally substituted heteroaryl, optionally substituted cycloalkyl, optionally substituted heterocycloalkyl, optionally substituted aryl, and optionally substituted heteroaryl, and the other of R³⁸ and R³⁹ is hydrogen or lower alkyl, and wherein R³⁴, R³⁵, R⁴⁰, and R⁴¹ are independently selected from the group consisting of lower alkyl, wherein lower alkyl is substituted with optionally substituted aryl or optionally substituted heteroaryl, optionally substituted cycloalkyl, optionally substituted heterocycloalkyl, optionally substituted aryl, and optionally substituted heteroaryl.

In one embodiment of compounds of Formula II, R³⁰ and R³¹ are independently selected from the group consisting of hydrogen, halogen, optionally substituted lower alkyl, optionally substituted lower alkoxy, optionally substituted aryloxy, optionally substituted heteroaryloxy, optionally substituted cycloalkyl, optionally substituted heterocycloalkyl, optionally substituted aryl and optionally substituted heteroaryl, or R³⁰ and R³¹ combine to form a fused ring wherein E and F are O, t is 1 or 2, and each R²⁹ is hydrogen. In one embodiment, R³⁰ and R³¹ are independently selected from the group consisting of hydrogen, halogen, and optionally substituted lower alkoxy, preferably wherein R³¹ is hydrogen and R³⁰ is halogen or optionally fluoro substituted lower alkoxy, preferably lower alkoxy.

In one embodiment of compounds of Formula II, D is —CR⁵¹R⁵²—, wherein each R⁵¹ and R⁵² are independently halogen or optionally substituted lower alkyl, or any two of R⁵¹ and R⁵² on the same carbon or on adjacent carbons combine to form an optionally substituted 3-7 membered monocyclic cycloalkyl or optionally substituted 3-7 membered monocyclic heterocycloalkyl, R³³ is substituted heteroaryl, and R³⁰ and R³¹ are independently selected from the group consisting of hydrogen, halogen, and optionally substituted lower alkoxy, preferably wherein D is —CH₂—, R³¹ is hydrogen and R³⁰ is halogen or optionally fluoro substituted lower alkoxy, preferably lower alkoxy.

In one embodiment of compounds of Formula II, D is —S(O)₂—, R³³ is substituted heteroaryl, and R³⁰ and R³¹ are independently selected from the group consisting of hydrogen, halogen, and optionally substituted lower alkoxy, preferably wherein R³¹ is hydrogen and R³⁰ is halogen or optionally fluoro substituted lower alkoxy, preferably lower alkoxy.

Further to any of the above embodiments of compounds of Formula II, R⁶⁰ and R⁶¹ are hydrogen and R³² is —C(O)OR²⁶, preferably —COOH.

In some embodiments, the invention involves compounds of Formula III as follows: 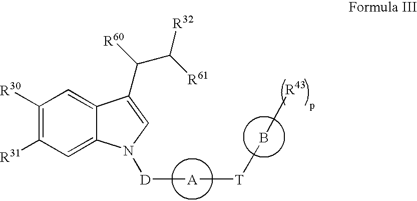
all salts, prodrugs, tautomers and isomers thereof,
wherein:
D, R³⁰, R³¹, R³², R⁶⁰, and R³¹ are as defined in formula II;
A is arylene or heteroarylene, wherein arylene or heteroarylene are optionally substituted with one or more substituents selected from the group consisting of halogen, —OH, lower alkyl, lower alkoxy, and lower alkylthio, wherein lower alkyl and the lower alkyl chains of lower alkoxy and lower alkylthio are optionally substituted with one or more substituents selected from the group consisting of fluoro, —OH, lower alkoxy, and lower alkylthio, provided, however, that any substitution of the carbon bound to the lower alkoxy O or lower alkylthio S is fluoro; T is a covalent bond or is selected from the group consisting of —(CR⁵¹R⁵²)ₘ—, —(CR⁵¹R⁵²)_{q}O(CR⁵¹R⁵²)ᵣ—, —(CR⁵¹R⁵²)_{q}S(CR⁵¹R⁵²)ᵣ—, —(CR⁵¹R⁵²)_{q}NR⁵³(CR⁵¹R⁵²)ᵣ—, —(CR⁵¹R⁵²)_{q}C(Z) (CR⁵¹R⁵²)ᵣ—, —(CR⁵¹R⁵²)_{q}S(O)ₙ(CR⁵¹R⁵²)ᵣ—, —(CR⁵¹R⁵²)_{q}C(Z)NR⁵⁴(CR⁵¹R⁵²)ᵣ—, —(CR⁵¹R⁵²)_{q}NR⁵⁴C(Z) (CR⁵¹R⁵²)ᵣ—, —(CR⁵¹R⁵²)_{q}NR⁵⁴C(Z)NR⁵⁴(CR⁵¹R⁵²)ᵣ—, —(CR⁵¹R⁵²)_{q}NR⁵⁴S(O)₂(CR⁵¹R⁵²)ᵣ—, —(CR⁵¹R⁵²)_{q}S(O)₂NR⁵⁴(CR⁵¹R⁵²)ᵣ—, and ——(CR⁵¹R⁵²)_{q}NR⁵⁴S(O)₂NR⁵⁴(CR⁵¹R⁵²)ᵣ—, wherein R⁵¹, R⁵² and m are as defined in Formula II above;
q and r are independently 0, 1, or 2;
B is selected from the group consisting of cycloalkyl, heterocycloalkyl, aryl, and heteroaryl;
R⁴³ at each occurence is independently selected from the group consisting of halogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted heteroaryl, —OH, —OR³⁴, —SR³⁵, —NR³⁶R³⁷, —C(Z)NR³⁸R³⁹, —C(Z)R⁴⁰, —S(O)₂NR³⁸R³⁹, and —S(O)R ⁴¹;
R⁵³ is selected from the group consisting of hydrogen, optionally substituted lower alkyl, optionally substituted C₃₋₆ alkenyl, provided, however, that when R⁵³ is optionally substituted C₃₋₆ alkenyl, no alkene carbon thereof is bound to the N of —NR⁵³—, optionally substituted C₃₋₆ alkynyl, provided, however, that when R⁵³ is optionally substituted C₃₋₆ alkynyl, no alkyne carbon thereof is bound to the N of —NR⁵³ —, optionally substituted cycloalkyl, optionally substituted heterocyclyoalkyl, optionally substituted aryl, optionally substituted heteroaryl,—C(Z)NR³⁸R³⁹, —C(Z)R⁴⁰, —S(O)₂NR³⁸R³⁹, and —S(O)₂R⁴¹;
R⁵⁴ at each occurrence is independently selected from the group consisting of hydrogen, optionally substituted lower alkyl, optionally substituted C₃₋₆ alkenyl, provided, however, that when R⁵⁴ is optionally substituted C₃₋₆ alkenyl, no alkene carbon thereof is bound to the N of —NR⁵⁴—, optionally substituted C₃₋₆ alkynyl, provided, however, that when R⁵⁴ is optionally substituted C₃₋₆ alkynyl, no alkyne carbon thereof is bound to the N of —NR⁵⁴—, optionally substituted cycloalkyl, optionally substituted heterocycloalkyl, optionally substituted aryl, and optionally substituted heteroaryl; p is 0, 1,2 or3; and
n, Z R³⁴, R³⁵ , R³⁶, R³⁷, R³⁸, R³⁹, R⁴⁰, and R⁴¹ are as defined for Formula II above, provided, however, the compound is not 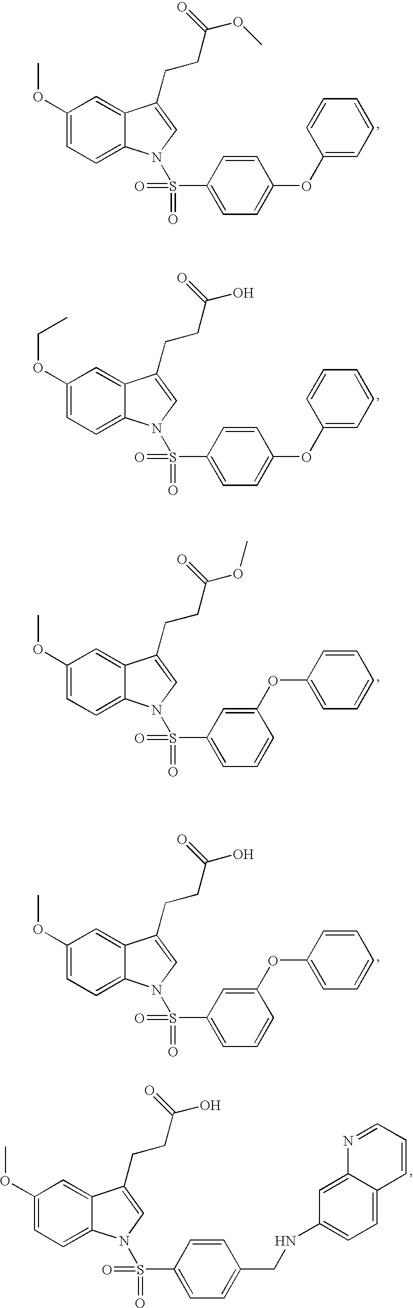 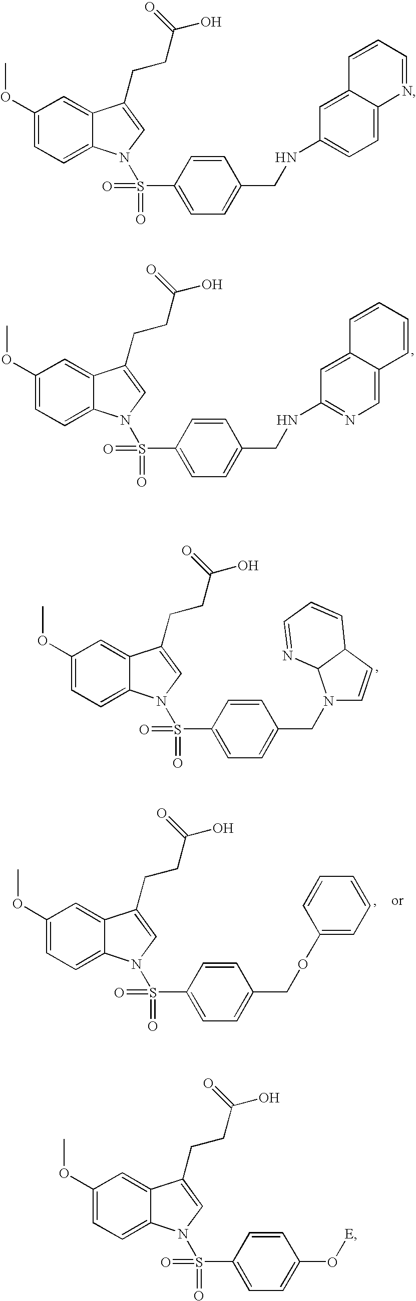
   wherein E is 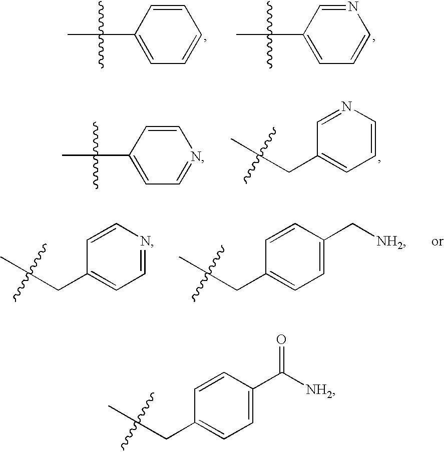
   wherein 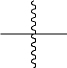
   indicates the point of attachment of E to 0.

In one embodiment of compounds of Formula III, when A is phenyl, T is meta or para to D, and B is phenyl, pyridinyl, 7-azaindolyl, or quinolinyl, then p is 1, 2 or 3, provided, however, that when T is —OCR⁵¹R⁵²— and para to D, B is phenyl, p is 1, and R⁴³ is para to T, R⁴³ is not CH₂NH₂ or C(O)NH₂. In another embodiment, A is other than phenyl. In another embodiment, B is other than phenyl, pyridinyl, 7-azaindolyl, or quinolinyl.

In one embodiment of compounds of Formula III, A is heteroaryl optionally substituted with halogen, —OH, lower alkyl, lower alkoxy, or lower alkylthio, wherein lower alkyl and the lower alkyl chains of lower alkoxy and lower alkylthio are optionally substituted with fluoro, —OH, lower alkoxy, or lower alkylthio, provided, however, that any substitution of the carbon bound to the lower alkoxy O or lower alkylthio S is fluoro. In one embodiment R⁴³ is selected from the group consisting of halogen, —OH, optionally substituted lower alkyl, optionally substituted lower alkenyl, optionally substituted lower alkynyl, —OR³⁴, —SR³⁵, —NR⁶³R³⁷, —C(Z)NR³⁸R³⁹, —C(Z)R⁴⁰, —S(O)₂NR³⁸R³⁹, and —S(O)ₙR⁴¹, wherein R³⁴, R³⁵, R³⁶, R³⁷, R³⁸, R³⁹, R⁴⁰ and R⁴¹ are not optionally substituted cycloalkyl, optionally substituted heterocycloalkyl, optionally substituted aryl, optionally substituted heteroaryl, or lower alkyl substituted with optionally substituted cycloalkyl, optionally substituted heterocycloalkyl, optionally substituted aryl, or optionally substituted heteroaryl. In one embodiment, A is heteroaryl optionally substituted with halogen, —OH, lower alkyl, lower alkoxy, or lower alkylthio, wherein lower alkyl and the lower alkyl chains of lower alkoxy and lower alkylthio are optionally substituted with fluoro, —OH, lower alkoxy, or lower alkylthio, provided, however, that any substitution of the carbon bound to the lower alkoxy O or lower alkylthio S is fluoro, and R⁴³ is selected from the group consisting of halogen, —OH, optionally substituted lower alkyl, optionally substituted lower alkenyl, optionally substituted lower alkynyl, —OR³⁴, —SR³⁵, —NR³⁶R³⁷, —C(Z)NR³⁸R³⁹, —C(Z)R⁴⁰, —S(O)₂NR³⁸R³⁹, and —S(O)ₙR⁴¹, wherein R³⁴, R³⁵, R³⁶, R³⁷, R³⁸, R³⁹, R⁴⁰ and R⁴¹ are not optionally substituted cycloalkyl, optionally substituted heterocycloalkyl, optionally substituted aryl, optionally substituted heteroaryl, or lower alkyl substituted with optionally substituted cycloalkyl, optionally substituted heterocycloalkyl, optionally substituted aryl, or optionally substituted heteroaryl.

In one embodiment of compounds of Formula III, R³⁰ and R³¹ are selected from the group consisting of hydrogen, halogen, optionally substituted lower alkyl, optionally substituted lower alkoxy, optionally substituted aryloxy, optionally substituted heteroaryloxy, optionally substituted cycloalkyl, optionally substituted heterocycloalkyl, optionally substituted aryl and optionally substituted heteroaryl, or R³⁰ and R³¹ combine to form a fused ring wherein E and F are O, t is 1 or 2, and each R²⁹ is hydrogen. In one embodiment, R³⁰ and R³¹ are independently optionally substituted lower alkoxy, or R³⁰ and R³¹ combine to form a fused ring wherein E and F are O, t is 1 or 2, and each R²⁹ is hydrogen. In one embodiment, R³⁰ and R³¹ are independently selected from the group consisting of hydrogen, halogen, and optionally substituted lower alkoxy, preferably wherein R³¹ is hydrogen and R³⁰ is halogen or optionally fluoro substituted lower alkoxy, preferably lower alkoxy.

In another embodiment of compounds of Formula III, A is phenyl and T-B is ortho to D. In one embodiment, A is heteroaryl optionally substituted with halogen, lower alkyl, —OH, lower alkoxy, or lower alkylthio, wherein lower alkyl and the lower alkyl chains of lower alkoxy and lower alkylthio are optionally substituted with fluoro, —OH, lower alkoxy, or lower alkylthio, provided, however, that any substitution of the carbon bound to the lower alkoxy O or lower alkylthio S is fluoro. In one embodiment, A is phenyl optionally substituted with halogen, lower alkyl, —OH, lower alkoxy, or lower alkylthio, wherein lower alkyl and the lower alkyl chains of lower alkoxy or lower alkylthio are optionally substituted with fluoro, —OH, lower alkoxy, or lower alkylthio, provided, however, that any substitution of the carbon bound to the lower alkoxy 0 or lower alkylthio S is fluoro, and T-B is ortho to D.

In another embodiment of compounds of Formula III, R⁵³ and R⁵⁴ are independently selected from the group consisting of hydrogen, optionally substituted lower alkyl, optionally substituted aryl, and optionally substituted heteroaryl. In another embodiment, R⁵³ and R⁵⁴ are independently hydrogen or optionally substituted lower alkyl, where lower alkyl is preferably optionally substituted with fluoro, —OH, lower alkoxy, or lower alkylthio, provided, however, that substitution of the carbon that is bound to the N of —NR⁵³— or —NR⁵⁴— is fluoro.

In one embodiment of compounds of Formula III, D is —S(O)₂—, R60 and R⁶¹ are hydrogen, R³¹ is hydrogen, R³⁰ is halogen or optionally fluoro substituted lower alkoxy, preferably lower alkoxy, A is optionally substituted with halogen, lower alkyl, —OH, lower alkoxy, or lower alkylthio, wherein lower alkyl and the lower alkyl chains of lower alkoxy and lower alkylthio are optionally substituted with fluoro, —OH, lower alkoxy, or lower alkylthio, provided, however, that any substitution of the carbon bound to the lower alkoxy O or lower alkylthio S is fluoro, and T is a covalent bond, —O—, or —NCH₃—. In one embodiment, D is —S(O)₂—, R⁶⁰ and R⁶¹ are hydrogen, R³¹ is hydrogen, R³⁰ is halogen or optionally fluoro substituted lower alkoxy, preferably lower alkoxy, A is optionally substituted with halogen, lower alkyl, —OH, lower alkoxy, or lower alkylthio, wherein lower alkyl and the lower alkyl chains of lower alkoxy and lower-alkylthio are optionally substituted with fluoro, —OH, lower alkoxy, or lower alkylthio, provided, however, that any substitution of the carbon bound to the lower alkoxy O or lower alkylthio S is fluoro, T is a covalent bond, —O—, or —NCH₃—, and each R⁴³ is independently selected from the group consisting of halogen, —OH, optionally substituted lower alkyl, optionally substituted lower alkoxy, and optionally substituted lower alkylthio, preferably halogen, lower alkyl, —OH, lower alkoxy, or lower alkylthio, wherein lower alkyl and the lower alkyl chains of lower alkoxy and lower alkylthio are optionally substituted with fluoro, —OH, lower alkoxy, or lower alkylthio, provided, however, that any substitution of the carbon bound to the lower alkoxy O or lower alkylthio S is fluoro.

In one embodiment of compounds of Formula III, D is —S(O)₂—, R⁶⁰ and R⁶¹ are hydrogen, R³¹ is hydrogen, R³⁰ is halogen or optionally fluoro substituted lower alkoxy, preferably lower alkoxy, A is phenyl, thiophenyl, pyridinyl, thiazolyl, or oxazolyl, wherein phenyl, thiophenyl, pyridinyl, thiazolyl, or oxazolyl are optionally substituted with halogen, lower alkyl, —OH, lower alkoxy, or lower alkylthio, wherein lower alkyl and the lower alkyl chains of lower alkoxy and lower alkylthio are optionally substituted with fluoro, —OH, lower alkoxy, or lower alkylthio, provided, however, that any substitution of the carbon bound to the lower alkoxy O or lower alkylthio S is fluoro, T is a covalent bond, —O—, or —NCH₃—, and B is phenyl, pyridinyl, pyrazolyl, or isoxazolyl. In one embodiment, D is —S(O)₂—, R⁶⁰ and R⁶¹ are hydrogen, R³¹ is hydrogen, R³⁰ is halogen or optionally fluoro substituted lower alkoxy, preferably lower alkoxy, A is phenyl, thiophenyl, pyridinyl, thiazolyl, or oxazolyl, wherein phenyl, thiophenyl, pyridinyl, thiazolyl, or oxazolyl are optionally substituted with halogen, lower alkyl, —OH, lower alkoxy, or lower alkylthio, wherein lower alkyl and the lower alkyl chains of lower alkoxy and lower alkylthio are optionally substituted with fluoro, —OH, lower alkoxy, or lower alkylthio, provided, however, that any substitution of the carbon bound to the lower alkoxy O or lower alkylthio S is fluoro, T is a covalent bond, —O—, or —NCH₃—, B is phenyl, pyridinyl, pyrazolyl, or isoxazolyl, and each R⁴³ is independently selected from the group consisting of halogen, —OH, optionally substituted lower alkyl, optionally substituted lower alkoxy, and optionally substituted lower alkylthio, preferably halogen, lower alkyl, —OH, lower alkoxy, or lower alkylthio, wherein lower alkyl and the lower alkyl chains of lower alkoxy and lower alkylthio are optionally substituted with fluoro, —OH, lower alkoxy, or lower alkylthio, provided, however, that any substitution of the carbon bound to the lower alkoxy O or lower alkylthio S is fluoro.

In one embodiment of compounds of Formula III, D is —S(O)₂—, R60 and R⁶¹ are hydrogen, R³¹ is hydrogen, R³⁰ is halogen or optionally fluoro substituted lower alkoxy, preferably lower alkoxy, A is phenyl, thiophenyl, pyridinyl, thiazolyl, or oxazolyl, wherein phenyl, thiophenyl, pyridinyl, thiazolyl, or oxazolyl are optionally substituted with fluoro, chloro, optionally fluoro substituted lower alkyl, or optionally fluoro substituted lower alkoxy, T is a covalent bond, —O—, or —NCH₃—, B is phenyl, pyridinyl, pyrazolyl, or isoxazolyl, and each R⁴³ is independently selected from the group consisting of fluoro, chloro, optionally fluoro substituted lower alkyl, and optionally fluoro substituted lower alkoxy.

In one embodiment of compounds of Formula III, D is —CR⁵¹R⁵²—, preferably —CH₂—, R⁶⁰ and R⁶¹ are hydrogen, R is hydrogen, R³⁰ is halogen or optionally fluoro substituted lower alkoxy, preferably lower alkoxy, A is optionally substituted with halogen, lower alkyl, —OH, lower alkoxy, or lower alkylthio, wherein lower alkyl and the lower alkyl chains of lower alkoxy and lower alkylthio are optionally substituted with fluoro, —OH, lower alkoxy, or lower alkylthio, provided, however, that any substitution of the carbon bound to the lower alkoxy O or lower alkylthio S is fluoro, and T is a covalent bond, —O—, or —NCH₃—. In one embodiment, D is —CR⁵¹R⁵²—, preferably —CH₂—, R⁶⁰ and R are hydrogen, R³ 'is hydrogen, R³⁰ is halogen or optionally fluoro substituted lower alkoxy, preferably lower alkoxy, A is optionally substituted with halogen, lower alkyl, —OH, lower alkoxy, or lower alkylthio, wherein lower alkyl and the lower alkyl chains of lower alkoxy and lower alkylthio are optionally substituted with fluoro, —OH, lower alkoxy, or lower alkylthio, provided, however, that any substitution of the carbon bound to the lower alkoxy O or lower alkylthio S is fluoro, T is a covalent bond, —O—, or —NCH₃—, and each R⁴³ is independently selected from the group consisting of halogen, —OH, optionally substituted lower alkyl, optionally substituted lower alkoxy, and optionally substituted lower alkylthio, preferably halogen, lower alkyl, —OH, lower alkoxy, or lower alkylthio, wherein lower alkyl and the lower alkyl chains of lower alkoxy and lower alkylthio are optionally substituted with fluoro, —OH, lower alkoxy, or lower alkylthio, provided, however, that any substitution of the carbon bound to the lower alkoxy O or lower alkylthio S is fluoro.

In one embodiment of compounds of Formula III, D is —CR⁵R⁵²—, preferably —CH₂—, R⁶⁰ and R⁶¹ are hydrogen, R³¹ is hydrogen, R³⁰ is halogen or optionally fluoro substituted lower alkoxy, preferably lower alkoxy, A is phenyl, thiophenyl, pyridinyl, thiazolyl, or oxazolyl, wherein phenyl, thiophenyl, pyridinyl, thiazolyl, or oxazolyl are optionally substituted with halogen, lower alkyl, —OH, lower alkoxy, or lower alkylthio, wherein lower alkyl and the lower alkyl chains of lower alkoxy and lower alkylthio are optionally substituted with fluoro, —OH, lower alkoxy, or lower alkylthio, provided, however, that any substitution of the carbon bound to the lower alkoxy O or lower alkylthio S is fluoro, T is a covalent bond, —O—, or —NCH₃—, and B is phenyl, pyridinyl, pyrazolyl, or isoxazolyl. In one embodiment, D is —CR⁵¹R⁵²—, preferably —CH₂—, R⁶⁰ and R⁶¹ are hydrogen, R³¹ is hydrogen, R³⁰ is halogen or optionally fluoro substituted lower alkoxy, preferably lower alkoxy, A is phenyl, thiophenyl, pyridinyl, thiazolyl, or oxazolyl, wherein phenyl, thiophenyl, pyridinyl, thiazolyl, or oxazolyl are optionally substituted with halogen, lower alkyl, —OH, lower alkoxy, or lower alkylthio, wherein lower alkyl and the lower alkyl chains of lower alkoxy and lower alkylthio are optionally substituted with fluoro, —OH, lower alkoxy, or lower alkylthio, provided, however, that any substitution of the carbon bound to the lower alkoxy O or lower alkylthio S is fluoro, T is a covalent bond, —O—, or —NCH₃—, B is phenyl, pyridinyl, pyrazolyl, or isoxazolyl, and each R⁴³ is independently selected from the group consisting of halogen, —OH, optionally substituted lower alkyl, optionally substituted lower alkoxy, and optionally substituted lower alkylthio, preferably halogen, lower alkyl, —OH, lower alkoxy, or lower alkylthio, wherein lower alkyl and the lower alkyl chains of lower alkoxy and lower alkylthio are optionally substituted with fluoro, —OH, lower alkoxy, or lower alkylthio, provided, however, that any substitution of the carbon bound to the lower alkoxy O or lower alkylthio S is fluoro.

In one embodiment of compounds of Formula III, D is —CR⁵R⁵²—, preferably —CH₂—, R⁶⁰ and R⁶¹ are hydrogen, R³¹ is hydrogen, R³⁰ is halogen or optionally fluoro substituted lower alkoxy, preferably lower alkoxy, A is phenyl, thiophenyl, pyridinyl, thiazolyl, or oxazolyl optionally substituted with fluoro, chloro, optionally fluoro substituted lower alkyl, or optionally fluoro substituted lower alkoxy, T is a covalent bond, —O—, or —NCH₃—, B is phenyl, pyridinyl, pyrazolyl, or isoxazolyl, and each R⁴³ is independently selected from the group consisting of fluoro, chloro, optionally fluoro substituted lower alkyl, and optionally fluoro substituted lower alkoxy.

Further to any of the above embodiments of compounds of Formula III, R³² is —C(O)OR²⁶, preferably —COOH.

In some embodiments of the above compounds, compounds are excluded where N (except where N is a heteroaryl ring atom), O, or S is bound to a carbon that is also bound to N (except where N is a heteroaryl ring atom), O, or S; or where N (except where N is a heteroaryl ring atom), O, C(S), C(O), or S(O)ₙ(n is 0-2) is bound to an alkene carbon of an alkenyl group or bound to an alkyne carbon of an alkynyl group; accordingly, in some embodiments compounds that include linkages such as the following are excluded from the present invention: —NR—CH₂—NR—, —O—CH₂—NR—, —S—CH₂—NR—, —NR—CH₂—O—, —O—CH₂—O—, —S—CH₂—O—, —NR—CH₂—S—, —O—CH₂—S—, —S—CH₂—S—, —NR—CH═CH—, —CH═CH—NR—, —NR—C≡C—, —C≡C—NR—, —O—CH═CH—, —CH═CH—O—, —O—C≡C—, —C≡C—O—, —S(O)₀₋₂—CH═CH—, —CH═CH—S(O)₀₋₂—, —S(O)₀₋₂—C≡C—, —C≡C—S(O)₀₋₂—, —C(O)—CH═CH—, —CH═CH—C(O)—, —C≡C—C(O)—, —C(O)—C≡C—, —C(S)—CH═CH—, —CH═CH—C(S)—, —C≡C—C(S)—, or —C(S)—C≡C—.

Reference to compounds of Formulae I, II and III herein includes specific reference to sub-groups and species of compounds of Formulae I, II and III described herein (including all embodiments as described above, e.g. reference to Formula I includes reference to Formulae Ia and Ib) unless indicated to the contrary. In specifying a compound or compounds of Formulae I, II, or III, unless clearly indicated to the contrary, specification of such compound(s) includes pharmaceutically acceptable salts of the compound(s).

Another aspect of the invention relates to novel use of compounds of Formulae I, Ia, lb, II, or III for the treatment of diseases associated with PPARs.

Another aspect of this invention provides compositions that include a therapeutically effective amount of a compound of Formulae II or III and at least one pharmaceutically acceptable carrier, excipient, and/or diluent. The composition can include a plurality of different pharmacalogically active compounds, including one or more compounds of Formulae I, II or III.

In another aspect, compounds of Formulae II or III can be used in the preparation of a medicament for the treatment of a PPAR-mediated disease or condition or a disease or condition in which modulation of a PPAR provides a therapeutic benefit. In a further aspect, the disease or condition is selected from the group consisting of weight disorders (e.g. obesity, overweight condition, bulimia, and anorexia nervosa), lipid disorders (e.g. hyperlipidemia, dyslipidemia including associated diabetic dyslipidemia and mixed dyslipidemia hypoalphalipoproteinemia, hypertriglyceridemia, hypercholesterolemia, and low HDL (high density lipoprotein)), metabolic disorders (e.g. Metabolic Syndrome, Type II diabetes mellitus, Type I diabetes, hyperinsulinemia, impaired glucose tolerance, insulin resistance, diabetic complication including neuropathy, nephropathy, retinopathy, diabetic foot ulcer and cataracts), cardiovascular disease (e.g. hypertension, coronary heart disease, heart failure, congestive heart failure, atherosclerosis, arteriosclerosis, stroke, cerebrovascular disease, myocardial infarction, peripheral vascular disease), inflammatory diseases (e.g. autoimmune diseases such as vitiligo, uveitis, pemphigus foliaceus, inclusion body myositis, polymyositis, dermatomyositis, scleroderma, Grave's disease, Hashimoto's disease, chronic graft versus host disease, rheumatoid arthritis, inflammatory bowel syndrome, Crohn's disease, systemic lupus erythematosis, Sjogren's Syndrome, and multiple sclerosis, diseases involving airway inflammation such as asthma and chronic obstructive pulmonary disease, and inflammation in other organs, such as polycystic kidney disease (PKD), polycystic ovary syndrome, pancreatitis, nephritis, and hepatitis), skin disorders (e.g. epithelial hyperproliferative diseases such as eczema and psoriasis, dermatitis, including atopic dermatitis, contact dermatitis, allergic dermatitis and chronic dermatitis, and impaired wound healing), neurodegenerative disorders (e.g. Alzheimer's disease, Parkinson's disease, amyotrophic lateral sclerosis, spinal cord injury, and demyelinating disease, including acute disseminated encephalomyelitis and Guillain-Barre syndrome), coagulation disorders (e.g. thrombosis), gastrointestinal disorders (e.g. infarction of the large or small intestine), genitourinary disorders (e.g. renal insufficiency, erectile dysfunction, urinary incontinence, and neurogenic bladder), ophthalmic disorders (e.g. ophthalmic inflammation, macular degeneration, and pathologic neovascularization), infections (e.g. HCV, HIV, and Helicobacter pylori), neuropathic or inflammatory pain, infertility, and cancer. In some embodiments, the disease or condition is selected from the group consisting of obesity, overweight condition, bulimia, anorexia nervosa, hyperlipidemia, dyslipidemia, hypoalphalipoproteinemia, hypertriglyceridemia, hypercholesterolemia, Metabolic Syndrome, Type II diabetes mellitus, Type I diabetes, hyperinsulinemia, impaired glucose tolerance, insulin resistance, a diabetic complication of neuropathy, nephropathy, retinopathy, cataracts, hypertension, coronary heart disease, heart failure, congestive heart failure, atherosclerosis, arteriosclerosis, rheumatoid arthritis, inflammatory bowel syndrome, Crohn's disease, multiple sclerosis, asthma, chronic obstructive pulmonary disease, eczema, psoriasis, Alzheimer's disease, Parkinson's disease, amyotrophic lateral sclerosis, thrombosis, macular degeneration, infertility, and cancer. In some embodiments, the disease or condition is selected from the group consisting of vitiligo, uveitis, pemphigus foliaceus, inclusion body myositis, polymyositis, dermatomyositis, scleroderma, Grave's disease, Hashimoto's disease, chronic graft versus host disease, rheumatoid arthritis, inflammatory bowel syndrome, Crohn's disease, systemic lupus erythematosis, Sjogren's Syndrome, multiple sclerosis, asthma, chronic obstructive pulmonary disease, polycystic kidney disease, polycystic ovary syndrome, pancreatitis, nephritis, and hepatitis), dermatitis, impaired wound healing, Alzheimer's disease, Parkinson's disease, amyotrophic lateral sclerosis, spinal cord injury, acute disseminated encephalomyelitis, Guillain-Barre syndrome, infarction of the large or small intestine, renal insufficiency, erectile dysfunction, urinary incontinence, neurogenic bladder, ophthalmic inflammation, macular degeneration, pathologic neovascularization, HCV infection, HIV infection, Helicobacter pylori infection, neuropathic pain, inflammatory pain, and infertility. In some embodiments, the disease or condition is selected from the group consisting of Alzheimer's disease, Parkinson's disease, amyotrophic lateral sclerosis, rheumatoid arthritis, inflammatory bowel syndrome, Crohn's disease, multiple sclerosis, infertility, asthma, chronic obstructive pulmonary disease, and macular degeneration.

In another aspect, the invention provides kits that include a compound or composition as described herein. In some embodiments, the compound or composition is packaged, e.g., in a vial, bottle, flask, which may be further packaged, e.g., within a box, envelope, or bag; the compound or composition is approved by the U.S. Food and Drug Administration or similar regulatory agency for administration to a mammal,e.g., a human; the compound or composition is approved for administration to a mammal, e.g., a human for a PPAR-mediated disease or condition; the kit includes written instructions or other indication that the compound or composition is suitable or approved for administration to a mammal, e.g., a human, for a PPAR-mediated disease or condition; the compound or composition is packaged in unit does or single dose form, e.g., single dose pills, capsules, or the like. In some embodiments, the compound or composition of the kits of the invention are approved for a medical indication selected from the group consisting of obesity, overweight condition, bulimia, anorexia nervosa, hyperlipidemia, dyslipidemia, hypoalphalipoproteinemia, hypertriglyceridemia, hypercholesterolemia, low HDL, Metabolic Syndrome, Type II diabetes mellitus, Type I diabetes, hyperinsulinemia, impaired glucose tolerance, insulin resistance, a diabetic complication of neuropathy, nephropathy, retinopathy, diabetic foot ulcer or cataracts, hypertension, coronary heart disease, heart failure, congestive heart failure, atherosclerosis, arteriosclerosis, stroke, cerebrovascular disease, myocardial infarction, peripheral vascular disease, vitiligo, uveitis, pemphigus foliaceus, inclusion body myositis, polymyositis, dermatomyositis, scleroderma, Grave's disease, Hashimoto's disease, chronic graft versus host disease, rheumatoid arthritis, inflammatory bowel syndrome, Crohn's disease, systemic lupus erythematosis, Sjogren's Syndrome, multiple sclerosis, asthma, chronic obstructive pulmonary disease, polycystic kidney disease, polycystic ovary syndrome, pancreatitis, nephritis, hepatitis, eczema, psoriasis, dermatitis, impaired wound healing, Alzheimer's disease, Parkinson's disease, amyotrophic lateral sclerosis, spinal cord injury, acute disseminated encephalomyelitis, Guillain-Barre syndrome, thrombosis, infarction of the large or small intestine, renal insufficiency, erectile dysfunction, urinary incontinence, neurogenic bladder, ophthalmic inflammation, macular degeneration, pathologic neovascularization, HCV infection, HIV infection, Helicobacter pylori infection, neuropathic or inflammatory pain, infertility, and cancer. In some embodiments, the compound or composition of the kits of the invention are approved for a medical indication selected from the group consisting of obesity, overweight condition, bulimia, anorexia nervosa, hyperlipidemia, dyslipidemia, hypoalphalipoproteinemia, hypertriglyceridemia, hypercholesterolemia, Metabolic Syndrome, Type II diabetes mellitus, Type I diabetes, hyperinsulinemia, impaired glucose tolerance, insulin resistance, a diabetic complication of neuropathy, nephropathy, retinopathy, cataracts, hypertension, coronary heart disease, heart failure, congestive heart failure, atherosclerosis, arteriosclerosis, rheumatoid arthritis, inflammatory bowel syndrome, Crohn's disease, multiple sclerosis, asthma, chronic obstructive pulmonary disease, eczema, psoriasis, Alzheimer's disease, Parkinson's disease, amyotrophic lateral sclerosis, thrombosis, macular degeneration, infertility, and cancer. In some embodiments, the compound or composition of the kits of the invention are approved for a medical indication selected from the group consisting of vitiligo, uveitis, pemphigus foliaceus, inclusion body myositis, polymyositis, dermatomyositis, scleroderma, Grave's disease, Hashimoto's disease, chronic graft versus host disease, rheumatoid arthritis, inflammatory bowel syndrome, Crohn's disease, systemic lupus erythematosis, Sjogren's Syndrome, multiple sclerosis, asthma, chronic obstructive pulmonary disease, polycystic kidney disease, polycystic ovary syndrome, pancreatitis, nephritis, and hepatitis), dermatitis, impaired wound healing, Alzheimer's disease, Parkinson's disease, amyotrophic lateral sclerosis, spinal cord injury, acute disseminated encephalomyelitis, Guillain-Barre syndrome, infarction of the large or small intestine, renal insufficiency, erectile dysfunction, urinary incontinence, neurogenic bladder, ophthalmic inflammation, macular degeneration, pathologic neovascularization, HCV infection, HIV infection, Helicobacter pylori infection, neuropathic pain, inflammatory pain, and infertility. In some embodiments, the compound or composition of the kits of the invention are approved for a medical indication selected from the group consisting of Alzheimer's disease, Parkinson's disease, amyotrophic lateral sclerosis, rheumatoid arthritis, inflammatory bowel syndrome, Crohn's disease, multiple sclerosis, infertility, asthma, chronic obstructive pulmonary disease, and macular degeneration.

In another aspect, the invention provides a method of treating or prophylaxis of a disease or condition in an animal subject, e.g., a PPAR-mediated disease or condition or a disease or condition in which modulation of a PPAR provides a therapeutic benefit, by administering to the subject a therapeutically effective amount of a compound of Formulae I, II, or III, a prodrug of such compound, or a pharmaceutically acceptable salt of such compound or prodrug. The compound can be administered alone or can be administered as part of a composition. In one aspect, the method involves administering to the subject an effective amount of a compound of Formulae I, II, or III, in combination with one or more other therapies for the disease or condition.

In another aspect, the invention provides a method of treating or prophylaxis of a PPAR-mediated disease or condition or a disease or condition in which modulation of a PPAR provides a therapeutic benefit, wherein the method involves administering to the subject a therapeutically effective amount of a composition including a compound of Formulae I, II or III.

In aspects and embodiments involving treatment or prophylaxis of a disease or condition, the disease or condition is selected from the group consisting of weight disorders (e.g. obesity, overweight condition, bulimia, and anorexia nervosa), lipid disorders (e.g. hyperlipidemia, dyslipidemia including associated diabetic dyslipidemia and mixed dyslipidemia hypoalphalipoproteinemia, hypertriglyceridemia, hypercholesterolemia, and low HDL (high density lipoprotein)), metabolic disorders (e.g. Metabolic Syndrome, Type II diabetes mellitus, Type I diabetes, hyperinsulinemia, impaired glucose tolerance, insulin resistance, diabetic complication including neuropathy, nephropathy, retinopathy, diabetic foot ulcer and cataracts), cardiovascular disease (e.g. hypertension, coronary heart disease, heart failure, congestive heart failure, atherosclerosis, arteriosclerosis, stroke, cerebrovascular disease, myocardial infarction, peripheral vascular disease), inflammatory diseases (e.g. autoimmune diseases such as vitiligo, uveitis, pemphigus foliaceus, inclusion body myositis, polymyositis, dermatomyositis, scleroderma, Grave's disease, Hashimoto's disease, chronic graft versus host disease, rheumatoid arthritis, inflammatory bowel syndrome, Crohn's disease, systemic lupus erythematosis, Sjogren's Syndrome, and multiple sclerosis, diseases involving airway inflammation such as asthma and chronic obstructive pulmonary disease, and inflammation in other organs, such as polycystic kidney disease (PKD), polycystic ovary syndrome, pancreatitis, nephritis, and hepatitis), skin disorders (e.g. epithelial hyperproliferative diseases such as eczema and psoriasis, dermatitis, including atopic dermatitis, contact dermatitis, allergic dermatitis and chronic dermatitis, and impaired wound healing), neurodegenerative disorders (e.g. Alzheimer's disease, Parkinson's disease, amyotrophic lateral sclerosis, spinal cord injury, and demyelinating disease, including acute disseminated encephalomyelitis and Guillain-Barre syndrome), coagulation disorders (e.g. thrombosis), gastrointestinal disorders (e.g. infarction of the large or small intestine), genitourinary disorders (e.g. renal insufficiency, erectile dysfunction, urinary incontinence, and neurogenic bladder), ophthalmic disorders (e.g. ophthalmic inflammation, macular degeneration, and pathologic neovascularization), infections (e.g. HCV, HIV, and Helicobacter pylori), neuropathic or inflammatory pain, infertility, and cancer. In some embodiments, the disease or condition is selected from the group consisting of obesity, overweight condition, bulimia, anorexia nervosa, hyperlipidemia, dyslipidemia, hypoalphalipoproteinemia, hypertriglyceridemia, hypercholesterolemia, Metabolic Syndrome, Type II diabetes mellitus, Type I diabetes, hyperinsulinemia, impaired glucose tolerance, insulin resistance, a diabetic complication of neuropathy, nephropathy, retinopathy, cataracts, hypertension, coronary heart disease, heart failure, congestive heart failure, atherosclerosis, arteriosclerosis, rheumatoid arthritis, inflammatory bowel syndrome, Crohn's disease, multiple sclerosis, asthma, chronic obstructive pulmonary disease, eczema, psoriasis, Alzheimer's disease, Parkinson's disease, amyotrophic lateral sclerosis, thrombosis, macular degeneration, infertility, and cancer. In some embodiments, the disease or condition is selected from the group consisting of vitiligo, uveitis, pemphigus foliaceus, inclusion body myositis, polymyositis, dermatomyositis, scleroderma, Grave's disease, Hashimoto's disease, chronic graft versus host disease, rheumatoid arthritis, inflammatory bowel syndrome, Crohn's disease, systemic lupus erythematosis, Sjogren's Syndrome, multiple sclerosis, asthma, chronic obstructive pulmonary disease, polycystic kidney disease, polycystic ovary syndrome, pancreatitis, nephritis, and hepatitis), dermatitis, impaired wound healing, Alzheimer's disease, Parkinson's disease, amyotrophic lateral sclerosis, spinal cord injury, acute disseminated encephalomyelitis, Guillain-Barre syndrome, infarction of the large or small intestine, renal insufficiency, erectile dysfunction, urinary incontinence, neurogenic bladder, ophthalmic inflammation, macular degeneration, pathologic neovascularization, HCV infection, HIV infection, Helicobacter pylori infection, neuropathic pain, inflammatory pain, and infertility. In some embodiments, the disease or condition is selected from the group consisting of Alzheimer's disease, Parkinson's disease, amyotrophic lateral sclerosis, rheumatoid arthritis, inflammatory bowel syndrome, Crohn's disease, multiple sclerosis, infertility, asthma, chronic obstructive pulmonary disease, and macular degeneration.

In certain aspects and embodiments, compounds of Formulae II or III are used in the treatment or prophylaxis of a disease or condition selected from the group consisting of weight disorders (e.g. obesity, overweight condition, bulimia, and anorexia nervosa), lipid disorders (e.g. hyperlipidemia, dyslipidemia including associated diabetic dyslipidemia and mixed dyslipidemia hypoalphalipoproteinemia, hypertriglyceridemia, hypercholesterolemia, and low HDL (high density lipoprotein)), metabolic disorders (e.g. Metabolic Syndrome, Type II diabetes mellitus, Type I diabetes, hyperinsulinemia, impaired glucose tolerance, insulin resistance, diabetic complication including neuropathy, nephropathy, retinopathy, diabetic foot ulcer and cataracts), cardiovascular disease (e.g. hypertension, coronary heart disease, heart failure, congestive heart failure, atherosclerosis, arteriosclerosis, stroke, cerebrovascular disease, myocardial infarction, peripheral vascular disease), inflammatory diseases (e.g. autoimmune diseases such as vitiligo, uveitis, pemphigus foliaceus, inclusion body myositis, polymyositis, dermatomyositis, scleroderma, Grave's disease, Hashimoto's disease, chronic graft versus host disease, rheumatoid arthritis, inflammatory bowel syndrome, Crohn's disease, systemic lupus erythematosis, Sjogren's Syndrome, and multiple sclerosis, diseases involving airway inflammation such as asthma and chronic obstructive pulmonary disease, and inflammation in other organs, such as polycystic kidney disease (PKD), polycystic ovary syndrome, pancreatitis, nephritis, and hepatitis), skin disorders (e.g. epithelial hyperproliferative diseases such as eczema and psoriasis, dermatitis, including atopic dermatitis, contact dermatitis, allergic dermatitis and chronic dermatitis, and impaired wound healing), neurodegenerative disorders (e.g. Alzheimer's disease, Parkinson's disease, amyotrophic lateral sclerosis, spinal cord injury, and demyelinating disease, including acute disseminated encephalomyelitis and Guillain-Barre syndrome), coagulation disorders (e.g. thrombosis), gastrointestinal disorders (e.g. infarction of the large or small intestine), genitourinary disorders (e.g. renal insufficiency, erectile dysfunction, urinary incontinence, and neurogenic bladder), ophthalmic disorders (e.g. ophthalmic inflammation, macular degeneration, and pathologic neovascularization), infections (e.g. HCV, HIV, and Helicobacter pylori), neuropathic or inflammatory pain, infertility, and cancer. In some embodiments, the disease or condition is selected from the group consisting of obesity, overweight condition, bulimia, anorexia nervosa, hyperlipidemia, dyslipidemia, hypoalphalipoproteinemia, hypertriglyceridemia, hypercholesterolemia, Metabolic Syndrome, Type II diabetes mellitus, Type I diabetes, hyperinsulinemia, impaired glucose tolerance, insulin resistance, a diabetic complication of neuropathy, nephropathy, retinopathy, cataracts, hypertension, coronary heart disease, heart failure, congestive heart failure, atherosclerosis, arteriosclerosis, rheumatoid arthritis, inflammatory bowel syndrome, Crohn's disease, multiple sclerosis, asthma, chronic obstructive pulmonary disease, eczema, psoriasis, Alzheimer's disease, Parkinson's disease, amyotrophic lateral sclerosis, thrombosis, macular degeneration, infertility, and cancer. In some embodiments, the disease or condition is selected from the group consisting of vitiligo, uveitis, pemphigus foliaceus, inclusion body myositis, polymyositis, dermatomyositis, scleroderma, Grave's disease, Hashimoto's disease, chronic graft versus host disease, rheumatoid arthritis, inflammatory bowel syndrome, Crohn's disease, systemic lupus erythematosis, Sjogren's Syndrome, multiple sclerosis, asthma, chronic obstructive pulmonary disease, polycystic kidney disease, polycystic ovary syndrome, pancreatitis, nephritis, and hepatitis), dermatitis, impaired wound healing, Alzheimer's disease, Parkinson's disease, amyotrophic lateral sclerosis, spinal cord injury, acute disseminated encephalomyelitis, Guillain-Barre syndrome, infarction of the large or small intestine, renal insufficiency, erectile dysfunction, urinary incontinence, neurogenic bladder, ophthalmic inflammation, macular degeneration, pathologic neovascularization, HCV infection, HIV infection, Helicobacter pylori infection, neuropathic pain, inflammatory pain, and infertility. In some embodiments, the disease or condition is selected from the group consisting of Alzheimer's disease, Parkinson's disease, amyotrophic lateral sclerosis, rheumatoid arthritis, inflammatory bowel syndrome, Crohn's disease, multiple sclerosis, infertility, asthma, chronic obstructive pulmonary disease, and macular degeneration.

In certain aspects and embodiments, compounds of Formulae I, Ia, Ib, II, or III are used in the treatment or prophylaxis of a disease or condition selected from the group consisting of inflammatory diseases (e.g. autoimmune diseases such as vitiligo, uveitis, pemphigus foliaceus, inclusion body myositis, polymyositis, dermatomyositis, scleroderma, Grave's disease, Hashimoto's disease, chronic graft versus host disease, rheumatoid arthritis, inflammatory bowel syndrome, Crohn's disease, systemic lupus erythematosis, Sjogren's Syndrome, and multiple sclerosis, diseases involving airway inflammation such as asthma and chronic obstructive pulmonary disease, and inflammation in other organs, such as polycystic kidney disease (PKD), polycystic ovary syndrome, pancreatitis, nephritis, and hepatitis), skin disorders (e.g. dermatitis, including atopic dermatitis, contact dermatitis, allergic dermatitis and chronic dermatitis, and impaired wound healing), neurodegenerative disorders (e.g. Alzheimer's disease, Parkinson's disease, amyotrophic lateral sclerosis, spinal cord injury, and demyelinating disease, including acute disseminated encephalomyelitis and Guillain-Barre syndrome), gastrointestinal disorders (e.g. infarction of the large or small intestine), genitourinary disorders (e.g. renal insufficiency, erectile dysfunction, urinary incontinence, and neurogenic bladder), ophthalmic disorders (e.g. ophthalmic inflammation, macular degeneration, and pathologic neovascularization), infections (e.g. HCV, HIV, and Helicobacter pylori), neuropathic or inflammatory pain, and infertility. In some aspects and embodiments, PPAR modulators with chemical structure of Formulae I, Ia, or Ib are used in the treatment or prophylaxis of a disease or condition selected from the group consisting of neurodegenerative diseases, Alzheimer's disease, Parkinson's disease, amyotrophic lateral sclerosis, rheumatoid arthritis, inflammatory bowel syndrome, Crohn's disease, multiple sclerosis, infertility, asthma, chronic obstructive pulmonary disease, and macular degeneration.

In some embodiments of aspects involving compounds of Formulae I, II, or III the compound is specific for any one or any two of PPARα, PPARγ and PPARδ, e.g. specific for PPARα; specific for PPARδ; specific for PPARγ; specific for PPARα and PPARδ; specific for PPARα and PPARγ; or specific for PPARδ and PPARγ. Such specificity means that the compound has at least 5-fold greater activity (preferably at least 5-, 10-, 20-, 50-, or 100-fold or more greater activity) on the specific PPAR(s) than on the other PPAR(s), where the activity is determined using a biochemical assay suitable for determining PPAR activity, e.g., any assay known to one skilled in the art or as described herein. In another embodiment, compounds have significant activity on all three of PPARα, PPARδ, and PPARγ.

In some embodiments, a compound of Formulae I, II, or III will have an EC₅₀ Of less than 100 nM, less than 50 nM, less than 20 nM, less than 10 nM, less than 5 nM, or less than 1 nM with respect to at least one of PPARα, PPARγ and PPARδ as determined in a generally accepted PPAR activity assay. In one embodiment, a compound of any of Formulae I, II, or III will have an EC₅₀ of less than 100 nM, less than 50 nM, less than 20 nM, less than 10 nM, less than 5 nM, or less than 1 nM with respect to at least any two of PPARα, PPARγ and PPARδ. In one embodiment, a compound of any of Formulae I, II, or III will have an EC₅₀ of less than 100 nM, less than 50 nM, less than 20 nM, less than 10 nM, less than 5 nM, or less than 1 nM with respect to all three of PPARα, PPARγ and PPARδ. Further to any of the above embodiments, a compound of the invention may be a specific agonist of any one of PPARα, PPARγ and PPARδ, or any two of PPARα, PPARγ and PPARδ. A specific agonist of one of PPARα, PPARγ and PPARδ is such that the EC₅₀ for one of PPARα, PPARγ and PPARδ will be at least about 5-fold, also 10-fold, also 20-fold, also 50-fold, or at least about 100-fold less than the EC₅₀ for the other two of PPARα, PPARγ and PPARδ. A specific agonist of two of PPARα, PPARγ and PPARδ is such that the EC₅₀ for each of two of PPARα, PPARγ and PPARδ will be at least about 5-fold, also 10-fold, also 20-fold, also 50-fold, or at least about 100-fold less than the EC₅₀ for the other of PPARα, PPARγ and PPARδ.

In some embodiments of the invention, the compounds of Formulae I, II, or III active on PPARs also have desireable pharmacologic properties. In some embodiments the desired pharmacologic property is PPAR pan-activity, PPAR selectivity for any individual PPAR (PPARα, PPARδ, or PPARγ), selectivity on any two PPARs (PPARα and PPARδ, PPARα and PPARδ, or PPARδ and PPARγ), or any one or more of serum half-life longer than 2 hr, also longer than 4 hr, also longer than 8 hr, aqueous solubility, and oral bioavailability more than 10%, also more than 20%.

Additional embodiments will be apparent from the Detailed Description and from the claims.

### DETAILED DESCRIPTION OF THE INVENTION

As indicated in the Summary above, the present invention concerns the peroxisome proliferator-activated receptors (PPARs), which have been identified in humans and other mammals. A group of compounds have been identified, corresponding to Formulae I, II, or III, that are active on one or more of the PPARs, in particular compounds that are active on one or more human PPARs. Such compounds can be used for a variety of applications, e.g., as agonists on PPARs, including agonists of at least one of PPARα, PPARδ, and PPARγ , as well as dual PPAR agonists and pan-agonist, such as agonists of both PPARα and PPARγ, both PPARα and PPARδ, both PPARγ and PPARδ, or agonists of PPARα, PPARγ and PPARδ.

As used herein the following definitions apply unless otherwise indicated:

“Halogen”—alone or in combination refers to all halogens, that is, chloro (Cl), fluoro (F), bromo (Br), or iodo (I).

“Hydroxyl” or “hydroxy” refer to the group —OH.

“Thiol” refers to the group —SH.

“Lower alkyl” alone or in combination means an alkane-derived radical containing from 1 to 6 carbon atoms (unless specifically defined) that includes a straight chain alkyl or branched alkyl. The straight chain or branched alkyl group is attached at any available point to produce a stable compound. In many embodiments, a lower alkyl is a straight or branched alkyl group containing from 1-6, 1-4, or 1-2, carbon atoms, such as methyl, ethyl, propyl, isopropyl, butyl, t-butyl, and the like. “Substituted lower alkyl” denotes lower alkyl that is independently substituted, unless indicated otherwise, with one or more, preferably 1, 2, 3, 4 or 5, also 1, 2, or 3 substituents, attached at any available atom to produce a stable compound, wherein the substituents are selected from the group consisting of —F, —NO₂, —CN, —OR^{a}, —SR^{a}, —OC(O)R^{a}, —OC(S)R^{a}, —C(O)R^{a}, C(S)R^{a}, —C(O)OR^{a}, —C(S)OR^{a}, —S(O)R^{a}, —S(O)₂R^{a}, —C(O)NR^{a}R^{a}, —C(S)NR^{a}R^{a}, —S(O)₂NR^{a}R^{a}, —C(NH)NR^{b}R^{c}, —NR^{a}C(O)R^{a}, —NR^{a}C(S)R^{a}, —NR^{a}S(O)₂R^{a}, —NR^{a}C(O)NR^{a}R^{a}, —NR^{a}C(S)NR^{a}R^{a}, —NR^{a}S(O)₂NR^{a}R^{a}, —NR^{a}R^{a}, —R^{e}, and —R^{f}. Furthermore, possible substitutions include subsets of these substitutions, such as are indicated herein, for example, in the description of compounds of Formulae I, II, or III, attached at any available atom to produce a stable compound. For example “fluoro substituted lower alkyl” denotes a lower alkyl group substituted with one or more fluoro atoms, such as perfluoroalkyl, where preferably the lower alkyl is substituted with 1, 2, 3, 4 or 5 fluoro atoms, also 1, 2, or 3 fluoro atoms. It is understood that substitutions are attached at any available atom to produce a stable compound, when optionally substituted lower alkyl is an R group of a moiety such as —OR (e.g. lower alkoxy), —SR (e.g. lower alkylthio), —NHR (e.g. mono-alkylamino), —C(O)NHR, and the like, substitution of the lower alkyl R group is such that substitution of the lower alkyl carbon bound to any O, S, or N of the moiety (except where N is a heteroaryl ring atom) excludes substituents that would result in any O, S, or N of the substituent (except where N is a heteroaryl ring atom) being bound to the lower alkyl carbon bound to any O, S, or N of the moiety.

“Lower alkenyl” alone or in combination means a straight or branched hydrocarbon containing 2-6 carbon atoms (unless specifically defined) and at least one, preferably 1-3, more preferably 1-2, most preferably one, carbon to carbon double bond. Carbon to carbon double bonds may be contained within either a straight chain or branched portion. Examples of lower alkenyl groups include ethenyl, propenyl, isopropenyl, butenyl, and the like. “Substituted lower alkenyl” denotes lower alkenyl that is independently substituted, unless indicated otherwise, with one or more, preferably 1, 2, 3, 4 or 5, also 1, 2, or 3 substituents, attached at any available atom to produce a stable compound, wherein the substituents are selected from the group consisting of —F, —NO₂, —CN, —OR^{a}, —SR^{a}, —OC(O)R^{a}, —OC(S)R^{a}, —C(O)R^{a}, —C(S)R^{a}, —C(O)OR^{a}, —C(S)OR^{a}, —S(O)R^{a}, —S(O)₂R^{a}, —C(O)NR^{a}R^{a}, —C(S)NR^{a}R^{a}, —S(O)₂NR^{a}R^{a}, —C(NH)NR^{b}R^{c}, —NR^{a}C(O)R^{a}, —NR^{a}C(S)R^{a}, —NR^{a}S(O)₂R^{a}, —NR^{a}C(O)NR^{a}R^{a}, —NR^{a}C(S)NR^{a}R^{a}, —NR^{a}S(O)₂NR^{a}R^{a}, —NR^{a}R^{a}, —R^{d}, and —R^{f}. Further, possible substitutions include subsets of these substitutions, such as are indicated herein, for example, in the description of compounds of Formulae I, II, or III, attached at any available atom to produce a stable compound. It is understood that substitutions are attached at any available atom to produce a stable compound, substitution of lower alkenyl groups are such that F, C(O), C(S), C(NH), S(O), S(O)₂, O, S, or N (except where N is a heteroaryl ring atom), are not bound to an alkene carbon thereof. Further, where lower alkenyl is a substituent of another moiety or an R group of a moiety such as —OR, —NHR, —C(O)R, and the like, substitution of the moiety is such that any C(O), C(S), S(O), S(O)₂, O, S, or N thereof (except where N is a heteroaryl ring atom) are not bound to an alkene carbon of the lower alkenyl substituent or R group. Further, where lower alkenyl is a substituent of another moiety or an R group of a moiety such as —OR, —NHR, —C(O)NHR, and the like, substitution of the lower alkenyl R group is such that substitution of the lower alkenyl carbon bound to any O, S, or N of the moiety (except where N is a heteroaryl ring atom) excludes substituents that would result in any O, S, or N of the substituent (except where N is a heteroaryl ring atom) being bound to the lower alkenyl carbon bound to any O, S, or N of the moiety. An “alkenyl carbon” refers to any carbon within a lower alkenyl group, whether saturated or part of the carbon to carbon double bond. An “alkene carbon” refers to a carbon within a lower alkenyl group that is part of a carbon to carbon double bond. “C₃₋₆ alkenyl” denotes lower alkenyl containing 3-6 carbon atoms. A “substituted C₃₋₆ alkenyl” denotes optionally substituted lower alkenyl containing 3-6 carbon atoms.

“Lower alkynyl” alone or in combination means a straight or branched hydrocarbon containing 2-6 carbon atoms (unless specifically defined) containing at least one, preferably one, carbon to carbon triple bond. Examples of lower alkynyl groups include ethynyl, propynyl, butynyl, and the like. “Substituted lower alkynyl” denotes lower alkynyl that is independently substituted, unless indicated otherwise, with one or more, preferably 1, 2, 3, 4 or 5, also 1, 2, or 3 substituents, attached at any available atom to produce a stable compound, wherein the substituents are selected from the group consisting of —F, —NO₂, —CN, —OR^{a}, —SR^{a}, —OC(O)R^{a}, —OC(S)R^{a}, —C(O)R^{a}, —C(S)R^{a}, —C(O)OR^{a}, —C(S)OR^{a}, —S(O)R^{a}, —S(O)₂R^{a}, —C(O)NR^{a}R^{a}, —C(S)NR^{a}R^{a}, —S(O)₂NR^{a}R^{a}, —C(NH)NR^{b}R^{c}, —NR^{a}C(O)R^{a}, —NR^{a}C(S)R^{a}, —NR^{a}S(O)₂R^{a}, —NR^{a}C(O)NR^{a}R^{a}, —NR^{a}C(S)NR^{a}R^{a}, —NR^{a}S(O)₂NR^{a}R^{a}, —NR^{a}R^{a}, —R^{d}, and —R^{f}. Further, possible substitutions included subsets of these substitutions, such as are indicated herein, for example, in the description of compounds of Formulae I, II, or III, attached at any available atom to produce a stable compound. It is understood that substitutions are attached at any available atom to produce a stable compound, substitution of lower alkynyl groups are such that F, C(O), C(S), C(NH), S(O), S(O)₂, O, S, or N (except where N is a heteroaryl ring atom) are not bound to an alkyne carbon thereof. Further, where lower alkynyl is a substituent of another moiety or an R group of a moiety such as —OR, —NHR, —C(O)R, and the like, substitution of the moiety is such that any C(O), C(S), S(O), S(O)₂, O, S, or N thereof (except where N is a heteroaryl ring atom) are not bound to an alkyne carbon of the lower alkynyl substituent or R group. Further, where lower alkynyl is a substituent of another moiety or an R group of a moiety such as —OR, —NHR, —C(O)NHR, and the like, substitution of the lower alkynyl R group is such that substitution of the lower alkynyl carbon bound to any O, S, or N of the moiety (except where N is a heteroaryl ring atom) excludes substituents that would result in any O, S, or N of the substituent (except where N is a heteroaryl ring atom) being bound to the lower alkynyl carbon bound to any O, S, or N of the moiety. An “alkynyl carbon” refers to any carbon within a lower alkynyl group, whether saturated or part of the carbon to carbon triple bond. An “alkyne carbon” refers to a carbon within a lower alkynyl group that is part of a carbon to carbon triple bond. “C₃₋₆ alkynyl” denotes lower alkynyl containing 3-6 carbon atoms. A “substituted C₃₋₆ alkynyl” denotes optionally substituted lower alkynyl containing 3-6 carbon atoms.

“Carboxylic acid isostere” refers to a moiety selected from the group consisting of thiazolidine dione (i.e. 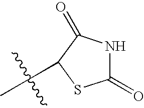
), hydroxamic acid (i.e. —C(O)NHOH), acyl-cyanamide (i.e. —C(O)NHCN), tetrazole (i.e. 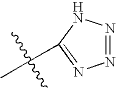
), 3- or 5-hydroxy isoxazole (i.e. 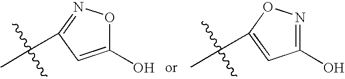
), 3- or 5-hydroxy isothiazole (i.e. 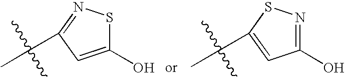
), sulphonate (i.e. —S(O)₂OH), and sulfonamide (i.e. —S(O)₂NH₂). In functional terms, carboxylic acid isosteres mimic carboxylic acids by virtue of similar physical properties, including but not limited to molecular size, charge distribution or molecular shape. 3- or 5-hydroxy isoxazole or 3- or 5-hydroxy isothiazole may be optionally substituted with lower alkyl or lower alkyl substituted with 1, 2 or 3 substituents selected from the group consisting of fluoro, aryl and heteroaryl, wherein aryl or heteroaryl may further be optionally substituted with 1, 2, or 3 substituents selected from the group consisting of halogen, lower alkyl, fluoro substituted lower alkyl, lower alkoxy, fluoro substituted lower alkoxy, lower alkylthio, and fluoro substituted lower alkylthio. The nitrogen of the sulfonamide may be optionally substituted with a substituent selected from the group consisting of lower alkyl, fluoro substituted lower alkyl, acetyl (i.e. —C(O)CH₃), aryl and heteroaryl, wherein aryl or heteroaryl may further be optionally substituted with 1, 2, or 3 substituents selected from the group consisting of halogen, lower alkyl, fluoro substituted lower alkyl, lower alkoxy, fluoro substituted lower alkoxy, lower alkylthio, and fluoro substituted lower alkylthio.

“Aryl” alone or in combination refers to a monocyclic or bicyclic ring system containing aromatic hydrocarbons such as phenyl or naphthyl, which may be optionally fused with a cycloalkyl or heterocycloalkyl of preferably 5-7, more preferably 5-6, ring members. “Arylene” refers to a divalent aryl.

“Heteroaryl” alone or in combination refers to a monocyclic aromatic ring structure containing 5 or 6 ring atoms, or a bicyclic aromatic group having 8 to 10 atoms, containing one or more, preferably 1-4, more preferably 1-3, even more preferably 1-2, heteroatoms independently selected from the group consisting of O, S, and N. Heteroaryl is also intended to include oxidized S or N, such as sulfinyl, sulfonyl and N-oxide of a tertiary ring nitrogen. A carbon or nitrogen atom is the point of attachment of the heteroaryl ring structure such that a stable compound is produced. Examples of heteroaryl groups include, but are not limited to, pyridinyl, pyridazinyl, pyrazinyl, quinoxalinyl, indolizinyl, benzo[b]thienyl, quinazolinyl, purinyl, indolyl, quinolinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, and indolyl. “Nitrogen containing heteroaryl” refers to heteroaryl wherein any heteroatoms are N. “Heteroarylene” refers to a divalent heteroaryl.

“Cycloalkyl” refers to saturated or unsaturated, non-aromatic monocyclic, bicyclic or tricyclic carbon ring systems of 3-10, also 3-8, more preferably 3-6, ring members per ring, such as cyclopropyl, cyclopentyl, cyclohexyl, adamantyl, and the like.

“Heterocycloalkyl” refers to a saturated or unsaturated non-aromatic cycloalkyl group having from 5 to 10 atoms in which from 1 to 3 carbon atoms in the ring are replaced by heteroatoms of O, S or N, and are optionally fused with benzo or heteroaryl of 5-6 ring members. Heterocycloalkyl is also intended to include oxidized S or N, such as sulfinyl, sulfonyl and N-oxide of a tertiary ring nitrogen. Heterocycloalkyl is also intended to include compounds in which one of the ring carbons is oxo substituted, i.e. the ring carbon is a carbonyl group, such as lactones and lactams. The point of attachment of the heterocycloalkyl ring is at a carbon or nitrogen atom such that a stable ring is retained. Examples of heterocycloalkyl groups include, but are not limited to, morpholino, tetrahydrofuranyl, dihydropyridinyl, piperidinyl, pyrrolidinyl, pyrrolidonyl, piperazinyl, dihydrobenzofuryl, and dihydroindolyl.

“Optionally substituted aryl”, “optionally substituted heteroaryl”, “optionally substituted cycloalkyl”, and “optionally substituted heterocycloalkyl”, refers to aryl, heteroaryl, cycloalkyl and heterocycloalkyl groups, respectively, which are optionally independently substituted, unless indicated otherwise, with one or more, preferably 1, 2, 3, 4 or 5, also 1, 2, or 3 substituents, attached at any available atom to produce a stable compound, wherein the substituents are selected from the group consisting of halogen, —NO₂, —CN, —OR^{a}, —SR^{a}, —OC(O)R^{a}, —OC(S)R^{a}, —C(O)R^{a}, —C(S)R^{a}, —C(O)OR^{a}, —C(S)OR^{a}, —NO₂, —CN, —OR^{a}, —SR^{a}, —OC(O)R^{a}, —OC(S)R^{a}, —C(O)R^{a}, —C(S)R^{a}, —C(O)OR^{a}, —C(S)OR^{a}, —(O)R^{a}, —S(O)₂R^{a}, —C(O)NR^{a}R^{a}, —C(S)NR^{a}R^{a}, —S(O)₂NR^{a}R^{a}, —C(NH)NR^{b}R^{c}, —NR^{a}C(O)R^{a}, —NR^{a}C(S)R^{a}, —NR^{a}S(O)₂R^{a}, —NR^{a}C(O)NR^{a}R^{a}, —NR^{a}C(S)NR^{a}R^{a}, —NR^{a}S(O)₂NR^{a}R^{a}, NR^{a}R^{a}, —R^{d}, —R^{e}, and —R^{f}.

The variables as used in the description of optional substituents for lower alkyl, lower alkenyl, lower alkynyl, cycloalkyl, heterocycloalkyl, aryl and heteroaryl are defined as follows:
—R^{a}, —R^{b}, and R^{c} at each occurrence are independently selected from the group consisting of hydrogen, —R^{d}, —R^{e}, and —R^{f}, provided, however, that R^{a}bound to S, S(O), S(O)₂, C(O) or C(O) is not hydrogen, or
—R^{b} and —R^{c} combine with the nitrogen to which they are attached form a 5-7 membered heterocycloalkyl or a 5 or 7 membered nitrogen containing heteroaryl, wherein the 5-7 membered heterocycloalkyl or 5 or 7 membered nitrogen containing heteroaryl are optionally substituted with one or more, preferably 1, 2, 3, 4 or 5, also 1, 2, or 3 substituents selected from the group consisting of halogen, cycloalkylamino, —NO₂, —CN, —OR^{k}, —SR^{k}, —NR^{k}R^{k}, —R^{m}, and ^{R}^{o};
—R^{d} at each occurrence is independently lower alkyl optionally substituted with one or more, preferably 1, 2, 3, 4 or 5, also 1, 2 or 3 substituents selected from the group consisting of fluoro, —OR^{g}, —SR^{g}, —NR^{g}R^{g}, —C(O)R^{g}, —C(S)R^{g}, —S(O)R^{g}, —S(O)₂R^{g}, —C(O)NR^{g}R^{g}, —C(S)NR^{g}R^{g}, —S(O)₂NR^{g}R^{g}, —NR^{g}C(O)R^{g}, —NR^{g}C(S)R^{g}, —NR^{g}S(O)₂R^{g}, —NR^{g}C(O)NR^{g}R^{g}, —NR^{g}C(S)NR^{g}R^{g}, —NR^{g}S(O)₂NR^{g}R^{g}, and —R^{f};
—R^{e} at each occurrence is independently selected from the group consisting of lower alkenyl and lower alkynyl, wherein lower alkenyl or lower alkynyl are optionally substituted with one or more, preferably 1, 2, 3, 4 or 5, also 1, 2 or 3 substituents selected from the group consisting of fluoro, —OR^{g9}, —SR^{g9}, —NR^{g}R^{g}, —C(O)R^{g}, —C(S)R^{g}, —S(O)R^{g}, —S(O)₂R^{g}, —C(O)NR^{g}R^{g}, —C(S)NR^{g}R^{g}, —S(O)₂NR^{g}R^{g}, —NR^{g}C(O)R^{g}, —NR^{g}C(S)R^{g}, —NR^{g}S(O)₂R^{g}, —NR^{g}C(O)NR^{g}R^{g}, —NR^{g}C(S)NR^{g}R^{g}, —NR^{g}S(O)₂NR^{g}R^{g}, —R^{d}, and —R^{f};
—R^{f} at each occurrence is independently selected from the group consisting of cycloalkyl, heterocycloalkyl, aryl, and heteroaryl, wherein cycloalkyl, heterocycloalkyl, aryl, and heteroaryl are optionally substituted with one or more, preferably 1, 2, 3, 4 or 5, also 1, 2 or 3 substituents selected from the group consisting of halogen, —NO₂, —CN, —OR⁹, —SR⁹, —NR^{g}R^{g}, —C(O)R^{g}, —C(S)R^{g}, —S(O)R^{g}, —S(O)₂R^{g}, —C(O)NR^{g}R^{g}, —C(S)NR^{g}R^{g}, —S(O)₂NR^{g}R^{g}, —NR^{g}C(O)R^{g}, —NR^{g}C(S)R^{g}, —NR^{g}S(O)₂R^{g}, —NR^{g}C(O)NR^{g}R^{g}, —NR^{g}C(S)NR^{g}R^{g}, —NR^{g}S(O)₂NR^{g}R^{g}, —R^{m}, and —R^{o};
—R^{g} at each occurrence is independently selected from the group consisting of hydrogen, —R^{h}, —Rⁱ, and —R^{j}, provided, however, that R^{g}bound to S, S(O), S(O)₂, C(S) or C(O) is not hydrogen;
—R^{h} at each occurrence is independently lower alkyl optionally substituted with one or more, preferably 1, 2, 3, 4 or 5, also 1, 2, or 3 substituents selected from the group consisting of fluoro, —OR^{k}, —SR^{k}, —NR^{k}R^{k}, —C(O)R^{k}, —C(S)R^{k}, —S(O)R^{k}, —S(O)₂R^{k}, —C(O)NR^{k}R^{k}, —C(S)NR^{k}R^{k}, —S(O)₂NR^{k}R^{k}, —NR^{k}C(O)R^{k}, —NR^{k}C(S)R^{k}, —NR^{k}S(O)₂R^{k}, —NR^{k}C(O)NR^{k}R^{k}, —NR^{k}C(S)NR^{k}R^{k}, —NR^{k}S(O)₂NR^{k}R^{k}, and —R^{o}, provided, however, that any substitution on the lower alkyl carbon bound to any O, S, or N of any OR^{h}, SR^{h}, or NR^{h} is selected from the group consisting of fluoro and —R^{o};
—Rⁱ at each occurrence is independently selected from the group consisting of C₃₋₆ alkenyl and C₃₋₆ alkynyl, wherein C₃₋₆ alkenyl or C₃₋₆ alkynyl are optionally substituted with one or more, preferably 1, 2, 3, 4 or 5, also 1, 2, or 3 substituents selected from the group consisting of fluoro, —OR^{k}, —SR^{k}, —NR^{k}R^{k}, —C(O)R^{k}, —C(S)R^{k}, —S(O)R^{k}, —S(O)₂R^{k}, —C(O)NR^{k}R^{k}, —C(S)NR^{k}R^{k}, —S(O)₂NR^{k}R^{k}, —NR^{k}C(O)R^{k}, —NR^{k}C(S)R^{k}, —NR^{k}S(O)₂R^{k}, —NR^{k}C(O)NR^{k}R^{k}, —NR^{k}C(S)NR^{k}R^{k}, —NR^{k}S(O)₂NR^{k}R^{k}, —R^{m} and —R^{o}, provided, however, that any substitution on the alkenyl or alkynyl carbon bound to any O, S, or N of any ORⁱ, SRⁱ, or NRⁱ is selected from the group consisting of fluoro, —R^{m} and —R^{o};
—R^{j} at each occurrence is independently selected from the group consisting of cycloalkyl, heterocycloalkyl, aryl, and heteroaryl, wherein cycloalkyl, heterocycloalkyl, aryl, and heteroaryl are optionally substituted with one or more, preferably 1, 2, 3, 4 or 5, also 1, 2, or 3 substituents selected from the group consisting of halogen, —NO₂, —CN, —OR^{k}, —SR^{k}, —NR^{k}R^{k}, —C(O)R^{k}, —C(S)R , —S(O)R^{k}, —S(O)₂R^{k}, —C(O)NR^{k}R^{k}, —C(S)NR^{k}R^{k}, —S(O)₂NR^{k}R^{k}, —NR^{k}C(O)R^{k}, —NR^{k}C(S)R^{k}, —NR^{k}S(O)₂R^{k}, —NR^{k}C(O)NR^{k}R^{k}, —NR^{k}C(S)NR^{k}R^{k}, —NR^{k}S(O)₂NR^{k}R^{k}, —R^{m}, and —R^{o};
—R^{m} at each occurrence is independently selected from the group consisting of lower alkyl, lower alkenyl and lower alkynyl, wherein lower alkyl is optionally substituted with one or more, preferably 1, 2, 3, 4 or 5, also 1, 2, or 3 substituents selected from the group consisting of —R^{o}, fluoro, lower alkoxy, fluoro substituted lower alkoxy, lower alkylthio, fluoro substituted lower alkylthio, mono-alkylamino, di-alkylamino, and cycloalkylamino, and wherein lower alkenyl or lower alkynyl are optionally substituted with one or more, preferably 1, 2, 3, 4 or 5, also 1, 2, or 3 substituents selected from the group consisting of —R^{o}, fluoro, lower alkyl, fluoro substituted lower alkyl, lower alkoxy, fluoro substituted lower alkoxy, lower alkylthio, fluoro substituted lower alkylthio, mono-alkylamino, di-alkylamino, and cycloalkylamino;

—R^{k}at each occurrence is independently selected from the group consisting of hydrogen, —Rⁿ, and —R^{o}, provided, however, that R^{k}bound to S, S(O), S(O)₂, C(S) or C(O) is not hydrogen;
—Rⁿ at each occurrence is independently selected from the group consisting of lower alkyl, C₃₋₆ alkenyl and C₃₋₆ alkynyl, wherein lower alkyl is optionally substituted with one or more, preferably 1, 2, 3, 4 or 5, also 1, 2, or 3 substituents selected from the group consisting of —R^{o}, fluoro, lower alkoxy, fluoro substituted lower alkoxy, lower alkylthio, fluoro substituted lower alkylthio, mono-alkylamino, di-alkylamino, and cycloalkylamino, provided, however, that any substitution of the lower alkyl carbon bound to the O of ORⁿ, S of SRⁿ, or N of any NRⁿ is fluoro or —R^{o}, and wherein C₃₋₆ alkenyl or C₃₋₆ alkynyl are optionally substituted with one or more, preferably 1, 2, 3, 4 or 5, also 1, 2, or 3 substituents selected from the group consisting of —R^{o}, fluoro, lower alkyl, fluoro substituted lower alkyl, lower alkoxy, fluoro substituted lower alkoxy, lower alkylthio, fluoro substituted lower alkylthio, mono-alkylamino, di-alkylamino, and cycloalkylamino, provided, however, that any substitution of the C₃₋₆ alkenyl or C₃₋₆ alkynyl carbon bound to the the O of ORⁿ, S of SRⁿ, or N of any NRⁿ is fluoro, lower alkyl, fluoro substituted lower alkyl, or —R^{o};
—R^{o} at each occurrence is independently selected from the group consisting of cycloalkyl, heterocycloalkyl, aryl, and heteroaryl, wherein cycloalkyl, heterocycloalkyl, aryl, and heteroaryl are optionally substituted with one or more, preferably 1, 2, 3, 4 or 5, also 1, 2, or 3 substituents selected from the group consisting of halogen, —OH, —NH₂, —NO₂, —CN, lower alkyl, fluoro substituted lower alkyl, lower alkoxy, fluoro substituted lower alkoxy, lower alklthio, fluoro substituted lower alkylthio, mono-alkylamino, di-alkylamino, and cycloalkylamino.

“Lower alkoxy” denotes the group —OR^{p}, where R^{p} is lower alkyl. “Optionally substituted lower alkoxy” denotes lower alkoxy in which R^{p} is optionally substituted lower alkyl. Preferably, substitution of lower alkoxy is with 1, 2, 3, 4, or 5 substituents, also 1, 2, or 3 substituents. For example “fluoro substituted lower alkoxy” denotes lower alkoxy in which the lower alkyl is substituted with one or more fluoro atoms, where preferably the lower alkoxy is substituted with 1, 2, 3, 4 or 5 fluoro atoms, also 1, 2, or 3 fluoro atoms. It is understood that substitutions on lower alkoxy are attached at any available atom to produce a stable compound, substitution of lower alkoxy is such that O, S, or N (except where N is a heteroaryl ring atom), are not bound to the lower alkyl carbon bound to the lower alkoxy O. Further, where lower alkoxy is described as a substituent of another moiety, the lower alkoxy oxygen is not bound to a carbon atom that is bound to an O, S, or N of the other moiety (except where N is a heteroaryl ring atom), or to an alkene or alkyne carbon of the other moiety.

“Aryloxy” denotes the group —OR^{q}, where R^{q} is aryl. “Optionally substituted aryloxy” denotes aryloxy in which R^{q} is optionally substituted aryl. “Heteroaryloxy” denotes the group —OR^{r}, where R^{r} is heteroaryl. “Optionally substituted heteroaryloxy” denotes heteroaryloxy in which R^{r} is optionally substituted heteroaryl.

“Lower alkylthio” denotes the group —SR^{s}, where R^{s} is lower alkyl. “Substituted lower alkylthio” denotes lower alkylthio in which R^{s} is optionally substituted lower alkyl. Preferably, substitution of lower alkylthio is with 1, 2, 3, 4, or 5 substituents, also 1, 2, or 3 substituents. For example “fluoro substituted lower alkylthio” denotes lower alkylthio in which the lower alkyl is substituted with one or more fluoro atoms, where preferably the lower alkylthio is substituted with 1, 2, 3, 4 or 5 fluoro atoms, also 1, 2, or 3 fluoro atoms. It is understood that substitutions on lower alkylthio are attached at any available atom to produce a stable compound, substitution of lower alkylthio is such that O, S, or N (except where N is a heteroaryl ring atom), are not bound to the lower alkyl carbon bound to the lower alkylthio S. Further, where lower alkylthio is described as a substituent of another moiety, the lower alkylthio sulfur is not bound to a carbon atom that is bound to an O, S, or N of the other moiety (except where N is a heteroaryl ring atom), or to an alkene or alkyne carbon of the other moiety.

“Amino” or “amine” denotes the group —NH₂. “Mono-alkylamino” denotes the group —NHR^{t} where R^{t} is lower alkyl. “Di-alkylamino” denotes the group —NR^{t}R^{u}, where R^{t} and R^{u} are independently lower alkyl. “Cycloalkylamino” denotes the group —NR^{v}R^{w}, where R^{v} and R^{w} combine with the nitrogen to form a 5-7 membered heterocycloalkyl, where the heterocycloalkyl may contain an additional heteroatom within the ring, such as O, N, or S, and may also be further substituted with lower alkyl. Examples of cycloalkylamino include, but are not limited to, piperidine, piperazine, 4-methylpiperazine, morpholine, and thiomorpholine. It is understood that when mono-alkylamino, di-alkylamino, or cycloalkylamino are substituents on other moieties that are attached at any available atom to produce a stable compound, the nitrogen of mono-alkylamino, di-alkylamino, or cycloalkylamino as substituents is not bound to a carbon atom that is bound to an O, S, or N of the other moiety (except where N is a heteroaryl ring atom) or to an alkene or alkyne carbon of the other moiety.

As used herein in connection with PPAR modulating compound, binding compounds or ligands, the term “specific for PPAR” and terms of like import mean that a particular compound binds to a PPAR to a statistically greater extent than to other biomolecules that may be present in or originally isolated from a particular organism, e.g., at least 2, 3, 4, 5, 10, 20, 50, 100, or 1000-fold greater binding. Also, where biological activity other than binding is indicated, the term “specific for PPAR” indicates that a particular compound has greater biological activity associated with binding to a PPAR than to other biomolecules (e.g., at a level as indicated for binding specificity). Similarly, the specificity can be for a specific PPAR with respect to other PPARs that may be present in or originally isolated from a particular organism.

Also in the context of compounds binding to a biomolecular target, the term “greater specificity” indicates that a compound binds to a specified target to a greater extent than to another biomolecule or biomolecules that may be present under relevant binding conditions, where binding to such other biomolecules produces a different biological activity than binding to the specified target. In some cases, the specificity is with reference to a limited set of other biomolecules, e.g., in the case of PPARs, in some cases the reference may be other receptors, or for a particular PPAR, it may be other PPARs. In some embodiments, the greater specificity is at least 2, 3, 4, 5, 8, 10, 50, 100, 200, 400, 500, or 1000-fold greater specificity. In the context of ligands interacting with PPARs, the terms “activity on”, “activity toward,” and like terms mean that such ligands have IC₅₀ EC₅₀ less than 10 μM, less than 1 μM, less than 100 nM, less than 50 nM, less than 20 nM, less than 10 nM, less than 5 nM, or less than 1 nM with respect to at least one PPAR as determined in a generally accepted PPAR activity assay.

The term “composition” or “pharmaceutical composition” refers to a formulation suitable for administration to an intended animal subject for therapeutic purposes. The formulation includes a therapeutically significant quantity (i.e. a therapeutically effective amount) of at least one active compound and at least one pharmaceutically acceptable carrier or excipient, which is prepared in a form adapted for administration to a subject. Thus, the preparation is “pharmaceutically acceptable”, indicating that it does not have properties that would cause a reasonably prudent medical practitioner to avoid administration of the material to a patient, taking into consideration the disease or conditions to be treated and the respective route of administration. In many cases, such a pharmaceutical composition is a sterile preparation, e.g. for injectibles.

The term “PPAR-mediated” disease or condition and like terms refer to a disease or condition in which the biological function of a PPAR affects the development and/or course of the disease or condition, and/or in which modulation of PPAR alters the development, course, and/or symptoms of the disease or condition. Similarly, the phrase “PPAR modulation provides a therapeutic benefit” indicates that modulation of the level of activity of PPAR in a subject indicates that such modulation reduces the severity and/or duration of the disease, reduces the likelihood or delays the onset of the disease or condition, and/or causes an improvement in one or more symptoms of the disease or condition. In some cases the disease or condition may be mediated by any one or more of the PPAR isoforms, e.g., PPARγ, PPARα, PPARδ, PPARγ and PPARα, PPARγ and PPARδ, PPARα and PPARδ, or PPARγ, PPARα, and PPARδ.

The term “therapeutically effective” or “effective amount” indicates that the materials or amount of material is effective to prevent, alleviate, or ameliorate one or more symptoms of a disease or medical condition, and/or to prolong the survival of the subject being treated.

The term “PPAR” refers to a peroxisome proliferator-activated receptor as recognized in the art. As indicated above, the PPAR family includes PPARα (also referred to as PPARa or PPARalpha), PPARδ (also referred to as PPARd or PPARdelta), and PPARγ (also referred to as PPARg or PPARgamma). The individual PPARs can be identified by their sequences, where exemplary reference sequence accession numbers are as follows:

| | | | |
|---|---|---|---|
| Receptor | Sequence | Accession No. | SEQ ID NO: |

| | | |
|---|---|---|
| hPPARa | cDNA | NM_005036 |
| hPPARa | protein | NP_005027 |
| hPPARg isoform 2 | cDNA | NM_015869 |
| hPPARg isoform 2 | protein | NP_056953 |
| hPPARd | cDNA | NM_006238 |
| hPPARd | protein | NP_006229 |

One of ordinary skill in the art will recognize that sequence differences will exist due to allelic variation, and will also recognize that other animals, particularly other mammals have corresponding PPARs, which have been identified or can be readily identified using sequence alignment and confirmation of activity. Such homologous PPARs can also be used in the present invention, which homologous PPARs have sequence identity of, for example, at least 50%, 60%, 70%, 80%, 90%, 95%, 99%, or even 100%, over a region spanning 50, 100, 150, 200, 250, 300, 350, 400, 450, 500, or even more amino acids or nucleotides for proteins or nucleic acids, respectively. One of ordinary skill in the art will also recognize that modifications can be introduced in a PPAR sequence without destroying PPAR activity. Such modified PPARs can also be used in the present invention, e.g., if the modifications do not alter the binding site conformation to the extent that the modified PPAR lacks substantially normal ligand binding.

As used herein in connection with the design or development of ligands, the term “bind” and “binding” and like terms refer to a non-convalent energetically favorable association between the specified molecules (i.e., the bound state has a lower free energy than the separated state, which can be measured calorimetrically). For binding to a target, the binding is at least selective, that is, the compound binds preferentially to a particular target or to members of a target family at a binding site, as compared to non-specific binding to unrelated proteins not having a similar binding site. For example, BSA is often used for evaluating or controlling for non-specific binding. In addition, for an association to be regarded as binding, the decrease in free energy going from a separated state to the bound state must be sufficient so that the association is detectable in a biochemical assay suitable for the molecules involved.

By “assaying” is meant the creation of experimental conditions and the gathering of data regarding a particular result of the experimental conditions. For example, enzymes can be assayed based on their ability to act upon a detectable substrate. Likewise, for example, a compound or ligand can be assayed based on its ability to bind to a particular target molecule or molecules and/or to modulate an activity of a target molecule.

By “background signal” in reference to a binding assay is meant the signal that is recorded under standard conditions for the particular assay in the absence of a test compound, molecular scaffold, or ligand that binds to the target molecule. Persons of ordinary skill in the art will realize that accepted methods exist and are widely available for determining background signal.

By “clog P” is meant the calculated log P of a compound, “P” referring to the partition coefficient of the compound between a lipophilic and an aqueous phase, usually between octanol and water.

In the context of compounds binding to a target, the term “greater affinity” indicates that the compound binds more tightly than a reference compound, or than the same compound in a reference condition, i.e., with a lower dissociation constant. In some embodiments, the greater affinity is at least 2, 3, 4, 5, 8, 10, 50, 100, 200, 400, 500, 1000, or 10,000-fold greater affinity.

By binding with “moderate affinity” is meant binding with a K_{D} of from about 200 nM to about 1 μM under standard conditions. By “moderately high affinity” is meant binding at a K_{D} of from about 1 nM to about 200 nM. By binding at “high affinity” is meant binding at a K_{D} of below about 1 nM under standard conditions. The standard conditions for binding are at pH 7.2 at 37° C. for one hour. For example, typical binding conditions in a volume of 100 μl/well would comprise a PPAR, a test compound, HEPES 50 mM buffer at pH 7.2, NaCl 15 mM, ATP 2 μM, and bovine serum albumin (1 ug/well), at 37° C. for one hour.

Binding compounds can also be characterized by their effect on the activity of the target molecule. Thus, a “low activity” compound has an inhibitory concentration (IC₅₀) (for inhibitors or antagonists) or effective concentration (EC₅₀) (applicable to agonists) of greater than 1 μM under standard conditions. By “moderate activity” is meant an IC₅₀ or EC₅₀ of 200 nM to 1 μM under standard conditions. By “moderately high activity” is meant an IC₅₀ or EC₅₀ of 1 nM to 200 nM. By “high activity” is meant an IC₅₀ or EC₅₀ of below 1 nM under standard conditions. The IC₅₀ (or EC₅₀) is defined as the concentration of compound at which 50% of the activity of the target molecule (e.g., enzyme or other protein) activity being measured is lost (or gained) relative to activity when no compound is present. Activity can be measured using methods known to those of ordinary skill in the art, e.g., by measuring any detectable product or signal produced by occurrence of an enzymatic reaction, or other activity by a protein being measured. For PPAR agonists, activities can be determined as described in the Examples, or using other such assay methods known in the art.

By “protein” is meant a polymer of amino acids. The amino acids can be naturally or non-naturally occurring. Proteins can also contain modifications, such as being glycosylated, phosphorylated, or other common modifications.

By “protein family” is meant a classification of proteins based on structural and/or functional similarities. For example, kinases, phosphatases, proteases, and similar groupings of proteins are protein families. Proteins can be grouped into a protein family based on having one or more protein folds in common, a substantial similarity in shape among folds of the proteins, homology, or based on having a common function. In many cases, smaller families will be specified, e.g., the PPAR family.

By “specific biochemical effect” is meant a therapeutically significant biochemical change in a biological system causing a detectable result. This specific biochemical effect can be, for example, the inhibition or activation of an enzyme, the inhibition or activation of a protein that binds to a desired target, or similar types of changes in the body's biochemistry. The specific biochemical effect can cause alleviation of symptoms of a disease or condition or another desirable effect. The detectable result can also be detected through an intermediate step.

By “standard conditions” is meant conditions under which an assay is performed to obtain scientifically meaningful data. Standard conditions are dependent on the particular assay, and can be generally subjective. Normally the standard conditions of an assay will be those conditions that are optimal for obtaining useful data from the particular assay. The standard conditions will generally minimize background signal and maximize the signal sought to be detected.

By “standard deviation” is meant the square root of the variance. The variance is a measure of how spread out a distribution is. It is computed as the average squared deviation of each number from its mean. For example, for the numbers 1, 2, and 3, the mean is 2 and the variance is:

$${σ}^{2} = \frac{{( 1 - 2 )}^{2} + {( 2 - 2 )}^{2} + {( 3 - 2 )}^{2}}{3} = 0.667 .$$

In the context of this invention, by “target molecule” is meant a molecule that a compound, molecular scaffold, or ligand is being assayed for binding to. The target molecule has an activity that binding of the molecular scaffold or ligand to the target molecule will alter or change. The binding of the compound, scaffold, or ligand to the target molecule can preferably cause a specific biochemical effect when it occurs in a biological system. A “biological system” includes, but is not limited to, a living system such as a human, animal, plant, or insect. In most but not all cases, the target molecule will be a protein or nucleic acid molecule.

By “pharmacophore” is meant a representation of molecular features that are considered to be responsible for a desired activity, such as interacting or binding with a receptor. A pharmacophore can include 3-dimensional (hydrophobic groups, charged/ionizable groups, hydrogen bond donors/acceptors), 2D (substructures), and 1D (physical or biological) properties.

As used herein in connection with numerical values, the terms “approximately” and “about” mean ±10% of the indicated value.

I. Applications of PPAR Agonists

The PPARs have been recognized as suitable targets for a number of different diseases and conditions. Some of those applications are described briefly below. Additional applications are known and the present compounds can also be used for those diseases and conditions.

(a) Insulin Resistance and Diabetes

In connection with insulin resistance and diabetes, PPARγ is necessary and sufficient for the differentiation of adipocytes in vitro and in vivo. In adipocytes, PPARγ increases the expression of numerous genes involved in lipid metabolism and lipid uptake. In contrast, PPARγ down-regulates leptin, a secreted, adipocyte-selective protein that has been shown to inhibit feeding and augment catabolic lipid metabolism. This receptor activity could explain the increased caloric uptake and storage noted in vivo upon treatment with PPARγ agonists. Clinically, TZDs, including troglitazone, rosiglitazone, and pioglitazone, and non-TZDs, including farglitazar, have insulin-sensitizing and antidiabetic activity. (Berger et al., 2002, Diabetes Tech. And Ther. 4:163-174.)

PPARγ has been associated with several genes that affect insulin action. TNFα, a proinflammatory cytokine that is expressed by adipocytes, has been associated with insulin resistance. PPARγ agonists inhibit expression of TNFα in adipose tissue of obese rodents, and ablate the actions of TNFα in adipocytes in vitro. PPARγ agonists were shown to inhibit expression of 11β-hydroxysteroid dehydrogenase 1 (11β-HSD-1), the enzyme that converts cortisone to the glucocorticoid agonist cortisol, in adipocytes and adipose tissue of type 2 diabetes mouse models. This is noteworthy since hypercortico-steroidism exacerbates insulin resistance. Adipocyte Complement-Related Protein of 30 kDa (Acrp30 or adiponectin) is a secreted adipocyte-specific protein that decreases glucose, triglycerides, and free fatty acids. In comparison to normal human subjects, patients with type 2 diabetes have reduced plasma levels of Acrp30. Treatment of diabetic mice and nondiabetic human subjects with PPARγ agonists increases plasma levels of Acrp30. Induction of Acrp30 by PPARγ agonists might therefore also play a key role in the insulin-sensitizing mechanism of PPARγ agonists in diabetes. (Berger et al., 2002, Diabetes Tech. And Ther. 4:163-174.)

PPARγ is expressed predominantly in adipose tissue. Thus, it is believed that the net in vivo efficacy of PPARγ agonists involves direct actions on adipose cells with secondary effects in key insulin responsive tissues such as skeletal muscle and liver. This is supported by the lack of glucose-lowering efficacy of rosiglitazone in a mouse model of severe insulin resistance where white adipose tissue was essentially absent. Furthermore, in vivo treatment of insulin resistant rats produces acute (<24 h) normalization of adipose tissue insulin action whereas insulin-mediated glucose uptake in muscle was not improved until several days after the initiation of therapy. This is consistent with the fact that PPARγ agonists can produce an increase in adipose tissue insulin action after direct in vitro incubation, whereas no such effect could be demonstrated using isolated in vitro incubated skeletal muscles. The beneficial metabolic effects of PPARγ agonists on muscle and liver may be mediated by their ability to (a) enhance insulin-mediated adipose tissue uptake, storage (and potentially catabolism) of free fatty acids; (b) induce the production of adipose-derived factors with potential insulin sensitizing activity (e.g., Acrp30); and/or (c) suppress the circulating levels and/or actions of insulin resistance-causing adipose-derived factors such as TNFα or resistin. (Berger et al., 2002, Diabetes Tech. And Ther. 4:163-174.)

(b) Dyslipidemia and Atherosclerosis

In connection with dyslipidemia and atherosclerosis, PPARα has been shown to play a critical role in the regulation of cellular uptake, activation, and β-oxidation of fatty acids. Activation of PPARα induces expression of fatty acid transport proteins and enzymes in the peroxisomal β-oxidation pathway. Several mitochondrial enzymes involved in the energy-harvesting catabolism of fatty acids are robustly upregulated by PPARα agonists. Peroxisome proliferators also activate expression of the CYP4As, a subclass of cytochrome P450 enzymes that catalyze the ω-hydroxylation of fatty acids, a pathway that is particularly active in the fasted and diabetic states. In sum, it is clear that PPARα is an important lipid sensor and regulator of cellular energy-harvesting metabolism. (Berger et al., 2002, Diabetes Tech. And Ther. 4:163-174.)

Atherosclerosis is a very prevalent disease in Westernized societies. In addition to a strong association with elevated LDL cholesterol, “dyslipidemia” characterized by elevated triglyceride-rich particles and low levels of HDL cholesterol is commonly associated with other aspects of a metabolic syndrome that includes obesity, insulin resistance, type 2 diabetes, and an increased risk of coronary artery disease. Thus, in 8,500 men with known coronary artery disease, 38% were found to have low HDL (<35 mg/dL) and 33% had elevated triglycerides (>200 mg/dL). In such patients, treatment with fibrates resulted in substantial triglyceride lowering and modest HDL-raising efficacy. More importantly, a recent large prospective trial showed that treatment with gemfibrozil produced a 22% reduction in cardiovascular events or death. Thus PPARα agonists can effectively improve cardiovascular risk factors and have a net benefit to improve cardiovascular outcomes. In fact, fenofibrate was recently approved in the United States for treatment of type IIA and IIB hyper-lipidemia. Mechanisms by which PPARα activation cause triglyceride lowering are likely to include the effects of agonists to suppress hepatic apo-CIII gene expression while also stimulating lipoprotein lipase gene expression. Dual PPARγ/α agonists, including KRP-297 and DRF 2725, possess potent lipid-altering efficacy in addition to antihyperglycemic activity in animal models of diabetes and lipid disorders.

The presence of PPARα and/or PPARγ expression in vascular cell types, including macrophages, endothelial cells, and vascular smooth muscle cells, suggests that direct vascular effects might contribute to potential antiatherosclerosis efficacy. PPARα and PPARα activation have been shown to inhibit cytokine-induced vascular cell adhesion and to suppress monocyte-macrophage migration. Several additional studies have also shown that PPARγ-selective compounds have the capacity to reduce arterial lesion size and attenuate monocyte-macrophage homing to arterial lesions in animal models of atherosclerosis. PPARγ is present in macrophages in human atherosclerotic lesions, and may play a role in regulation of expression of matrix metalloproteinase-9 (MMP-9), which is implicated in atherosclerotic plaque rupture (Marx et al., Am J Pathol. 1998, 153(1):17-23). Downregulation of LPS induced secretion of MMP-9 was also observed for both PPARα and PPARγ agonists, which may account for beneficial effects observed with PPAR agonists in animal models of atherosclerosis (Shu et al., Biochem Biophys Res Commun. 2000, 267(1):345-9). PPARγ is also shown to have a role in intercellular adhesion molecule-1 (ICAM-1) protein expression (Chen et al., Biochem Biophys Res Commun. 2001, 282(3):717-22) and vascular cell adhesion molecule-1 (VCAM-1) protein expression (Jackson et al., Arterioscler Thromb Vasc Biol. 1999, 19(9):2094-104) in endothelial cells, both of which play a role in the adhesion of monocytes to endothelial cells. In addition, two recent studies have suggested that either PPARα or PPARγ activation in macrophages can induce the expression of a cholesterol efflux “pump” protein.

It has been found that relatively selective PPARδ agonists produce minimal, if any, glucose- or triglyceride-lowering activity in murine models of type 2 diabetes in comparison with efficacious PPARγ or PPARα agonists. Subsequently, a modest increase in HDL-cholesterol levels was detected with PPARδ agonists in db/db mice. Recently, Oliver et al. (supra) reported that a potent, selective PPARδ agonist could induce a substantial increase in HDL-cholesterol levels while reducing triglyceride levels and insulin resistance in obese rhesus monkeys.

Thus, via multifactorial mechanisms that include improvements in circulating lipids, systemic and local antiinflammatory effects, and, inhibition of vascular cell proliferation, PPARα, PPARγ, and PPARδ agonists can be used in the treatment or prevention of atherosclerosis (Berger et al., supra).

(c) Inflammation

Monocytes and macrophages are known to play an important part in the inflammatory process through the release of inflammatory cytokines and the production of nitric oxide by inducible nitric oxide synthase. Rosiglitazone has been shown to induce apoptosis of macrophages at concentrations that parallel its affinity for PPARγ. This ligand has also been shown to block inflammatory cytokine synthesis in colonic cell lines. This latter observation suggests a mechanistic explanation for the observed anti-inflammatory actions of TZDs in rodent models of colitis.

Additional studies have examined the relationship between macrophages, cytokines and PPARγ and agonists thereof (Jiang et al., Nature 1998, 391(6662):82-6., Ricote et al., Nature 1998, 391(6662):79-82, Hortelano et al., J Immunol. 2000, 165(11):6525-31, and Chawla et al., Nat Med. 2001, 7(1):48-52) suggesting a role for PPARγ agonists in treating inflammatory responses, for example in autoimmune diseases.

The migration of monocytes and macrophages plays a role in the development of inflammatory responses as well. PPAR ligands have been shown to have an effect on a variety of chemokines. Monocyte chemotactic protein-1 (MCP-1) directed migration of monocytes is attenuated by PPARγ and PPARα ligands in a monocytic leukemia cell line (Kintscher et al., Eur J Pharmacol. 2000, 401(3):259-70). MCP-1 gene expression was shown to be suppressed by PPARγ ligand 15-deoxy-Delta(12,14)PGJ2 (15d-PGJ2) in two monocytic cell lines, which also showed induction of IL-8 gene expression ( Zhang et al., J Immunol. 2001, 166(12):7104-11).

Anti-inflammatory actions have been described for PPARα ligands that can be important in the maintenance of vascular health. Treatment of cytokine-activated human macrophages with PPARα agonists induced apoptosis of the cells. It was reported that PPARα agonists inhibit activation of aortic smooth muscle cells in response to inflammatory stimuli. (Staels et al., 1998, Nature 393:790-793.) In hyperlipidemic patients, fenofibrate treatment decreases the plasma concentrations of the inflammatory cytokine interleukin-6.

Anti-inflammatory pathways in airway smooth muscle cells were investigated with respect to PPARα and PPARγ (Patel et al., 2003, The Journal of Immunology, 170:2663-2669). This study demonstrated and anti-inflammatory effect of a PPARγ ligand that may be useful in the treatment of COPD and steroid-insensitive asthma.

The anti-inflammatory effects of PPAR modulators have also been studied with respect to autoimmune diseases, such as chronic inflammatory bowel syndrome, arthritis, Crohn's disease and multiple sclerosis, and in neuronal diseases such as Alzheimer's disease and Parkinson's disease.

(d) Hypertension

Hypertension is a complex disorder of the cardiovascular system that has been shown to be associated with insulin resistance. Type 2 diabetes patients demonstrate a 1.5-2-fold increase in hypertension in comparison with the general population. Troglitazone, rosiglitazone, and pioglitazone therapy have been shown to decrease blood pressure in diabetic patients as well as troglitazone therapy in obese, insulin-resistant subjects. Since such reductions in blood pressure were shown to correlate with decreases in insulin levels, they can be mediated by an improvement in insulin sensitivity. However, since TZDs also lowered blood pressure in one-kidney one-clip Sprague Dawley rats, which are not insulin resistant, it was proposed that the hypotensive action of PPARγ agonists is not exerted solely through their ability to improve insulin sensitivity. Other mechanisms that have been invoked to explain the antihypertensive effects of PPARγ agonists include their ability to (a) downregulate expression of peptides that control vascular tone such as PAI-I, endothelin, and type-c natriuretic peptide C or (b) alter calcium concentrations and the calcium sensitivity of vascular cells (Berger et al., supra).

(e) Cancer

PPAR modulation has also been correlated with cancer treatment. (Burstein et al.; Breast Cancer Res. Treat. 2003 79(3):391-7; Alderd et al.; Oncogene, 2003, 22(22):3412-6).

(f) Weight Control

Administration of PPARα agonists can induce satiety, and thus are useful in weight loss or maintenance. Such PPARα agonists can act preferentially on PPARα, or can also act on another PPAR, or can be PPAR pan-agonists. Thus, the satiety inducing effect of PPARα agonists can be used for weight control or loss.

(g) Autoimmune Diseases

PPAR agonists may provide benefits in the treatment of autoimmune diseases. Agonists of PPAR isoforms may be involved in T cell and B cell trafficking or activity, the altering of oligodendrocyte function or differentiation, the inhibition of macrophage activity, the reduction of inflammatory responses, and neuroprotective effects, some or all of which may be important in a variety of autoimmune diseases.

Multiple sclerosis (MS) is a neurodegenerative autoimmune disease that involves the demyelination of axons and formation of plaques. PPARδ mRNA has been shown to be strongly expressed in immature oligodendrocytes (Granneman et al., J Neurosci Res. 1998, 51(5):563-73). PPARδ selective agonists or pan- agonists were shown to accelerate differentiation of oligodendrocytes, with no effect on differentiation observed with a PPARγ selective agonist. An alteration in the myelination of corpus callosum was observed in PPARδ null mice (Peters et al., Mol Cell Biol. 2000, 20(14):5119-28). It was also shown that PPARδ mRNA and protein is expressed throughout the brain in neurons and oligodendrocytes, but not in astrocytes (Woods et al., Brain Res. 2003, 975(1-2):10-21). These observations suggest that PPARδ has a role in myelination, where modulation of such a role could be used to treat multiple sclerosis by altering the differentiation of oligodendrocytes, which may result in slowing of the demyelination, or even promoting the remyelination of axons. It has also been shown that oligodendrocyte-like B12 cells, as well as isolated spinal cord oligodendrocytes from rat, are affected by PPARγ agonists. Alkyl-dihydroxyacetone phosphate synthase, a key peroxisomal enzyme involved in the synthesis of plasmologens, which are a key component of myelin, is increased in PPARγ agonist treated B12 cells, while the number of mature cells in isolated spinal cord oligodendrocytes increases with PPARγ agonist treatment.

The role of PPAR in the regulation of B and T cells may also provide therapeutic benefits in diseases such as MS. For example, it has been shown that PPARγ agonists can inhibit the secretion of IL-2 by T cells (Clark et al., J Immunol. 2000, 164(3): 1364-71) or may induce apoptosis in T cells (Harris et al., Eur J Immunol. 2001, 31(4):1098-105), suggesting an important role in cell-mediated immune responses. An antiproliferative and cytotoxic effect on B cells by PPARγ agonists has also been observed (Padilla et al., Clin Immunol. 2002, 103(1):22-33).

The anti-inflammatory effects of PPAR modulators, as discussed herein, may also be useful in treating MS, as well as a variety of other autoimmune diseases such as Type-1 diabetes mellitus, psoriasis, vitiligo, uveitis, Sjogren's disease, pemphigus foliaceus, inclusion body myositis, polymyositis, dermatomyositis, scleroderma, Grave's disease, Hashimoto's disease, chronic graft-versus host disease, rheumatoid arthritis, inflammatory bowel syndrome, and Crohn's disease. Using a mouse model, the PPARα agonists gemfibrozil and fenofibrate were shown to inhibit clinical signs of experimental autoimmune encephalomyelitis, suggesting that PPARα agonists may be useful in treating inflammatory conditions such as multiple sclerosis (Lovett-Racke et al., J Immunol. 2004, 172(9):5790-8).

Neuroprotective effects that appear to be associated with PPARs may also aid in the treatment of MS. The effects of PPAR agonists on LPS induced neuronal cell death were studied using cortical neuron-glial co-cultures. PPARγ agonists 15d-PGJ2, ciglitazone and troglitazone were shown to prevent the LPS-induced neuronal cell death, as well as abolish NO and PGE2 release and a reduction in iNOS and COX-2 expression (Kim et al., Brain Res. 2002, 941(1-2):1-10).

Rheumatoid arthritis (RA) is an autoimmune inflammatory disease that results in the destruction of joints. In addition to chronic inflammation and joint damage due in part to mediators such as IL-6 and TNF-alpha, osteoclast differentiation is also implicated in damage to the joints. PPAR agonists may regulate these pathways, providing therapeutic benefits in treatment of RA. In studies using PPARγ agonist troglitazone in fibroblast-like synovial cells (FLS) isolated from patients with rheumatoid arthritis, an inhibition of cytokine mediated inflammatory responses was observed (Yamasaki et al., Clin Exp Immunol., 2002, 129(2):379-84). PPARγ agonists have also demonstrated beneficial effects in a rat or mouse model of RA (Kawahito et al., J Clin Invest. 2000, 106(2): 189-97; Cuzzocrea et al., Arthritis Rheum. 2003, 48(12):3544-56). The effects of the PPARα ligand fenofibrate on rheumatoid synovial fibroblasts from RA patients also showed inhibition of cytokine production, as well as NF-KappaB activation and osteoclast differentiation. Fenofibrate was also shown to inhibit the development of arthritis in a rat model (Okamoto et al., Clin Exp Rheumatol. 2005, 23(3):323-30).

Psoriasis is a T cell mediated autoimmune disease, where T cell activation leads to release of cytokines and resulting proliferation of keratinocytes. In addition to anti-inflammatory effects, the differentiation of keratinocytes may also be a therapeutic target for PPAR agonists. Studies in a PPARδ null mouse model suggest using PPARδ ligand to selectively induce keratinocyte differentiation and inhibit cell proliferation (Kim et al., Cell Death Differ. 2005). Thiazolidinedione ligands of PPARγ have been shown to inhibit the proliferation of psoriatic keratinocytes in monolayer and organ culture, and when applied topically inhibit epidermal hyperplasia of human psoriatic skin transplanted to SCID mice (Bhagavathula et al., J Pharmacol Exp Ther. 2005,315(3):996-1004).

(h) Neurodegenerative Diseases:

The modulation of the PPARs may provide benefits in the treatment of neuronal diseases. For example, the anti-inflammatory effects of PPAR modulators discussed herein have also been studied with respect to neuronal diseases such as Alzheimer's disease and Parkinson's disease.

In addition to inflammatory processes, Alzheimer's disease is characterized by deposits of amyloid-beta (Abeta) peptides and neurofibrillary tangles. A decrease in the levels of Abeta peptide in neuronal and non-neuronal cells was observed with induced expression of PPARγ, or by activation of PPARγ using a thiazolidinedione (Camacho et al., J Neurosci. 2004, 24(48):10908-17). Treatment of APP7171 mice with PPARγ agonist pioglitazone showed several beneficial effects, including reduction in activated microglia and reactive astrocytes in the hippocampus and cortex, reduction in proinflammatory cyclooxygenase 2 and inducible nitric oxide synthase, decreased β-secretase-1 mRNA and protein levels, and a reduction in the levels of soluble Abetal -42 peptide (Heneka et al., Brain. 2005, 128(Pt 6):1442-53).

Regions of degeneration of dopamine neurons in Parkinson's disease have been associated with increased levels of inflammatory cytokines (Nagatsu et al., J Neural Transm Suppl. 2000;(60):277-90). The effect of PPARγ agonist pioglitazone on dopaminergic nerve cell death and glial activation was studied in an MPTP mouse model of Parkinson's disease, wherein orally administered pioglitazone resulted in reduced glial activation as well as prevention of dopaminergic cell loss (Breidert et al. Journal of Neurochemistry, 2002, 82: 615).

(i) Other indications

PPARγ modulators have shown inhibition of VEGF-induced choroidal angiogenesis as well as repression of choroidal neovascularization effects, suggesting potential for treatment of retinal disorders. PPARδ has been shown to be expressed in implantation asites and in decidual cells in rats, suggesting a role in pregnancy, such as to enhance fertility. These studies were reviewed in Kota et al., 2005, Pharmacological Research 51: 85-94.

The management of pain, either neuropathic or inflammatory, is also suggested as a possible target for PPAR modulators. Burstein, S., Life Sci. 2005, 77(14):1674-84, suggests that PPARγ provides a receptor function for the activity of some cannabinoids. Lo Verme et al., Mol Pharmacol. 2005, 67(1):15-9, identifies PPARα as a target responsible for pain and inflammation reducing effects of palmitoylethanolamide (PEA). PEA selectively activates PPARα in vitro, and induces expression of PPARα mRNA when applied topically to mice. In animal models of carrageenan-induced paw edema and phorbol ester-induced ear edema, inflammation in wild type mice is attenuated by PEA, which has no effect in PPARα deficient mice. PPARα agonists OEA, GW7647 and Wy-14643 demonstrate similar effects. Benani et al., Neurosci Lett. 2004, 369(1):59-63, uses a model of inflammation in rats to assess the PPAR response in the rat spinal cord following injection of complete Freund's adjuvant into the hind paw. It was shown that PPARα was activated, suggesting a role in pain pathways.

PPARs are also involved in some infections, and may be targeted in treating such infections. Dharancy et al. report that HCV infection is related to altered expression and function of the anti-inflammatory nuclear receptor PPARalpha, and identify hepatic PPARalpha as one mechanism underlying the pathogenesis of HCV infection, and as a new therapeutic target in traditional treatment of HCV-induced liver injury (Dharancy et al., Gastroenterology 2005, 128(2):334-42). J Raulin reports that among other effects, HIV infection induces alteration of cellular lipids, including deregulation of PPARγ (J. Raulin, Prog Lipid Res 2002, 41(1):27-65). Slomiany and Slomiany report that PPARgamma activation leading to the impedance of Helicobacter pylori lipopolysaccharide (LPS) inhibitory effect on salivary mucin synthesis requires epidermal growth factor receptor (EGFR) participation. Further, they showed the impedance by ciglitazone was blunted in a concentration dependent fashion by a PPAR gamma agonist. (Slomiany and Slomiany, Inflammopharmacology 2004, 12(2):177-88).

Muto et al. (Human Molecular Genetics 2002, 11(15):1731-1742) showed that molecular defects observed in Pkd1⁻¹⁻ embryos contribute to the pathogenesis of autosomal dominant polycystic kidney disease (ADPKD)and that thiazolidindiones have a compensatory effect on the pathway affected by the loss of polycystin-1. Thus pathways activated by thiazolidinediones may provide new therapeutic targets in ADPKD (Muto et al., supra). Glintborg et al. show an increase in growth hormone levels in subjects with polycystic ovary syndrome treated with pioglitazone (Glintborg et al., J Clin Endocrinol Metab 2005, 90(10):5605-12).

In accordance with the description above, isoforms of the PPAR family of nuclear receptors are clearly involved in the systemic regulation of lipid metabolism and serve as “sensors” for fatty acids, prostanoid metabolites, eicosanoids and related molecules. These receptors function to regulate a broad array of genes in a coordinate fashion. Important biochemical pathways that regulate insulin action, lipid oxidation, lipid synthesis, adipocyte differentiation, peroxisome function, cell apoptosis, and inflammation can be modulated through the individual PPAR isoforms. Strong therapeutic effects of PPARα and PPARγ agonists to favorably influence systemic lipid levels, glucose homeostasis, and atherosclerosis risk (in the case of PPARα activation in humans) have recently been discovered. PPARα and PPARγ agonists are presently used clinically to favorably alter systemic lipid levels and glucose homeostasis, respectively. Recent observations made using PPARS ligands suggest that this isoform is also an important therapeutic target for dyslipidemia and insulin resistance, as well.

Thus, PPAR modulators, such as those described herein, can be used in the prophylaxis and/or therapeutic treatment of a variety of different disease and conditions, such as weight disorders (e.g. obesity, overweight condition, bulimia, and anorexia nervosa), lipid disorders (e.g. hyperlipidemia, dyslipidemia including associated diabetic dyslipidemia and mixed dyslipidemia hypoalphalipoproteinemia, hypertriglyceridemia, hypercholesterolemia, and low HDL (high density lipoprotein)), metabolic disorders (e.g. Metabolic Syndrome, Type II diabetes mellitus, Type I diabetes, hyperinsulinemia, impaired glucose tolerance, insulin resistance, diabetic complication including neuropathy, nephropathy, retinopathy, diabetic foot ulcer and cataracts), cardiovascular disease (e.g. hypertension, coronary heart disease, heart failure, congestive heart failure, atherosclerosis, arteriosclerosis, stroke, cerebrovascular disease, myocardial infarction, peripheral vascular disease), inflanmmatory diseases (e.g. autoimmune diseases such as vitiligo, uveitis, pemphigus foliaceus, inclusion body myositis, polymyositis, dermatomyositis, scleroderma, Grave's disease, Hashimoto's disease, chronic graft versus host disease, rheumatoid arthritis, inflammatory bowel syndrome, Crohn's disease, systemic lupus erythematosis, Sjogren's Syndrome, and multiple sclerosis, diseases involving airway inflammation such as asthma and chronic obstructive pulmonary disease, and inflammation in other organs, such as polycystic kidney disease (PKD), polycystic ovary syndrome, pancreatitis, nephritis, and hepatitis), skin disorders (e.g. epithelial hyperproliferative diseases such as eczema and psoriasis, dermatitis, including atopic dermatitis, contact dermatitis, allergic dermatitis and chronic dermatitis, and impaired wound healing), neurodegenerative disorders (e.g. Alzheimer's disease, Parkinson's disease, amyotrophic lateral sclerosis, spinal cord injury, and demyelinating disease, including acute disseminated encephalomyelitis and Guillain-Barre syndrome), coagulation disorders (e.g. thrombosis), gastrointestinal disorders (e.g. infarction of the large or small intestine), genitourinary disorders (e.g. renal insufficiency, erectile dysfunction, urinary incontinence, and neurogenic bladder), ophthalmic disorders (e.g. ophthalmic inflammation, macular degeneration, and pathologic neovascularization), infections (e.g. HCV, HIV, and Helicobacter pylori), neuropathic or inflammatory pain, infertility, and cancer.

II. PPAR Active Compounds

As indicated in the Summary and in connection with applicable diseases and conditions, a number of different PPAR agonists have been identified. In addition, the present invention provides PPAR agonist compounds described by Formulae I, Ia, lb, II, or III as provided in the Summary above. Included within Formula I are sub-groups and compounds described in U.S. patent application Ser. No. 10/937,791, the disclosure of which is hereby incorporated by reference herein in its entirety. These compounds can be used in the treatment or prophylaxis of a disease or condition selected from the group consisting of inflammatory diseases (e.g. autoimmune diseases such as vitiligo, uveitis, pemphigus foliaceus, inclusion body myositis, polymyositis, dermatomyositis, scleroderma, Grave's disease, Hashimoto's disease, chronic graft versus host disease, rheumatoid arthritis, inflammatory bowel syndrome, Crohn's disease, systemic lupus erythematosis, Sjogren's Syndrome, and multiple sclerosis, diseases involving airway inflammation such as asthma and chronic obstructive pulmonary disease, and inflammation in other organs, such as polycystic kidney disease (PKD), polycystic ovary syndrome, pancreatitis, nephritis, and hepatitis), skin disorders (e.g. dermatitis, including atopic dermatitis, contact dermatitis, allergic dermatitis and chronic dermatitis, and impaired wound healing), neurodegenerative disorders (e.g. Alzheimer's disease, Parkinson's disease, amyotrophic lateral sclerosis, spinal cord injury, and demyelinating disease, including acute disseminated encephalomyelitis and Guillain-Barre syndrome), gastrointestinal disorders (e.g. infarction of the large or small intestine), genitourinary disorders (e.g. renal insufficiency, erectile dysfunction, urinary incontinence, and neurogenic bladder), ophthalmic disorders (e.g. ophthalmic inflammation, macular degeneration, and pathologic neovascularization), infections (e.g. HCV, HIV, and Helicobacter pylori), neuropathic or inflammatory pain, and infertility, preferably neurodegenerative diseases, such as Alzheimer's disease, Parkinson's disease, and amyotrophic lateral sclerosis, autoimmune diseases such as rheumatoid arthritis, inflammatory bowel syndrome, Crohn's disease and multiple sclerosis, infertility, diseases involving airway smooth muscle cells such as asthma and chronic obstructive pulmonary disease, and angiogenesis related conditions, such as macular degeneration. Compounds of Formulae II or III may also be used in the treatment of these diseases, as well as in the treatment or prophylaxis of a disease or condition selected from the group consisting of weight disorders (e.g. obesity, overweight condition, bulimia, and anorexia nervosa), lipid disorders (e.g. hyperlipidemia, dyslipidemia including associated diabetic dyslipidemia and mixed dyslipidemia hypoalphalipoproteinemia, hypertriglyceridemia, hypercholesterolemia, and low HDL (high density lipoprotein)), metabolic disorders (e.g. Metabolic Syndrome, Type II diabetes mellitus, Type I diabetes, hyperinsulinemia, impaired glucose tolerance, insulin resistance, diabetic complication including neuropathy, nephropathy, retinopathy, diabetic foot ulcer and cataracts), cardiovascular disease (e.g. hypertension, coronary heart disease, heart failure, congestive heart failure, atherosclerosis, arteriosclerosis, stroke, cerebrovascular disease, myocardial infarction, peripheral vascular disease), skin disorders (e.g. epithelial hyperproliferative diseases such as eczema and psoriasis, coagulation disorders (e.g. thrombosis), and cancer. Exemplary compounds described by Formulae II and III are provided in the Examples below. Additional compounds within Formulae I, Ia, lb, II, or III can also be prepared and tested to confirm activity using conventional methods and the guidance provided herein.

The activity of the compounds can be assessed using methods known to those of skill in the art, as well as methods described herein. Screening assays may include controls for purposes of calibration and confirmation of proper manipulation of the components of the assay. Blank wells that contain all of the reactants but no member of the chemical library are usually included. As another example, a known inhibitor (or activator) of an enzyme for which modulators are sought, can be incubated with one sample of the assay, and the resulting decrease (or increase) in the enzyme activity used as a comparator or control. It will be appreciated that modulators can also be combined with the enzyme activators or inhibitors to find modulators which inhibit the enzyme activation or repression that is otherwise caused by the presence of the known the enzyme modulator. Similarly, when ligands to a target are sought, known ligands of the target can be present in control/calibration assay wells.

(a) Enzymatic Activity Assays

A number of different assays can be utilized to assess activity of PPAR modulators and/or determine specificity of a modulator for a particular PPAR. In addition to the assays mentioned in the Examples below, one of ordinary skill in the art will know of other assays that can be utilized and can modify an assay for a particular application. For example, the assay can utilize AlphaScreen (amplified luminescent proximity homogeneous assay) format, e.g., AlphaScreening system (Packard BioScience). AlphaScreen is generally described in Seethala and Prabhavathi, Homogenous Assays: AlphaScreen, Handbook of Drug Screeni, Marcel Dekkar Pub. 2001, pp. 106-110. Applications of the technique to PPAR receptor ligand binding assays are described, for example, in Xu, et al., Nature, 2002, 415:813-817.

(b) Assessment of efficacy of compounds in disease model systems.

The utility of compounds of Formula I for the treatment of diseases such as autoimmune diseases and neurological diseases can be readily assessed using model systems known to those of skill in the art. For example, efficacy of PPAR modulators in models of Alzheimer's disease can be tested by mimicking inflammatory injury to neuronal tissues and measuring recovery using molecular and pharmacological markers (Heneka, et al., J Neurosci.,2000, 20:6862-6867). Efficacy of PPAR modulators in multiple sclerosis has been monitored using the accepted model of experimental autoimmune encephalomyelitis (EAE) (Storer, et al., J Neuroimmunol., 2004, 161:113-122. See also: Niino, et al., J Neuroimmunol., 2001, 116:4048; Diab, et al. J Immunol., 2002, 168:2508-2515; Natarajan, et al., Genes Immun., 2002, 3, 59-70; Feinstein, et al., Ann. Neurol., 2002, 51:694-702.)

(c) Isomers, Prodrugs, and Active Metabolites

Compounds contemplated herein are described with reference to both generic formulae and specific compounds. In addition, the invention compounds may exist in a number of different forms or derivatives, all within the scope of the present invention. These include, for example, tautomers, stereoisomers, racemic mixtures, regioisomers, salts, prodrugs (e.g., carboxylic acid esters), solvated forms, different crystal forms or polymorphs, and active metabolites.

(d) Tautomers, Stereoisomers, Regioisomers, and Solvated Forms

It is understood that some compounds may exhibit tautomerism. In such cases, the formulae provided herein expressly depict only one of the possible tautomeric forms. It is therefore to be understood that the formulae provided herein are intended to represent any tautomeric form of the depicted compounds and are not to be limited merely to the specific tautomeric form depicted by the drawings of the formulae.

Likewise, some of the compounds according to the present invention may exist as stereoisomers, i.e. having the same atomic connectivity of covalently bonded atoms yet differing in the spatial orientation of the atoms. For example, compounds may be optical stereoisomers, which contain one or more chiral centers, and therefore, may exist in two or more stereoisomeric forms (e.g. enantiomers or diastereomers). Thus, such compounds may be present as single stereoisomers (i.e., essentially free of other stereoisomers), racemates, and/or mixtures of enantiomers and/or diastereomers. As another example, stereoisomers include geometric isomers, such as cis- or trans- orientation of substituents on adjacent carbons of a double bond. All such single stereoisomers, racemates and mixtures thereof are intended to be within the scope of the present invention. Unless specified to the contrary, all such steroisomeric forms are included within the formulae provided herein.

In some embodiments, a chiral compound of the present invention is in a form that contains at least 80% of a single isomer (60% enantiomeric excess (“e.e.”) or diastereomeric excess (“d.e.”)), or at least 85% (70% e.e. or d.e.), 90% (80% e.e. or d.e.), 95% (90% e.e. or d.e.), 97.5% (95% e.e. or d.e.), or 99% (98% e.e. or d.e.). As generally understood by those skilled in the art, an optically pure compound having one chiral center is one that consists essentially of one of the two possible enantiomers (i.e., is enantiomerically pure), and an optically pure compound having more than one chiral center is one that is both diastereomerically pure and enantiomerically pure. In some embodiments, the compound is present in optically pure form.

For compounds in which synthesis involves addition of a single group at a double bond, particularly a carbon-carbon double bond, the addition may occur at either of the double bond-linked atoms. For such compounds, the present invention includes both such regioisomers.

Additionally, the formulae are intended to cover solvated as well as unsolvated forms of the identified structures. For example, the indicated structures include both hydrated and non-hydrated forms. Other examples of solvates include the structures in combination with a suitable solvent, such as isopropanol, ethanol, methanol, DMSO, ethyl acetate, acetic acid, or ethanolamine.

(e) Prodrugs and Metabolites

In addition to the present formulae and compounds described herein, the invention also includes prodrugs (generally pharmaceutically acceptable prodrugs), active metabolic derivatives (active metabolites), and their pharmaceutically acceptable salts.

Prodrugs are compounds or pharmaceutically acceptable salts thereof which, when metabolized under physiological conditions or when converted by solvolysis, yield the desired active compound. Prodrugs include, without limitation, esters, amides, carbamates, carbonates, ureides, solvates, or hydrates of the active compound. Typically, the prodrug is inactive, or less active than the active compound, but may provide one or more advantageous handling, administration, and/or metabolic properties. For example, some prodrugs are esters of the active compound; during metabolysis, the ester group is cleaved to yield the active drug. Also, some prodrugs are activated enzymatically to yield the active compound, or a compound which, upon further chemical reaction, yields the active compound. In this context, a common example is an alkyl ester of a carboxylic acid.

As described in The Practice of Medicinal Chemistry, Ch. 31-32 (Ed. Wermuth, Academic Press, San Diego, Cailf., 2001), prodrugs can be conceptually divided into two non-exclusive categories, bioprecursor prodrugs and carrier prodrugs. Generally, bioprecursor prodrugs are compounds that are inactive or have low activity compared to the corresponding active drug compound, that contain one or more protective groups and are converted to an active form by metabolism or solvolysis. Both the active drug form and any released metabolic products should have acceptably low toxicity. Typically, the formation of active drug compound involves a metabolic process or reaction that is one of the follow types:

Oxidative reactions: Oxidative reactions are exemplified without limitation to reactions such as oxidation of alcohol, carbonyl, and acid functionalities, hydroxylation of aliphatic carbons, hydroxylation of alicyclic carbon atoms, oxidation of aromatic carbon atoms, oxidation of carbon-carbon double bonds, oxidation of nitrogen-containing functional groups, oxidation of silicon, phosphorus, arsenic, and sulfur, oxidative N-dealkylation, oxidative O-and S-dealkylation, oxidative deamination, as well as other oxidative reactions.

Reductive reactions: Reductive reactions are exemplified without limitation to reactions such as reduction of carbonyl functionalitites, reduction of alcohol functionalities and carbon-carbon double bonds, reduction of nitrogen-containing functional groups, and other reduction reactions.

Reactions without change in the oxidation state: Reactions without change in the state of oxidation are exemplified without limitation to reactions such as hydrolysis of esters and ethers, hydrolytic cleavage of carbon-nitrogen single bonds, hydrolytic cleavage of non-aromatic heterocycles, hydration and dehydration at multiple bonds, new atomic linkages resulting from dehydration reactions, hydrolytic dehalogenation, removal of hydrogen halide molecule, and other such reactions.

Carrier prodrugs are drug compounds that contain a transport moiety, e.g., that improves uptake and/or localized delivery to a site(s) of action. Desirably for such a carrier prodrug, the linkage between the drug moiety and the transport moiety is a covalent bond, the prodrug is inactive or less active than the drug compound, the prodrug and any release transport moiety are acceptably non-toxic. For prodrugs where the transport moiety is intended to enhance uptake, typically the release of the transport moiety should be rapid. In other cases, it is desirable to utilize a moiety that provides slow release, e.g., certain polymers or other moieties, such as cyclodextrins. (See, e.g., Cheng et al., U.S. Patent Publ. No. 20040077595, application Ser. No. 10/656,838, incorporated herein by reference.) Such carrier prodrugs are often advantageous for orally administered drugs. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g., stability, water solubility, suppression of an undesirable organoleptic or physiochemical property). For example, lipophilicity can be increased by esterification of hydroxyl groups with lipophilic carboxylic acids, or of carboxylic acid groups with alcohols, e.g., aliphatic alcohols. Wermuth, supra.

Prodrugs may proceed from prodrug form to active form in a single step or may have one or more intermediate forms which may themselves have activity or may be inactive.

Metabolites, e.g., active metabolites, overlap with prodrugs as described above, e.g., bioprecursor prodrugs. Thus, such metabolites are pharmacologically active compounds or compounds that further metabolize to pharmacologically active compounds that are derivatives resulting from metabolic processes in the body of a subject. Of these, active metabolites are such pharmacologically active derivative compounds. For prodrugs, the prodrug compound is generally inactive or of lower activity than the metabolic product. For active metabolites, the parent compound may be either an active compound or may be an inactive prodrug. Metabolites of a compound may be identified using routine techniques known in the art, and their activities determined using tests such as those described herein. For example, in some compounds, one or more alkoxy groups can be metabolized to hydroxyl groups while retaining pharmacologic activity and/or carboxyl groups can be esterified, e.g., glucuronidation. In some cases, there can be more than one metabolite, where an intermediate metabolite(s) is further metabolized to provide an active metabolite. For example, in some cases a derivative compound resulting from metabolic glucuronidation may be inactive or of low activity, and can be further metabolized to provide an active metabolite.

Prodrugs and active metabolites may be identified using routine techniques known in the art. See, e.g., Bertolini et al., 1997, J Med. Chem., 40:2011-2016; Shan et al., 1997, J Pharm Sci 86(7):756-757; Bagshawe, 1995, Drug Dev. Res., 34:220-230; Wermuth, supra.

(f) Pharmaceutically Acceptable Salts

Compounds can be formulated as or be in the form of pharmaceutically acceptable salts. Contemplated pharmaceutically acceptable salt forms include, without limitation, mono, bis, tris, tetrakis, and so on. Pharmaceutically acceptable salts are non-toxic in the amounts and concentrations at which they are administered. The preparation of such salts can facilitate the pharmacological use by altering the physical characteristics of a compound without preventing it from exerting its physiological effect. Useful alterations in physical properties include lowering the melting point to facilitate transmucosal administration and increasing the solubility to facilitate administering higher concentrations of the drug. A compound of the invention may possess a sufficiently acidic, a sufficiently basic, or both functional groups, and accordingly react with any of a number of inorganic or organic bases, and inorganic and organic acids, to form a pharmaceutically acceptable salt.

Pharmaceutically acceptable salts include acid addition salts such as those containing sulfate, pyrosulfate, bisulfate, sulfite, bisulfite, chloride, bromide, iodide, hydrochloride, fumarate, maleate, phosphate, monohydrogenphosphate, dihydrogenphosphate, metaphosphate, pyrophosphate, sulfamate, acetate, citrate, lactate, tartrate, sulfonate, methanesulfonate, propanesulfonate, ethanesulfonate, benzenesulfonate, p-toluenesulfonate, naphthalene- 1-sulfonate, naphthalene-2-sulfonate, xylenesulfonates, cyclohexylsulfamate, quinate, propionate, decanoate, caprylate, acrylate, formate, isobutyrate, caproate, heptanoate, propiolate, oxalate, malonate, succinate, suberate, sebacate, fumarate, maleate, butyne-1,4 dioate, hexyne-1,6-dioate, benzoate, chlorobenzoate, methylbenzoate, dinitrobenzoate, hydroxybenzoate, methoxybenzoate, phthalate, phenylacetate, phenylpropionate, phenylbutyrate, gamma-hydroxybutyrate, glycollate, and mandelate. Pharmaceutically acceptable salts can be obtained from acids such as hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid.

Pharmaceutically acceptable salts also include basic addition salts such as those containing benzathine, chloroprocaine, choline, diethanolamine, ethanolamine, t-butylamine, ethylenediamine, meglumine, procaine, aluminum, calcium, lithium, magnesium, potassium, sodium, ammonium, alkylamine, and zinc, when acidic functional groups, such as carboxylic acid or phenol are present. For example, see Remington's Pharmaceutical Sciences, 19th ed., Mack Publishing Co., Easton, Pa., Vol. 2, p. 1457, 1995. Such salts can be prepared using the appropriate corresponding bases.

Pharmaceutically acceptable salts can be prepared by standard techniques. For example, the free-base form of a compound can be dissolved in a suitable solvent, such as an aqueous or aqueous-alcohol solution containing the appropriate acid and then isolated by evaporating the solution. In another example, a salt can be prepared by reacting the free base and acid in an organic solvent.

Thus, for example, if the particular compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid, and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

Similarly, if the particular compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as L-glycine, L-lysine, and L-arginine, ammonia, primary, secondary, and tertiary amines, and cyclic amines, such as hydroxyethylpyrrolidine, piperidine, morpholine or piperazine, and inorganic salts derived from sodium, calcium, potassium, magnesium, manganese, iron, copper, zinc, aluminum and lithium.

The pharmaceutically acceptable salt of the different compounds may be present as a complex. Examples of complexes include 8-chlorotheophylline complex (analogous to, e.g., dimenhydrinate: diphenhydramine 8-chlorotheophylline (1:1) complex; Dramamine) and various cyclodextrin inclusion complexes.

Unless specified to the contrary, specification of a compound herein includes pharmaceutically acceptable salts of such compound.

(g) Polymorphic Forms

In the case of agents that are solids, it is understood by those skilled in the art that the compounds and salts may exist in different crystal or polymorphic forms, all of which are intended to be within the scope of the present invention and specified formulae.

III. Administration

The methods and compounds will typically be used in therapy for human subjects. However, they may also be used to treat similar or identical indications in other animal subjects. In this context, the terms “subject”, “animal subject”, and the like refer to human and non-human vertebrates, e.g., mammals such as non-human primates, sports and commercial animals, e.g., bovines, equines, porcines, ovines, rodents, and pets e.g., canines and felines.

Suitable dosage forms, in part, depend upon the use or the route of administration, for example, oral, transdermal, transmucosal, inhalant, or by injection (parenteral). Such dosage forms should allow the compound to reach target cells. Other factors are well known in the art, and include considerations such as toxicity and dosage forms that retard the compound or composition from exerting its effects. Techniques and formulations generally may be found in Remington: The Science and Practice of Pharmacy, 21^{st} edition, Lippincott, Williams and Wilkins, Philadelphia, Pa., 2005 (hereby incorporated by reference herein).

Compounds of the present invention (i.e. Formula I, including Formulae Ia-Im, and all sub-embodiments disclosed herein) can be formulated as pharmaceutically acceptable salts.

Carriers or excipients can be used to produce compositions. The carriers or excipients can be chosen to facilitate administration of the compound. Examples of carriers include calcium carbonate, calcium phosphate, various sugars such as lactose, glucose, or sucrose, or types of starch, cellulose derivatives, gelatin, vegetable oils, polyethylene glycols and physiologically compatible solvents. Examples of physiologically compatible solvents include sterile solutions of water for injection (WFI), saline solution, and dextrose.

The compounds can be administered by different routes including intravenous, intraperitoneal, subcutaneous, intramuscular, oral, transmucosal, rectal, transdermal, or inhalant. In some embodiments, oral administration is preferred. For oral administration, for example, the compounds can be formulated into conventional oral dosage forms such as capsules, tablets, and liquid preparations such as syrups, elixirs, and concentrated drops.

Pharmaceutical preparations for oral use can be obtained, for example, by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethylcellulose (CMC), and/or polyvinylpyrrolidone (PVP: povidone). If desired, disintegrating agents may be added, such as the cross-linked polyvinylpyrrolidone, agar, or alginic acid, or a salt thereof such as sodium alginate.

Dragee cores are provided with suitable coatings. For this purpose, concentrated sugar solutions may be used, which may optionally contain, for example, gum arabic, talc, poly-vinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin (“gelcaps”), as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

Alternatively, injection (parenteral administration) may be used, e.g., intramuscular, intravenous, intraperitoneal, and/or subcutaneous. For injection, the compounds of the invention are formulated in sterile liquid solutions, preferably in physiologically compatible buffers or solutions, such as saline solution, Hank's solution, or Ringer's solution. In addition, the compounds may be formulated in solid form and redissolved or suspended immediately prior to use. Lyophilized forms can also be produced.

Administration can also be by transmucosal, topical, transdermal, or inhalant means. For transmucosal, topical or transdermal administration, penetrants appropriate to the barrier to be permeated are used in the formulation. Such penetrants are generally known in the art, and include, for example, for transmucosal administration, bile salts and fusidic acid derivatives. In addition, detergents may be used to facilitate permeation. Transmucosal administration, for example, may be through nasal sprays or suppositories (rectal or vaginal).

The topical compositions of this invention are formulated preferably as oils, creams, lotions, ointments, and the like by choice of appropriate carriers known in the art. Suitable carriers include vegetable or mineral oils, white petrolatum (white soft paraffin), branched chain fats or oils, animal fats and high molecular weight alcohol (greater than C₁₂). The preferred carriers are those in which the active ingredient is soluble. Emulsifiers, stabilizers, humectants and antioxidants may also be included as well as agents imparting color or fragrance, if desired. Creams for topical application are preferably formulated from a mixture of mineral oil, self-emulsifying beeswax and water in which mixture the active ingredient, dissolved in a small amount solvent (e.g., an oil), is admixed. Additionally, administration by transdermal means may comprise a transdermal patch or dressing such as a bandage impregnated with an active ingredient and optionally one or more carriers or diluents known in the art. To be administered in the form of a transdermal delivery system, the dosage administration will, of course, be continuous rather than intermittent throughout the dosage regimen.

For inhalants, compounds of the invention may be formulated as dry powder or a suitable solution, suspension, or aerosol. Powders and solutions may be formulated with suitable additives known in the art. For example, powders may include a suitable powder base such as lactose or starch, and solutions may comprise propylene glycol, sterile water, ethanol, sodium chloride and other additives, such as acid, alkali and buffer salts. Such solutions or suspensions may be administered by inhaling via spray, pump, atomizer, or nebulizer, and the like. The compounds of the invention may also be used in combination with other inhaled therapies, for example corticosteroids such as fluticasone proprionate, beclomethasone dipropionate, triamcinolone acetonide, budesonide, and mometasone furoate; beta agonists such as albuterol, salmeterol, and formoterol; anticholinergic agents such as ipratroprium bromide or tiotropium; vasodilators such as treprostinal and iloprost; enzymes such as DNAase; therapeutic proteins; immunoglobulin antibodies; an oligonucleotide, such as single or double stranded DNA or RNA, siRNA; antibiotics such as tobramycin; muscarinic receptor antagonists; leukotriene antagonists; cytokine antagonists; protease inhibitors; cromolyn sodium; nedocril sodium; and sodium cromoglycate.

The amounts of various compounds to be administered can be determined by standard procedures taking into account factors such as the compound EC₅₀, the biological half-life of the compound, the age, size, and weight of the subject, and the disorder associated with the subject. The importance of these and other factors are well known to those of ordinary skill in the art. Generally, a dose will be between about 0.01 and 50 mg/kg, preferably 0.1 and 20 mg/kg of the subject being treated. Multiple doses may be used.

The compounds of the invention may also be used in combination with other therapies for treating the same disease. Such combination use includes administration of the compounds and one or more other therapeutics at different times, or co-administration of the compound and one or more other therapies. In some embodiments, dosage may be modified for one or more of the compounds of the invention or other therapeutics used in combination, e.g., reduction in the amount dosed relative to a compound or therapy used alone, by methods well known to those of ordinary skill in the art.

It is understood that use in combination includes use with other therapies, drugs, medical procedures etc., where the other therapy or procedure may be administered at different times (e.g. within a short time, such as within hours (e.g. 1, 2, 3, 4-24 hours), or within a longer time (e.g. 1-2 days, 2-4 days, 4-7 days, 1-4 weeks)) than a compound of the present invention, or at the same time as a compound of the invention. Use in combination also includes use with a therapy or medical procedure that is administered once or infrequently, such as surgery, along with a compound of the invention administered within a short time or longer time before or after the other therapy or procedure. In some embodiments, the present invention provides for delivery of compounds of the invention and one or more other drug therapeutics delivered by a different route of administration or by the same route of administration. The use in combination for any route of administration includes delivery of compounds of the invention and one or more other drug therapeutics delivered by the same route of administration together in any formulation, including formulations where the two compounds are chemically linked in such a way that they maintain their therapeutic activity when administered. In one aspect, the other drug therapy may be co-administered with one or more compounds of the invention. Use in combination by co-administration includes administration of co-formulations or formulations of chemically joined compounds, or administration of two or more compounds in separate formulations within a short time of each other (e.g. within an hour, 2 hours, 3 hours, up to 24 hours), administered by the same or different routes. Co-administration of separate formulations includes co-administration by delivery via one device, for example the same inhalant device, the same syringe, etc., or administration from separate devices within a short time of each other. Co-formulations of compounds of the invention and one or more additional drug therapies delivered by the same route includes preparation of the materials together such that they can be administered by one device, including the separate compounds combined in one formulation, or compounds that are modified such that they are chemically joined, yet still maintain their biological activity. Such chemically joined compounds may have a linkage that is substantially maintained in vivo, or the linkage may break down in vivo, separating the two active components.

IV. Synthesis of Compounds

Compounds with the chemical structure of Formulae I, Ia and Ib can be prepared for example, by the synthetic schemes described in U.S. patent application Ser. No. 10/937,791 (see also PCT publication WO 2005/009958). Compounds with the chemical structure of Formulae II and III can be prepared by a number of synthetic routes, including, for example, the synthetic schemes described herein. Additional synthetic routes can be utilized by one skilled in chemical synthesis.

One method to prepare the indole precursor with 3-propionic acid side chain (VII) involves the use of indole with Meldrum's acid to afford the propionic acid ester through a two step process in one pot as shown in Scheme I.

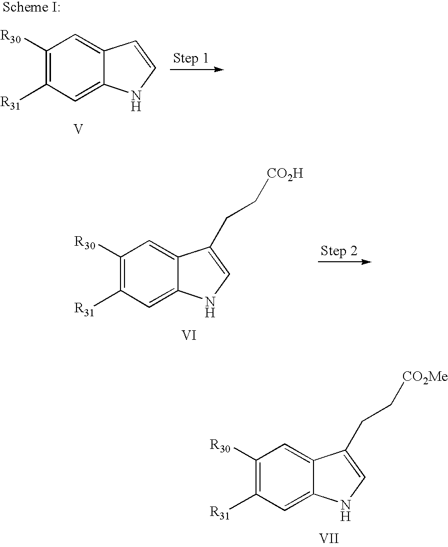

Step 1: Preparation of the Indole-3-Propionic Acid, VI

Into a microwave vessel, indole (1 equivalent), paraformaldehyde (1.1 equivalent), 2,2-dimethyl-1,3-dioxane-4,6-dione (1.1 equivalent), triethylamine (1.1 equivalent) are dissolved in acetonitrile (2 ml/mmol). The reaction is heated at 150° C. for 3 minutes in a microwave reactor. The reaction is then diluted with acidified water ( pH˜5 with acetic acid), and the aqueous layer was extracted with ethyl acetate. The organic layer is then washed with water, brine, and then dried over magnesium sulfate. Evaporation of solvent leads to a solid. The crude product is then purified via flash chromatography with step gradient of 2, 4, and 6% methanol in chloroform on silica to obtain the desired compound, VI, as an oil.

Step 2: Preparation of Compound VII.

Compound VI is stirred at ambient temperature with aqueous HCl (4M), with methanol and dioxane (1:1 equivalent) for 1 hour. The reaction mixture is then extracted with xylenes. The organic layer is evaporated, and compound VII is purified via flash chromatography on silica eluting with chloroform to obtain a solid.

The resulting propionic acid ester can be used to prepare the 1-sulfone substituted indole IX in two steps as shown in Scheme II.

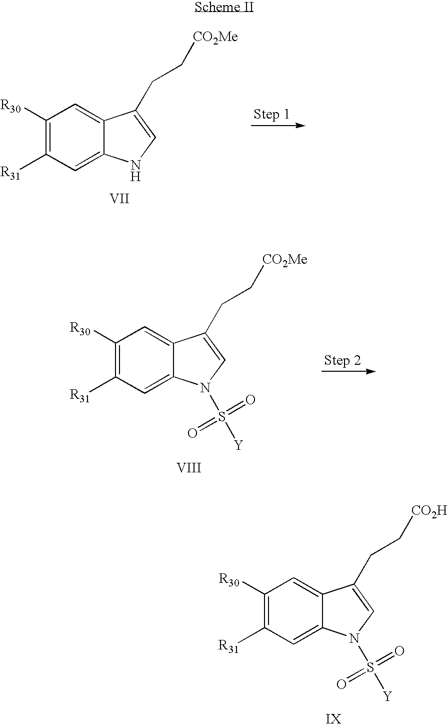

Step 1: Preparation of Compound VIII

Compound VII (1 mmol) in THF (5 ml), is combined with BEMP (1.1 mmol), and substituted sulfonyl chloride (1.05 mmol) and mixed at room temperature for 2 hours. The crude product VIII is supplied directly to the saponification step next.

Step 2: Preparation of Compound IX, Deprotection of the Methyl Ester.

Into a flask, the crude reactant VIII is dissolved in 1 M NaOH, and stirred for 4 hours at ambient temperature. The hydrolysis can be monitored via LC-MS. Upon full transformation, the basic solution is neutralized with acetic acid. Next, solvent is removed under reduced pressure to yield a crude solid. The crude material is then taken up in DMSO, and purified via reverse phase HPLC with a 20-100% Acetonitrile gradient (12 minute gradient). The purified material is then analyzed via HPLC to identify the pure fractions. The fractions are combined and concentrated to afford the desired compound IX as a solid.

Compounds having an optionally substitituted aryl sulfone on the indole nitrogen can be prepared in three steps as shown in Scheme III.

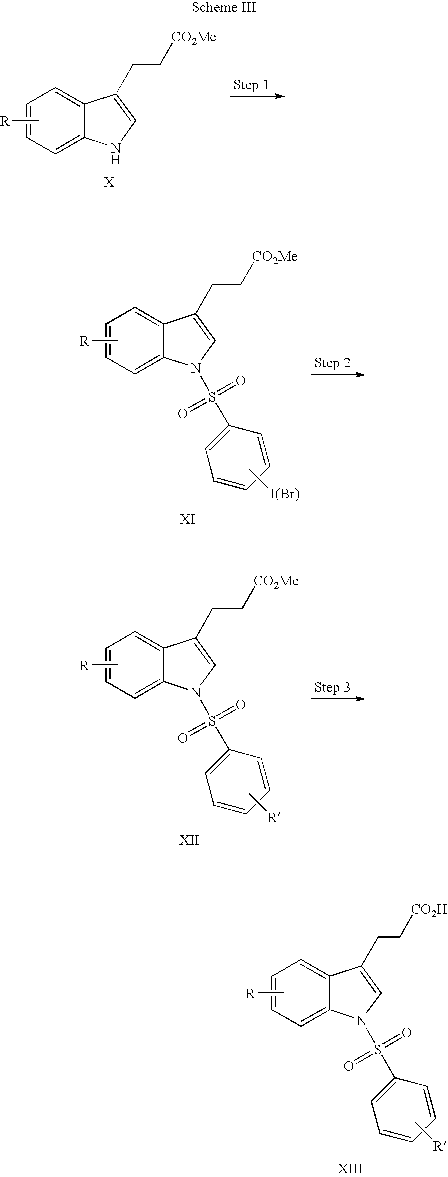

Step 1: Preparation of Compound XI

Compound XI is prepared by deprotonation of the indole nitrogen of compound X with the use of a base, such as for example, sodium hydride, and coupling with a halogen substituted aryl sulfonyl chloride in an inert solvent such as N,N -dimethylformamide.

Step 2: Preparation of Compound XII

Compound XII is prepared through metal catalyzed (such as palladium) biaryl coupling of a boronic acid with halogen (iodo or bromo) substituted aromatic ring, under basic conditions (i.e., Suzuki Cross Coupling).

Step 3: Preparation of Compound XIII

The final step of the synthesis of compound XIII involves the deprotection of the ester (methyl or ethyl) under saponification conditions with an aqueous hydroxide solution and an inert solvent such as tetrahydrofuran (THF).

Similarly, the Suzuki Cross Coupling reaction can also be extended to halogenated thiophenes. As illustrated in Scheme IV, bi-aryl substituted thiophenes can be generated through the same synthetic route as illustrated in Scheme III.

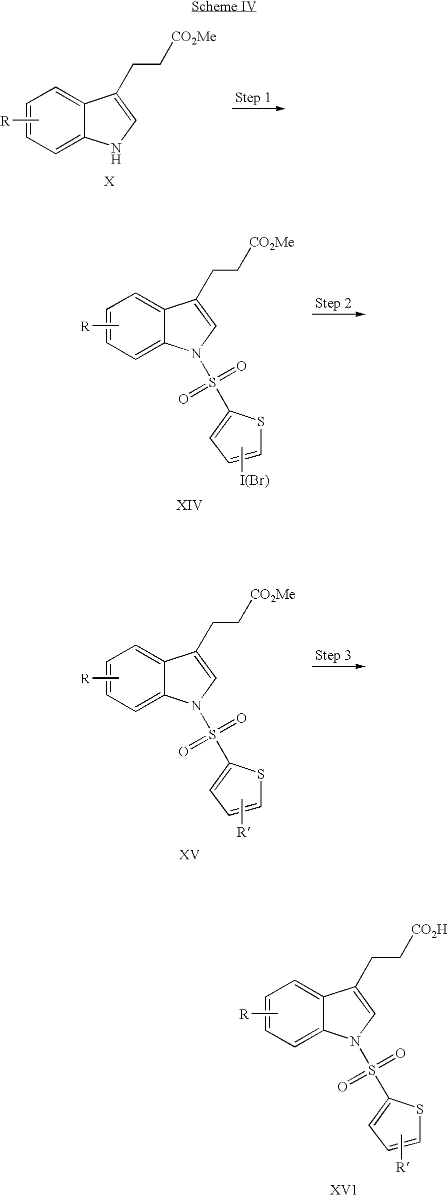

Step 1: Preparation of Compound XIV

Compound XIV is prepared through deprotonation of the indole nitrogen with the use of a base, such as for example, sodium hydride, and coupling with a halogen substituted thiophenyl sulfonyl chloride in an inert solvent such as N,N-dimethylformamide.

Step 2: Preparation of Compound XV

Compound XV is prepared through metal catalyzed (such as palladium) biaryl coupling of a boronic acid with a halogen (iodo or bromo) substituted aromatic ring, under basic conditions.

Step 3: Preparation of Compound XVI

In the final step of the synthesis of compound XVI the ester (methyl or ethyl) is deprotected under saponification conditions with an aqueous hydroxide solution with an inert solvent such as tetrahydrofuran (THF).

An alternative approach to generation of the biaryl linkage of the indole-1-sulfonamides contemplates reversing the order of the boronic acid/ester of the reagents. This synthetic strategy is illustrated in Scheme V using a thiophene as an example. Compounds of this type can be prepared in five synthetic steps.

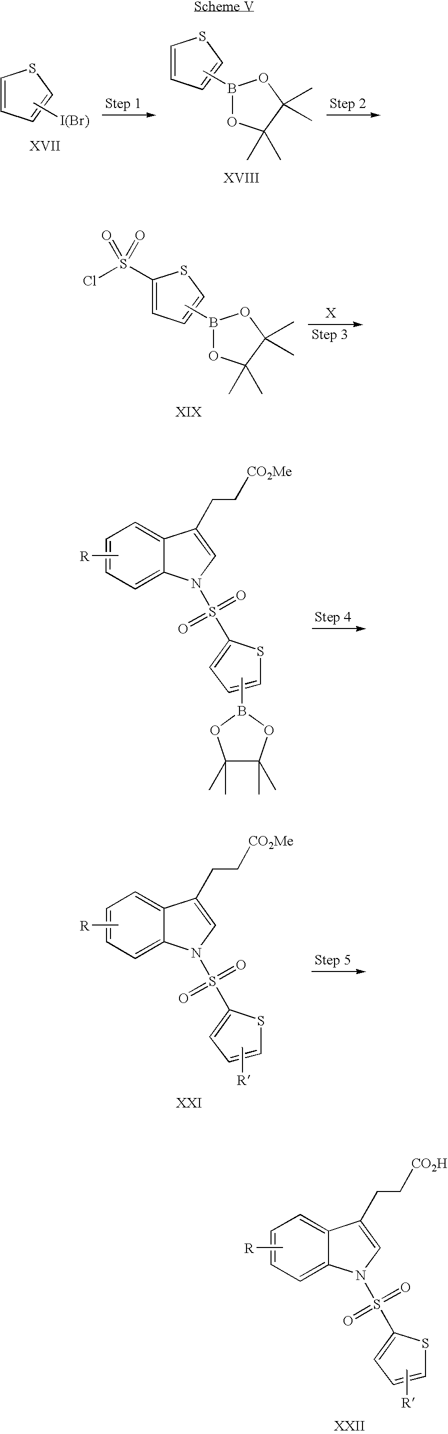

Step 1: Preparation of Compound XVIII

From the halogenated thiophene XVII, a lithium exchange can occur, using reagents such as n-butyl lithium at −78° C. The thienyl lithium can be coupled with boron trichloride. Subsequent hydrolysis of the dichloride with an alcohol or a 1,2-dihydroxyalkane, such as for example, pinacol would generate the desired boronic ester.

Step 2: Preparation of Compound XIX

Compound XVIII is treated with chlorosulfonic acid under cold conditions (define temperature) to add the sulfonyl chloride to the thiophene boronic ester.

Step 3: Preparation of Compound XX

Compound XIX is coupled to the indole X as described in Schemes III and IV using a base for deprotonation of the indole nitrogen, followed by coupling with the sulfonyl chloride in an inert solvent such as N,N-dimethylformamide.

Step 4: Preparation of Compound XXI

Compound XXI is prepared through metal catalyzed (such as palladium) biaryl coupling of a boronic acid with a halogen (lodo or Bromo) substituted aromatic ring, under basic conditions.

Step 5: Preparation of Compound XXII

The final step of the synthesis involves the deprotection of the ester (methyl or ethyl) under saponification conditions with an aqueous hydroxide solution with an inert solvent such as tetrahydrofuran (THF).

### EXAMPLES

### Example 1

### Synthesis of 3-{5-methoxy-1-[(E)-2-(4-trifluoromethyl-phenyl)-ethanesulfonyl]-1H-indol-3-yl}-propionic acid (P-0026)

Compound P-0026 was synthesized in three steps from 5-methoxy indole 1 as shown in Scheme 1.

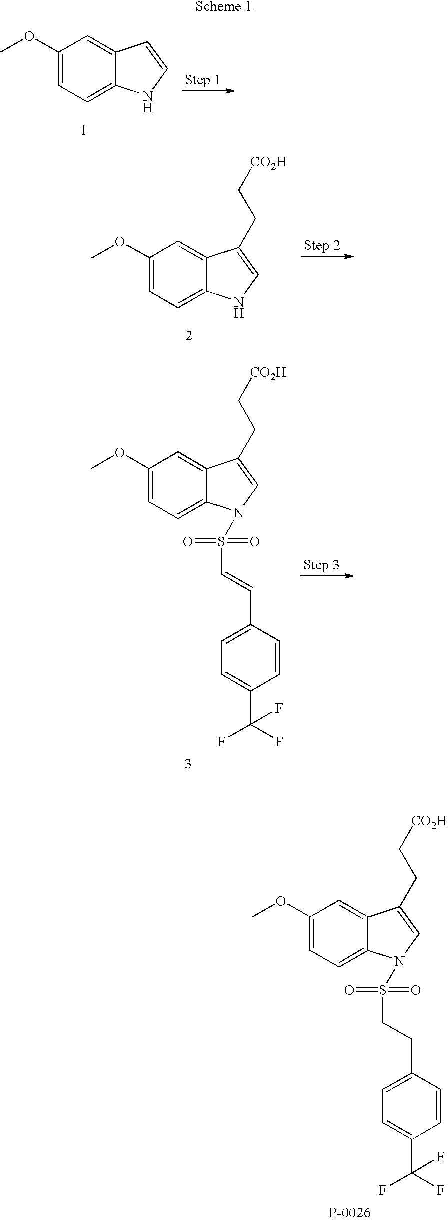

### Step 1: Preparation of 3-(5-methoxy-1H-indol-3-yl)-propionic acid (2)

To a solution of 5-methoxyindole (1, 4.00 g, 0.0272 mol) dissolved in acetic acid (13 mL, 0.22 mol) containing acetic anhydride (5 mL, 0.06 mol), acrylic acid (4.06 mL, 0.0592 mol) was added and the reaction was heated to 90° C. for 3 hrs. The reaction mixture was then concentrated, 3 ml NaOH (2M) was added and the mixture stirred for 5 min. The insoluble material was removed by filtration. The filtrate was acidified with 6M HCl. The precipitate was filtered off to yield 2 (1.95 g, 33%).

### Step 2: Preparation of 3-5-Methoxy-1-[(E)-2-(4-trifluoromethyl-phenyl)-ethenesulfonyl]-1H-indol-3-yl-propionic acid (3)

Into a dried and argon bled round bottom flask was dissolved 3-(5-Methoxy-1H-indol-3-yl)-propionic acid (2, 90.0 mg, 0.000410 mol) in dry Tetrahydrofuran (4 mL, 0.06 mol). The solution was cooled to −76° C. and 2.5 M of n-Butyllithium in Hexane (328 uL) was added drop wise. After 15 minutes (E)-2-(4-Trifluoromethyl-phenyl)-ethenesulfonyl chloride (167 mg, 0.000616 mol) dissolved in 0.5 ml dry THF was added drop wise. The reaction was stirred overnight. EtOAc was added to the mixture and then acidified with HCl (1M). The mixture was stirred for 1 hour. The organic phase was separated and the aqueous phase was extracted 3 times with EtOAc. The pooled organic phase was dried (Na2SO4) and the mixture was concentrated, then put on silica and purified with flash chromatography (0.5% MeOH in DCM) to yield 33 mg, 18% of 3.

### Step 3: Preparation of 3-5-Methoxy-1-[(E)-2-(4-trifluoromethyl-phenyl)-ethanesulfonyl]-1H-indol-3-yl-propionic acid. (P-0026)

To 3-5-Methoxy- 1-[(E)-2-(4-trifluoromethyl-phenyl)-ethenesulfonyl]- 1H-indol-3-yl-propionic acid (3, 8 mg, 0.00002 mol) dissolved in Tetrahydrofuran (2.0 mL, 0.025 mol), 5% Pd/C (5:95, Palladium:Carbon, 7 mg) was added to the solution. The mixture was stirred overnight under an atmosphere of hydrogen gas. The palladium was filtered off, and the solution evaporated to give P-0026 (8.0 mg, 100%). Calculated molecular weight 455.45, MS (ESI) [M+H+]+=454.0.

### Example 2

### Synthesis of 3-{5-methoxy-1[-5-(4-methoxy-phenyl)-thiophene-2-sulfonyl]-1H-indol-3-yl}-propionic acid (P-0016)

Compound P-0016 was synthesized in five steps from 5-methoxyindole-3-carboxaldehyde 4 as shown in Scheme 2.

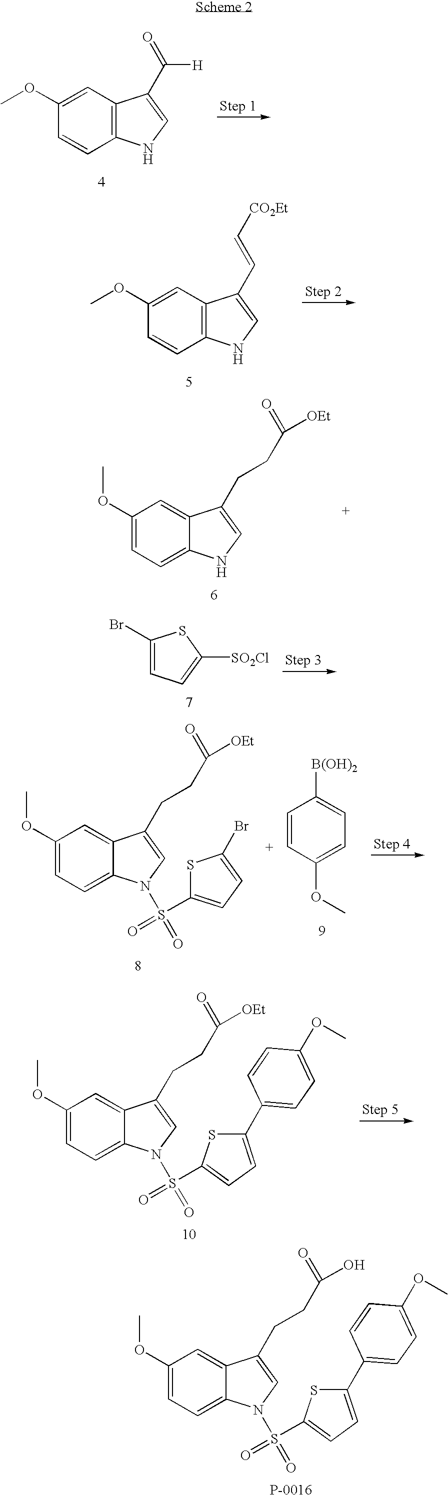

### Step 1: Preparation of 3-(5-methoxy-1H-indol-3-yl)-acrylic acid ethyl ester (5)

To a cold solution (ice bath) of ethyl diethylphosphonoacetate (30.11 g, 0.134 mol) in tetrahydrofuran (300 mL) under nitrogen, was added sodium hydride (6.44 g, 0.161 mol, 60%) in four portions, and stirred until hydrogen evolution ceased (caution: very vigorous evolution of gas). A solution of 5-methoxyindole-3-carboxyaldehyde (4,19.61 g, 0.112 mol) in 350 mL tetrahydrofuran was added over a period of 60 minutes to the phosphonate solution. The reaction mixture was heated to 55° C. for 24 hours, after which the mixture was diluted with 650 mL dichloromethane and washed with water (200 mL; 3X). The organic layer was washed once with brine, dried over anhydrous sodium sulfate, and evaporated under reduced pressure to give a yellow-tinted oil, which was purified by filtering through a silica plug. The filtrate was evaporated to afford compound 5 as an off white solid. ¹H NMR is consistent with the compound structure set forth above.

### Step 2—Preparation of 3-(5-methoxy-1H-indol-3-yl)-propionic acid ethyl ester (6)

To a solution of 3-(5-methoxy-1H-indol-3-yl)-acrylic acid ethyl ester 5 in 250 mL ethyl acetate was added palladium on activated carbon (10%; 3 g). The solution was deoxygenated under vacuum and hydrogen was introduced to the reaction flask from a balloon filled with hydrogen. The process was repeated three times and the reaction mixture was stirred for 16 hours at room temperature. The mixture was filtered through celite and the filtrate was evaporated under reduced pressure to yield compound 6 as a while solid (18.9 g, 68% yield). IH NMR is consistent with the compound structure set forth above.

### Step 3: Preparation of 3-[1-(5-bromo-thiophen-2-ylmethyl)-5-methoxy-1H-indol-3-yl]-propionic acid ethyl ester (8)

To a dry round bottom flask, 3-(5-methoxy-1H-indol-3-yl)-propionic acid ethyl ester (6, 492.0 mg, 1.9 mmol) was dissolved with dichloromethane (12 mL). Tetrabutylammonium hydrogen sulfate (30 mg) and 50% KOH solution (5 mL) were added next. After about 5 minutes of stirring, 5-bromo thiophene-2-sulfonyl chloride (7, 774.0 mg, 2.9 mmol) was added. This reaction was allowed to stir at ambient temperature overnight, after which 50 mL water and 150 mL ethyl acetate were added to the reaction. The layers were separated and the organic layer was washed with saturated bicarbonate (3×75 mL) and water (2×75 mL) to remove the hydroxide and sulfonate salt, then washed with brine (1×75 mL) and dried over anhydrous sodium sulfate. Evaporation under reduced pressure afforded compound 8 as a brown oil. (820 mg, 87%). ¹H NMR is consistent with the compound structure set forth above.

### Step 4: Preparation of 3-{5-methoxy-1-[5-(4-methoxy-phenyl)-thiophene-2-sulfonyl]-1H-indol-3-yl}-propionic acid ethyl ester (10)

Into a 50 mL oven dried round bottom flask, 3-[1-(5-bromo-thiophen-2-ylmethyl)-5-methoxy-1H-indol-3-yl]-propionic acid ethyl ester (8, 300 mg, 0.064 mmol) was dissolved in dry tetrahydrofuran (8 mL) under an argon flow. 4-Methoxy-phenyl boronic acid (9, 24.0 mg, 0.16 mmol), tetrakis(triphenylphosphine) palladium(0) (7.2 mg, 0.006 mmol) and 1 N K₂CO₃ (0.4 mL) were added. A condenser equipped with argon gas line was attached and the reaction heated at 48° C. for 3 days. The solvent was removed under reduced pressure, and the crude product was purified by flash chromatography on silica gel, using a gradient of 0-10% ethyl acetate/hexane to provide compound 10. ¹H NMR is consistent with the compound structure set forth above.

### Step 5: Synthesis of 3-{5-methoxy-1-[5-(4-methoxy-phenyl)-thiophene-2-sulfonyl]-1H-indol-3-yl}-propionic acid (P-0016)

To a solution of 3-{5-methoxy-1-[5-(4-methoxy-phenyl)-thiophene-2-sulfonyl]-1H-indol-3-yl}-propionic acid ethyl ester 10 in tetrahydrofuran (4 mL) was added an aqueous solution of potassium hydroxide (1 mL of 1 M) and stirred at room temperature overnight. The acid product was isolated by neutralizing the reaction mixture with aqueous hydrochloric acid, extracting the product with ethyl acetate, drying over anhydrous magnesium sulfate, evaporating under reduced pressure, and triturating with diethyl ether to afford P-0016 as a white solid (10 mg, 32%) Calculated molecular weight 471.55, MS(ESI) [M−H⁺]³¹ =470.11).

Additional compounds were prepared following the protocol of Scheme 2, replacing 4-methoxy-phenyl boronic acid 9 with an appropriate boronic acid in Step 4. The following compounds were prepared by this method:
3-{5-Methoxy-1-[5-(4-trifluoromethoxy-phenyl)-thiophene-2-sulfonyl]-1H-indol-3-yl}-propionic acid ethyl ester (P-0014, isolated after Step 4),
3-{5-Methoxy-1-[5-(4-trifluoromethoxy-phenyl)-thiophene-2-sulfonyl]-1H-indol-3-yl}-propionic acid (P-0015),
3 -{1-[5-(4-Ethoxy-phenyl)-thiophene-2-sulfonyl]-5-methoxy-1H-indol-3-yl}-propionic acid (P-0017),
3-{5-Methoxy-1-[5-(3-trifluoromethyl-phenyl)-thiophene-2-sulfonyl]-1H-indol-3-yl}-propionic acid (P-0019),
3-{5-Methoxy-1-[5-(3-trifluoromethoxy-phenyl)-thiophene-2-sulfonyl]-1 H-indol-3-yl}-propionic acid (P-0018),
3-{5-Methoxy-1-[5-(4-trifluoromethyl-phenyl)-thiophene-2-sulfonyl]-1H-indol-3-yl}-propionic acid (P-0020),
3-{5-Methoxy-1-[5-(4-propoxy-phenyl)-thiophene-2-sulfonyl]-1H-indol-3-yl}-propionic acid (P-0035),
3-{1-[5-(4-Isopropoxy-phenyl)-thiophene-2-sulfonyl]-5-methoxy-1H-indol-3-yl}-propionic acid ethyl ester (P-0036, isolated after Step 4), and
3-{1-[5-(4-Isopropoxy-phenyl)-thiophene-2-sulfonyl]-5-methoxy-1H-indol-3-yl}-propionic acid (P-0037).

These compounds are shown in the following Table 1, where the compound number is provided in Column 1, the boronic acid used in Step 4 in Column 2, the compound structure in Column 3, and the calculated and measured mass in Columns 4 and 5.

TABLE 1

| | | | Molecular weight | |
|---|---|---|---|---|
| Cmpd. | | | | Measured |
| number | Boronic acid | Compound structure | Calc. | MS (ESI) |
| P-0014 | 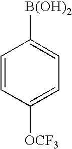 | 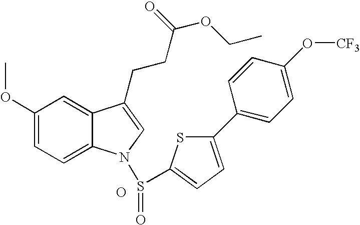 | 525.53 | [M + H⁺]⁺ = 526.16 |
| P-0015 | 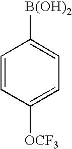 | 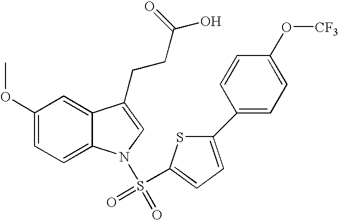 | 471.55 | [M − H⁺]⁻ = 470.11 |
| P-0017 | 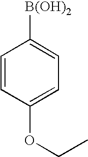 | 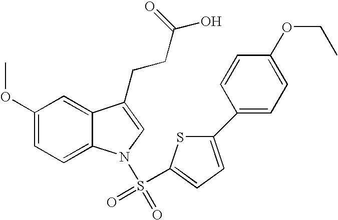 | 485.10 | [M − H⁺]⁻ = 484.17 |
| P-0019 | 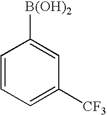 | 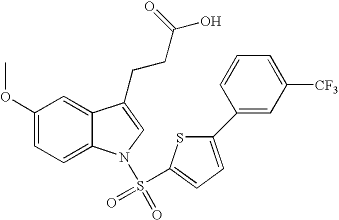 | 509.06 | [M − H⁺]⁻ = 508.12 |
| P-0018 | 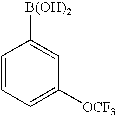 | 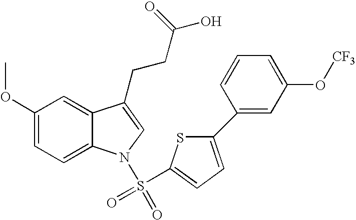 | 525.05 | |
| P-0020 | 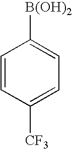 | 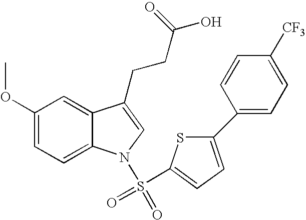 | 509.52 | [M − H⁺]⁻ = 508.12 [M + H⁺]⁺ = 510.20 |
| P-0035 | 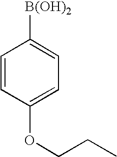 | 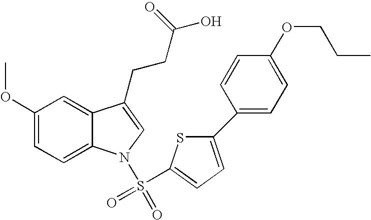 | 499.61 | |
| P-0036 | 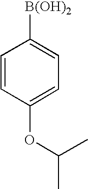 | 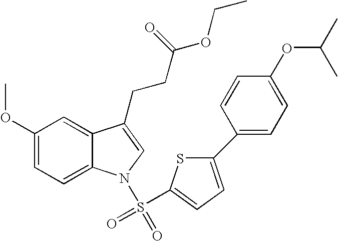 | 527.66 | |
| P-0037 | 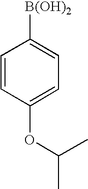 | 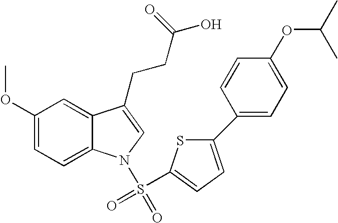 | 499.61 | |

Additional compounds were prepared following the protocol of Scheme 2, optionally replacing 5-methoxyindole-3-carboxyaldehyde 4 with an appropriate indole carboxyaldehyde in Step 1, and/or optionally replacing 5-bromo thiophene-2-sulfonyl chloride 7 with an appropriate sulfonyl chloride in Step 3, and taking the product of Step 3 directly on to Step 5 to form the propionic acid. The following compounds were prepared by this method, with the calculated molecular weight and measured mass (MS(ESI)) provided after the compound:
3-{5-Fluoro-1-[5-(1-methyl-5-trifluoromethyl-1H-pyrazol-3-yl)-thiophene-2-sulfonyl]-1H-indol-3-yl}-propionic acid (P-0002), calculated MW 501.47, [M−H+]⁻=500.08,
3-{5-Fluoro-1-[5-(5-trifluoromethyl-isoxazol-3-yl)-thiophene-2-sulfonyl]-1H-indol-3-yl}-propionic acid (P-0003), calculated MW 488.43, [M−H⁺]⁻=487.08,
3-{5-Chloro-1-[5-(5-trifluoromethyl-isoxazol-3-yl)-thiophene-2-sulfonyl]-1H-indol-3-yl}-propionic acid (P-0004), calculated MW 504.92, [M+H⁺]⁺=505.48, [M−H⁺]⁻=503.06,
3-{1-[4-(4-Trifluoromethyl-phenoxy)-benzenesulfonyl]-1H-indol-3-yl}-propionic acid (P-0006), calculated MW 489.47, [M+H⁺]⁺=488.32,
3-{1-[5-(1-Methyl-5-trifluoromethyl-1H-pyrazol-3-yl)-thiophene-2-sulfonyl]-1H-indol-3-yl}-propionic acid (P-0007), calculated MW 483.49, [M−H⁺]⁻=482.2,
3-{5-Methoxy-1-[5-(5-trifluoromethyl-isoxazol-3-yl)-thiophene-2-sulfonyl]-1H-indol-3-yl}-propionic acid (P-0008), calculated MW 500.47, [M−H⁺]⁻=499.1,
3-{5-Ethoxy-1-[5-(5-trifluoromethyl-isoxazol-3-yl)-thiophene-2-sulfonyl]-1H-indol-3yl}-propionic acid ethyl ester (P-0009), calculated MW 528.52, [M−H⁺]⁻=527.1,
3-{5-Chloro-1-[5-(1-methyl-5-trifluoromethyl-1H-pyrazol-3-yl)-thiophene-2-sulfonyl]-1H-indol-3-yl}-propionic acid (P-0033), calculated MW 517.0, [M+H⁺]⁺=518.15, [M−H⁺]⁻=516.07,
3-{5-Methoxy-1-[5-(2-methyl-5-trifluoromethyl-2H-pyrazol-3-yl)-thiophene-2-sulfonyl]-1H-indol-3-yl}propionic acid (P-0034), calculated MW 513.53, [M+H⁺]⁺=514.33, [M−H⁺]⁻=512.24,
3-{5-Methoxy-1 -[4-(pyridin-2-yloxy)-benzenesulfonyl]-1H-indol-3-yl }-propionic acid (P-0047), calculated MW 452.49, [M+H⁺]⁺=453.1,
3-{5-Methoxy-1-[4-(4-methoxy-phenoxy)-benzenesulfonyl]-1H-indol-3 -yl }-propionic acid (P-0048), calculated MW 481.53, [M+H⁺]⁺=482.3,
3-{1-[4-(3,4-Dichloro-phenoxy)-benzenesulfonyl]-5-methoxy-1H-indol-3-yl}-propionic acid (P-0049), calculated MW 520.39, [M−H⁺]⁻=519.9,
3-{1-[4-(3,5-Dichloro-phenoxy)-benzenesulfonyl]-5-methoxy-1H-indol-3-yl}-propionic acid (P-0050), calculated MW 520.39, [M−H⁺]⁻=519.9,
3-{5-Methoxy-1-[4-(4-trifluoromethyl-phenoxy)-benzenesulfonyl]-1H-indol-3-yl}-propionic acid (P-0051), calculated MW 519.50, [M+H⁺]⁺=519.9,
3-{1-[3-(3,4-Dichloro-phenoxy)-benzenesulfonyl]-5-ethoxy-1H-indol-3-yl }-propionic acid (P-0052), calculated MW 534.42, [M−H⁺]⁻=533.9,
3-{5-Ethoxy-1-[5-(2-methylsulfanyl-pyrimidin-4-yl)-thiophene-2-sulfonyl]-1H-indol -3-yl}-propionic acid (P-0053), calculated MW 503.62, [M+H⁺]⁺=520.3,
3-{5-Ethoxy-1-[4-(pyridin-2-yloxy)-benzenesulfonyl]-1H-indol-3-yl}-propionic acid (P-0055), calculated MW 466.52, [M+H⁺]⁺=467.1,
3-{5-Ethoxy-1 -[4-(pyridin-3-yloxy)-benzenesulfonyl]-1H-indol-3-yl}-propionic acid (P-0056), calculated MW 466.52, [M+H⁺]⁺=467.1,
3-{5-Ethoxy-1-[4-(4-methoxy-phenoxy)-benzenesulfonyl]-1H-indol-3-yl}-propionic acid (P-0057), calculated MW 495.56, [M+H⁺]⁺=496.3,
3-{1-[4-(3,4-Dichloro-phenoxy)-benzenesulfonyl]-5-ethoxy-1H-indol-3-yl}-propionic acid (P-0058), calculated MW 534.42, [M−H⁺]⁻=533.9,
3-{1-[4-(3,5-Dichloro-phenoxy)-benzenesulfonyl]-5-ethoxy-1H-indol-3-yl}-propionic acid (P-0059), calculated MW 534.42, [M−H⁺]⁻=533.9,
3-{5-Ethoxy-1-[4-(4-trifluoromethyl-phenoxy)-benzenesulfonyl]-1H-indol-3-yl}-propionic acid (P-0060), calculated MW 533.53, [M+H⁺]⁺=533.9,
3-{1-[4-(3 -Chloro-5-trifluoromethyl-pyridin-2-yloxy)-benzenesulfonyl]-5-ethoxy- 1H -indol-3-yl}-propionic acid (P-0061), calculated MW 568.96, [M+H⁺]⁺=569.2,
3-[5-Ethoxy-1-(4′-methoxy-biphenyl-4-sulfonyl)-1H-indol-3-yl]-propionic acid (P-0062), calculated MW 479.56, [M+H⁺]⁺=480.3,
3-[5-Ethoxy-1-(6-morpholin-4-yl-pyridine-3-sulfonyl)-1H-indol-3-yl]-propionic acid (P-0063), calculated MW 459.53, [M+H⁺]⁺=460.3,
3-[5-Ethoxy-1-(6-phenoxy-pyridine-3-sulfonyl)-1H-indol-3-yl]-propionic acid (P-0064), calculated MW 466.52, [M+H⁺]⁺=467.1,
3-[5-Ethoxy-1-(5-pyridin-2-yl-thiophene-2-sulfonyl)-1H-indol-3-yl]-propionic acid (P-0065), calculated MW 456.54, [M+H⁺]⁺=457.1,
3-{5-Ethoxy-1-[5-(1-methyl-5-trifluoromethyl-1H-pyrazol-3-yl)-thiophene-2-sulfonyl]-1H-indol-3-yl}-propionic acid (P-0066), calculated MW 527.55, [M+H⁺]⁺=527.9,
3-{5-Methoxy-1-[5-(2-methylsulfanyl-pyrimidin-4-yl)-thiophene-2-sulfonyl]-1H-indol-3-yl}-propionic acid (P-0067), calculated MW 489.59, [M−H⁺]⁻=489.1,
3-{1-[4-(3-Chloro-5-trifluoromethyl-pyridin-2-yloxy)-benzenesulfonyl]-5-methoxy-1H-indol-3-yl}-propionic acid (P-0068), calculated MW 554.93, [M+H⁺]⁺=555.2,
3-{5-Methoxy-1-[5-(1-methyl-5-trifluoromethyl-1H-pyrazol-3-yl)-thiophene-2-sulfonyl]-1H-indol-3-yl}-propionic acid (P-0069), calculated MW 513.52, [M−H⁺]⁻=512.09,
3-{1-[3-(3,4-Dichloro-phenoxy)-benzenesulfonyl]-5-methoxy-1H-indol-3-yl}-propionic acid (P-0071), calculated MW 520.39, [M−H⁺]⁻=520.3,
3-[5-Methoxy-1-(4′-methoxy-biphenyl-4-sulfonyl)-1H-indol-3-yl]-propionic acid (P-0072), calculated MW 465.53, [M+H⁺]⁺=466.3,3-[5-Methoxy-1-(5-methyl-1-phenyl-1H-pyrazole-4-sulfonyl)-1H-indol-3-yl]-propionic acid (P-0146), calculated MW 439.49, [M+H⁺]⁺=440.3,
3-{5-Methoxy-1-[3-(pyridine-2-carbonyl)-benzenesulfonyl]-1H-indol-3-yl}-propionic acid (P-0150), calculated MW 464.50, [M+H⁺]⁺=465.1,
3-{5-Methoxy-1-[3-(pyridine-4-carbonyl)-benzenesulfonyl]-1H-indol-3-yl}-propionic acid (P-0151), calculated MW 464.50, [M+H⁺]⁺=465.1,
3-[1-(Biphenyl-2-sulfonyl)-5-methoxy-1H-indol-3-yl]-propionic acid (P-0152), calculated MW 435.50, [M+H⁺]⁺=436.3,
3-[5-Methoxy-1 -(4-pyrazol-1-yl-benzenesulfonyl)-1H-indol-3-yl]-propionic acid (P-0155), calculated MW 425.47, [M+H⁺]⁺=426.3,
3-[5-Methoxy-1-(2-phenoxy-benzenesulfonyl)-1H-indol-3-yl]-propionic acid (P-0162), calculated MW 451.50, [M+H⁺]⁺=451.9,
3-{5-Ethoxy-1-[3-(pyridine-4-carbonyl)-benzenesulfonyl]-1H-indol-3-yl}-propionic acid (P-0168), calculated MW 478.53, [M+H⁺]⁺=479.1,
3-[5-Methoxy-1-(6-morpholin-4-yl-pyridine-3-sulfonyl)-1H-indol-3-yl]-propionic acid (P-0214), calculated MW 445.50, [M+H⁺]⁺=446.3,
3-[5-Methoxy-1-(6-phenoxy-pyridine-3-sulfonyl)-1H-indol-3-yl]-propionic acid (P-0215), calculated MW 452.49, [M+H⁺]⁺=453.1,
3-[5-Methoxy-1-(5-pyridin-2-yl-thiophene-2-sulfonyl)-1H-indol-3-yl]-propionic acid (P-0216), calculated MW 442.52, [M+H⁺]⁺=443.5,
3-{5-Isopropoxy-1-[5-(2-methylsulfanyl-pyrimidin-4-yl)-thiophene-2-sulfonyl]-1H-indol-3-yl}-propionic acid (P-0311), calculated MW 517.46, [M+H⁺]⁺=517.9,
3-{5-Isopropoxy-1-[4-(pyridin-2-yloxy)-benzenesulfonyl]-1H-indol-3-yl}-propionic acid (P-0316), calculated MW 480.54, [M+H⁺]⁺=481.1,
3-{5-Isopropoxy-1-[4-(pyridin-4-yloxy)-benzenesulfonyl]-1H-indol-3-yl}-propionic acid (P-0317), calculated MW 480.54, [M+H⁺]⁺=481.1,
3-{5-Isopropoxy-1-[4-(4-methoxy-phenoxy)-benzenesulfonyl]-1H-indol-3-yl}-propionic acid (P-0318), calculated MW 509.98, [M+H⁺]⁺=510.3,
3-{5-Isopropoxy-1-[4-(4-trifluoromethyl-phenoxy)-benzenesulfonyl]-1H-indol-3-yl}-propionic acid (P-0319), calculated MW 547.55, [M+H⁺]⁺=548.3,
3-{1-[4-(3-Chloro-5-trifluoromethyl-pyridin-2-yloxy)-benzenesulfonyl]-5-isopropoxy- 1H-indol-3-yl}-propionic acid (P-0320), calculated MW 582.99, [M+H⁺]⁺=583.2,
3-{1-[3-(3,4-Dichloro-phenoxy)-benzenesulfonyl]-5-isopropoxy-1H-indol-3-yl}-propionic acid (P-0321), calculated MW 548.45, [M−H⁺]⁻=547.9,
3-[5-Isopropoxy- 1 -(4′-methoxy-biphenyl-4-sulfonyl)-1H-indol-3-yl]-propionic acid (P-0322), calculated MW 493.58, [M+H⁺]⁺=494.3,
3-[5-Isopropoxy-1-(6-phenoxy-pyridine-3-sulfonyl)-1H-indol-3-yl]-propionic acid (P-0323), calculated MW 480.54, [M+H⁺]⁺=481.1,
3-[5-Isopropoxy-1-(5-pyridin-2-yl-thiophene-2-sulfonyl)-1H-indol-3-yl]-propionic acid (P-0324), calculated MW 470.57, [M+H⁺]⁺=471.1,
3-{5-Isopropoxy- 1-[5-(1-methyl-5-trifluoromethyl-1H-pyrazol-3-yl)-thiophene-2-sulfonyl]-1H-indol-3-yl}-propionic acid (P-0326), calculated MW 541.57, [M+H⁺]⁺=541.9,
3-[1-(Biphenyl-2-sulfonyl)-5-isopropoxy-1H-indol-3-yl]-propionic acid (P-0332), calculated MW 463.56, [M+H⁺]⁺=463.9,
3-[5-Isopropoxy-1-(4′-methyl-biphenyl-2-sulfonyl)-1H-indol-3-yl]-propionic acid (P-0338), calculated MW 477.58, [M+H⁺]⁺=478.3,
3-[5-Isopropoxy-1-(2-phenoxy-benzenesulfonyl)-1H-indol-3-yl]-propionic acid (P-0339), calculated MW 479.56, [M+H⁺]⁺=479.9,
3-(5-Ethoxy-1-{4-[(morpholine-4-carbonyl)-amino]-benzenesulfonyl}-1H-indol-3-yl)-propionic acid (P-0342), calculated MW 501.56, [M+H⁺]⁺=502.3,
3-{5-Ethoxy-1-[3-(pyridine-2-carbonyl)-benzenesulfonyl]-1H-indol-3-yl}-propionic acid (P-0344), calculated MW 478.53, [M+H⁺]⁺=479.1,
3-{5-Isopropoxy-1-[4-(pyridin-3-yloxy)-benzenesulfonyl]-1H-indol-3-yl}-propionic acid (P-0371), calculated MW 480.54, [M+H⁺]⁺=481.1,
3-[5-Isopropoxy-1-(4-pyrazol-1-yl-benzenesulfonyl)-1H-indol-3-yl]-propionic acid (P-0375), calculated MW 453.52, [M+H⁺]⁺=454.3,
3-[5-Ethoxy-1-(4′-methyl-biphenyl-2-sulfonyl)-1H-indol-3-yl]-propionic acid (P-0386), calculated MW 463.55, [M+H⁺]⁺=464.3,
3-[5-Methoxy-1-(4′-trifluoromethoxy-biphenyl-2-sulfonyl)-1H-indol-3-yl]-propionic acid (P-0391), calculated MW 519.49, [M−H⁺]⁻=518.26,
3-[5-Methoxy-1-(4′-trifluoromethyl-biphenyl-2-sulfonyl)-1H-indol-3-yl]-propionic acid (P-0392), calculated MW 503.50, [M−H⁺]⁻=502.25,
3-{1-[3-(3,5-Dichloro-phenoxy)-benzenesulfonyl]-5-methoxy-1H-indol-3-yl}-propionic acid (P-0560), calculated MW 520.39, [M−H⁺]⁻=518.02,
3-{5-Methoxy-1-[3-(4-methoxy-phenoxy)-benzenesulfonyl]-1H-indol-3-yl}-propionic acid (P-0561), calculated MW 481.52, [M−H⁺]⁻=480.09,
3-[5-Methoxy-1-(3-p-tolyloxy-benzenesulfonyl)-1H-indol-3-yl]-propionic acid ethyl ester (P-0562), calculated MW 493.58, [M+H⁺]⁺=494.2,
3-{1-[3-(4-Chloro-phenoxy)-benzenesulfonyl]-5-methoxy-1H-indol-3-yl}-propionic acid ethyl ester (P-0563), calculated MW 514.00, [M+H⁺]⁺=514.9,
3-{5-Methoxy-1-[3-(4-trifluoromethyl-phenoxy)-benzenesulfonyl]-1H-indol-3-yl}-propionic acid (P-0564), calculated MW 519.49, [M+H⁺]⁺=520.11, [M−H⁺]⁻=518.06,
3-{1-[3-(4-Fluoro-phenoxy)-benzenesulfonyl]-5-methoxy-1H-indol-3-yl}-propionic acid ethyl ester (P-0565), calculated MW 497.54, [M+H⁺]⁺=498.2,
3-[5-Methoxy-1-(3-p-tolyloxy-benzenesulfonyl)-1H-indol-3-yl]-propionic acid (P-0566), calculated MW 465.52, [M−H⁺]⁻=464.1,
3-{1-[3-(4-Chloro-phenoxy)-benzenesulfonyl]-5-methoxy-1H-indol-3-yl}-propionic acid (P-0567), calculated MW 485.94, [M−H⁺]⁻=484.3,
3-{1-[3-(4-Fluoro-phenoxy)-benzenesulfonyl]-5-methoxy-1H-indol-3-yl}-propionic acid (P-0568), calculated MW 469.49, [M−H⁺]⁻=468.1,
3-[5-Methoxy-1-(4′-trifluoromethyl-biphenyl-3-sulfonyl)-1H-indol-3-yl]-propionic acid (P-0570), calculated MW 503.50, and
3-[1-(4′-Trifluoromethyl-biphenyl-3-sulfonyl)-1H-indol-3-yl]-propionic acid (P-0572), calculated MW 473.47.

These compounds are shown in the following Table 2, where the compound number is provided in Column 1, the indole carboxyaldehyde used in Step 1 in Column 2, the sulfonyl chloride used in Step 3 in Column 3, with the compound structure in Column 4.

TABLE 2

| Cmpd. | | | |
|---|---|---|---|
| number | Indole-3-carboxyaldehyde | Sulfonyl chloride | Compound structure |
| P-0002 | 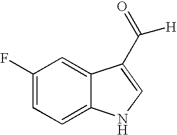 | 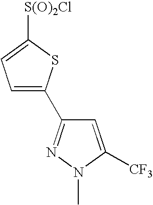 | 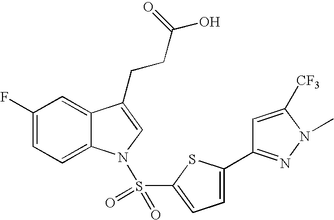 |
| P-0003 | 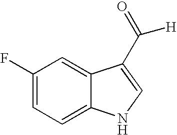 | 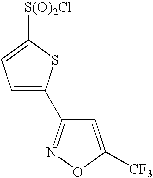 | 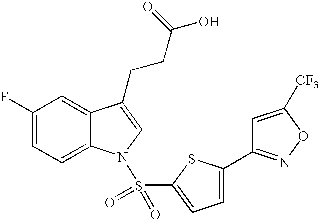 |
| P-0004 | 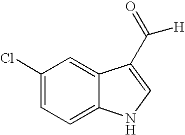 | 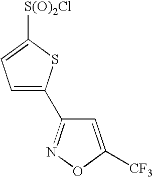 | 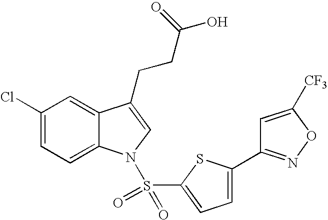 |
| P-0006 | 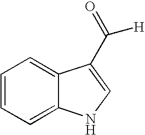 | 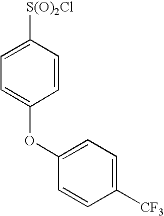 | 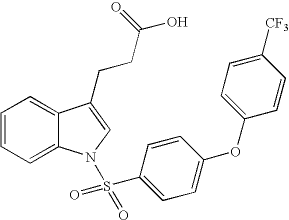 |
| P-0007 | 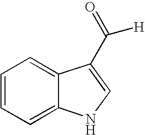 | 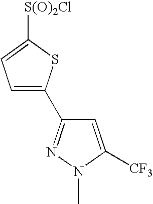 | 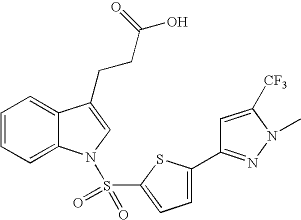 |
| P-0008 | 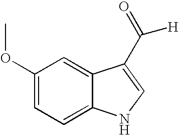 | 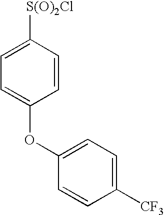 | 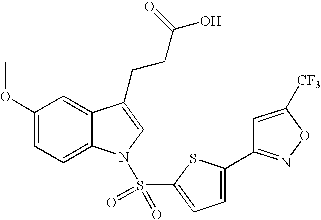 |
| P-0009* | 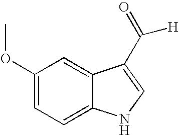 | 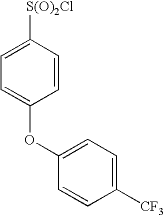 | 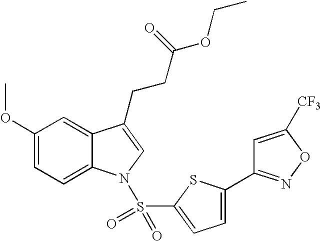 |
| P-0033 | 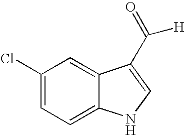 | 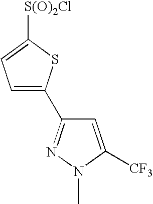 | 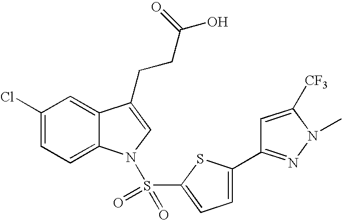 |
| P-0034 | 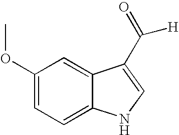 | 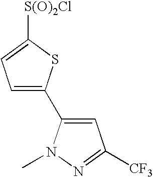 | 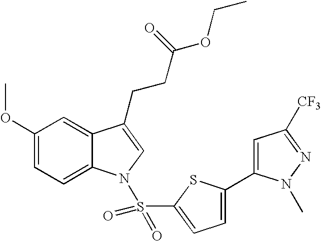 |
| P-0047 | 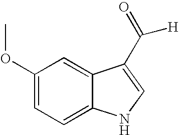 | 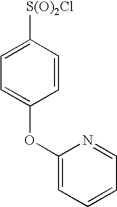 | 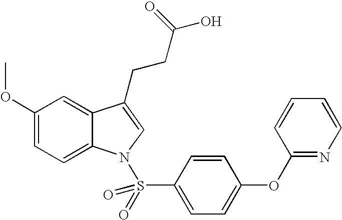 |
| P-0048 | 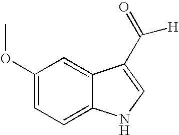 | 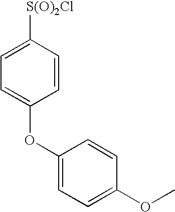 | 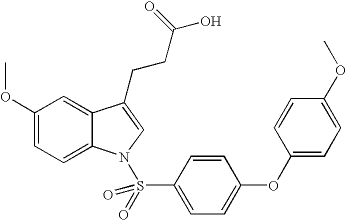 |
| P-0049 | 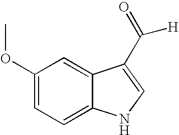 | 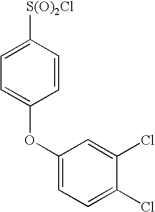 | 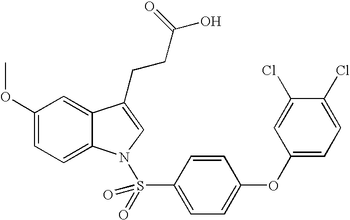 |
| P-0050 | 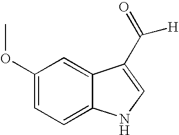 | 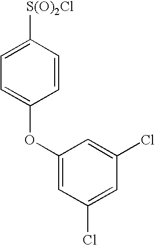 | 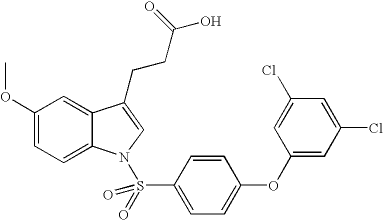 |
| P-0051 | 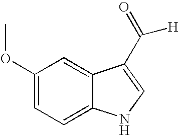 | 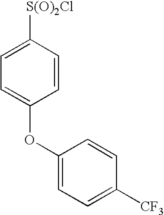 | 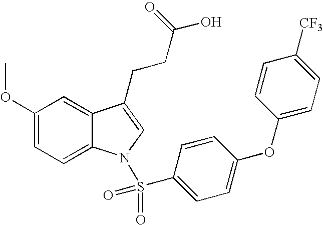 |
| P-0052 | 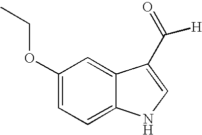 | 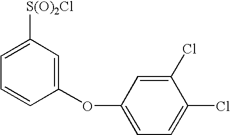 | 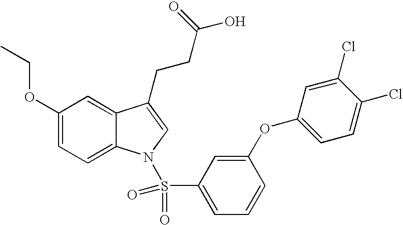 |
| P-0053 | 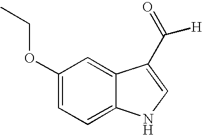 | 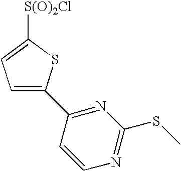 | 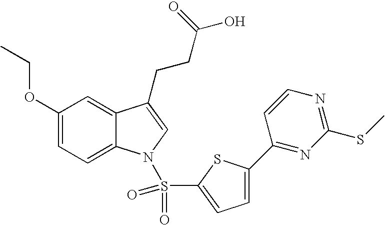 |
| P-0055 | 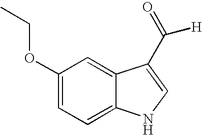 | 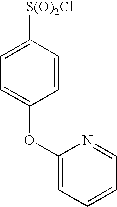 | 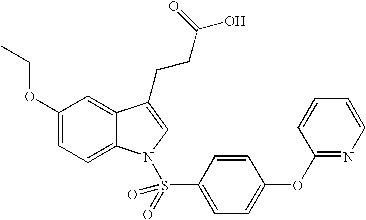 |
| P-0056 | 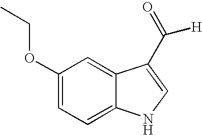 | 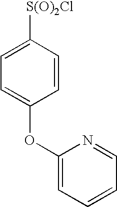 | 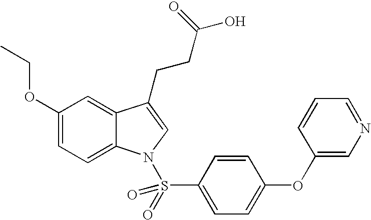 |
| P-0057 | 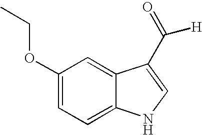 | 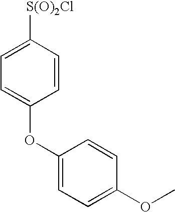 | 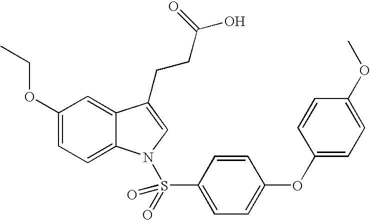 |
| P-0058 | 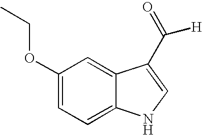 | 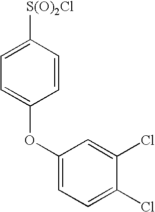 | 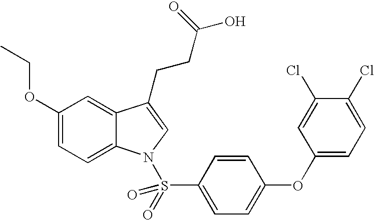 |
| P-0059 | 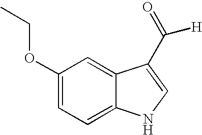 | 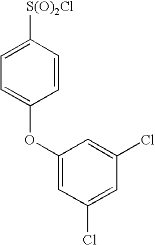 | 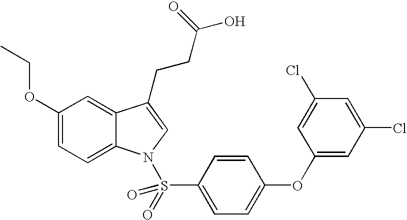 |
| P-0060 | 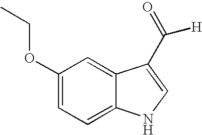 | 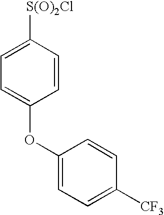 | 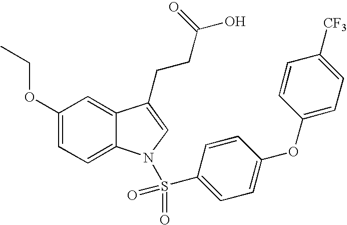 |
| P-0061 | 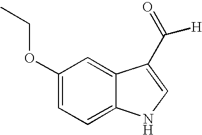 | 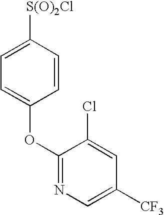 | 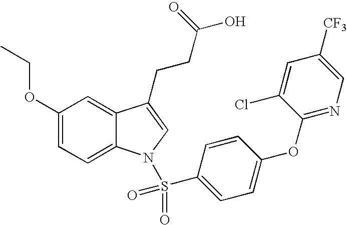 |
| P-0062 | 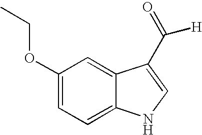 | 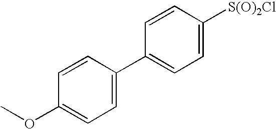 | 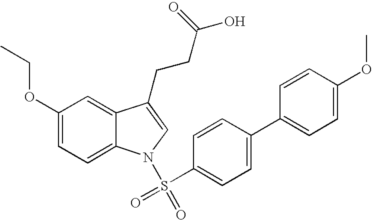 |
| P-0063 | 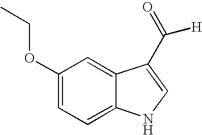 | 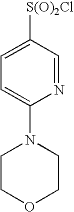 | 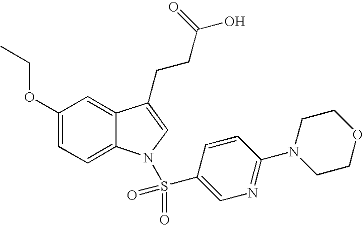 |
| P-0064 | 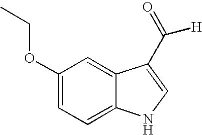 | 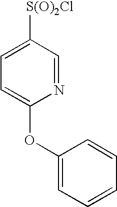 | 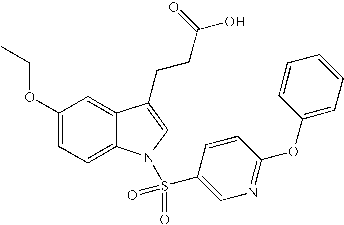 |
| P-0065 | 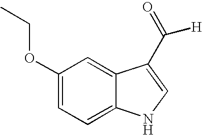 | 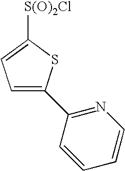 | 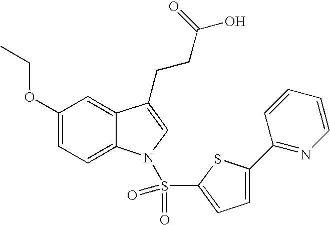 |
| P-0066 | 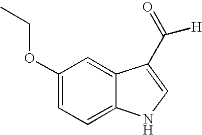 | 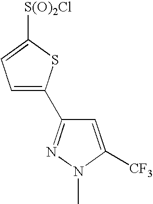 | 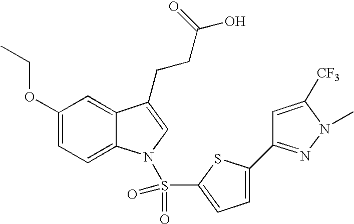 |
| P-0067 | 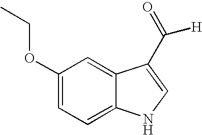 | 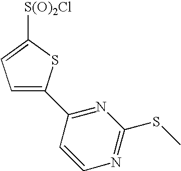 | 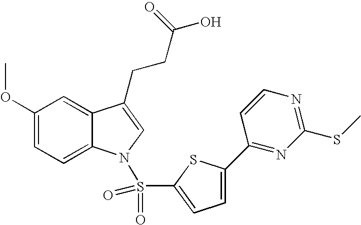 |
| P-0068 | 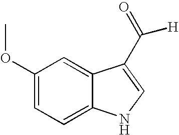 | 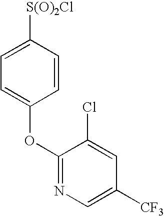 | 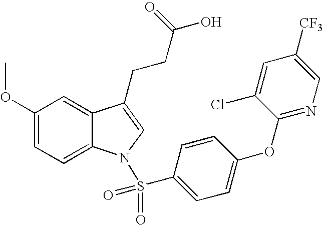 |
| P-0069 | 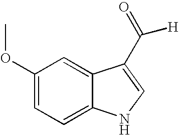 | 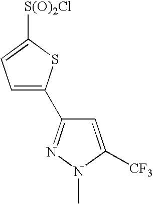 | 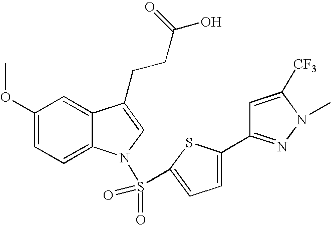 |
| P-0071 | 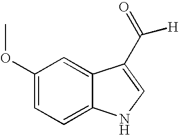 | 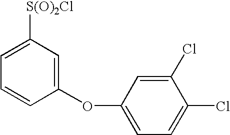 | 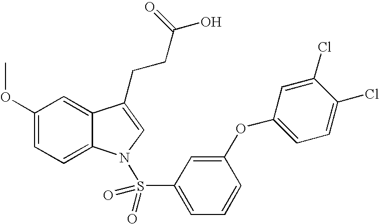 |
| P-0072 | 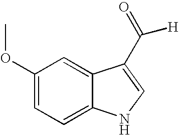 | 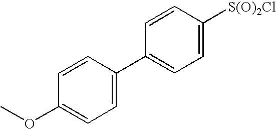 | 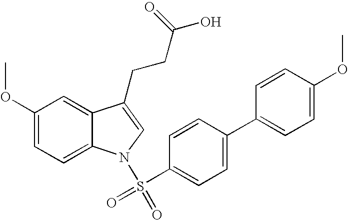 |
| P-0146 | 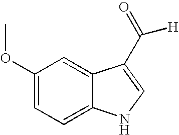 | 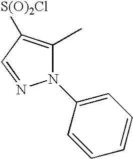 | 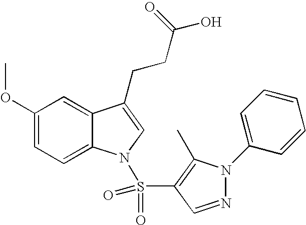 |
| P-0150 | 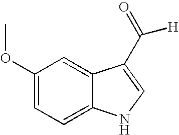 | 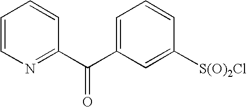 | 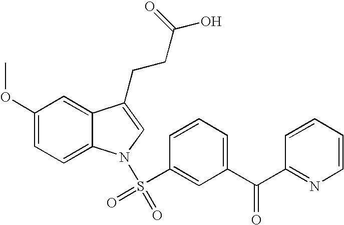 |
| P-0151 | 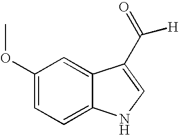 | 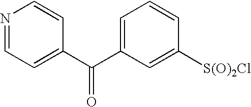 | 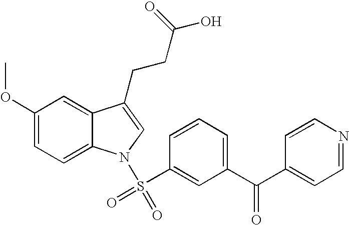 |
| P-0152 | 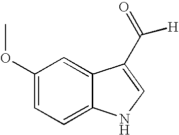 | 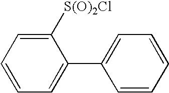 | 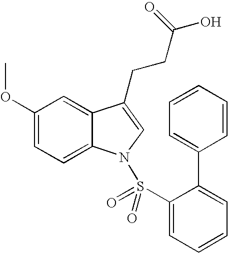 |
| P-0155 | 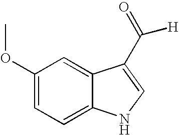 | 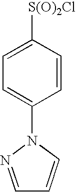 | 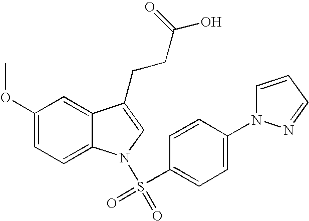 |
| P-0162 | 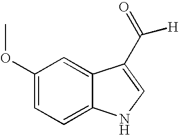 | 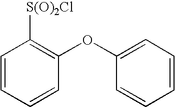 | 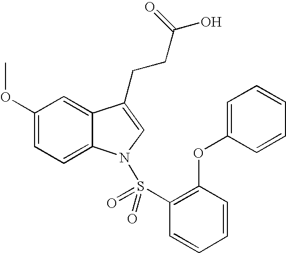 |
| P-0168 | 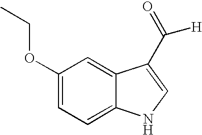 | 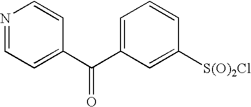 | 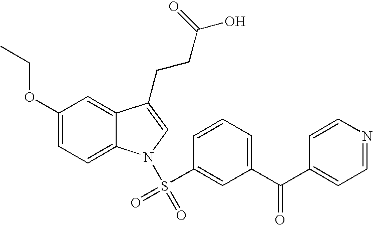 |
| P-0214 | 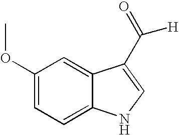 | 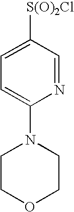 | 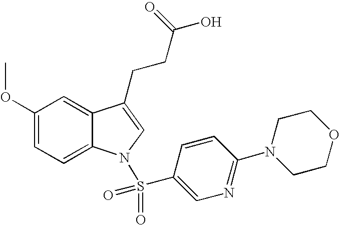 |
| P-0215 | 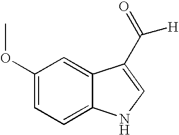 | 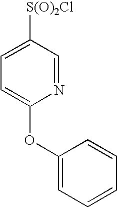 | 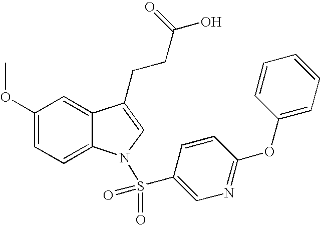 |
| P-0216 | 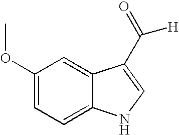 | 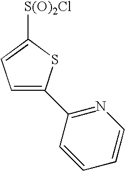 | 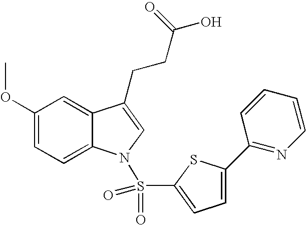 |
| P-0311 | 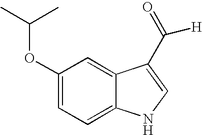 | 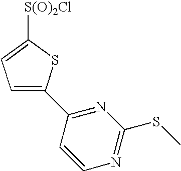 | 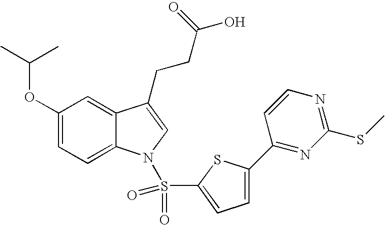 |
| P-0316 | 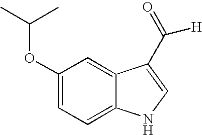 | 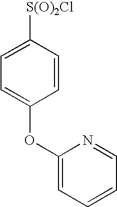 | 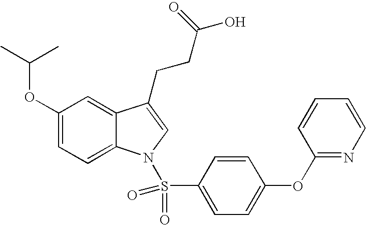 |
| P-0317 | 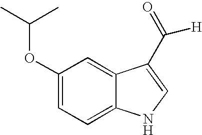 | 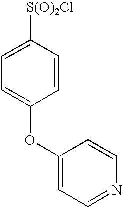 | 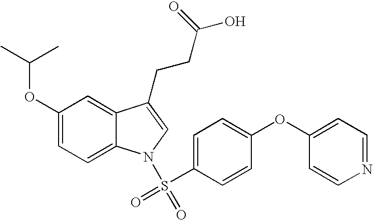 |
| P-0318 | 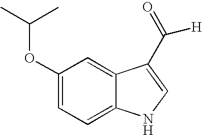 | 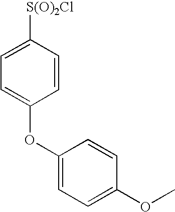 | 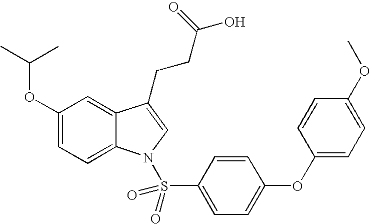 |
| P-0319 | 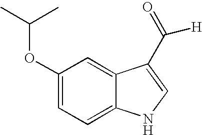 | 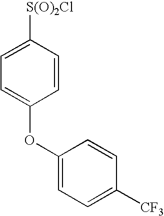 | 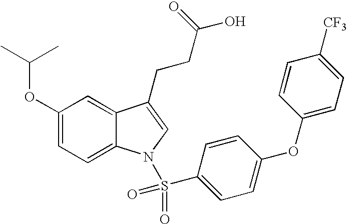 |
| P-0320 | 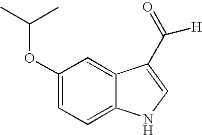 | 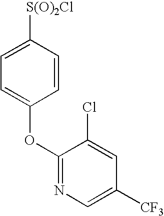 | 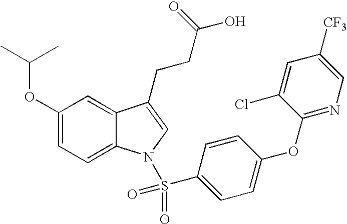 |
| P-0321 | 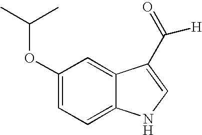 | 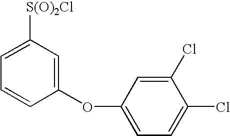 | 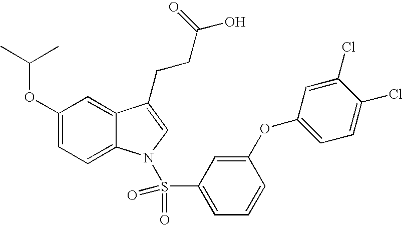 |
| P-0322 | 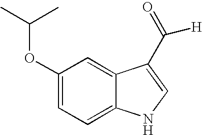 | 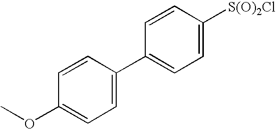 | 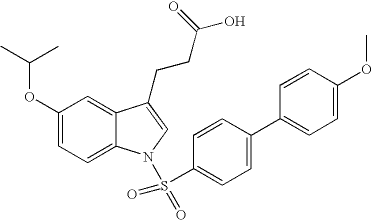 |
| P-0323 | 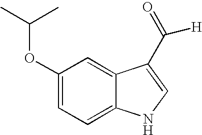 | 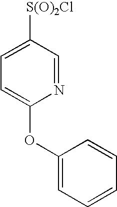 | 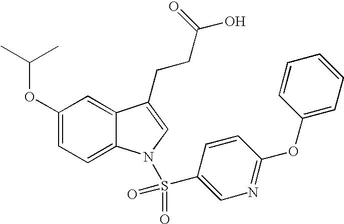 |
| P-0324 | 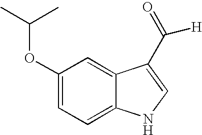 | 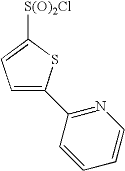 | 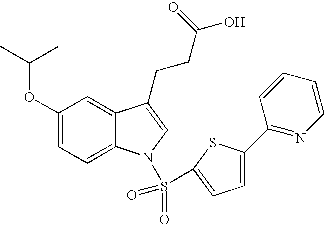 |
| P-0326 | 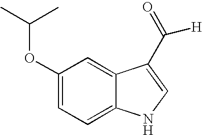 | 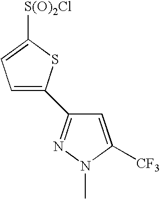 | 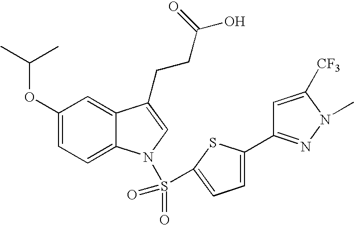 |
| P-0332 | 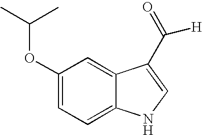 | 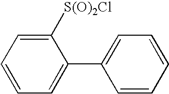 | 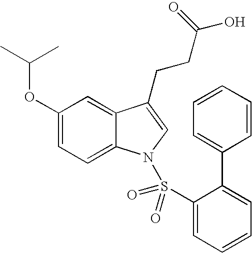 |
| P-0338 | 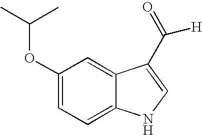 | 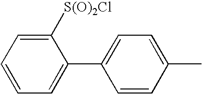 | 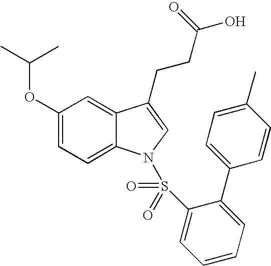 |
| P-0339 | 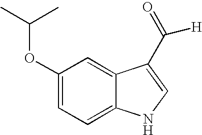 | 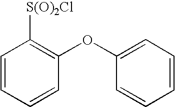 | 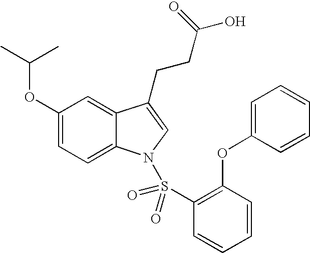 |
| P-0342 | 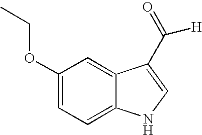 | 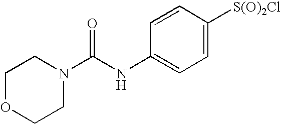 | 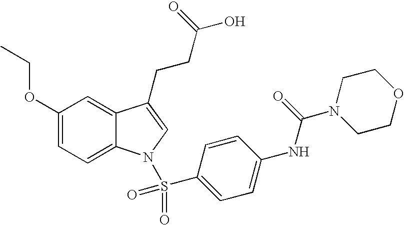 |
| P-0344 | 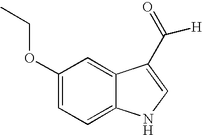 | 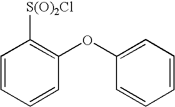 | 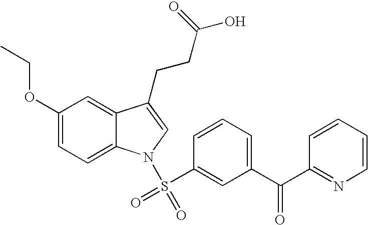 |
| P-0371 | 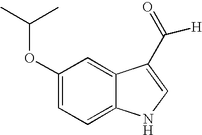 | 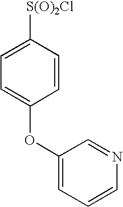 | 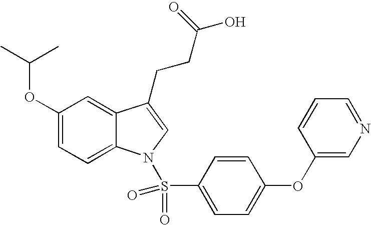 |
| P-0375 | 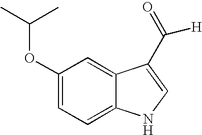 | 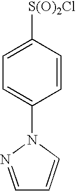 | 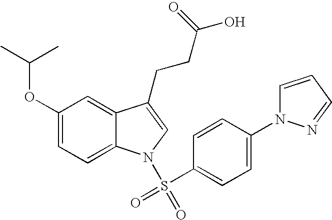 |
| P-0386 | 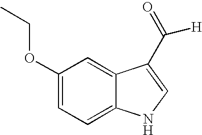 | 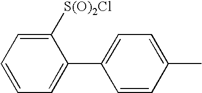 | 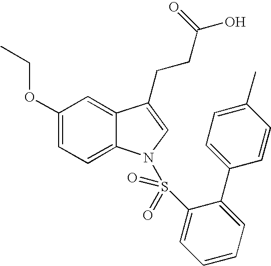 |
| P-0391 | 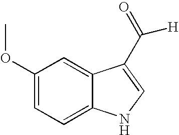 | 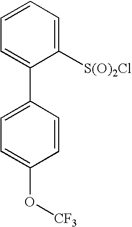 | 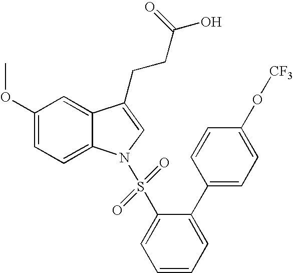 |
| P-0392 | 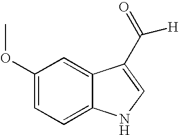 | 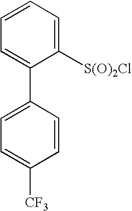 | 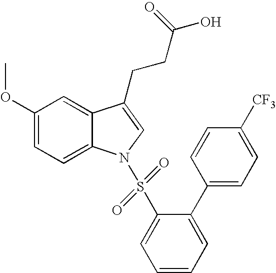 |
| P-0560 | 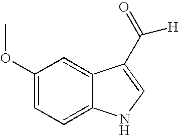 | 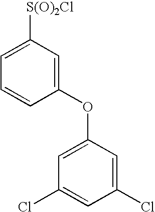 | 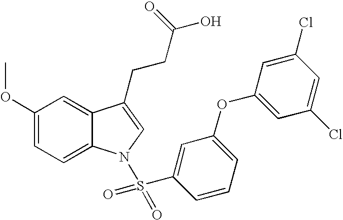 |
| P-0561 | 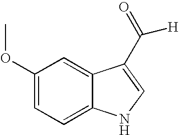 | 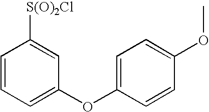 | 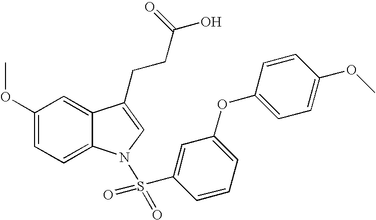 |
| P-0562* | 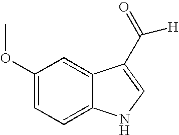 | 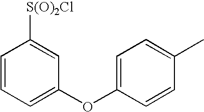 | 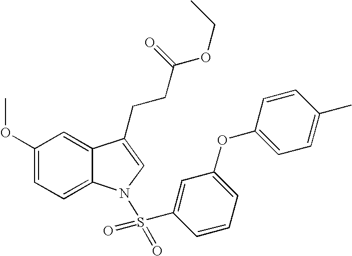 |
| P-0563* | 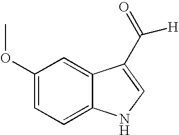 | 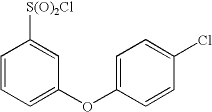 | 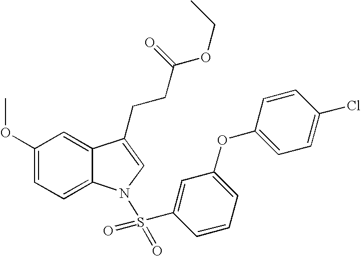 |
| P-0564 | 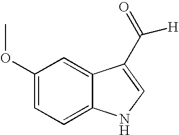 | 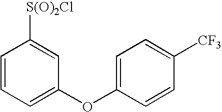 | 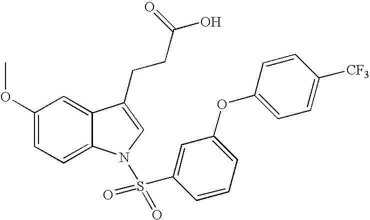 |
| P-0565* | 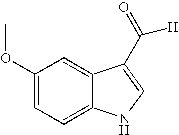 | 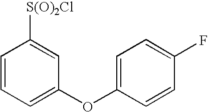 | 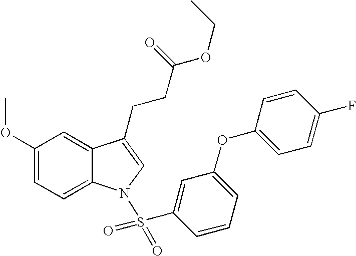 |
| P-0566 | 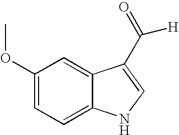 | 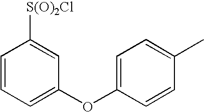 | 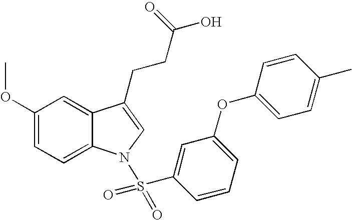 |
| P-0567 | 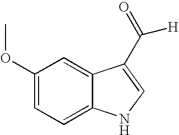 | 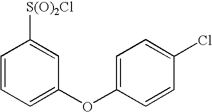 | 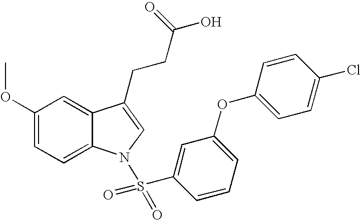 |
| P-0568 | 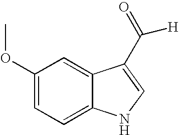 | 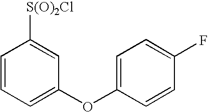 | 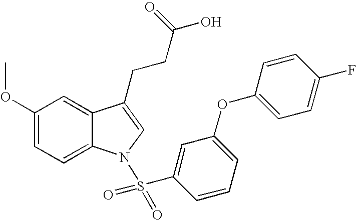 |
| P-0570 | 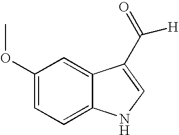 | 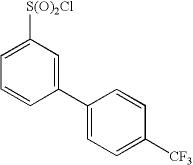 | 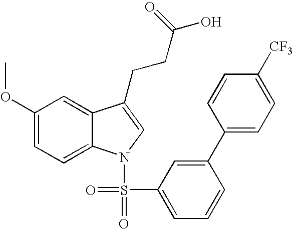 |
| P-0572 | 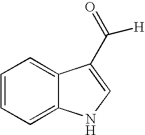 | 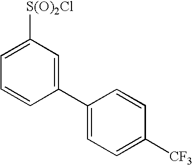 | 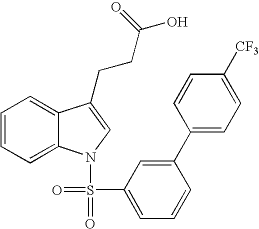 |

*Isolated after Step 3.

Compounds were also prepared by an alternative route to Steps 4 and 5 as shown in Scheme 2a.

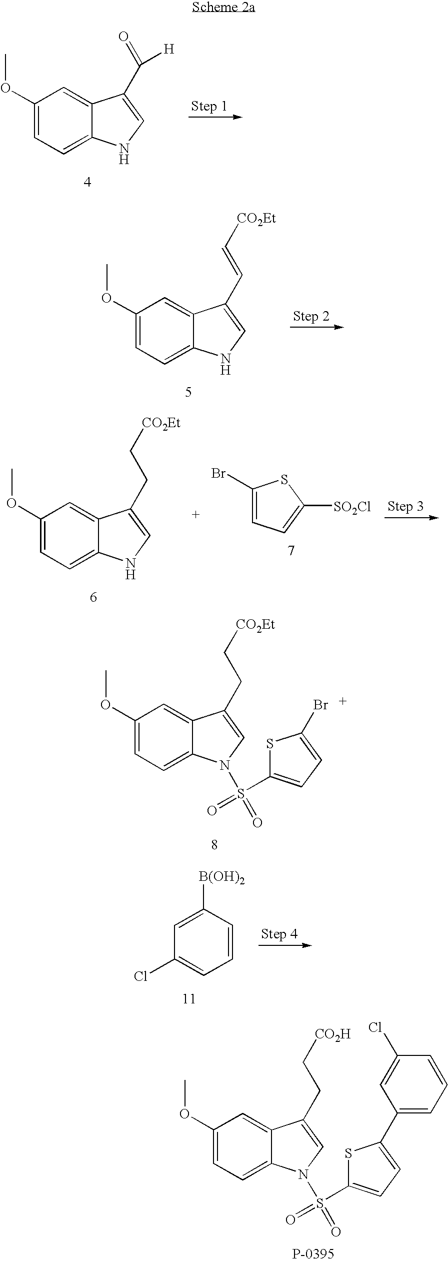

### Step-1 through Step-3: See Scheme 2 above

### Step-4: Synthesis of 3-{1-[5-(3-chloro-phenyl)-thiophene-2-sulfonyl]-5-methoxy-1H-indol-3-yl}-propionic acid (P-0395)

10 mg of 3-[1-(5-bromo-thiophen-2-ylmethyl)-5-methoxy-1H-indol-3-yl]-propionic acid ethyl ester 8 was dissolved in 400 μL of acetonitrile and 2 equivalents of the 3-chloro-phenyl boronic acid 11 was added. 200 μL of 1M K₂CO₃ was added and 10 μL of Pd(AOc)₂/di-tbutylbiphenylphosphine (0.2 M solution in toluene) was added. The reaction mixture was irradiated for 10 minutes at 160° C. in the microwave. The solution was neutralized with acetic acid and the solvents removed under vacuum. The crude material was dissolved in 500 μL of dimethyl sulfoxide and purified by reverse phase HPLC (C18 column), eluting with a water/0.1% trifluoro acetic acid and acetonitrile/0.1% trifluoro acetic acid gradient, 20-100% acetonitrile over 16 minutes. Calculated molecular weight 475.97, [M+H⁺]⁺=475.9.

Additional compounds were prepared following the protocol of Scheme 2a, optionally replacing 5-methoxyindole-3-carboxyaldehyde 4 with an appropriate indole-3-carboxyaldehyde in Step 1, and/or optionally replacing 3-chloro-phenyl boronic acid 11 with an appropriate boronic acid in Step 4. The following compounds were prepared by this method, with the calculated molecular weight and measured mass (MS(ESI)) provided after the compound:
3-{1-[5-(3,5-Bis-trifluoromethyl-phenyl)-thiophene-2-sulfonyl]-5-methoxy-1H-indol-3-yl}-propionic acid (P-0001), calculated MW 577.52, [M−H⁺]⁻=575.96,
3-{1-[5-(4-Trifluoromethoxy-phenyl)-thiophene-2-sulfonyl]-1H-indol-3-yl}-propionic acid methyl ester (P-0038),
3-{1-[5-(3-Trifluoromethoxy-phenyl)-thiophene-2-sulfonyl]-1H-indol-3-yl}-propionic acid methyl ester (P-0388), calculated MW 509.52, [M+H⁺]⁺=510.1,
3-{1-[5-(4-Trifluoromethoxy-phenyl)-thiophene-2-sulfonyl]-1H-indol-3-yl}-propionic acid (P-0393), calculated MW 495.50, [M−H⁺]⁻=494.2,
3-{1-[5-(3-Trifluoromethoxy-phenyl)-thiophene-2-sulfonyl]-1H-indol-3-yl}-propionic acid (P-0394), calculated MW 495.50, [M−H⁺]⁻=494.2,
3-{1-[5-(3-Chloro-phenyl)-thiophene-2-sulfonyl]-5-ethoxy-1H-indol-3-yl}-propionic acid (P-0396), calculated MW 490.00, [M+H⁺]⁺=490.3,
3-{5-Chloro-1-[5-(3-chloro-phenyl)-thiophene-2-sulfonyl]-1H-indol-3-yl}-propionic acid (P-0397), calculated MW 480.39, [M−H⁺]⁻=476.7,
3-{1-[5-(3-Chloro-phenyl)-thiophene-2-sulfonyl]-5-fluoro-1H-indol-3-yl}-propionic acid (P-0398), calculated MW 463.94, [M+H⁺]⁺=466.3,
3-{1-[5-(4-Chloro-phenyl)-thiophene-2-sulfonyl]-5-methoxy-1H-indol-3-yl}-propionic acid (P-0399), calculated MW 475.97, [M+H⁺]⁺=475.5,
3-{1-[5-(4-Chloro-phenyl)-thiophene-2-sulfonyl]-5-ethoxy-1H-indol-3-yl}-propionic acid (P-0400), calculated MW 490.00, [M−H⁺]⁻=489.9,
3-{5-Chloro-1-[5-(4-chloro-phenyl)-thiophene-2-sulfonyl]-1H-indol-3-yl}-propionic acid (P-0401), calculated MW 480.39, [M+H⁺]⁺=481.5,
3-{1-[5-(4-Chloro-phenyl)-thiophene-2-sulfonyl]-5-fluoro-1H-indol-3-yl}-propionic acid (P-0402), calculated MW 463.94, [M−H⁺]⁻=461.1,
3-[1-(5-Furan-2-yl-thiophene-2-sulfonyl)-5-methoxy-1H-indol-3-yl]-propionic acid (P-0403), calculated MW 431.49, [M+H⁺]⁺=432.3,
3-[5-Ethoxy-1-(5-furan-2-yl-thiophene-2-sulfonyl)-1H-indol-3-yl]-propionic acid (P-0404), calculated MW 445.51, [M+H^{+l ]}⁺=445.9,
3-[5-Chloro-1-(5-furan-2-yl-thiophene-2-sulfonyl)-1H-indol-3-yl]-propionic acid (P-0405), calculated MW 435.91, [M+H⁺]⁺=435.9,
3-[5-Fluoro-1-(5-furan-2-yl-thiophene-2-sulfonyl)-1H-indol-3-yl]-propionic acid (P-0406), calculated MW 419.45, [M+H⁺]⁺=419.9,
3-[1-(5-Furan-3-yl-thiophene-2-sulfonyl)-5-methoxy-1H-indol-3-yl]-propionic acid (P-0407), calculated MW 431.49, [M+H⁺]⁺=432.3,
3-[5-Ethoxy-1-(5-furan-3-yl-thiophene-2-sulfonyl)-1H-indol-3-yl]-propionic acid (P-0408), calculated MW 445.51, [M+H⁺]⁺=445.9,
3-[5-Chloro-1-(5-furan-3-yl-thiophene-2-sulfonyl)-1H-indol-3-yl]-propionic acid (P-0409), calculated MW 435.91, [M−H⁺]⁻=433.9,
3-[5-Fluoro-1-(5-furan-3-yl-thiophene-2-sulfonyl)-1H-indol-3-yl]-propionic acid (P-0410), calculated MW 419.45, [M+H⁺]⁺=420.3,
3-[5-Methoxy-1-(5-pyridin-3-yl-thiophene-2-sulfonyl)-1H-indol-3-yl]-propionic acid (P-0411), calculated MW 442.51, [M+H⁺]⁺=443.1,
3-[5-Ethoxy-1-(5-pyridin-3-yl-thiophene-2-sulfonyl)-1H-indol-3-yl]-propionic acid (P-0412), calculated MW 456.54, [M+H⁺]⁺=457.1,
3-[5-Chloro-1-(5-pyridin-3-yl-thiophene-2-sulfonyl)-1H-indol-3-yl]-propionic acid (P-0413), calculated MW 446.93, [M+H⁺]⁺=447.1,
3-[5-Fluoro-1-(5-pyridin-3-yl-thiophene-2-sulfonyl)-1H-indol-3-yl]-propionic acid (P-0414), calculated MW 430.48, [M+H⁺]⁺=431.1,
3-[5-Ethoxy-1-(5-pyridin-4-yl-thiophene-2-sulfonyl)-1H-indol-3-yl]-propionic acid (P-0415), calculated MW 456.54, [M+H⁺]⁺=457.1,
3-[5-Chloro-1-(5-pyridin-4-yl-thiophene-2-sulfonyl)-1H-indol-3-yl]-propionic acid (P-0416), calculated MW 446.93, [M+H⁺]⁺=447.1,
3-[5-Fluoro-1-(5-pyridin-4-yl-thiophene-2-sulfonyl)-1H-indol-3-yl]-propionic acid (P-0417), calculated MW 430.48, [M+H⁺]⁺=431.1,
3-{1-[5-(3,5-Dichloro-phenyl)-thiophene-2-sulfonyl]-5-methoxy-1H-indol-3-yl}-propionic acid (P-0418), calculated MW 510.42, [M−H⁺]⁻=509.9,
3-{1-[5-(3,5-Dichloro-phenyl)-thiophene-2-sulfonyl]-5-ethoxy-1H-indol-3-yl}-propionic acid (P-0419), calculated MW 524.44, [M+H⁺]⁺=524.3,
3-{5-Chloro-1-[5-(3,5-dichloro-phenyl)-thiophene-2-sulfonyl]-1H-indol-3-yl}-propionic acid (P-0420), calculated MW 514.84, [M−H⁺]⁻=507.1,
3-{1-[5-(3,5-Dichloro-phenyl)-thiophene-2-sulfonyl]-5-fluoro-1H-indol-3-yl}-propionic acid (P-0421), calculated MW 498.38, [M−H⁺]⁻=490.3,
3-{1-[5-(3,4-Difluoro-phenyl)-thiophene-2-sulfonyl]-5-methoxy-1H-indol-3-yl}-propionic acid (P-0422), calculated MW 477.51, [M+H⁺]⁺=478.3,
3-{1-[5-(3,4-Difluoro-phenyl)-thiophene-2-sulfonyl]-5-ethoxy-1H-indol-3-yl}-propionic acid (P-0423), calculated MW 491.53, [M+H⁺]⁺=492.3,
3-{5-Chloro-1-[5-(3,4-difluoro-phenyl)-thiophene-2-sulfonyl]-1H-indol-3-yl}-propionic acid (P-0424), calculated MW 481.93, [M+H⁺]⁺=481.1,
3-{1-[5-(3,4-Difluoro-phenyl)-thiophene-2-sulfonyl]-5-fluoro-1H-indol-3-yl}-propionic acid (P-0425), calculated MW 465.47, [M−H⁺]⁻=464.7,
3-{1-[5-(3,4-Dimethoxy-phenyl)-thiophene-2-sulfonyl]-5-methoxy-1H-indol-3-yl}-propionic acid (P-0426), calculated MW 501.58, [M+H⁺]⁺=501.9,
3-{1-[5-(3,4-Dimethoxy-phenyl)-thiophene-2-sulfonyl]-5-ethoxy-1H-indol-3-yl}-propionic acid (P-0427), calculated MW 515.60, [M+H⁺]⁺=516.3,
3-{5-Chloro-1-[5-(3,4-dimethoxy-phenyl)-thiophene-2-sulfonyl]-1H-indol-3-yl}-propionic acid (P-0428), calculated MW 506.00, [M+H⁺]⁺=507.5,
3-{1-[5-(3,4-Dimethoxy-phenyl)-thiophene-2-sulfonyl]-5-fluoro-1H-indol-3-yl}-propionic acid (P-0429), calculated MW 489.54, [M−H⁺]⁻=485.5,
3-{1-[5-(4-Fluoro-phenyl)-thiophene-2-sulfonyl]-5-methoxy-1H-indol-3-yl}-propionic acid (P-0430), calculated MW 459.52, [M+H⁺]⁺=459.9,
3-{5-Ethoxy-1-[5-(4-fluoro-phenyl)-thiophene-2-sulfonyl]-1H-indol-3-yl}-propionic acid (P-0431), calculated MW 473.54, [M+H⁺]⁺=473.9,
3-{5-Chloro-1-[5-(4-fluoro-phenyl)-thiophene-2-sulfonyl]-1H-indol-3-yl}-propionic acid (P-0432), calculated MW 463.94, [M−H⁺]⁻=458.7,
3-{5-Fluoro-1-[5-(4-fluoro-phenyl)-thiophene-2-sulfonyl]-1H-indol-3-yl}-propionic acid (P-0433), calculated MW 447.48, [M+H⁺]⁺=447.9,
3-{5-Methoxy-1-[5-(4-methylsulfanyl-phenyl)-thiophene-2-sulfonyl]-1H-indol-3-yl}-propionic acid (P-0434), calculated MW 487.62, [M+H⁺]⁺=487.9,
3-{5-Ethoxy-1-[5-(4-methylsulfanyl-phenyl)-thiophene-2-sulfonyl]-1H-indol-3-yl}-propionic acid (P-0435), calculated MW 501.65, [M+H⁺]⁺=501.9,
3-{5-Chloro-1-[5-(4-methylsulfanyl-phenyl)-thiophene-2-sulfonyl]-1H-indol-3-yl}-propionic acid (P-0436), calculated MW 492.04, [M−H⁺]⁻=487.9,
3-{5-Fluoro-1-[5-(4-methylsulfanyl-phenyl)-thiophene-2-sulfonyl]-1H-indol-3-yl}-propionic acid (P-0437), calculated MW 475.58, [M−H⁺]⁻=473.9,
3-{1-[5-(3-Chloro-4-fluoro-phenyl)-thiophene-2-sulfonyl]-5-methoxy-1H-indol-3-yl}-propionic acid (P-0438), calculated MW 493.96, [M+H⁺]⁺=493.9,
3-{1-[5-(3 -Chloro-4-fluoro-phenyl)-thiophene-2-sulfonyl]-5-ethoxy-1H-indol-3-yl}-propionic acid (P-0439), calculated MW 507.99, [M+H⁺]⁺=507.5,
3-{5-Chloro-1-[5-(3-chloro-4-fluoro-phenyl)-thiophene-2-sulfonyl]-1H-indol-3-yl}-propionic acid (P-0440), calculated MW 498.38, [M+H⁺]⁺=503.5,
3-{1-[5-(3-Chloro-4-fluoro-phenyl)-thiophene-2-sulfonyl]-5-fluoro-1H-indol-3-yl}-propionic acid (P-0441), calculated MW 481.93, [M−H⁺]⁻=479.1,
3-[5-Methoxy-1-(5-pyrimidin-5-yl-thiophene-2-sulfonyl)-1H-indol-3-yl]-propionic acid (P-0442), calculated MW 443.50, [M+H⁺]⁺=444.3,
3-[5-Ethoxy-1-(5-pyrimidin-5-yl-thiophene-2-sulfonyl)-1H-indol-3-yl]-propionic acid (P-0443), calculated MW 457.53, [M+H⁺]⁺=458.3,
3-[5-Chloro-1-(5-pyrimidin-5-yl-thiophene-2-sulfonyl)-1H-indol-3-yl]-propionic acid (P-0444), calculated MW 447.92, [M+H⁺]⁺=447.9,
3-[5-Fluoro-1-(5-pyrimidin-5-yl-thiophene-2-sulfonyl)-1H-indol-3-yl]-propionic acid (P-0445), calculated MW 431.47, [M+H⁺]⁺=432.3,
3-{5-Methoxy-1-[5-(6-methoxy-pyridin-3-yl)-thiophene-2-sulfonyl]-1H-indol-3-yl}-propionic acid (P-0446), calculated MW 472.54, [M+H⁺]⁺=473.1,
3-{5-Ethoxy-1-[5-(6-methoxy-pyridin-3-yl)-thiophene-2-sulfonyl]-1H-indol-3-yl}-propionic acid (P-0447), calculated MW 486.57, [M+H⁺]⁺=487.1,
3-{5-Chloro-1-[5-(6-methoxy-pyridin-3-yl)-thiophene-2-sulfonyl]-1H-indol-3-yl}-propionic acid (P-0448), calculated MW 476.96, [M+H⁺]⁺=477.1,
3-{5-Fluoro-1-[5-(6-methoxy-pyridin-3-yl)-thiophene-2-sulfonyl]-1H-indol-3-yl}-propionic acid (P-0449), calculated MW 460.50, [M+H⁺]⁺=461.1,
3-{5-Methoxy-1-[5-(1H-pyrazol-4-yl)-thiophene-2-sulfonyl]-1H-indol-3-yl}-propionic acid (P-0450), calculated MW 431.49, [M+H⁺]⁺=432.3,
3-{5-Ethoxy-1-[5-(1 H-pyrazol-4-yl)-thiophene-2-sulfonyl]-1H-indol-3-yl}-propionic acid (P-0451), calculated MW 445.52, [M+H⁺]⁺=445.9,
3-{5-Chloro-1-[5-(1H-pyrazol-4-yl)-thiophene-2-sulfonyl]-1H-indol-3-yl}-propionic acid (P-0452), calculated MW 435.91, [M+H⁺]⁺=435.9,
3-{5-Fluoro-1-[5-( 1H-pyrazol-4-yl)-thiophene-2-sulfonyl]-1H-indol-3-yl}-propionic acid (P-0453), calculated MW 419.46, [M+H⁺]⁺=419.9,
3-{5-Methoxy-1-[5-(1-methyl-1H-pyrazol-4-yl)-thiophene-2-sulfonyl]-1H-indol-3-yl}-propionic acid (P-0454), calculated MW 445.52, [M+H⁺]⁺=445.9,
3-{5-Ethoxy-1-[5-(1-methyl-1H-pyrazol-4-yl)-thiophene-2-sulfonyl]-1H-indol-3-yl}-propionic acid (P-0455), calculated MW 459.54, [M+H⁺]⁺=460.3,
3-{5-Chloro-1-[5-(1-methyl-1H-pyrazol-4-yl)-thiophene-2-sulfonyl]-1H-indol-3-yl}-propionic acid (P-0456), calculated MW 449.94, [M+H⁺]⁺=449.9,
3-{5-Fluoro-1-[5-(1-methyl-1H-pyrazol-4-yl)-thiophene-2-sulfonyl]-1H-indol-3-yl}-propionic acid (P-0457), calculated MW 433.48, [M+H⁺]⁺=434.3,
3-{1-[5-(3-Dimethylamino-phenyl)-thiophene-2-sulfonyl]-5-methoxy-1H-indol-3-yl}-propionic acid (P-0458), calculated MW 484.59, [M+H⁺]⁺=485.1,
3-{1-[5-(3 -Dimethylamino-phenyl)-thiophene-2-sulfonyl]-5-ethoxy-1H-indol-3-yl}-propionic acid (P-0459), calculated MW 498.62, [M+H⁺]⁺=499.1,
3-{5-Chloro-1-[5-(3-dimethylamino-phenyl)-thiophene-2-sulfonyl]-1H-indol-3-yl}-propionic acid (P-0460), calculated MW 489.01, [M+H⁺]⁺=489.1,
3-{1-[5-(3-Dimethylamino-phenyl)-thiophene-2-sulfonyl]-5-fluoro-1H-indol-3-yl}-propionic acid (P-0461), calculated MW 472.56, [M+H⁺]⁺=473.1,
3-{1-[5-(2,6-Dimethoxy-pyridin-3-yl)-thiophene-2-sulfonyl]-5-methoxy-1H-indol-3-yl}-propionic acid (P-0462), calculated MW 502.57, [M+H⁺]⁺=503.1,
3-{1-[5-(2,6-Dimethoxy-pyridin-3-yl)-thiophene-2-sulfonyl]-5-ethoxy-1H-indol-3-yl}-propionic acid (P-0463), calculated MW 516.59, [M+H⁺]⁺=517.1,
3-{5-Chloro-1-[5-(2,6-dimethoxy-pyridin-3-yl)-thiophene-2-sulfonyl]-1H-indol-3-yl}-propionic acid (P-0464), calculated MW 506.99, [M+H⁺]⁺=507.1,
3-{1-[5-(2,6-Dimethoxy-pyridin-3-yl)-thiophene-2-sulfonyl]-5-fluoro-1H-indol-3-yl}-propionic acid (P-0465), calculated MW 490.53, [M+H⁺]⁺=491.1,
3-{1-[5-(2,4-Dimethoxy-pyrimidin-5-yl)-thiophene-2-sulfonyl]-5-methoxy-1H-indol-3-yl}-propionic acid (P-0466), calculated MW 503.55, [M+H⁺]⁺=503.9,
3-{1-[5-(2,4-Dimethoxy-pyrimidin-5-yl)-thiophene-2-sulfonyl]-5-ethoxy-1H-indol-3-yl}-propionic acid (P-0467), calculated MW 517.58, [M+H⁺]⁺=517.9,
3-{5-Chloro-1-[5-(2,4-dimethoxy-pyrimidin-5-yl)-thiophene-2-sulfonyl]-1H-indol-3-yl}-propionic acid (P-0468), calculated MW 507.97, [M+H⁺]⁺=507.9,
3-{1-[5-(2,4-Dimethoxy-pyrimidin-5-yl)-thiophene-2-sulfonyl]-5-fluoro-1H-indol-3-yl}-propionic acid (P-0469), calculated MW 491.52, [M+H⁺]⁺=491.1,
3-{1-[5-(6-Benzyloxy-pyridin-3-yl)-thiophene-2-sulfonyl]-5-methoxy-1H-indol-3-yl}-propionic acid (P-0470), calculated MW 548.64, [M+H⁺]⁺=549.1,
3-{1-[5-(6-Benzyloxy-pyridin-3-yl)-thiophene-2-sulfonyl]-5-ethoxy-1H-indol-3-yl}-propionic acid (P-0471), calculated MW 562.66, [M+H⁺]⁺=563.2,
3-{1-[5-(6-Benzyloxy-pyridin-3-yl)-thiophene-2-sulfonyl]-5-chloro-1H-indol-3-yl}-propionic acid (P-0472), calculated MW 553.06, [M+H⁺]⁺=553.2,
3-{1-[5-(6-Benzyloxy-pyridin-3-yl)-thiophene-2-sulfonyl]-5-fluoro-1H-indol-3-yl}-propionic acid (P-0473), calculated MW 536.60, [M+H⁺]⁺=537.1,
3-{5-Ethoxy-1-[5-(4-ethoxy-phenyl)-thiophene-2-sulfonyl]-1H-indol-3-yl}-propionic acid (P-0474), calculated MW 499.61, [M+H⁺]⁺=499.9,
3-{5-Chloro-1-[5-(4-ethoxy-phenyl)-thiophene-2-sulfonyl]-1H-indol-3-yl }-propionic acid (P-0475), calculated MW 490.00, [M−H⁺]⁻=489.9,
3-{1-[5-(4-Ethoxy-phenyl)-thiophene-2-sulfonyl]-5-fluoro-1H-indol-3-yl}-propionic acid (P-0476), calculated MW 473.54, [M+H⁺]⁺=473.9,
3-{1-[5-(3-Fluoro-phenyl)-thiophene-2-sulfonyl]-5-methoxy-1H-indol-3-yl}-propionic acid (P-0477), calculated MW 459.52, [M+H⁺]⁺=460.3,
3-{5-Ethoxy-1-[5-(3 -fluoro-phenyl)-thiophene-2-sulfonyl]-1H-indol-3-yl }-propionic acid (P-0478), calculated MW 473.54, [M+H⁺]⁺=473.9,
3-{5-Chloro-1-[5-(3-fluoro-phenyl)-thiophene-2-sulfonyl]-1H-indol-3-yl}-propionic acid (P-0479), calculated MW 463.94, [M−H⁺]⁻=457.5,
3-{5-Fluoro-1-[5-(3-fluoro-phenyl)-thiophene-2-sulfonyl]-1H-indol-3-yl}-propionic acid (P-0480), calculated MW 447.48, [M+H⁺]⁺=447.9,
3-{5-Ethoxy-1-[5-(3-trifluoromethoxy-phenyl)-thiophene-2-sulfonyl]-1H-indol-3-yl}-propionic acid (P-0481), calculated MW 539.55, [M+H⁺]⁺=539.9,
3-{5-Chloro-1-[5-(3-trifluoromethoxy-phenyl)-thiophene-2-sulfonyl]-1H-indol-3-yl}-propionic acid (P-0482), calculated MW 529.94, [M+H⁺]⁺=525.9,
3-{5-Fluoro-1-[5-(3-trifluoromethoxy-phenyl)-thiophene-2-sulfonyl]-1H-indol-3-yl}-propionic acid (P-0483), calculated MW 513.49, [M+H⁺]⁺=514.3,
3-{1-[5-(3,4-Dichloro-phenyl)-thiophene-2-sulfonyl]-5-methoxy-1H-indol-3-yl}-propionic acid (P-0484), calculated MW 510.42, [M−H⁺]⁻=509.9,
3-{1-[5-(3,4-Dichloro-phenyl)-thiophene-2-sulfonyl]-5-ethoxy-1H-indol-3-yl}-propionic acid (P-0485), calculated MW 524.44, [M+H⁺]⁺=524.3,
3-{5-Chloro-1-[5-(3,4-dichloro-phenyl)-thiophene-2-sulfonyl]-1H-indol-3-yl}-propionic acid (P-0486), calculated MW 514.84, [M+H⁺]⁺=511.9,
3-{1-[5-(3,4-Dichloro-phenyl)-thiophene-2-sulfonyl]-5-fluoro-1H-indol-3-yl}-propionic acid (P-0487), calculated MW 498.38, [M−H⁺]⁻=496.3,
3-{5-Ethoxy-1-[5-(3 -trifluoromethyl-phenyl)-thiophene-2-sulfonyl]-1H-indol-3-yl}-propionic acid (P-0488), calculated MW 523.55, [M+H⁺]⁺=524.3,
3-{5-Chloro-1-[5-(3-trifluoromethyl-phenyl)-thiophene-2-sulfonyl]-1H-indol-3-yl}-propionic acid (P-0489), calculated MW 513.94, [M−H⁺]⁻=511.9,
3-{5-Fluoro-1-[5-(3-trifluoromethyl-phenyl)-thiophene-2-sulfonyl]-1H-indol-3-yl}-propionic acid (P-0490), calculated MW 497.49, [M+H⁺]⁺=497.9,
3-{1-[5-(4-Benzyloxy-phenyl)-thiophene-2-sulfonyl]-5-ethoxy-1H-indol-3-yl}-propionic acid (P-0491), calculated MW 561.68, [M+H⁺]⁺=562.0,
3-{1-[5-(4-Benzyloxy-phenyl)-thiophene-2-sulfonyl]-5-chloro-1H-indol-3-yl}-propionic acid (P-0492), calculated MW 552.07, [M+H⁺]⁺=553.6,
3-{1-[5-(4-Benzyloxy-phenyl)-thiophene-2-sulfonyl]-5-fluoro-1H-indol-3-yl}-propionic acid (P-0493), calculated MW 535.61, [M+H⁺]⁺=535.9,
3-{5-Ethoxy-1-[5-(4-trifluoromethyl-phenyl)-thiophene-2-sulfonyl]-1H-indol-3-yl}-propionic acid (P-0494), calculated MW 523.55, [M+H⁺]⁺=524.3,
3-{5-Chloro-1-[5-(4-trifluoromethyl-phenyl)-thiophene-2-sulfonyl]-1H-indol-3-yl}-propionic acid (P-0495), calculated MW 513.94, [M−H⁺]⁻=512.3,
3-{5-Fluoro-1-[5-(4-trifluoromethyl-phenyl)-thiophene-2-sulfonyl]-1H-indol-3-yl}-propionic acid (P-0496), calculated MW 497.49, [M−H⁺]⁻=490.3,
3-{1-[5-(3Fluoro-4-methoxy-phenyl)-thiophene-2-sulfonyl]-5-methoxy-1H-indol-3-yl}-propionic acid (P-0497), calculated MW 489.54, [M+H⁺]⁺=490.3,
3-{5-Ethoxy-1-[5-(3-fluoro-4-methoxy-phenyl)-thiophene-2-sulfonyl]-1H-indol-3-yl}-propionic acid (P-0498), calculated MW 503.57, [M+H⁺]⁺=503.9,
3-{5-Chloro-1-[5-(3-fluoro-4-methoxy-phenyl)-thiophene-2-sulfonyl]-1H-indol-3-yl}-propionic acid (P-0499), calculated MW 493.96, [M+H⁺]⁺=497.1,
3-{5-Fluoro-1-[5-(3-fluoro-4-methoxy-phenyl)-thiophene-2-sulfonyl]-1H-indol-3-yl}-propionic acid (P-0500), calculated MW 477.51, [M+H⁺]⁺=478.3,
3-{5-Methoxy-1-[5-(5-methyl-furan-2-yl)-thiophene-2-sulfonyl]-1H-indol-3-yl}-propionic acid (P-0501), calculated MW 445.51, [M+H⁺]⁺=445.9,
3-{5-Ethoxy-1-[5-(5-methyl-furan-2-yl)-thiophene-2-sulfonyl]-1H-indol-3-yl}-propionic acid (P-0502), calculated MW 459.54, [M+H⁺]⁺=459.9,
3-{5-Chloro-1-[5-(5-methyl-furan-2-yl)-thiophene-2-sulfonyl]-1H-indol-3-yl }-propionic acid (P-0503), calculated MW 449.93, [M+H⁺]⁺=449.5,
3-{1-[5-(3,5-Dimethyl-isoxazol-4-yl)-thiophene-2-sulfonyl]-5-methoxy-1H-indol-3-yl}-propionic acid (P-0504), calculated MW 460.53, [M+H⁺]⁺=461.1,
3-{1-[5-(3,5-Dimethyl-isoxazol-4-yl)-thiophene-2-sulfonyl]-5-ethoxy-1H-indol-3-yl}-propionic acid (P-0505), calculated MW 474.56, [M+H⁺]⁺=475.1,
3-{5-Chloro-1-[5-(3,5-dimethyl-isoxazol-4-yl)-thiophene-2-sulfonyl]-1H-indol-3-yl}-propionic acid (P-0506), calculated MW 464.95, [M+H⁺]⁺=464.7,
3-{1-[5-(3,5-Dimethyl-isoxazol-4-yl)-thiophene-2-sulfonyl]-5-fluoro-1H-indol-3-yl}-propionic acid (P-0507), calculated MW 448.49, [M+H⁺]⁺=448.7,
3-{1-[5-(4-Chloro-3-trifluoromethyl-phenyl)-thiophene-2-sulfonyl]-5-methoxy-1H-indol-3-yl}-propionic acid (P-0508), calculated MW 543.97, [M+H⁺]⁺=543.9,
3-{1-[5-(4-Chloro-3-trifluoromethyl-phenyl)-thiophene-2-sulfonyl]-5-ethoxy-1H-indol-3-yl}-propionic acid (P-0509), calculated MW 558.00, [M+H⁺]⁺=558.0,
3-{5-Chloro-1-[5-(4-chloro-3trifluoromethyl-phenyl)-thiophene-2-sulfonyl]-1H-indol-3-yl}-propionic acid (P-0510), calculated MW 548.39, [M−H⁺]⁻=546.7,
3-{1-[5-(4-Chloro-3-trifluoromethyl-phenyl)-thiophene-2-sulfonyl]-5-fluoro-1H-indol-3-yl}-propionic acid (P-0511), calculated MW 531.93, [M+H⁺]⁺=531.9,
3-{5-Methoxy-1-[5-(4-morpholin-4-yl-phenyl)-thiophene-2-sulfonyl]-1H-indol-3-yl}-propionic acid (P-0512), calculated MW 526.63, [M+H⁺]⁺=527.1,
3-{5-Ethoxy-1-[5-(4-morpholin-4-yl-phenyl)-thiophene-2-sulfonyl]-1H-indol-3-yl}-propionic acid (P-0513), calculated MW 540.66, [M+H⁺]⁺=541.1,
3-{5-Chloro-1-[5-(4-morpholin-4-yl-phenyl)-thiophene-2-sulfonyl]-1H-indol-3-yl}propionic acid (P-0514), calculated MW 531.05, [M+H⁺]⁺=531.1,
3-{1-[5-(2-Chloro-4-trifluoromethyl-phenyl)-thiophene-2-sulfonyl]-5-methoxy-1H-indol-3-yl}-propionic acid (P-0515), calculated MW 543.97, [M+H⁺]⁺=543.9,
3-{1-[5-(2-Chloro-4-trifluoromethyl-phenyl)-thiophene-2-sulfonyl]-5-ethoxy-1H-indol-3-yl}-propionic acid (P-0516), calculated MW 558.00, [M+H⁺]⁺=558.0,
3-{5-Chloro-1-[5-(2-chloro-4-trifluoromethyl-phenyl)-thiophene-2-sulfonyl]-1H-indol-3-yl}-propionic acid (P-0517), calculated MW 548.39, [M+H⁺]⁺=548.3,
3-{1-[5-(2-Chloro-4-trifluoromethyl-phenyl)-thiophene-2-sulfonyl]-5-fluoro-1H-indol-3-yl}-propionic acid (P-0518), calculated MW 531.93, [M+H⁺]⁺=531.9,
3-{5-Methoxy-1-[5-(1-propyl-1H-pyrazol-4-yl)-thiophene-2-sulfonyl]-1H-indol-3-yl}-propionic acid (P-0519), calculated MW 473.57, [M+H⁺]⁺=473.9,
3-{5-Ethoxy-1-[5-(1-propyl-1H-pyrazol-4-yl)-thiophene-2-sulfonyl]-1H-indol-3-yl}-propionic acid (P-0520), calculated MW 487.60, [M+H⁺]⁺=487.9,
3-{5-Chloro-1-[5-(1-propyl-1H-pyrazol-4-yl)-thiophene-2-sulfonyl]-1H-indol-3-yl}-propionic acid (P-0521), calculated MW 477.99, [M+H⁺]⁺=477.9,
3-{5-Fluoro-1-[5-(1-propyl-1H-pyrazol-4-yl)-thiophene-2-sulfonyl]-1H-indol-3-yl}-propionic acid (P-0522), calculated MW 461.54, [M+H⁺]⁺=462.3, and
3-(5-Methoxy-1-{5-[4-(2,2,2-trifluoro-ethoxy)-phenyl]-thiophene-2-sulfonyl}-1H-indol-3-yl)-propionic acid (P-0523), calculated MW 539.55, [M−H⁺]⁻=538.06.

These compounds are shown in the following Table 3, indicating the indole-3-carboxyaldehyde used in Step 1 (Column 2), the boronic acid used in Step 4 (Column 3), and the compound structure (Column 4), with the compound number provided in Column 1.

TABLE 3

| Cmpd. | | | |
|---|---|---|---|
| number | Indole-3-carboxyaldehyde | Boronic acid | Compound structure |
| P-0001 | 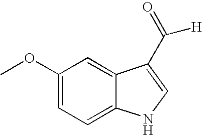 | 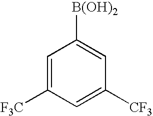 | 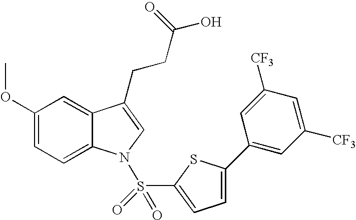 |
| P-0038 | 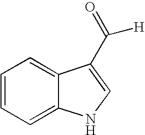 | 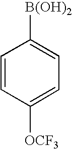 | 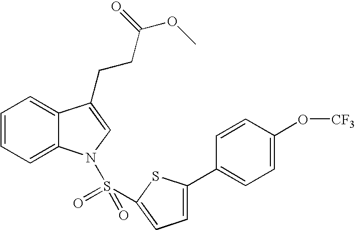 |
| P-0388 | 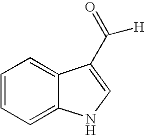 | 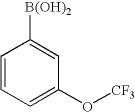 | 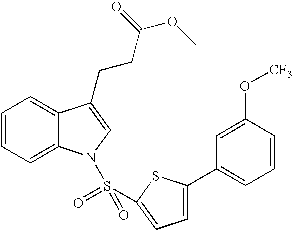 |
| P-0393 | 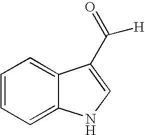 | 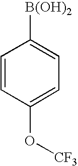 | 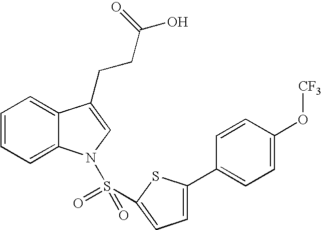 |
| P-0394 | 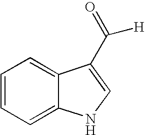 | 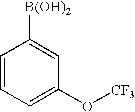 | 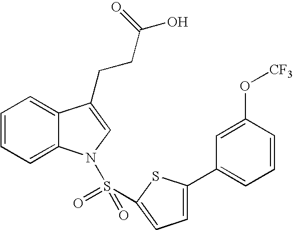 |
| P-0395 | | 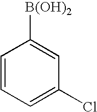 | 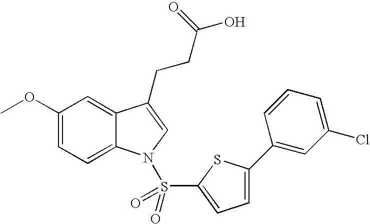 |
| P-0396 | 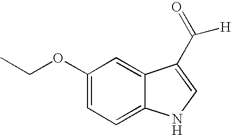 | 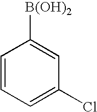 | 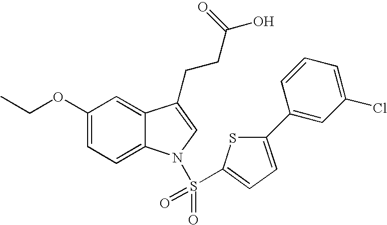 |
| P-0397 | 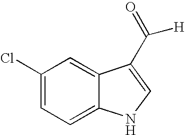 | 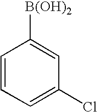 | 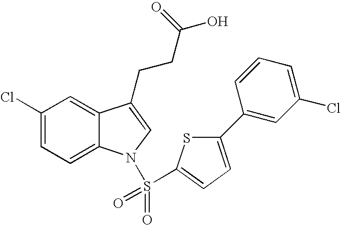 |
| P-0398 | 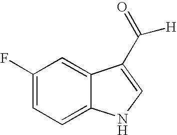 | 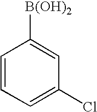 | 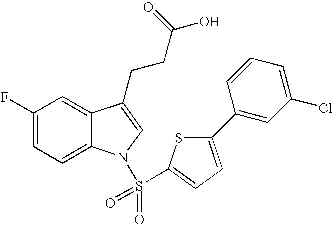 |
| P-0399 | 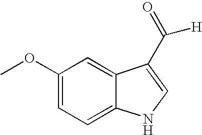 | 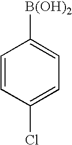 | 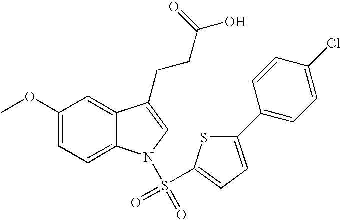 |
| P-0400 | 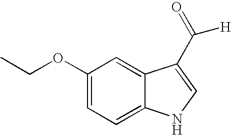 | 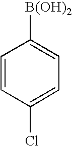 | 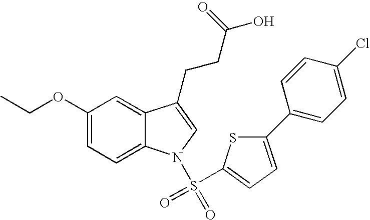 |
| P-0401 | 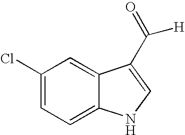 | 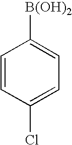 | 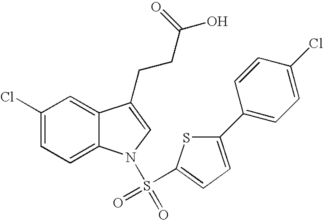 |
| P-0402 | 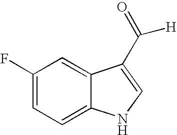 | 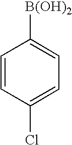 | 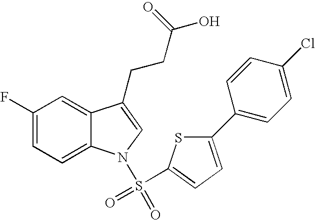 |
| P-0403 | 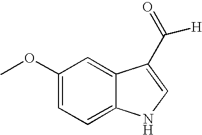 | 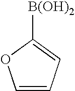 | 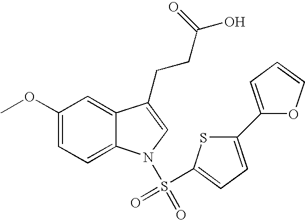 |
| P-0404 | 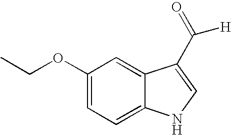 | 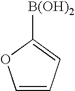 | 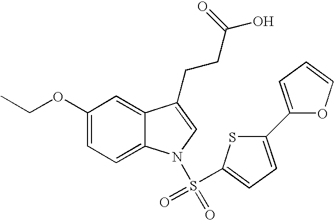 |
| P-0405 | 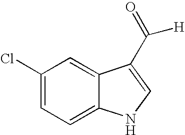 | 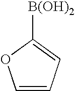 | 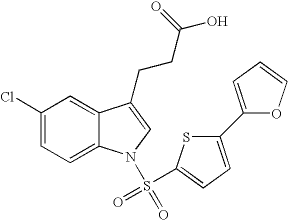 |
| P-0406 | 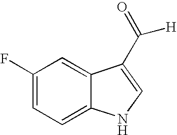 | 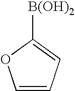 | 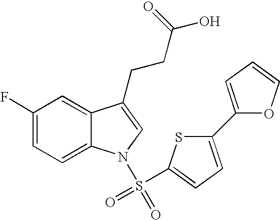 |
| P-0407 | 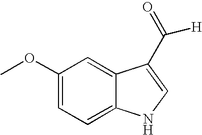 | 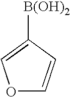 | 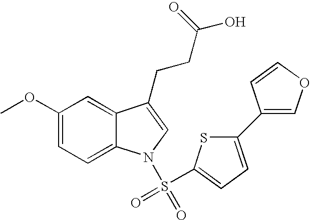 |
| P-0408 | 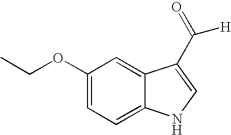 | 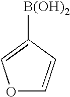 | 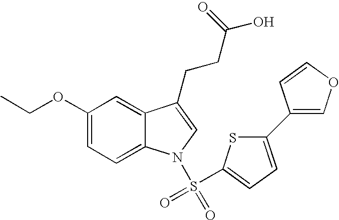 |
| P-0409 | 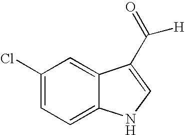 | 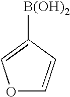 | 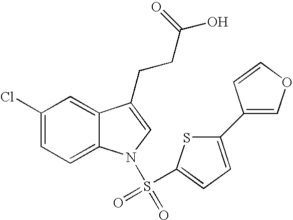 |
| P-0410 | 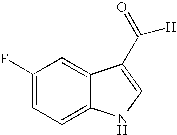 | 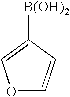 | 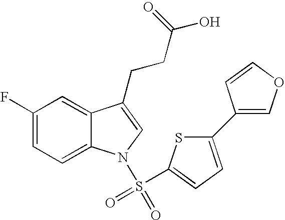 |
| P-0411 | 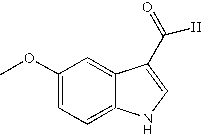 | 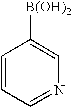 | 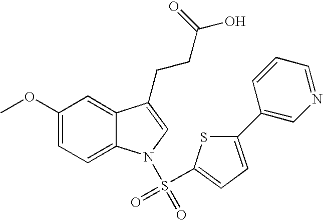 |
| P-0412 | 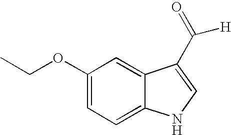 | 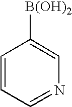 | 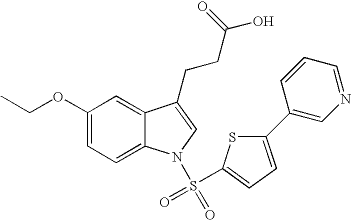 |
| P-0413 | 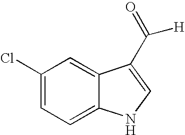 | 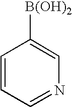 | 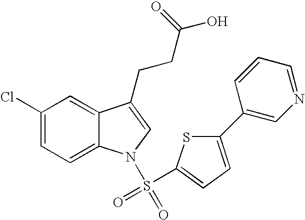 |
| P-0414 | 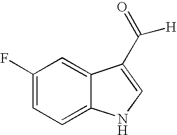 | 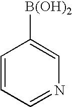 | 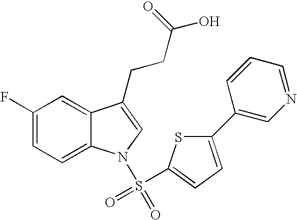 |
| P-0415 | 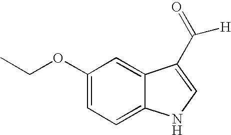 | 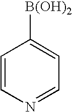 | 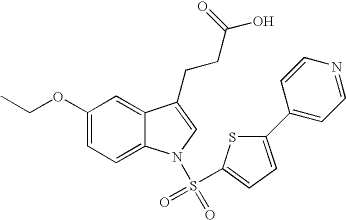 |
| P-0416 | 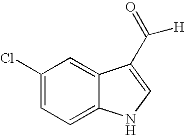 | 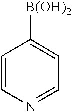 | 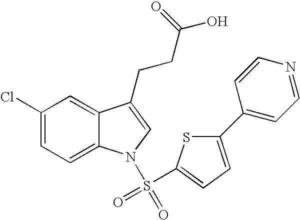 |
| P-0417 | 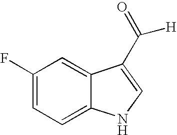 | 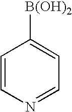 | 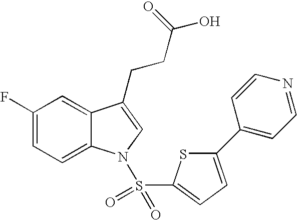 |
| P-0418 | 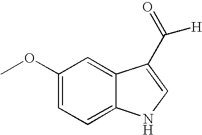 | 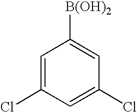 | 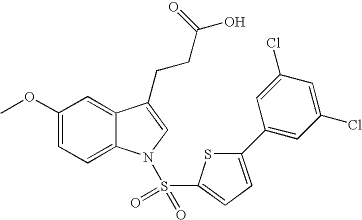 |
| P-0419 | 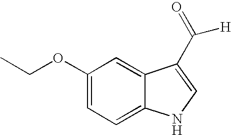 | 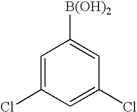 | 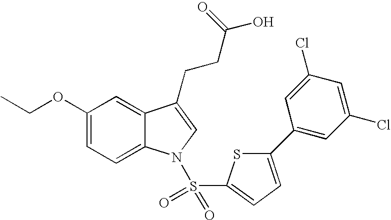 |
| P-0420 | 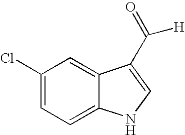 | 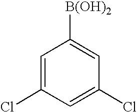 | 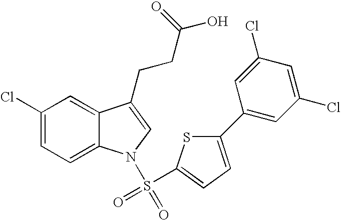 |
| P-0421 | 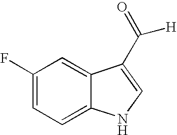 | 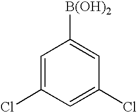 | 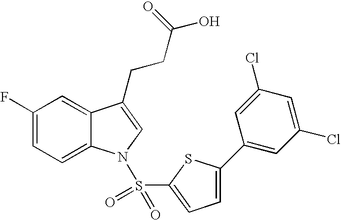 |
| P-0422 | 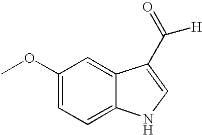 | 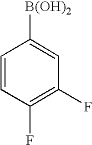 | 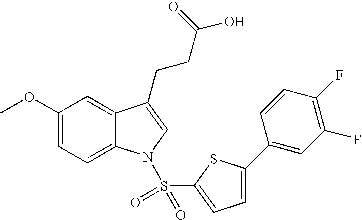 |
| P-0423 | 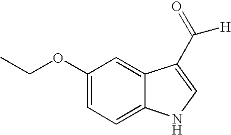 | 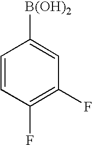 | 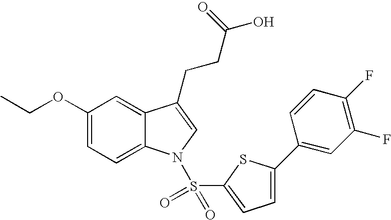 |
| P-0424 | 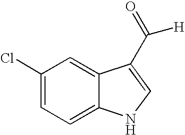 | 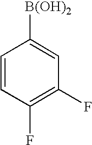 | 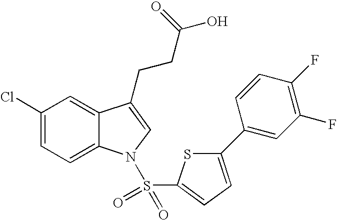 |
| P-0425 | 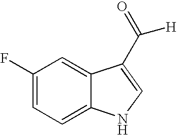 | 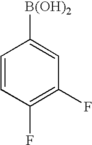 | 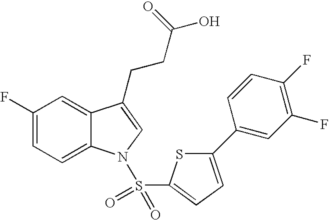 |
| P-0426 | 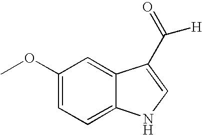 | 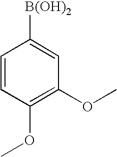 | 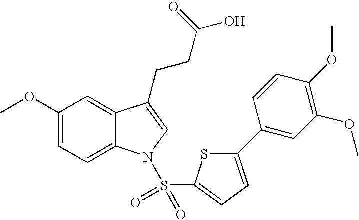 |
| P-0427 | 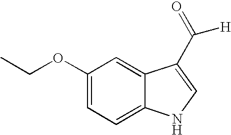 | 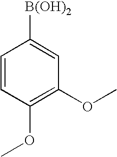 | 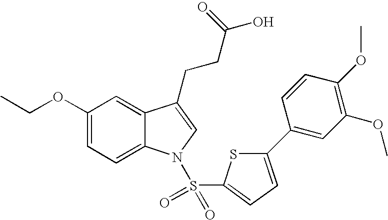 |
| P-0428 | 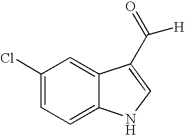 | 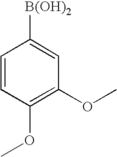 | 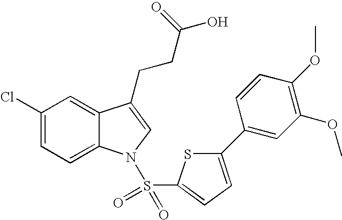 |
| P-0429 | 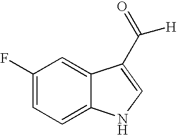 | 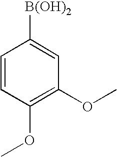 | 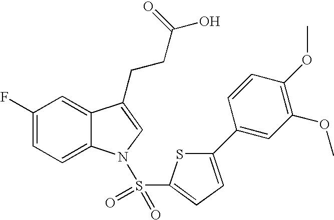 |
| P-0430 | 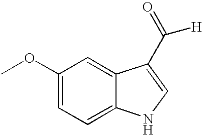 | 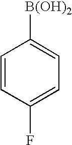 | 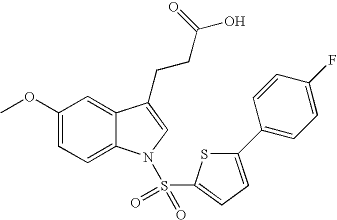 |
| P-0431 | 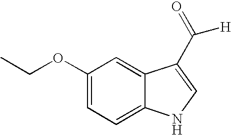 | 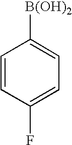 | 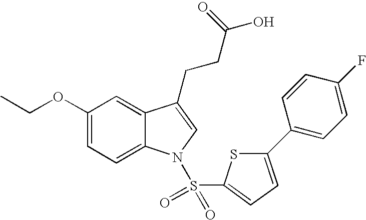 |
| P-0432 | 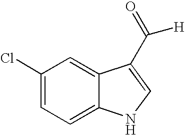 | 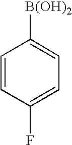 | 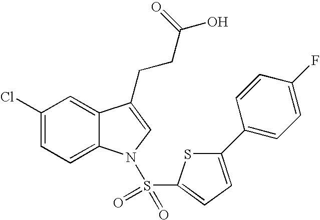 |
| P-0433 | 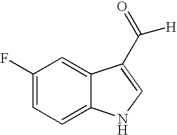 | 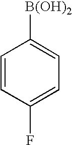 | 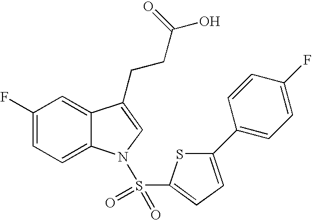 |
| P-0434 | 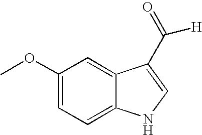 | 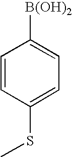 | 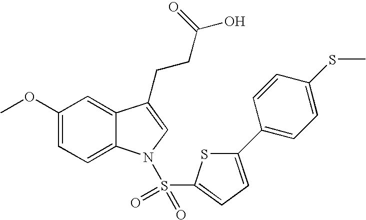 |
| P-0435 | 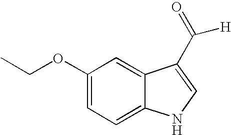 | 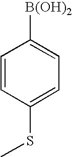 | 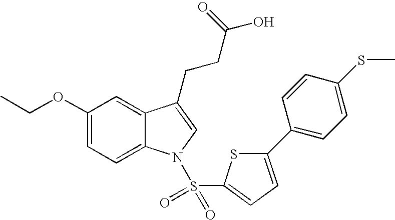 |
| P-0436 | 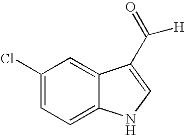 | 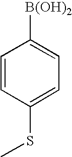 | 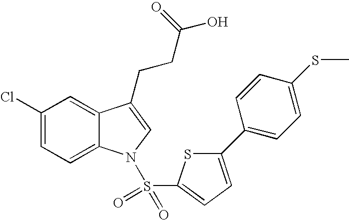 |
| P-0437 | 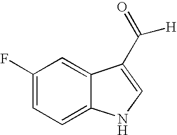 | 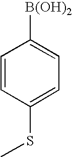 | 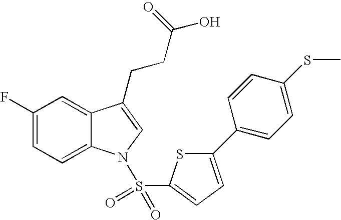 |
| P-0438 | 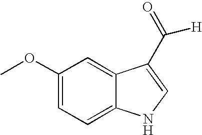 | 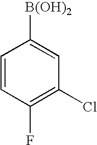 | 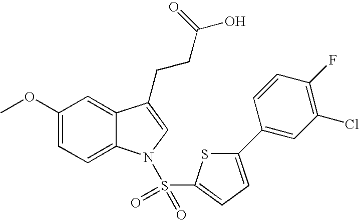 |
| P-0439 | 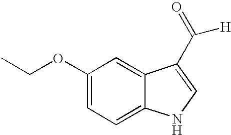 | 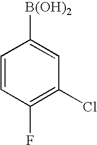 | 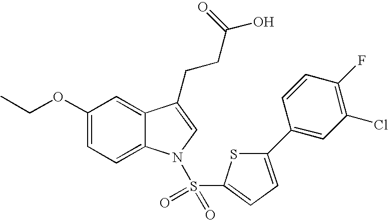 |
| P-0440 | 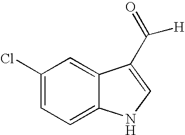 | 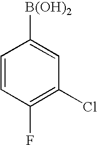 | 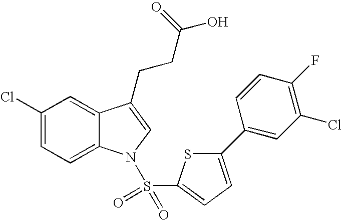 |
| P-0441 | 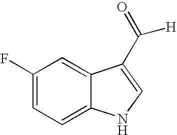 | 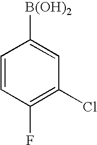 | 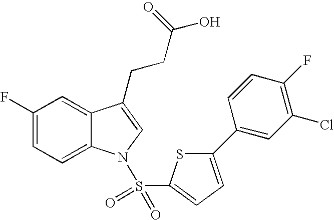 |
| P-0442 | 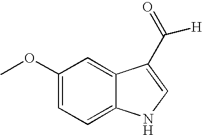 | 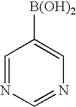 | 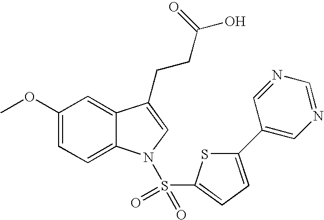 |
| P-0443 | 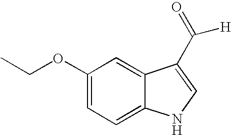 | 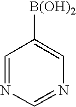 | 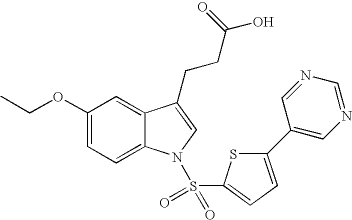 |
| P-0444 | 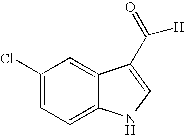 | 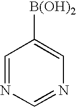 | 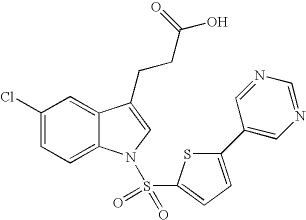 |
| P-0445 | 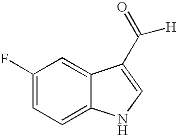 | 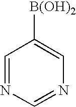 | 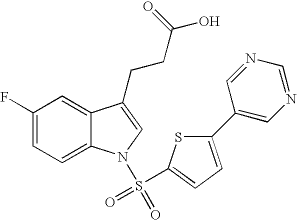 |
| P-0446 | 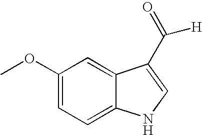 | 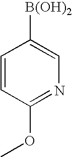 | 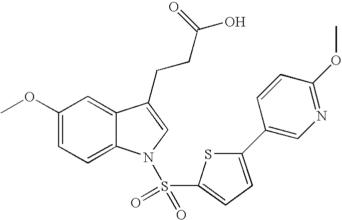 |
| P-0447 | 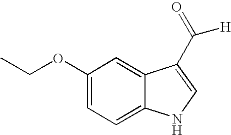 | 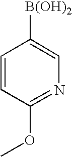 | 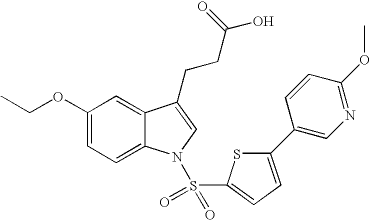 |
| P-0448 | 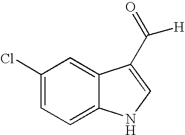 | 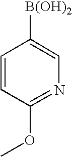 | 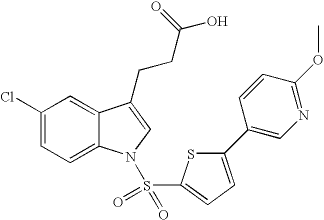 |
| P-0449 | 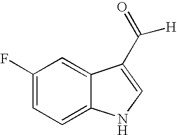 | 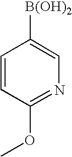 | 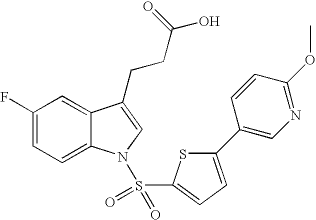 |
| P-0450 | 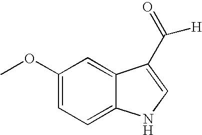 | 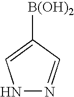 | 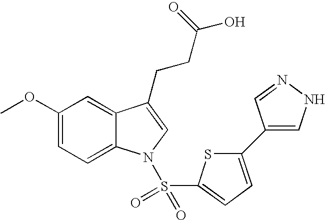 |
| P-0451 | 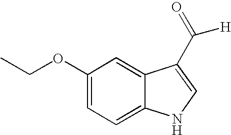 | 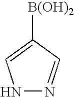 | 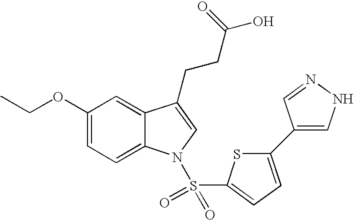 |
| P-0452 | 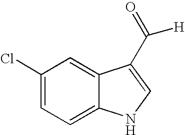 | 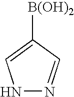 | 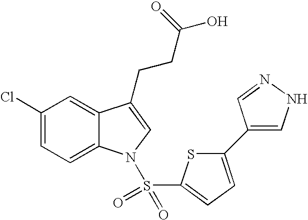 |
| P-0453 | 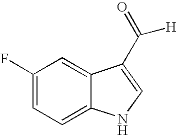 | 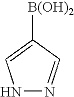 | 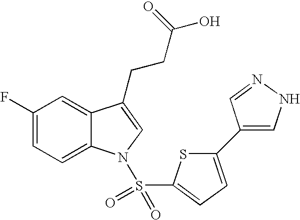 |
| P-0454 | 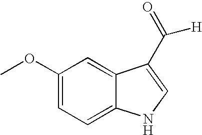 | 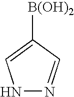 | 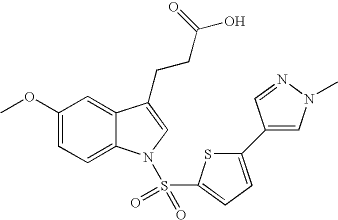 |
| P-0455 | 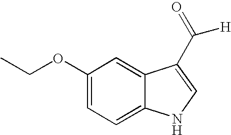 | 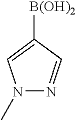 | 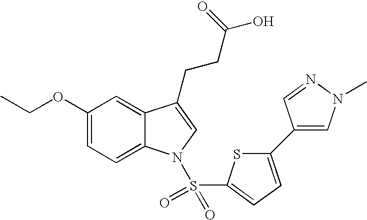 |
| P-0456 | 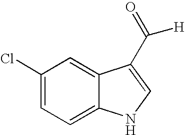 | 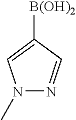 | 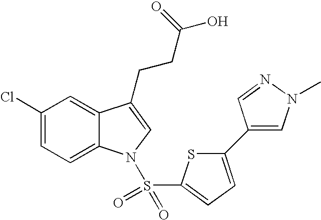 |
| P-0457 | 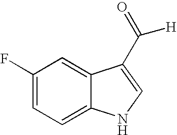 | 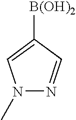 | 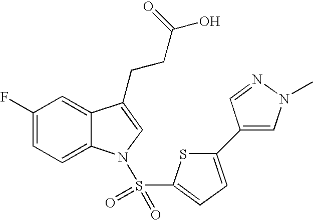 |
| P-0458 | 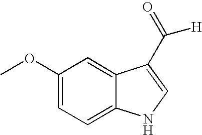 | 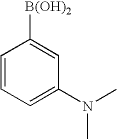 | 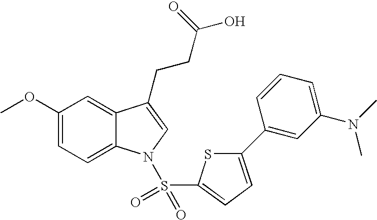 |
| P-0459 | 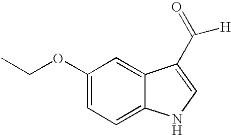 | 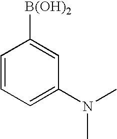 | 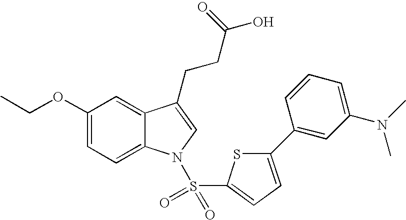 |
| P-0460 | 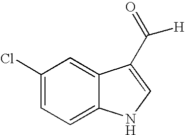 | 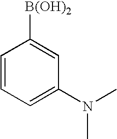 | 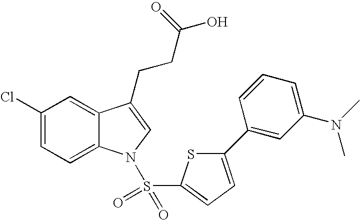 |
| P-0461 | 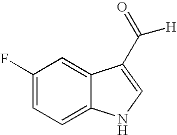 | 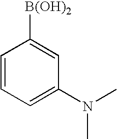 | 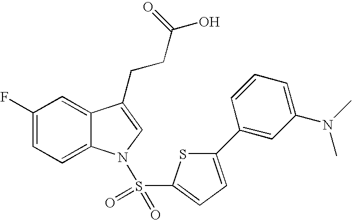 |
| P-0462 | 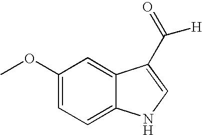 | 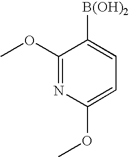 | 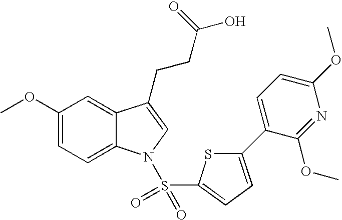 |
| P-0463 | 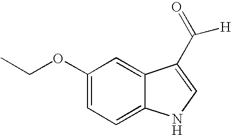 | 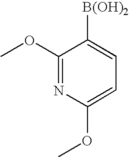 | 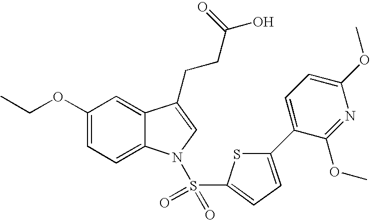 |
| P-0464 | 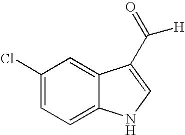 | 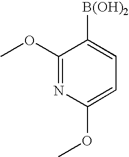 | 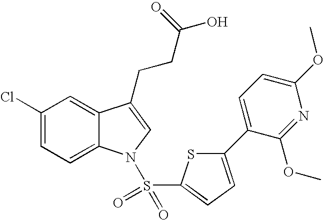 |
| P-0465 | 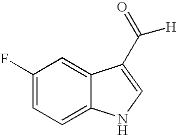 | 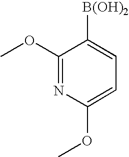 | 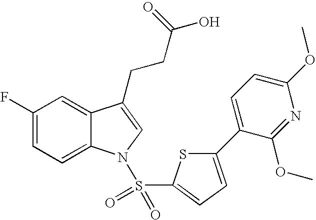 |
| P-0466 | 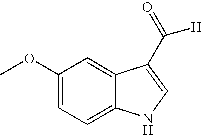 | 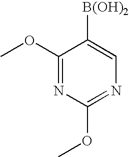 | 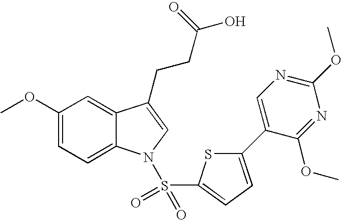 |
| P-0467 | 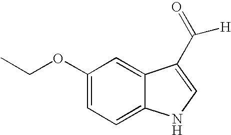 | 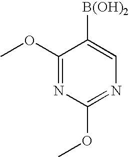 | 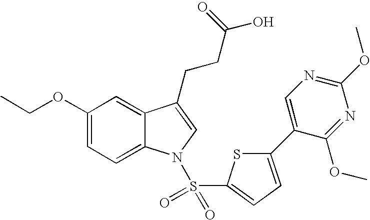 |
| P-0468 | 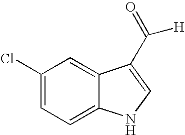 | 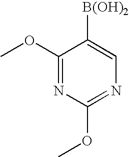 | 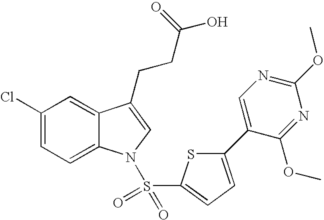 |
| P-0469 | 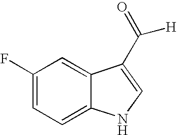 | 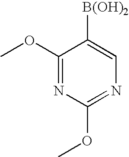 | 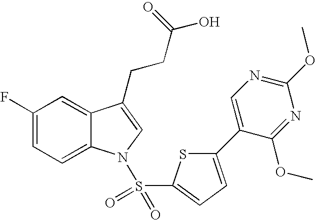 |
| P-0470 | 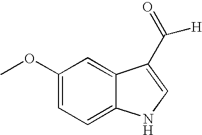 | 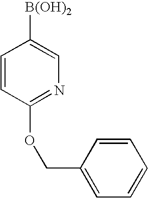 | 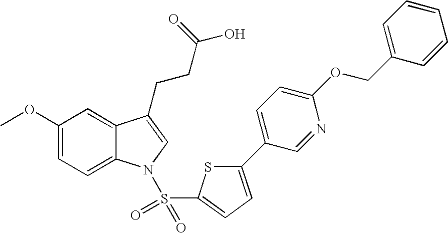 |
| P-0471 | 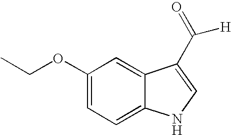 | 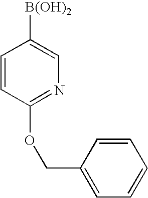 | 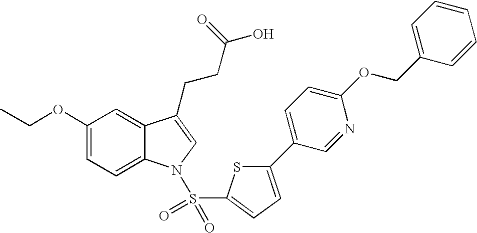 |
| P-0472 | 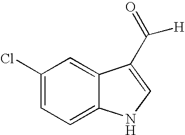 | 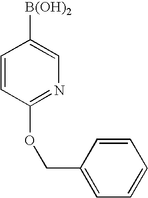 | 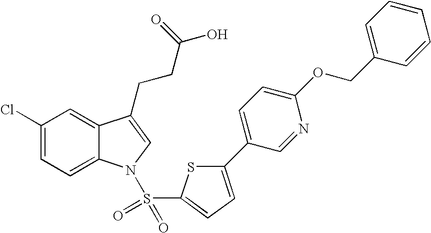 |
| P-0473 | 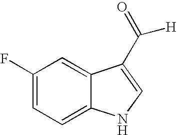 | 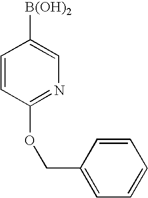 | 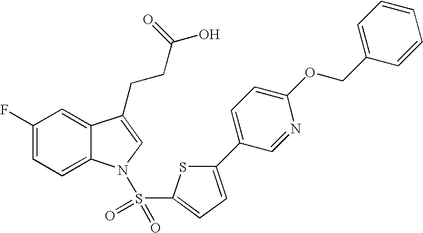 |
| P-0474 | 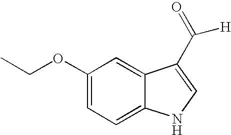 | 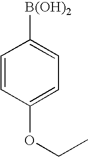 | 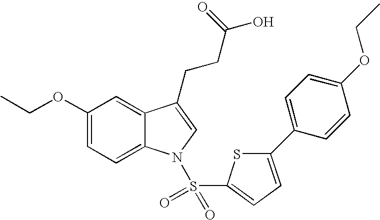 |
| P-0475 | 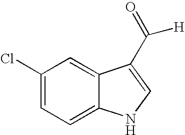 | 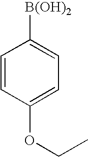 | 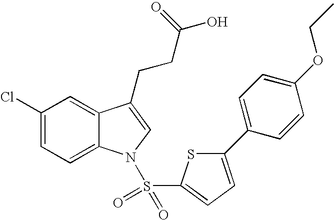 |
| P-0476 | 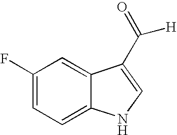 | 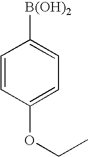 | 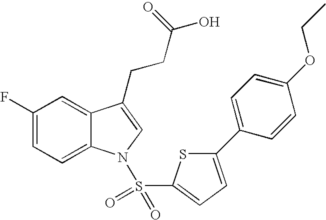 |
| P-0477 | 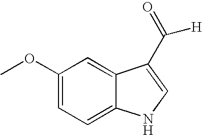 | 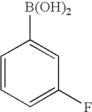 | 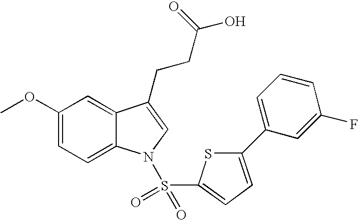 |
| P-0478 | 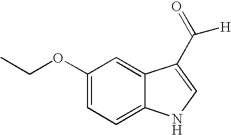 | 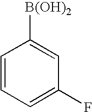 | 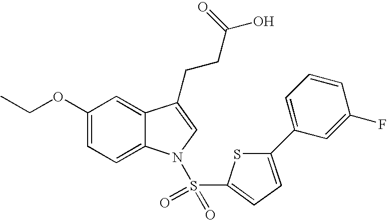 |
| P-0479 | 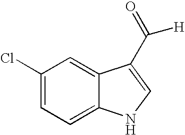 | 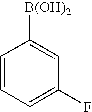 | 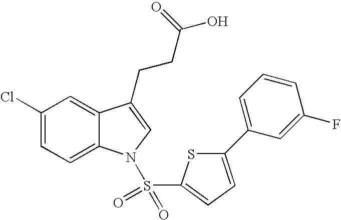 |
| P-0480 | 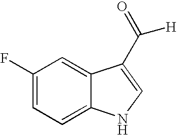 | 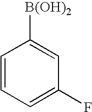 | 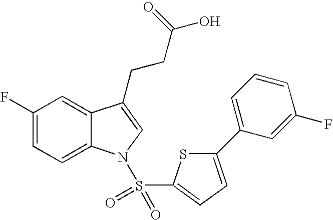 |
| P-0481 | 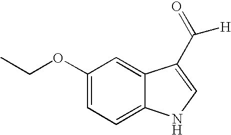 | 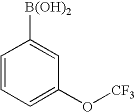 | 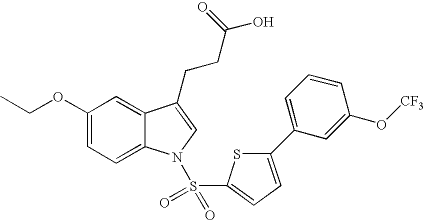 |
| P-0482 | 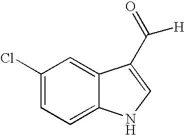 | 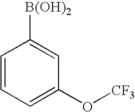 | 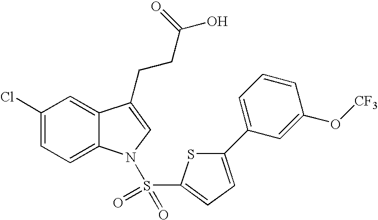 |
| P-0483 | 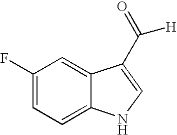 | 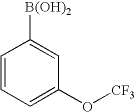 | 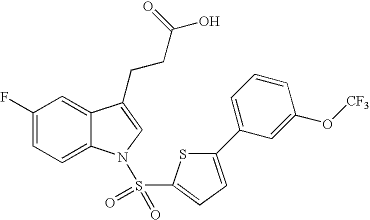 |
| P-0484 | 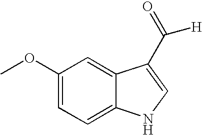 | 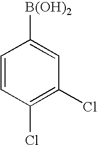 | 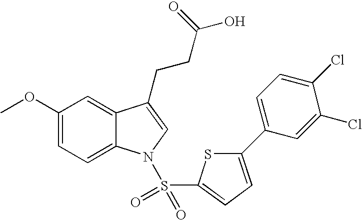 |
| P-0485 | 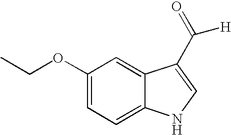 | 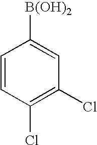 | 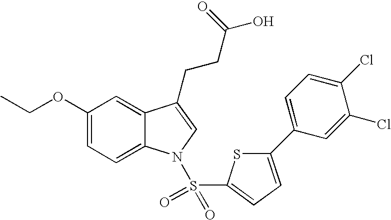 |
| P-0486 | 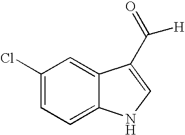 | 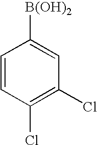 | 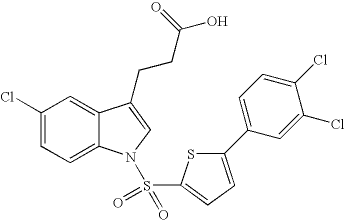 |
| P-0487 | 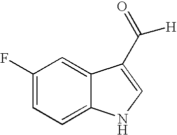 | 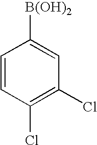 | 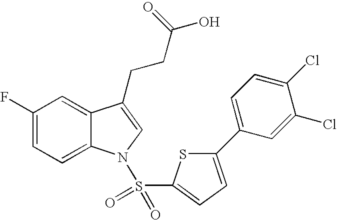 |
| P-0488 | 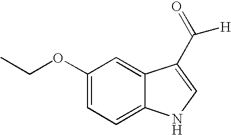 | 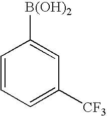 | 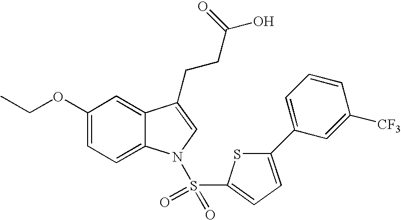 |
| P-0489 | 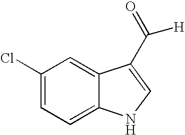 | 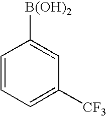 | 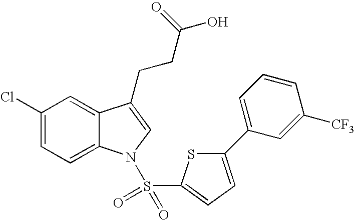 |
| P-0490 | 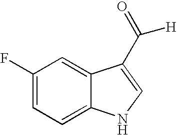 | 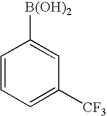 | 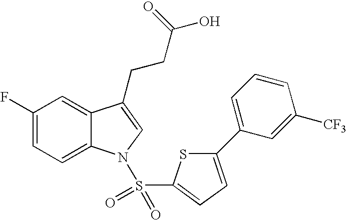 |
| P-0491 | 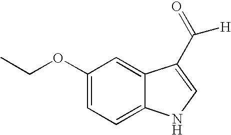 | 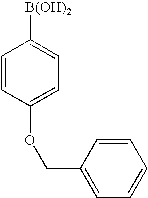 | 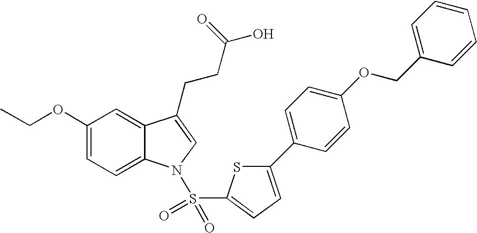 |
| P-0492 | 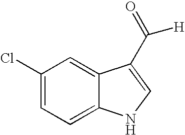 | 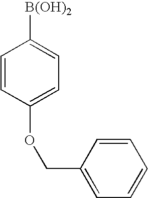 | 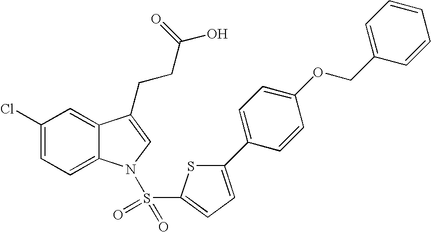 |
| P-0493 | 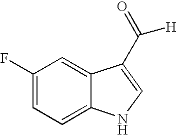 | 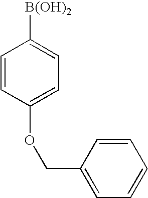 | 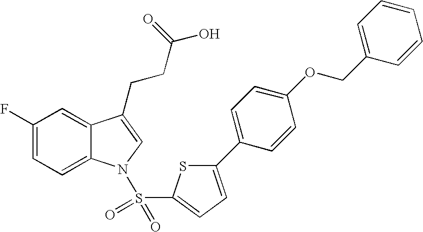 |
| P-0494 | 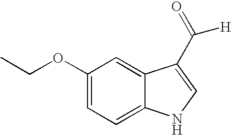 | 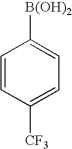 | 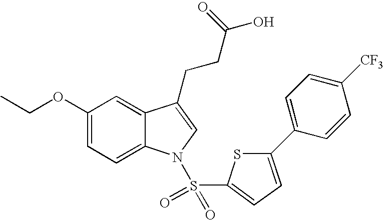 |
| P-0495 | 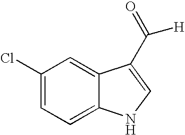 | 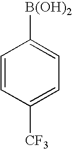 | 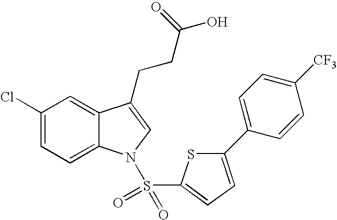 |
| P-0496 | 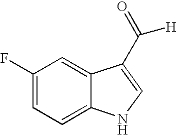 | 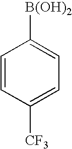 | 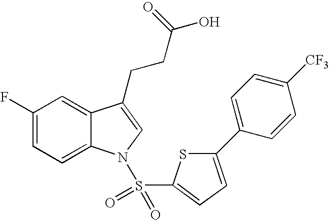 |
| P-0497 | 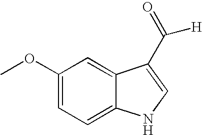 | 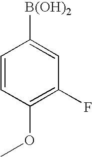 | 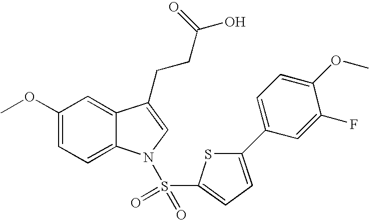 |
| P-0498 | 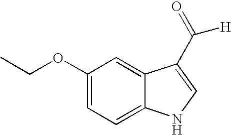 | 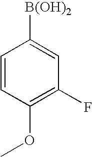 | 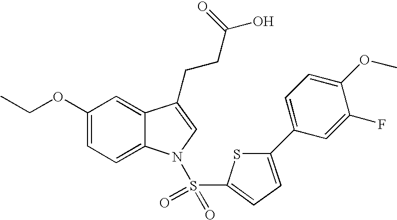 |
| P-0499 | 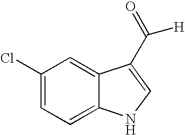 | 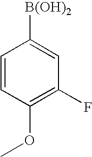 | 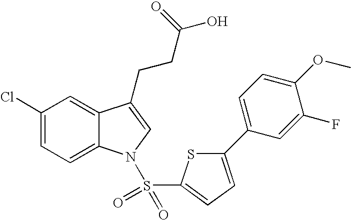 |
| P-0500 | 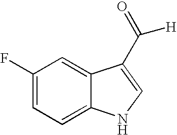 | 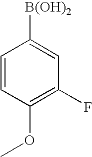 | 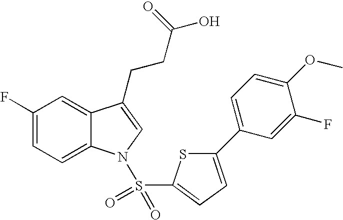 |
| P-0501 | 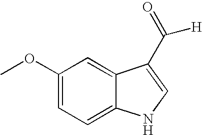 | 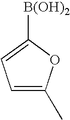 | 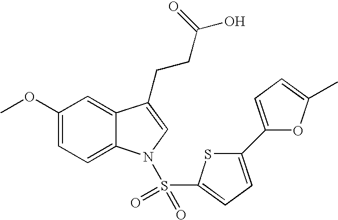 |
| P-0502 | 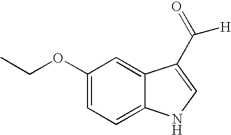 | 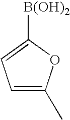 | 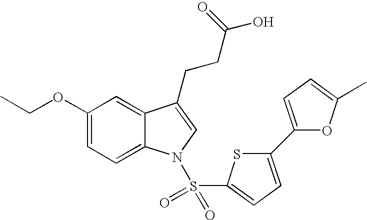 |
| P-0503 | 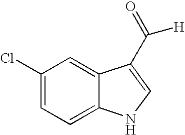 | 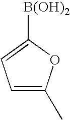 | 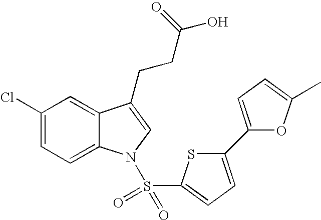 |
| P-0504 | 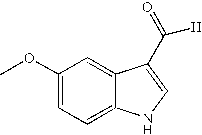 | 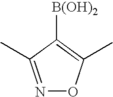 | 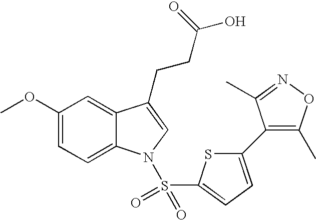 |
| P-0505 | 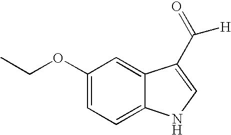 | 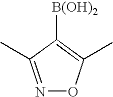 | 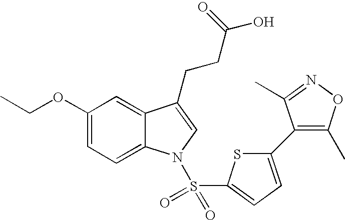 |
| P-0506 | 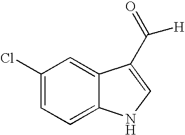 | 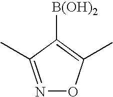 | 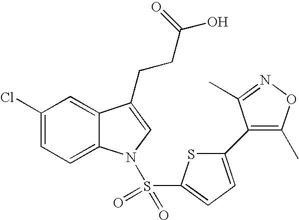 |
| P-0507 | 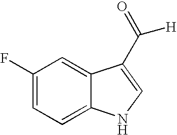 | 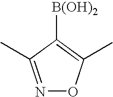 | 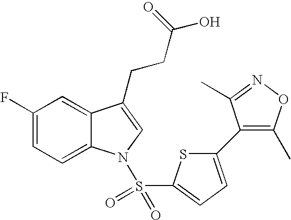 |
| P-0508 | 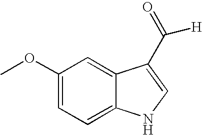 | 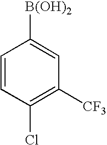 | 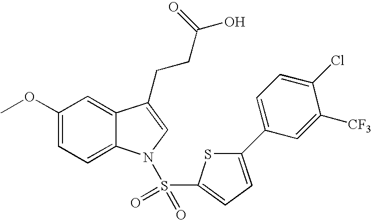 |
| P-0509 | 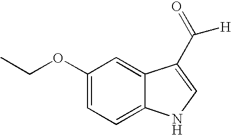 | 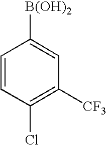 | 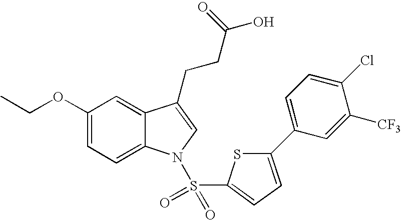 |
| P-0510 | 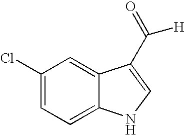 | 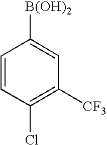 | 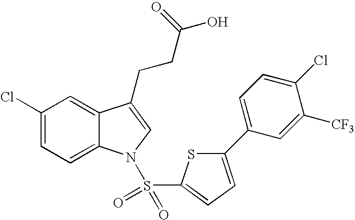 |
| P-0511 | 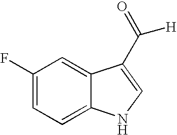 | 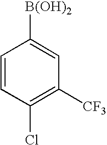 | 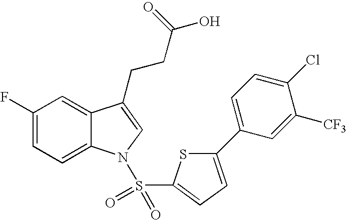 |
| P-0512 | 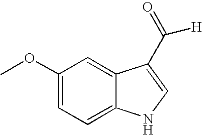 | 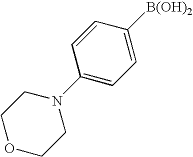 | 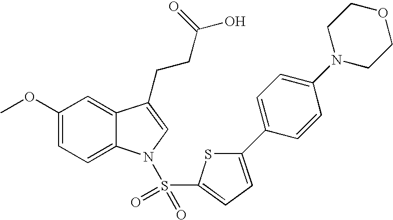 |
| P-0513 | 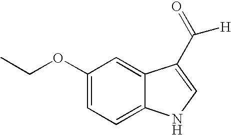 | 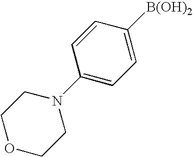 | 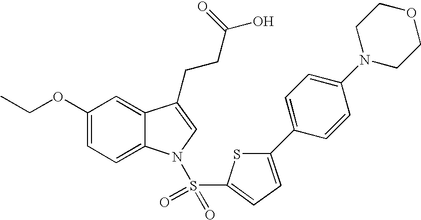 |
| P-0514 | 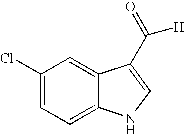 | 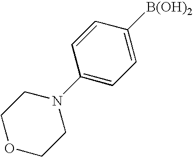 | 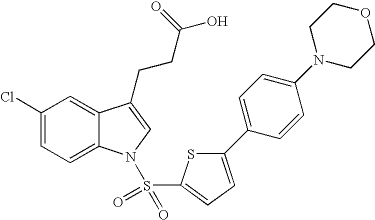 |
| P-0515 | 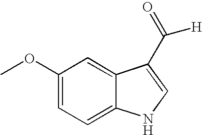 | 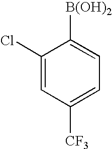 | 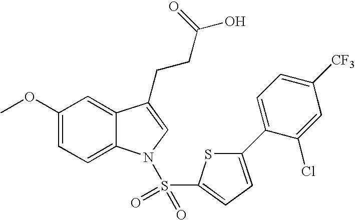 |
| P-0516 | 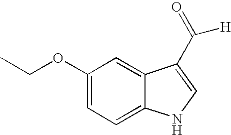 | 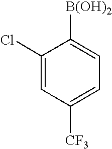 | 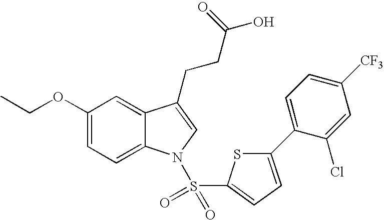 |
| P-0517 | 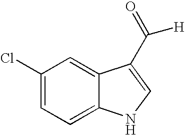 | 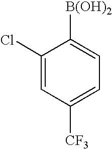 | 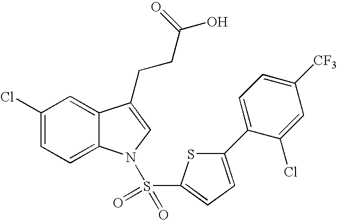 |
| P-0518 | 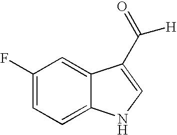 | 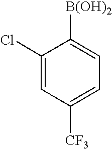 | 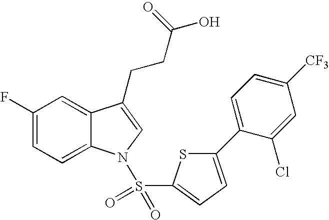 |
| P-0519 | 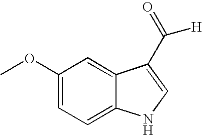 | 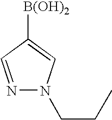 | 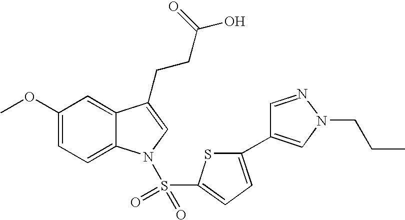 |
| P-0520 | 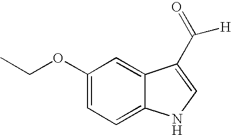 | 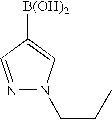 | 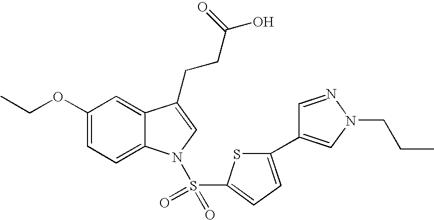 |
| P-0521 | 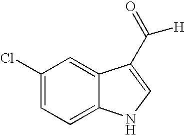 | 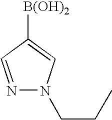 | 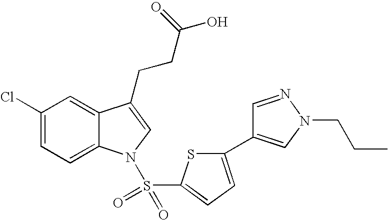 |
| P-0522 | 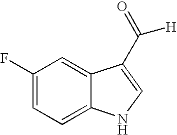 | 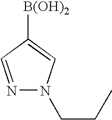 | 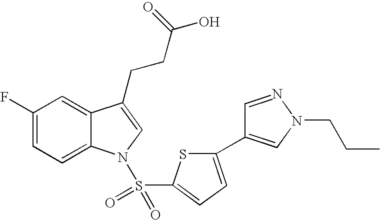 |
| P-0523 | 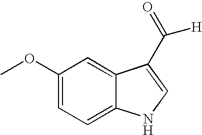 | 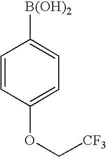 | 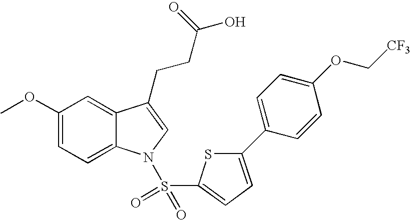 |

Additional compounds were prepared similarly, replacing 5-bromo thiophene-2-sulfonyl chloride 7 with either 2-bromo-benzenesulfonyl chloride or 3-bromo-5-methyl-thiophene-2-sulfonyl chloride in Step 3 and replacing 5-methoxyindole-3-carboxyaldehyde 4 with an appropriate 5-ethoxyindole-3-carboxyaldehyde in Step 1, and reacting the product of Step 3 with an appropriate boronic acid via a modified Step 4, wherein 10 mg of the product of Step 3 was dissolved in 400 μL of acetonitrile in a 2 mL microwave vial. To this was added 2 equivalents of the appropriate boronic acid and 3 mg of tetrakis(triphenylphosphine)palladium(0), then 400 μL of aqueous 1M potassium carbonate was added, the vial capped, and irradiate 10 minutes at 160° C. The solution was neutralized with acetic acid and the solvents removed under vacuum. The crude material was dissolved in dimethyl sulfoxide and purified by reverse phase HPLC (C18 column), eluting with a water/0.1% trifluoro acetic acid and acetonitrile/0.1% trifluoro acetic acid gradient, 20-100% acetonitrile over 16 minutes.

The following compounds were prepared using 5-ethoxyindole-3-carboxyaldehyde in Step 1, 2-bromo-benzenesulfonyl chloride or 3-bromo-5-methyl-thiophene-2-sulfonyl chloride in Step 3, and the appropriate boronic acid in Step 4, with the calculated molecular weight and measured mass (MS(ESI)) provided after the compound:
3-[1-(2-Bromo-benzenesulfonyl)-5-ethoxy-1H-indol-3-yl]-propionic acid ethyl ester (P-0528), calculated MW 480.38, [M+H⁺]⁺=480.1, 482.1,
3-[1-(4′-Chloro-biphenyl-2-sulfonyl)-5-ethoxy-1H-indol-3-yl]-propionic acid (P-0529), calculated MW 483.97, [M+H⁺]⁺=484.3,
3-[5-Ethoxy-1-(4′-methoxy-biphenyl-2-sulfonyl)-1H-indol-3-yl]-propionic acid (P-0530), calculated MW 479.55, [M+H⁺]⁺=479.9,
3-[5-Ethoxy-1-(4′-isopropyl-biphenyl-2-sulfonyl)-1H-indol-3-yl]-propionic acid (P-0531), calculated MW 491.61, [M+H⁺]⁺=492.3,
3-[5-Ethoxy-1-(4′-fluoro-biphenyl-2-sulfonyl)-1H-indol-3-yl]-propionic acid (P-0532), calculated MW 467.51, [M+H⁺]⁺=468.3,
3-[1-(4′-Dimethylamino-biphenyl-2-sulfonyl)-5-ethoxy-1H-indol-3-yl]-propionic acid (P-0533), calculated MW 492.59, [M+H⁺]⁺=492.3,
3-[1-(4′-Acetyl-biphenyl-2-sulfonyl)-5-ethoxy-1H-indol-3-yl]-propionic acid (P-0534), calculated MW 491.56, [M+H⁺]⁺=492.3,
3-[5-Ethoxy-1-(4′-ethoxy-biphenyl-2-sulfonyl)-1H-indol-3-yl]-propionic acid (P-0535), calculated MW 493.58, [M+H⁺]⁺=494.3,
3-[5-Ethoxy-1-(4′-trifluoromethoxy-biphenyl-2-sulfonyl)-1H-indol-3-yl]-propionic acid (P-0536), calculated MW 533.52, [M+H⁺]⁺=533.9,
3-[5-Ethoxy-1-(4′-ethyl-biphenyl-2-sulfonyl)-1H-indol-3-yl]-propionic acid (P-0537), calculated MW 477.58, [M+H⁺]⁺=477.9,
3-[5-Ethoxy-1-(4′-propyl-biphenyl-2-sulfonyl)-1H-indol-3-yl]-propionic acid (P-0538), calculated MW 491.61, [M+H⁺]⁺=492.3,
3-[1-(4′-Amino-biphenyl-2-sulfonyl)-5-ethoxy-1H-indol-3-yl]-propionic acid (P-0539), calculated MW 464.54, [M+H⁺]⁺=465.1,
3-[5-Ethoxy-1-(4′-trifluoromethyl-biphenyl-2-sulfonyl)-1H-indol-3-yl]-propionic acid (P-0552), calculated MW 517.52, [M+H⁺]⁺=517.9,
3-[1-(3′,4′-Difluoro-biphenyl-2-sulfonyl)-5-ethoxy-1H-indol-3-yl]-propionic acid (P-0553), calculated MW 517.52, [M+H⁺]⁺=517.9,
3-{5-Ethoxy-1-[2-(1H-indol-5-yl)-benzenesulfonyl]-1H-indol-3-yl}-propionic acid (P-0554), calculated MW 488.56, [M+H⁺]⁺=489.1,
3-[1-(3′,4′-Dimethyl-biphenyl-2-sulfonyl)-5-ethoxy-1H-indol-3-yl]-propionic acid (P-0555), calculated MW 477.58, [M+H⁺]⁺=478.3,
3-[5-Ethoxy-1-(5-methyl-3-p-tolyl-thiophene-2-sulfonyl)-1H-indol-3-yl]-propionic acid (P-0524),
3-{1-[3-(4-Benzyloxy-phenyl)-5-methyl-thiophene-2-sulfonyl]-5-ethoxy-1H-indol-3-yl}-propionic acid (P-0525),
3-{1-[3-(4-Amino-phenyl)-5-methyl-thiophene-2-sulfonyl]-5-ethoxy-1H-indol-3-yl}-propionic acid (P-0526),
3-{5-Ethoxy-1-[3-(4-hydroxy-phenyl)-5-methyl-thiophene-2-sulfonyl]-1H-indol-3-yl}-propionic acid (P-0527),
3-{1-[3-(4-Chloro-phenyl)-5-methyl-thiophene-2-sulfonyl]-5-ethoxy-1H-indol-3-yl}-propionic acid (P-0540),
3-{5-Ethoxy-1-[3-(4-methoxy-phenyl)-5-methyl-thiophene-2-sulfonyl]-1H-indol-3-yl}-propionic acid (P-0541),
3-{5-Ethoxy-1-[3-(4-isopropyl-phenyl)-5-methyl-thiophene-2-sulfonyl]-1H-indol-3-yl}-propionic acid (P-0542),
3-{5-Ethoxy-1-[3-(4-fluoro-phenyl)-5-methyl-thiophene-2-sulfonyl]-1H-indol-3-yl}-propionic acid (P-0543),
3-{1-[3-(4-Dimethylamino-phenyl)-5-methyl-thiophene-2-sulfonyl]-5-ethoxy-1H-indol-3-yl}-propionic acid (P-0544),
3-{1-[3-(4-Acetyl-phenyl)-5-methyl-thiophene-2-sulfonyl]-5-ethoxy-1H-indol-3-yl}propionic acid (P-0545),
3-{5-Ethoxy-1-[3-(4-ethoxy-phenyl)-5-methyl-thiophene-2-sulfonyl]-1H-indol-3-yl}propionic acid (P-0546)
3-{5-Ethoxy-1-[5-methyl-3-(4-trifluoromethoxy-phenyl)-thiophene-2-sulfonyl]-1H-indol-3-yl}-propionic acid (P-0547),
3-{5-Ethoxy-1-[5-methyl-3-(4-trifluoromethyl-phenyl)-thiophene-2-sulfonyl]-1H-indol-3-yl}-propionic acid (P-0548),
3-{5-Ethoxy-1-[3-(4-ethyl-phenyl)-5-methyl-thiophene-2-sulfonyl]-1H-indol-3-yl}-propionic acid (P-0549),
3-{5-Ethoxy-1-[5-methyl-3-(4-propyl-phenyl)-thiophene-2-sulfonyl]-1H-indol-3-yl}-propionic acid (P-0550),
3-{1-[3-(4-Aminomethyl-phenyl)-5-methyl-thiophene-2-sulfonyl]-5-ethoxy-1H-indol-3-yl}-propionic acid (P-0551),
3-{1-[3-(3,4-Difluoro-phenyl)-5-methyl-thiophene-2-sulfonyl]-5-ethoxy-1H-indol-3-yl}-propionic acid (P-0556),
3-{5-Ethoxy-1-[3-(1H-indol-5-yl)-5-methyl-thiophene-2-sulfonyl]-1H-indol-3-yl}-propionic acid (P-0557), and
3-{1-[3-(3,4-Dimethyl-phenyl)-5-methyl-thiophene-2-sulfonyl]-5-ethoxy-1H-indol-3-yl}-propionic acid (P-0558).

These compounds are shown in the following Table 4, indicating the sulfonyl chloride used in Step 3 (Column 2), the boronic acid used in the modified Step 4 (Column 3), and the compound structure (Column 4), with the compound number provided in Column 1.

TABLE 4

| Cmpd. | | | |
|---|---|---|---|
| number | Sulfonyl-chloride | Boronic acid | Compound structure |
| P-0528* | 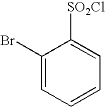 | N/A | 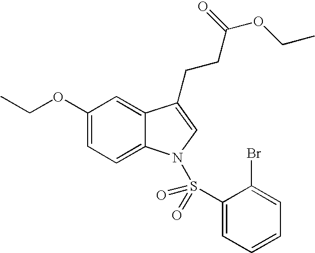 |
| P-0529 | 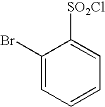 | 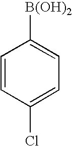 | 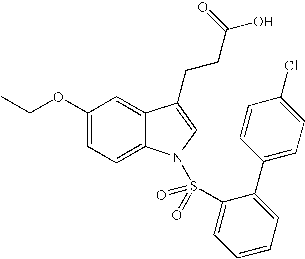 |
| P-0530 | 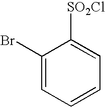 | 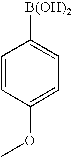 | 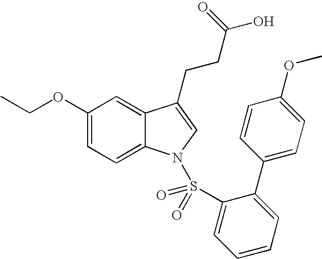 |
| P-0531 | 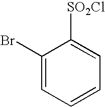 | 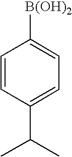 | 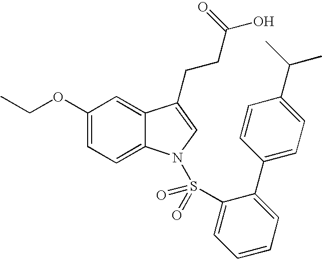 |
| P-0532 | 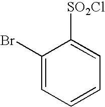 | 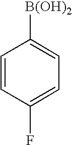 | 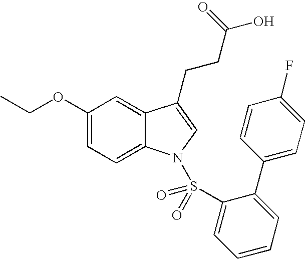 |
| P-0533 | 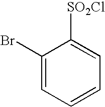 | 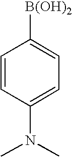 | 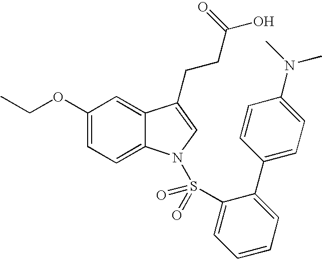 |
| P-0534 | 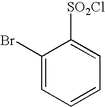 | 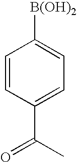 | 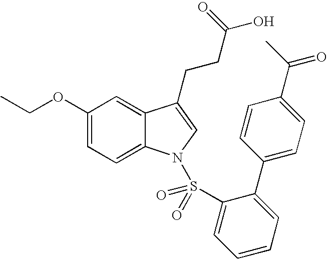 |
| P-0535 | 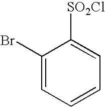 | 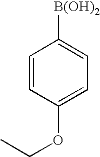 | 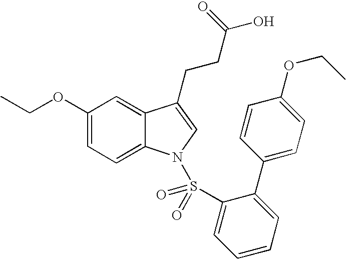 |
| P-0536 | 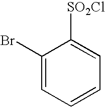 | 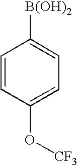 | 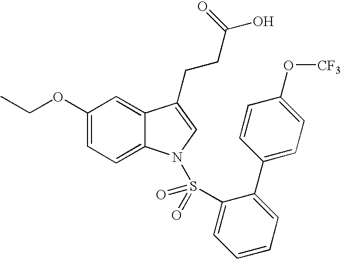 |
| P-0537 | 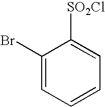 | 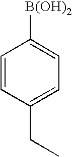 | 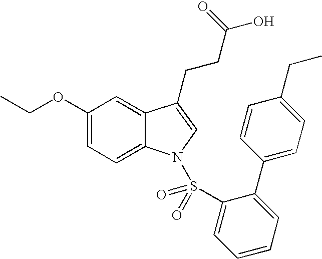 |
| P-0538 | 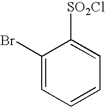 | 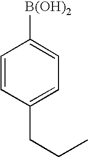 | 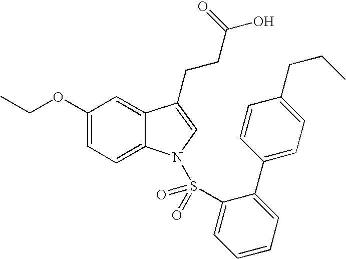 |
| P-0539 | 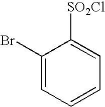 | 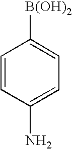 | 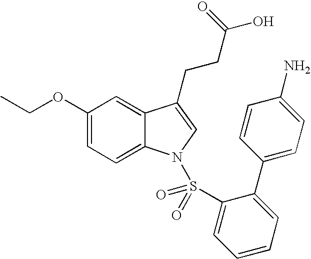 |
| P-0552 | 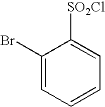 | 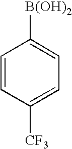 | 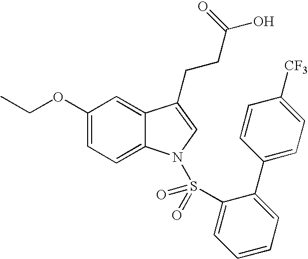 |
| P-0553 | 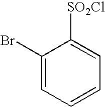 | 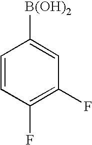 | 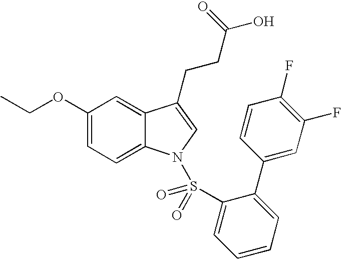 |
| P-0554 | 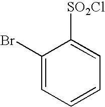 | 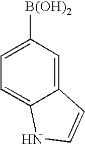 | 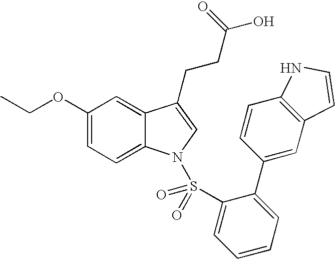 |
| P-0555 | 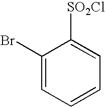 | 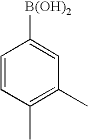 | 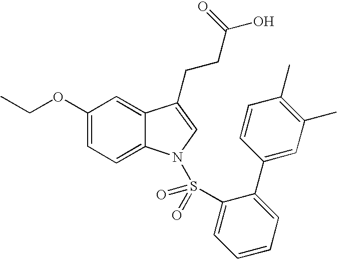 |
| P-0524 | 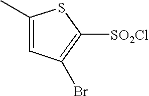 | 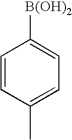 | 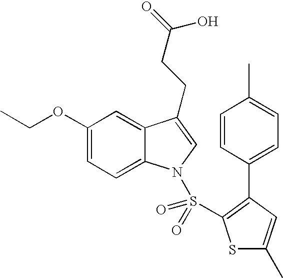 |
| P-0525 | 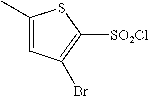 | 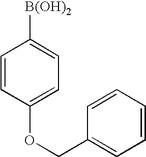 | 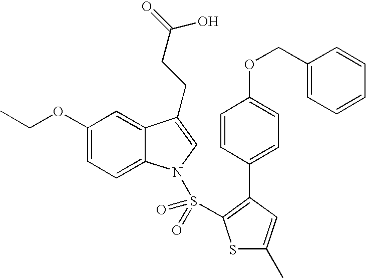 |
| P-0526 | 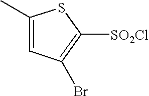 | 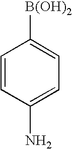 | 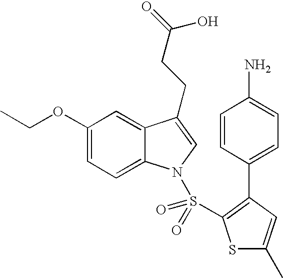 |
| P-0527 | 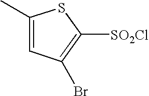 | 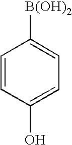 | 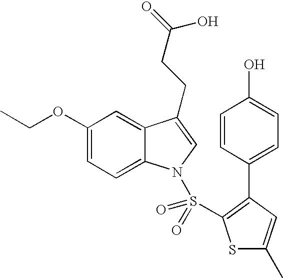 |
| P-0540 | 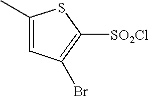 | 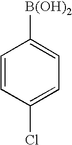 | 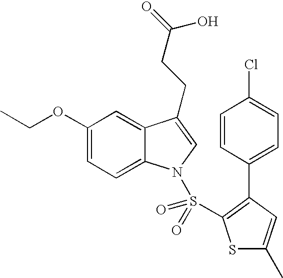 |
| P-0541 | 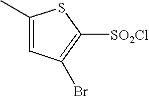 | 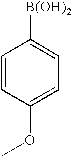 | 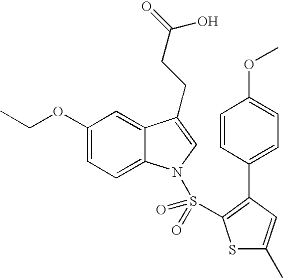 |
| P-0542 | 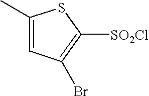 | 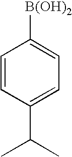 | 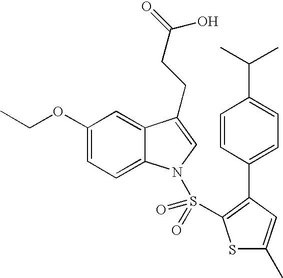 |
| P-0543 | 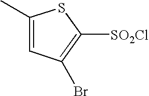 | 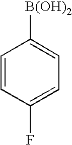 | 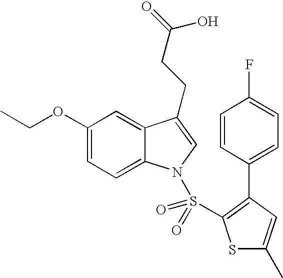 |
| P-0544 | 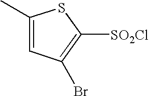 | 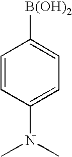 | 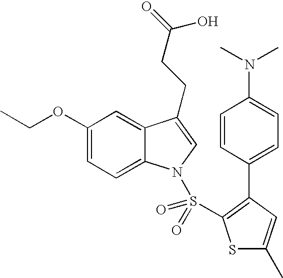 |
| P-0545 | 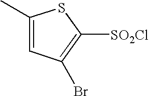 | 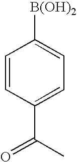 | 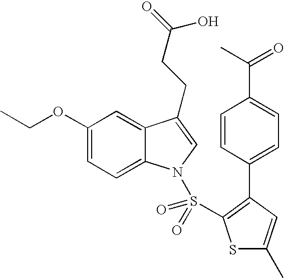 |
| P-0546 | 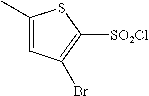 | 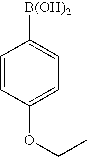 | 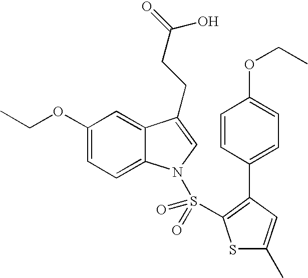 |
| P-0547 | 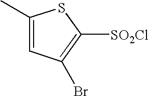 | 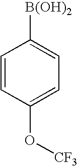 | 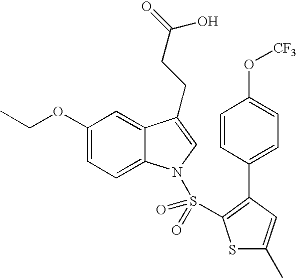 |
| P-0548 | 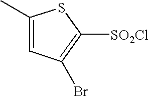 | 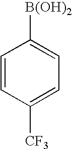 | 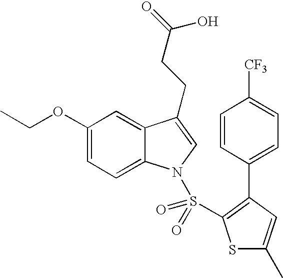 |
| P-0549 | 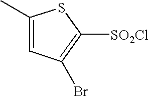 | 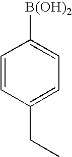 | 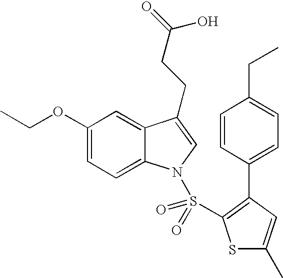 |
| P-0550 | 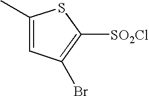 | 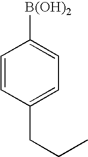 | 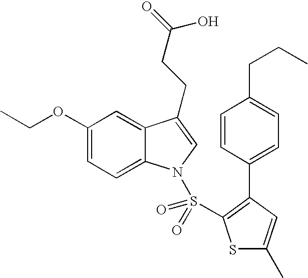 |
| P-0551 | 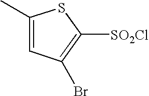 | 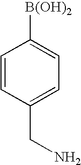 | 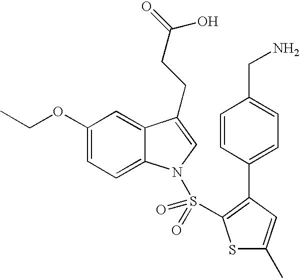 |
| P-0556 | 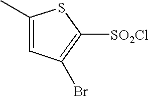 | 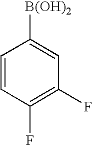 | 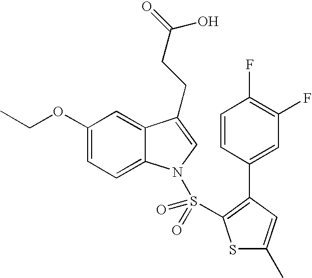 |
| P-0557 | 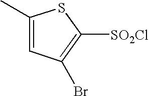 | 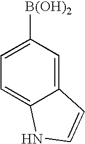 | 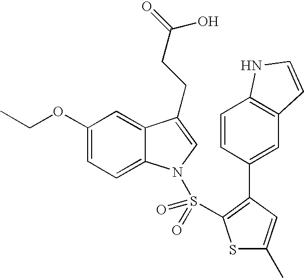 |
| P-0558 | 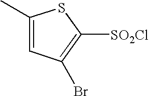 | 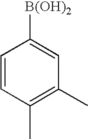 | 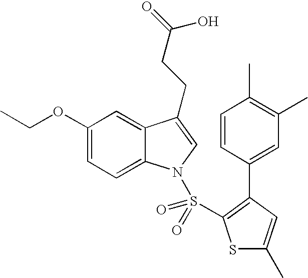 |

*isolated after Step 3.

### Example 3

### Synthesis of 2-[5-Methoxy-1-(4-methoxy-benzenesulfonyl)-1H-Indol-3-yl)-cyclopropane carboxylic acid (P-0012)

Compound P-0012 was synthesized in four steps as shown in Scheme 3.

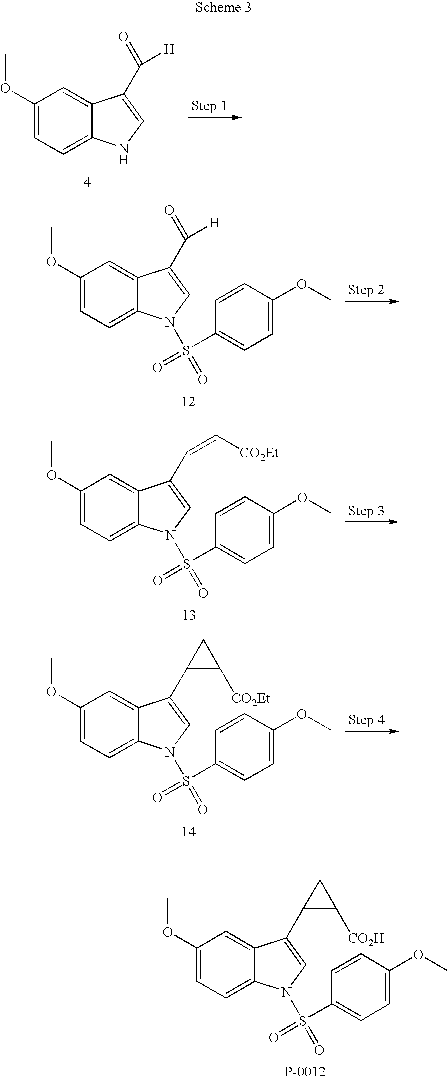

### Step-1: Preparation of 5-methoxy-1-(4-methoxy benzenesulfonyl)-1H-indole-3-carbaldehyde (12)

5-methoxyindole-3-carboxyaldehyde (4, 263 mg) and toluene (15 mL) were combined in a dry 100 mL round bottom flask. The mixture was stirred for 5 minutes, then an aqueous solution of 50% KOH (12 mL) was added followed by tetrabutyl ammonium hydrogen sulfate (8 mg). The solution was stirred at room temperature overnight, after which the resulting solid was collected by filtration using a medium grade fritted funnel. The solid was rinsed with cold water (10 mL) and ethyl ether (2×15 mL) to give the desired compound (2, 388 mg, 75%). 1H NMR is consistent with the compound structure set forth above.

### Step-2: Preparation of (Z)-3-[5-methoxy-1-(4-methoxy-benzenesulfonyl)-1H-Indol-3-yl)-acrylic acid ethyl ester (13)

Ethyl diphenylphosphonoacetate (549 mg, 1.71 mmol) in tetrahydrofuran (2 mL) was cooled to 0° C. and sodium hydride (47.9 mg, 1.99 mmol) was added. The mixture was stirred at 0° C. for 15 min and the solution of deprotonated ethyl diphenylphosphonoacetate was added dropwise to a stirring solution of 5-methoxy-1-(4-methoxy benzenesulfonyl)-1H-indole-3-carbaldehyde (12, 493 mg, 1.43 mmol) in tetrahydrofuran (11 mL) at 0° C. The reaction mixture was warmed slowly to 25° C. overnight. As conversion of 12 was incomplete, the reaction mixture was cooled to 0° C. and an additional equivalent of deprotonated ethyl diphenylphosphonoacetate was added. After slowly warming to 25° C. overnight, ethyl acetate was added to the reaction mixture and the organic layer was washed with saturated sodium bicarbonate, dried over magnesium sulfate and filtered. Concentration under reduced pressure resulted in a crude solid that was a 2:1 ratio of the Z and E isomers. The Z isomer was isolated using chromatography (gradient of hexanes to 20% ethyl acetate in hexanes) (13, 251 mg, 42% yield). MS(ESI) [M+H⁺]⁺=438.2.

### Step-3: Preparation of 2-[5-methoxy-1-(4-methoxy-benzenesulfonyl)-1H-Indol-3-yl)-cyclopropane carboxylic acid ethyl ester (14)

Trimethylsulfoxonium iodide (116 mg, 0.053 mmol) was dissolved in dimethyl sulfoxide (0.75 mL) and sodium hydride (14 mg, 0.058 mmol) was added. After 20 minutes of stirring at 25° C., (Z)-3-[5-methoxy-1-(4-methoxy-benzenesulfonyl)-1H-Indol-3-yl)-acrylic acid ethyl ester (13, 200 mg, 0.048 mmol) in tetrahydrofuran (0.78 mL) was added and the solution was heated to 60° C. under an atmosphere of nitrogen overnight. Water was added to the reaction mixture followed by the addition of ethyl acetate. The aqueous layer was washed with ethyl acetate and the organic layers were combined, dried over magnesium sulfate, filtered and concentrated at reduced pressure. Purification of the crude material was carried out using preparatory chromatography (30% ethyl acetate in hexanes) to obtain the desired compound as an off-white solid (14, 33 mg, 16% yield). MS(ESI) [M+H⁺]⁺=430.4.

### Step-4: Preparation of 2-[5-methoxy-1-(4-methoxy-benzenesulfonyl)-1H-Indol-3-yl)-cyclopropane carboxylic acid (P-0012)

2-[5-Methoxy-1-(4-methoxy-benzenesulfonyl)-1H-Indol-3-yl)-cyclopropane carboxylic acid ethyl ester (14, 25 mg, 0.006 mmol) was dissolved in tetrahydrofuran (1.0 mL) and 1M lithium hydroxide (0.25 mL) was added. After stirring for 4 days at 25° C., acetate was added and the mixture was acidified with 1M hydrochloric acid. The organic layer was dried over magnesium sulfate, filtered and concentrated at reduced pressure to yield a red solid. The crude material was triturated with tert-butyl methyl ether to afford the desired compound (P-0012, 2.6 mg, 11% yield). Calculated molecular weight 401.43, MS(ESI) [M−H⁺]⁻=400.2.

### Example 4

### Synthesis of 3-{5-phenyl-1-[4-(4-trifluoromethyl-phenoxy)-benzesulfonyl]-1H-indol-3-yl}-propionic acid (P-0082)

Compound P-0082 was synthesized in five Steps as shown in Scheme 4.

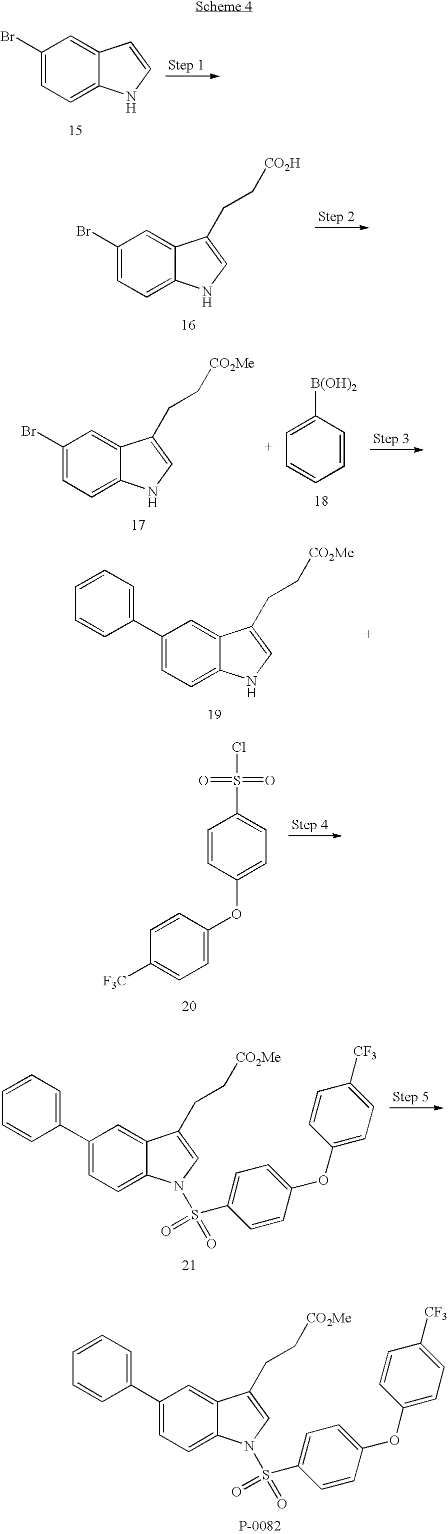

### Step 1: preparation of 5-bromoindole-3-propionic acid (16)

Into a microwave vessel, 5-bromo-indole (15, 1 equivalent), paraformaldehyde (1.1 equivalent), 2,2-dimethyl-1,3-dioxane-4,6-dione (1.1 equivalent), and triethylamine (1.1 equivalent) were dissolved in acetonitrile (2 mL/mmol). The reaction was heated at 150° C. for 3 minutes in a microwave reactor. The reaction was then diluted with acidified water to pH ˜5 with acetic acid and the aqueous layer was extracted with ethyl acetate. The organic layer was then washed with water 2×, brine 1×, and dried over magnesium sulfate. Evaporation of solvent afforded a solid. The crude solid was then purified via flash chromatography with a step gradient of 2 to 4 to 6% methanol in chloroform on silica to obtain the desired compound as a solid.

### Step 2: Preparation of 5-bromoindole-3-propionic acid methyl ester (17)

The 5-bromo-indole-3-propionic acid 16 was treated with an aqueous solution of 4M HCl:methanol:dioxane (1:1:1) for 1 hour. The reaction was then re-evaporated with xylene and purified via flash chromatography on silica (chloroform) to obtain the desired compound as an off-white solid.

### Step 3: Preparation of 3-(5-phenyl-1H-indol-3-yl)-propionic acid methyl ester (19)

Into a microwave tube containing intermediate 5-bromo-indole-3-propionic acid methyl ester (17, 0.05mmol), phenyl boronic acid (18, 0.1 mmol), 0.2 mL 1M K₂CO₃ (0.2 mmol), acetonitrile (0.4 ml) and a few mg of tetrakis(triphenylphosphine)palladium(0) were combined and heated in the microwave at 160° C. for 400 seconds. The crude material was then purified via flash chromatography with silica, eluting with a step gradient of 2 to 4 to 6% methanol in chloroform to isolate the desired compound as a solid.

### Step 4: Preparation of 3-{5-phenyl-1-[4-(4-trifluoromethyl-phenoxy)-benzenesulfonyl]-1H-indol-3-yl}-propionic acid methyl ester (21)

Into a flask containing 3-(5-phenyl-1H-indol-3-yl)-propionic acid methyl ester 19, Immol) dissolved in 5 mL tetrahydrofuran, BEMP (1.1 mmol), and 4-(4-trifluoromethyl-phenoxy)-benzenesulfonyl chloride (20, 1.05 mmol) were combined and at room temperature for 2 hours. The crude mixture was taken on to the next step.

### Step 5: Preparation of 3-{5-phenyl-1-[4-(4-trifluoromethyl-phenoxy)-benezenesulfonyl]-1H-indol-3-yl}-propionic acid (P-0082)

Into a flask, the crude mixture from Step 4 was dissolved in 1 M NaOH solution, and stirred for 4 hours at ambient temperature. The hydrolysis was monitored via LC-MS. Upon full transformation, the basic solution was neutralized with acetic acid, followed by removal of the solvent under reduced pressure to yield a crude solid. The crude material en taken up in dimethylsulfoxide and purified via reverse phase HPLC with a 20-100% acetonitrile gradient (12 minute gradient). The purified material was then analyzed via HPLC to identify the pure fractions. The fractions were then combined and concentrated down to afford the desired compound as a solid. Calculated molecular weight of 565.57, MS(ESI) [M+H⁺]⁺=566.4.

Additional compounds were prepared following the protocol of Scheme 4, optionally replacing phenyl boronic acid 18 with pyridine-3-boronic acid or thiophene-3-boric acid in Step 3, and/or optionally replacing 4-(4-trifluoromethyl-phenoxy)-benzenesulfonyl chloride 20 with an appropriate sulfonyl chloride in Step 4. The following compounds were prepared by this method, with the calculated molecular weight and measured mass (MS(ESI)) provided after the compound:
3-[1-(5-Isoxazol-3-yl-thiophene-2-sulfonyl)-5-phenyl-1H-indol-3-yl]-propionic acid (P-0076), calculated MW 478.55, [M+H⁺]⁺=N/A,
3-{1-[5-(2-Methyl-thiazol-4-yl)-thiophene-2-sulfonyl]-5-phenyl-1H-indol-3-yl}-propionic acid (P-0077), calculated MW 508.64, [M+H⁺]⁺=509.1,
3-{5-Phenyl-1-[4-(pyridin-2-yloxy)-benzenesulfonyl]-1H-indol-3-yl}-propionic acid (P-0078), calculated MW 498.56, [M+H⁺]⁺=499.1,
3-{1-[4-(4-Methoxy-phenoxy)-benzenesulfonyl]-5-phenyl-1H-indol-3-yl}-propionic acid (P-0079), calculated MW 527.60, [M+H⁺]⁺=528.3,
3-{1-[4-(3,4-Dichloro-phenoxy)-benzenesulfonyl]-5-phenyl-1H-indol-3-yl}-propionic acid (P-0080), calculated MW 566.46, [M−H⁺]⁻=566.4
3-{1-[4-(3,5-Dichloro-phenoxy)-benzenesulfonyl]-5-phenyl-1H-indol-3-yl}-propionic acid (P-0081), calculated MW 566.46, [M−H⁺]⁻=566.4,
3-{1-[4-(3-Chloro-5-trifluoromethyl-pyridin-2-yloxy)-benzenesulfonyl]-5-phenyl-1H-indol-3-yl}-propionic acid (P-0083), calculated MW 601.01, [M+H⁺]⁺=601.2,
3-{1-[3-(3,4-Dichloro-phenoxy)-benzenesulfonyl]-5-phenyl-1H-indol-3-yl}-propionic acid (P-0084), calculated MW 566.46, [M+H⁺]⁺=566.8,
3-[1-(4′-Methoxy-biphenyl-4-sulfonyl)-5-phenyl-1H-indol-3-yl]-propionic acid (P-0085), calculated MW 511.60, [M+H⁺]⁺=512.3,
3-[1-(6-Morpholin-4-yl-pyridine-3-sulfonyl)-5-phenyl-1H-indol-3-yl]-propionic acid (P-0086), calculated MW 491.57, [M+H⁺]⁺=492.3,
3-[1-(6-Phenoxy-pyridine-3-sulfonyl)-5-phenyl-1H-indol-3-yl]-propionic acid (P-0087), calculated MW 498.56, [M+H⁺]⁺=499.1,
3-[5-Phenyl-1-(5-pyridin-2-yl-thiophene-2-sulfonyl)-1H-indol-3-yl]-propionic acid (P-0088), calculated MW 488.59, [M+H⁺]⁺=489.1,
3-(1-{4-[(Morpholine-4-carbonyl)-amino]-benzenesulfonyl}-5-phenyl-1H-indol-3-yl)-propionic acid (P-0089), calculated MW 533.61, [M+H⁺]⁺=534.3,
3-{1-[5-(1-Methyl-5-trifluoromethyl-1H-pyrazol-3-yl)-thiophene-2-sulfonyl]-5-phenyl-1H-indol-3-yl}-propionic acid (P-0091), calculated MW 559.59, [M+H⁺]⁺=560.4,
3-{1-[5-(2-Methylsulfanyl-pyrimidin-4-yl)-thiophene-2-sulfonyl]-5-pyridin-3-yl-1H-indol-3-yl}-propionic acid (P-0095), calculated MW 536.65, [M+H⁺]⁺=537.1,
3-{5-Pyridin-3-yl-1-[4-(pyridin-2-yloxy)-benzenesulfonyl]-1H-indol-3-yl}-propionic acid (P-0097), calculated MW 499.55, [M+H⁺]⁺=500.3,
3-{1-[4-(4-Methoxy-phenoxy)-benzenesulfonyl]-5-pyridin-3-yl-1H-indol-3-yl}propionic acid (P-0098), calculated MW 528.59, [M+H⁺]⁺=529.1,
3-{1-[4-(3,4-Dichloro-phenoxy)-benzenesulfonyl]-5-pyridin-3-yl-1H-indol-3-yl}-propionic acid (P-0099), calculated MW 567.45, [M−H⁺]⁻=567.2,
3-{1-[4-(3,5-Dichloro-phenoxy)-benzenesulfonyl]-5-pyridin-3-yl-1H-indol-3-yl}-propionic acid (P-0100), calculated MW 567.45, [M−H⁺]⁻=567.2,
3-{5-Pyridin-3-yl-1-[4-(4-trifluoromethyl-phenoxy)-benzenesulfonyl]-1H-indol-3-yl}-propionic acid (P-0101), calculated MW 566.56, [M+H⁺]⁺=567.2,
3-{1-[4-(3-Chloro-5-trifluoromethyl-pyridin-2-yloxy)-benzenesulfonyl]-5-pyridin-3-yl -1H-indol-3-yl}-propionic acid (P-0102), calculated MW 601.99, [M+H⁺]⁺=602.4,
3-{1-[3-(3,4-Dichloro-phenoxy)-benzenesulfonyl]-5-pyridin-3-yl-1H-indol-3-yl}-propionic acid (P-0103), calculated MW 567.45, [M−H⁺]⁻=567.2,
3-[1-(4′-Methoxy-biphenyl-4-sulfonyl)-5-pyridin-3-yl-1H-indol-3-yl]-propionic acid (P0104), calculated MW 512.59, [M+H⁺]⁺=513.5,
3-[1-(6-Phenoxy-pyridine-3-sulfonyl)-5-pyridin-3-yl-1H-indol-3-yl]-propionic acid (P0105), calculated MW 499.55, [M+H⁺]⁺=500.3,
3-[5-Pyridin-3-yl-1-(5-pyridin-2-yl-thiophene-2-sulfonyl)-1H-indol-3-yl]-propionic acid (P-0106), calculated MW 489.58, [M+H⁺]⁺=490.3,
3-{1-[5-(1Methyl-5-trifluoromethyl-1H-pyrazol-3-yl)-thiophene-2-sulfonyl]-5-pyridin-3-yl-1H-indol-3-yl}-propionic acid (P-0107), calculated MW 560.58, [M+H⁺]⁺=561.2,
3-[1-(5-Methyl-1-phenyl-1H-pyrazole-4-sulfonyl)-5-pyridin-3-yl-1H-indol-3-yl]-propionic acid (P-0113), calculated MW 486.55, [M+H⁺]⁺=487.1,
3-[1-(6-Morpholin-4-yl-pyridine-3-sulfonyl)-5-pyridin-3-yl-1H-indol-3-yl]-propionic acid (P-0114), calculated MW 492.56, [M+H⁺]⁺=493.5,
3-(1-{4-[(Morpholine-4-carbonyl)-amino]-benzenesulfonyl}-5-pyridin-3-yl-1H-indol-3-yl)-propionic acid (P-0115), calculated MW 534.60, [M+H⁺]⁺=535.1,
3-{5-Pyridin-3-yl-1-[4-(pyridin-4-yloxy)-benzenesulfonyl]-1H-indol-3-yl}-propionic acid (P-0118), calculated MW 499.55, [M+H⁺]⁺=500.3,
3-{1-[3-(2-Methyl-pyrimidin-4-yl)-benzenesulfonyl]-5-pyridin-3-yl-1H-indol-3-yl}-propionic acid (P-0120), calculated MW 498.56, [M+H⁺]⁺=499.1,
3-[1-(Biphenyl-2-sulfonyl)-5-pyridin-3-yl-1H-indol-3-yl]-propionic acid (P-0125), calculated MW 482.56, [M+H⁺]⁺=483.1,
3-[1-(2-Phenoxy-benzenesulfonyl)-5-pyridin-3-yl-1H-indol-3-yl]-propionic acid (P0128), calculated MW 498.56, [M+H⁺]⁺=499.1,
3-{1-[3-(Pyridine-2-carbonyl)-benzenesulfonyl]-5-pyridin-3-yl-1H-indol-3-yl}-propionic acid (P-0131), calculated MW 511.56, [M+H⁺]⁺=512.3,
3-[1-(4-Pyrazol-1-yl-benzenesulfonyl)-5-pyridin-3-yl-1H-indol-3-yl]-propionic acid (P0137), calculated MW 472.53, [M+H⁺]⁺=473.1,
3-{1-[5-(2-Methylsulfanyl-pyrimidin-4-yl)-thiophene-2-sulfonyl]-5-thiophen-3-yl-1H-indol-3-yl}-propionic acid (P-0573), calculated MW 541.70,
3-{1-[4-(3,5-Dichloro-phenoxy)-benzenesulfonyl]-5-thiophen-3-yl-1H-indol-3-yl}-propionic acid (P-0574), calculated MW 572.49,
3-{1-[3-(3,4-Dichloro-phenoxy)-benzenesulfonyl]-5-thiophen-3-yl-1H-indol-3-yl}-propionic acid (P-0575), calculated MW 572.49,
3-{1-[4-(Pyridin-2-yloxy)-benzenesulfonyl]-5-thiophen-3-yl-1H-indol-3-yl}-propionic acid (P-0581), calculated MW 504.58,
3-{1-[4-(Pyridin-4-yloxy)-benzenesulfonyl]-5-thiophen-3-yl-1H-indol-3-yl}-propionic acid (P-0582), calculated MW 504.58,
3-{1-[4-(4-Methoxy-phenoxy)-benzenesulfonyl]-5-thiophen-3-yl-1H-indol-3-yl}-propionic acid (P-0583), calculated MW 533.62,
3-{1-[4-(3,4-Dichloro-phenoxy)-benzenesulfonyl]-5-thiophen-3-yl-1H-indol-3-yl}-propionic acid (P-0584), calculated MW 572.49,
3-{5-Thiophen-3-yl-1-[4-(4-trifluoromethyl-phenoxy)-benzenesulfonyl]-1H-indol-3-yl}-propionic acid (P-0585), calculated MW 571.59,
3-{1-[4-(3-Chloro-5-trifluoromethyl-pyridin-2-yloxy)-benzenesulfonyl]-5-thiophen-3-yl-1H-indol-3-yl}-propionic acid (P-0586), calculated MW 607.03,
3-[1-(4′-Methoxy-biphenyl-4-sulfonyl)-5-thiophen-3-yl-1H-indol-3-yl]-propionic acid (P0587), calculated MW 517.62,
3-[1-(6-Morpholin-4-yl-pyridine-3-sulfonyl)-5-thiophen-3-yl-1H-indol-3-yl]-propionic acid (P-0588), calculated MW 497.59,
3-[1-(6-Phenoxy-pyridine-3-sulfonyl)-5-thiophen-3-yl-1H-indol-3-yl]-propionic acid (P0589), calculated MW 504.58,
3-[1-(5-Pyridin-2-yl-thiophene-2-sulfonyl)-5-thiophen-3-yl-1H-indol-3-yl]-propionic acid(P-0590), calculated MW 494.61,
3-{1-[5-(1Methyl-5-trifluoromethyl-1H-pyrazol-3-yl)-thiophene-2-sulfonyl]-5-thiophen-3-yl-1H-indol-3-yl}-propionic acid (P-0592), calculated MW 565.62,
3-[1-(5-Methyl-1-phenyl-1H-pyrazole-4-sulfonyl)-5-thiophen-3-yl-1H-indol-3-yl]-propionic acid (P-0599), calculated MW 491.59,
3-{1-[3-(2-Methyl-pyrimidin-4-yl)-benzenesulfonyl]-5-thiophen-3-yl-1H-indol-3-yl}-propionic acid (P-0601), calculated MW 503.60,
3-{1-[3-(Pyridine-2-carbonyl)-benzenesulfonyl]-5-thiophen-3-yl-1H-indol-3-yl}-propionic acid (P-0607), calculated MW 516.60,
3-[1-(Biphenyl-2-sulfonyl)-5-thiophen-3-yl-1H-indol-3-yl]-propionic acid (P-0608), calculated MW 487.60,
3-[1-(4′-Methyl-biphenyl-2-sulfonyl)-5-thiophen-3-yl-1H-indol-3-yl]-propionic acid (P0617), calculated MW 501.62,
3-[1-(2-Phenoxy-benzenesulfonyl)-5-thiophen-3-yl-1H-indol-3-yl]-propionic acid (P0618), calculated MW 503.60,

These compounds are shown in the following Table 5, indicating the boronic acid used in Step 3 (Column 2), and the sulfonyl chloride used in the Step 4 (Column 3), with the compound structure provided in Column 4 and the compound number provided in Column 1.

TABLE 5

| Cmpd. | | | |
|---|---|---|---|
| number | Boronic acid | Sulfonyl chloride | Compound structure |
| P-0076 | 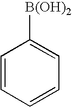 | 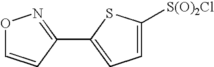 | 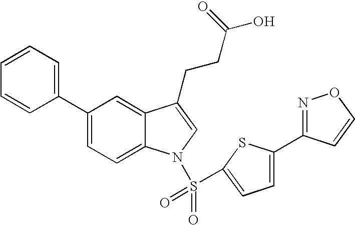 |
| P-0077 | 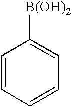 | 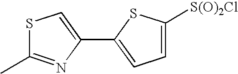 | 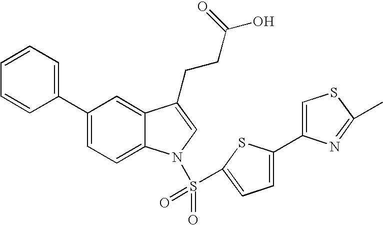 |
| P-0078 | 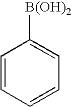 | 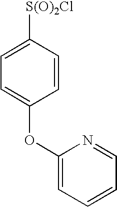 | 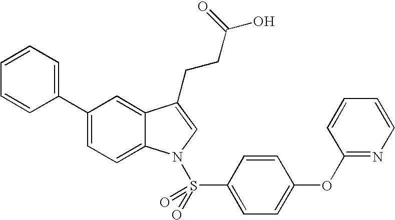 |
| P-0079 | 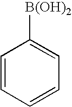 | 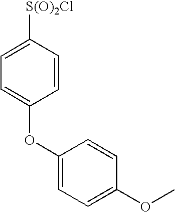 | 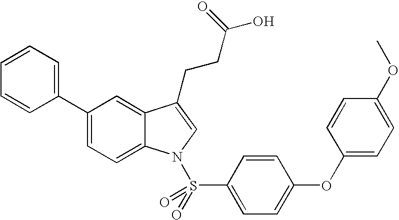 |
| P-0080 | 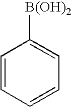 | 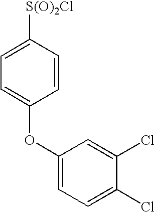 | 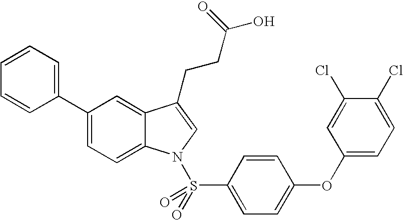 |
| P-0081 | 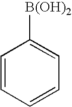 | 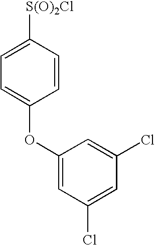 | 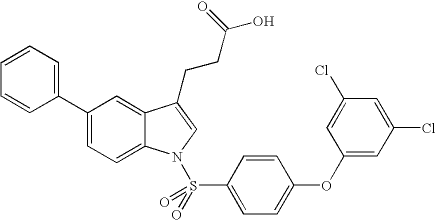 |
| P-0083 | 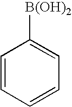 | 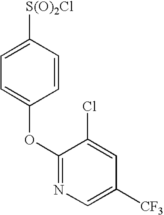 | 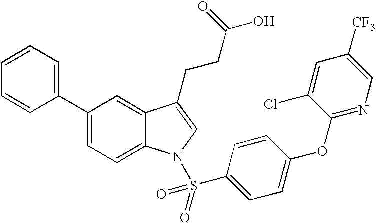 |
| P-0084 | 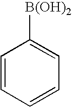 | 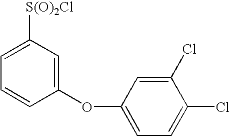 | 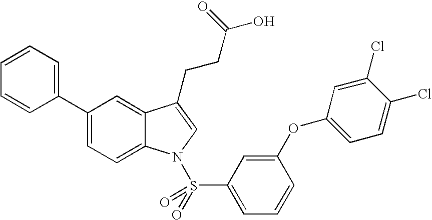 |
| P-0085 | 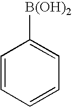 | 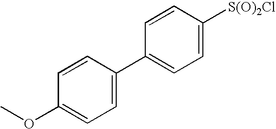 | 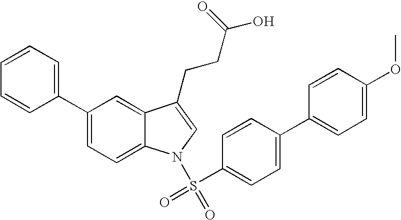 |
| P-0086 | 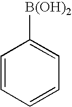 | 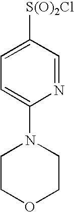 | 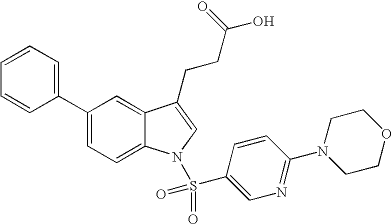 |
| P-0087 | 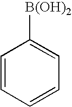 | 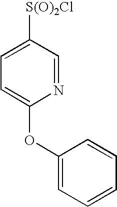 | 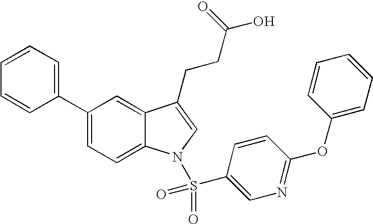 |
| P-0088 | 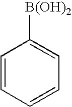 | 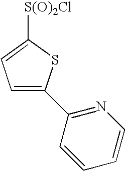 | 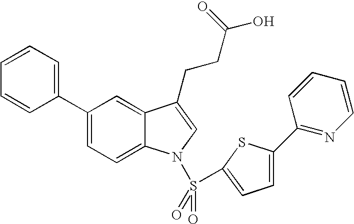 |
| P-0089 | 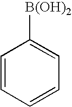 | 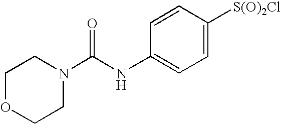 | 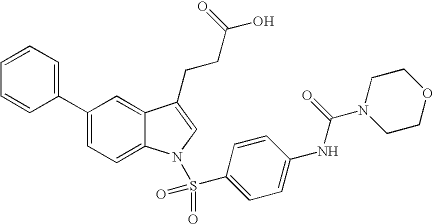 |
| P-0091 | 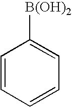 | 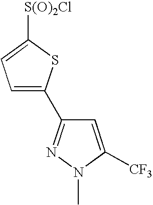 | 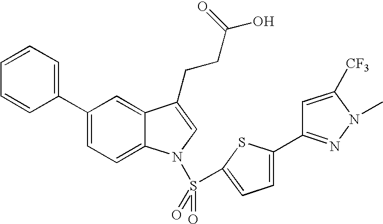 |
| P-0095 | 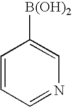 | 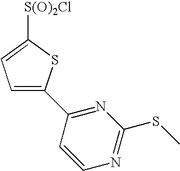 | 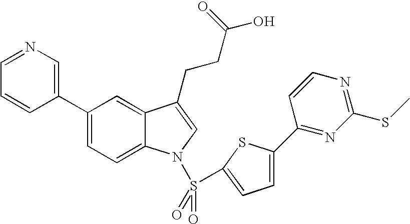 |
| P-0097 | 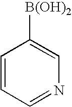 | 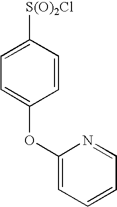 | 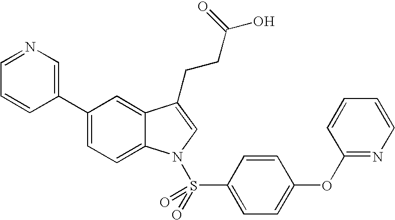 |
| P-0098 | 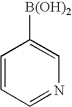 | 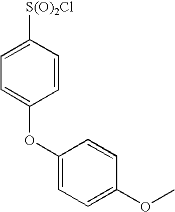 | 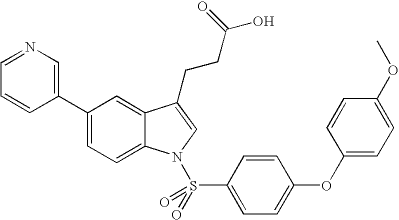 |
| P-0099 | 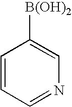 | 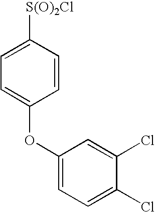 | 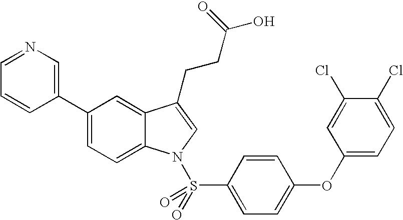 |
| P-0100 | 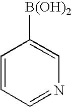 | 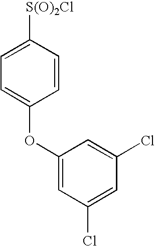 | 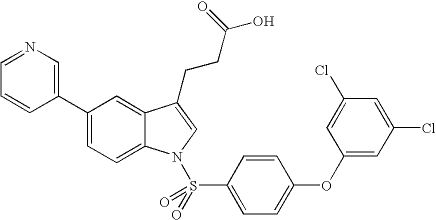 |
| P-0101 | 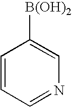 | 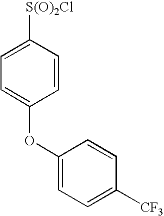 | 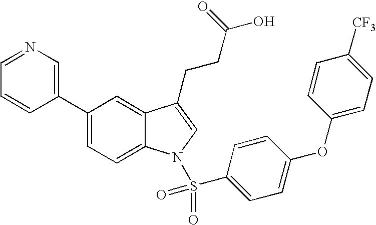 |
| P-0102 | 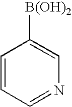 | 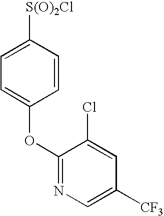 | 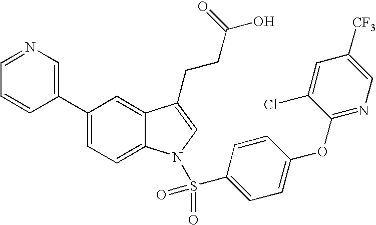 |
| P-0103 | 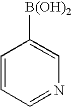 | 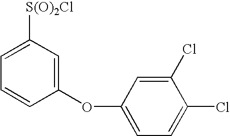 | 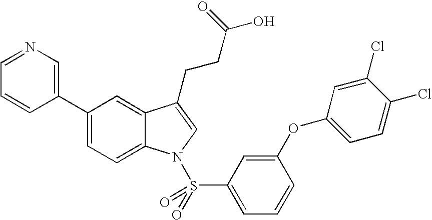 |
| P-0104 | 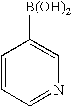 | 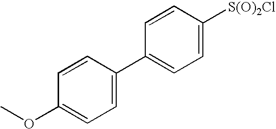 | 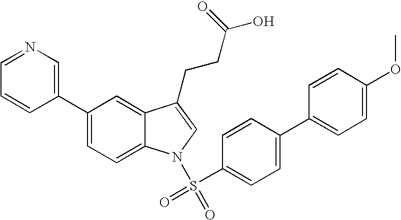 |
| P-0105 | 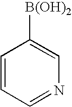 | 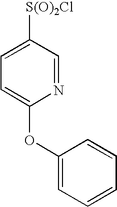 | 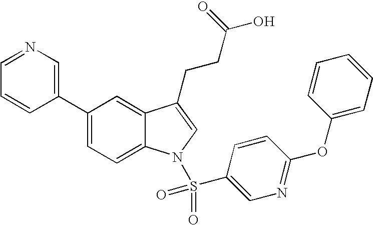 |
| P-0106 | 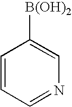 | 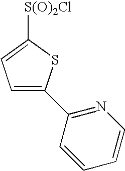 | 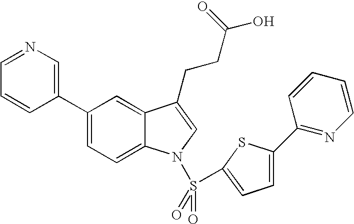 |
| P-0107 | 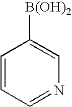 | 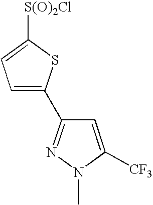 | 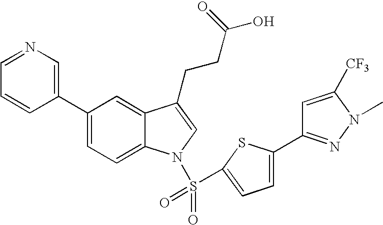 |
| P-0113 | 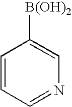 | 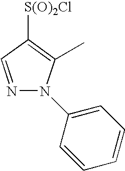 | 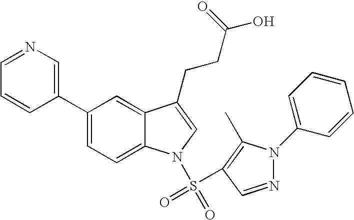 |
| P-0114 | 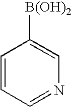 | 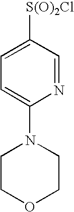 | 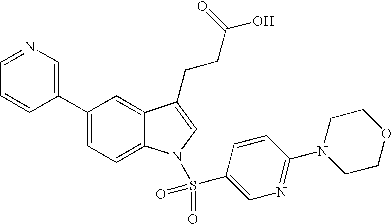 |
| P-0115 | 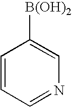 | 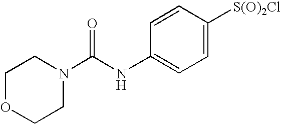 | 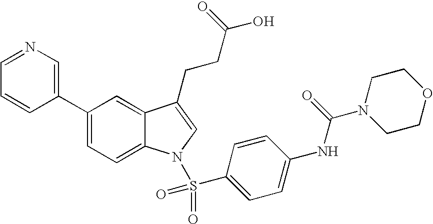 |
| P-0118 | 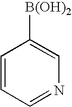 | 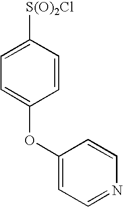 | 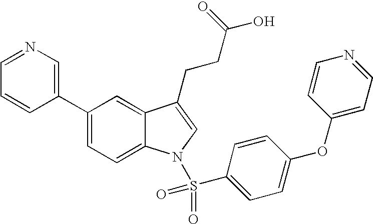 |
| P-0120 | 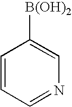 | 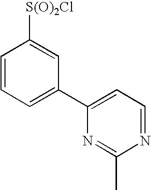 | 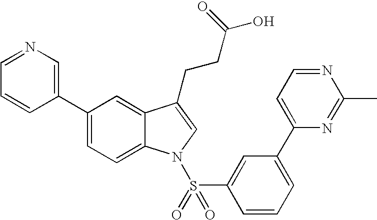 |
| P-0125 | 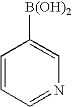 | 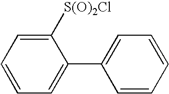 | 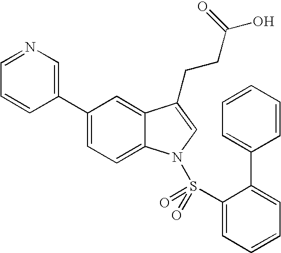 |
| P-0128 | 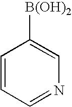 | 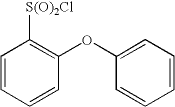 | 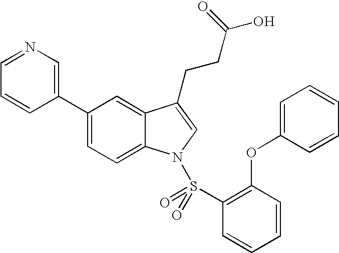 |
| P-0131 | 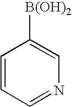 | 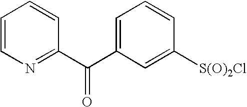 | 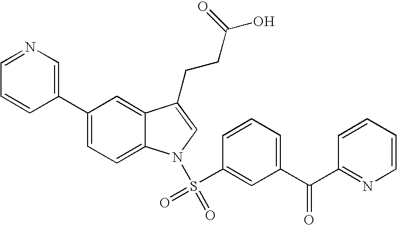 |
| P-0137 | 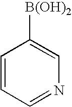 | 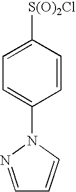 | 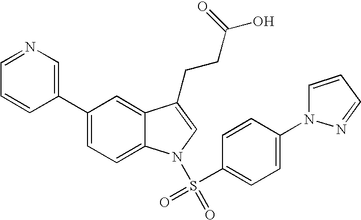 |
| P-0573 | 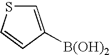 | 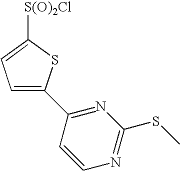 | 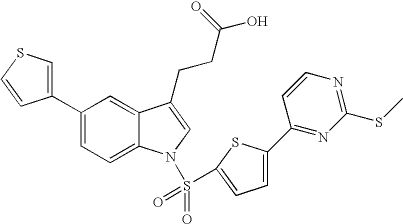 |
| P-0574 | 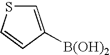 | 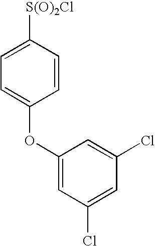 | 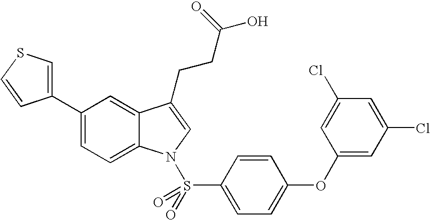 |
| P-0575 | 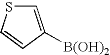 | 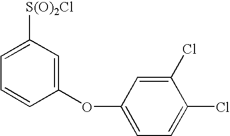 | 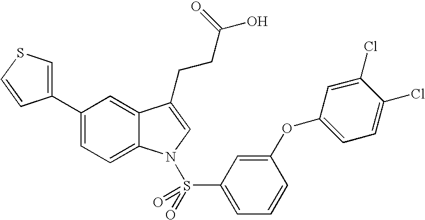 |
| P-0581 | 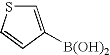 | 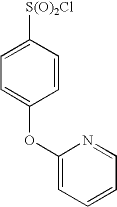 | 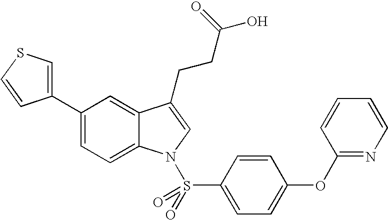 |
| P-0582 | 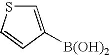 | 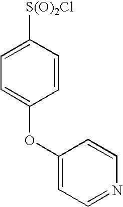 | 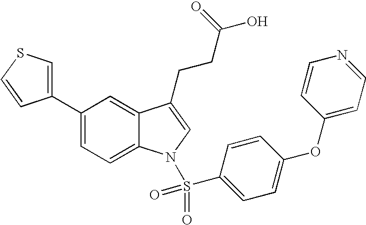 |
| P-0583 | 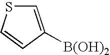 | 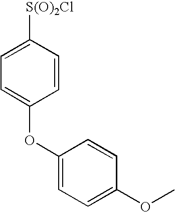 | 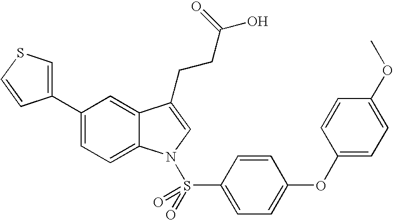 |
| P-0584 | 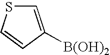 | 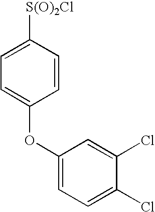 | 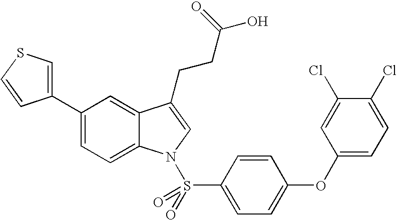 |
| P-0585 | 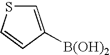 | 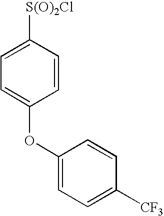 | 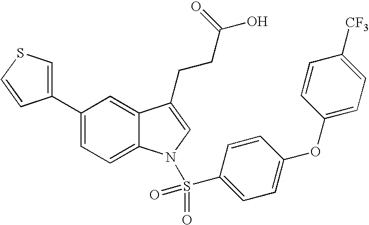 |
| P-0586 | 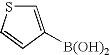 | 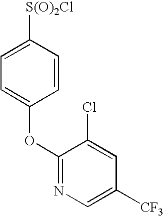 | 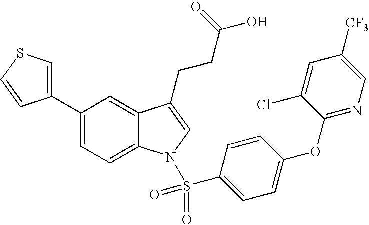 |
| P-0587 | 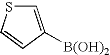 | 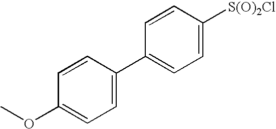 | 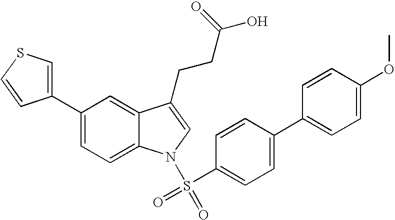 |
| P-0588 | 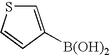 | 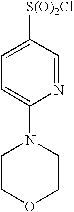 | 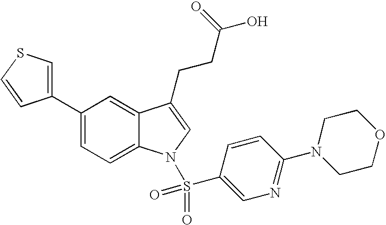 |
| P-0589 | 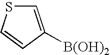 | 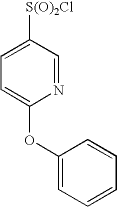 | 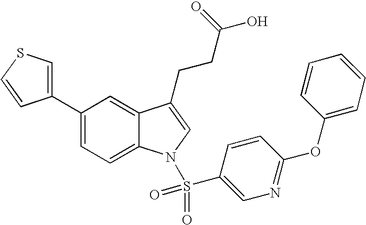 |
| P-0590 | 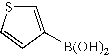 | 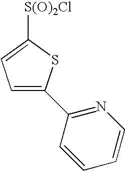 | 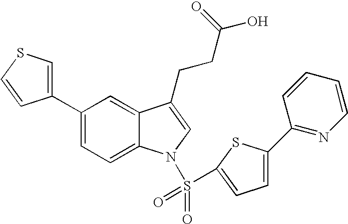 |
| P-0592 | 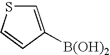 | 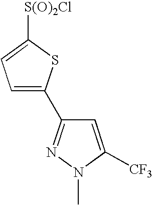 | 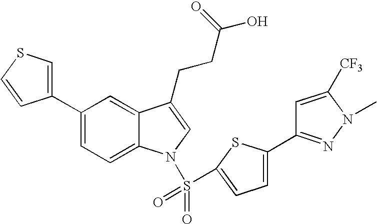 |
| P-0599 | 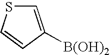 | 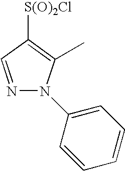 | 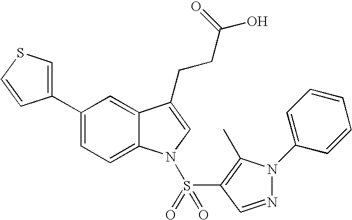 |
| P-0601 | 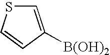 | 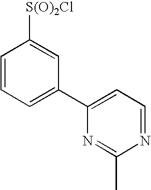 | 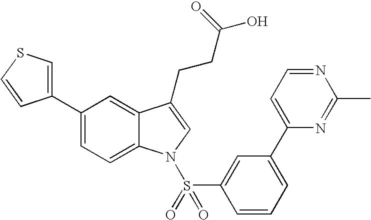 |
| P-0607 | 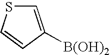 | 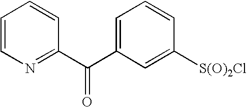 | 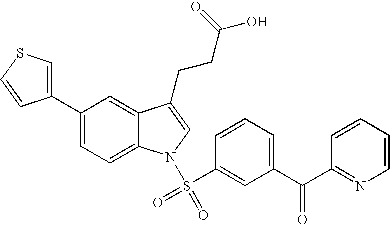 |
| P-0608 | 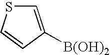 | 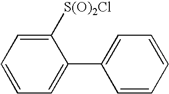 | 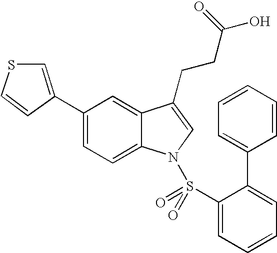 |
| P-0617 | 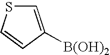 | 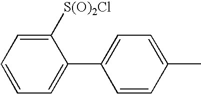 | 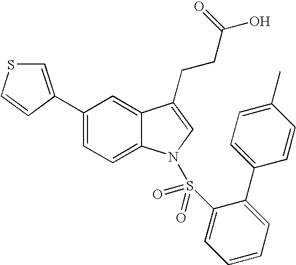 |
| P-0618 | 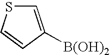 | 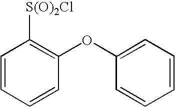 | 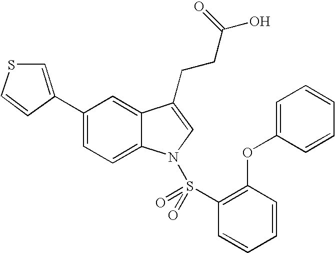 |

### Example 5

### Synthesis of 3-{6-Ethoxy-1-[4-(4-trifluoromethyl-phenoxy)-benzenesulfonyl]-1H-indol-3-yl}-propionic acid (P-0174).

Compound P-0174 was synthesized in five Steps as shown in Scheme 5.

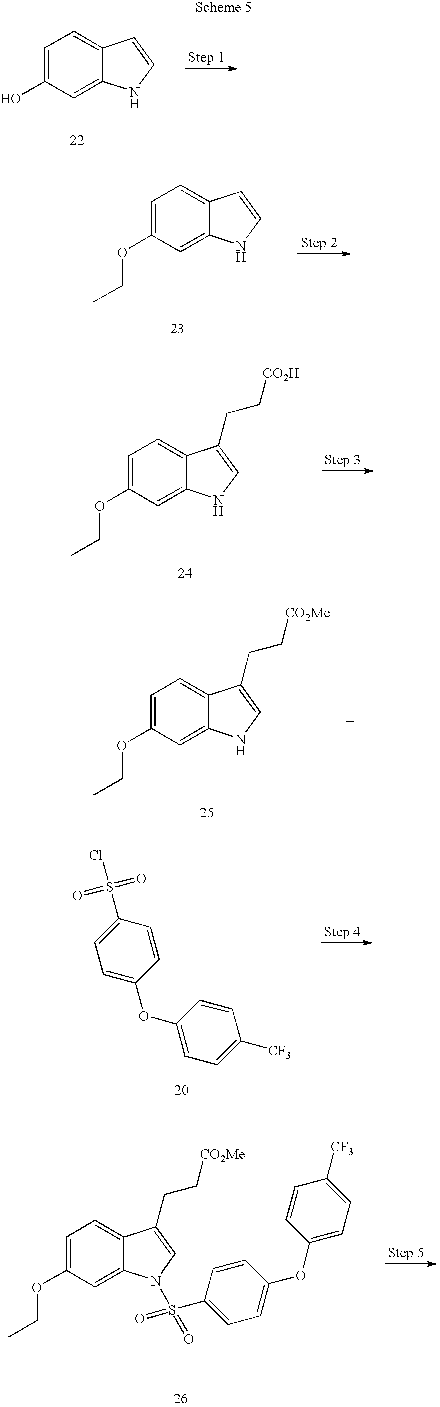

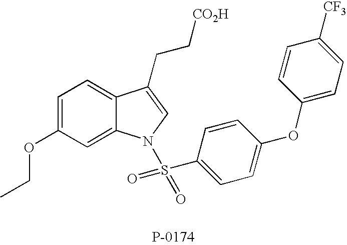

### Step: b 1: preparation of 6-ethoxyindole (23)

Into a round bottom flask 6-hydroxyindole (22, 2 g, 0.02 mol), potassium carbonate (4 g, 0.03 mol), acetonitrile (20 g, 0.5 mol), and iodoethane (4 g, 0.02 mol) were combined and stirred at ambient temperature for 3-4 days. The reaction was filtered and washed with dichloromethane. The organic layer was then washed with water twice, brine once, and dried over sodium sulfate. The evaporation of the solvent yielded an oil. The oil was then absorbed onto silica and purified via 80% hexane, 20% ethyl acetate to yield a yellow solid. ¹H NMR is consistent with the compound structure set forth above.

### Step 2: Preparation of the 6-ethoxy-indole-3-propionic acid (24)

Into a microwave vessel, 6-ethoxy indole (23, 1 equivalent), paraformaldehyde (1.1 equivalent), 2,2-dimethyl-1,3-dioxane-4,6-dione (1.1 equivalent), and triethylamine (1.1 equivalent) were dissolved in acetonitrile (2 mL/mmol). The reaction was heated at 150° C. for 3 minutes in a microwave reactor. The reaction was then diluted with acidified water to pH ˜5 with acetic acid and the aqueous layer was extracted with ethyl acetate. The organic layer was then washed with water 2×, brine 1×, and dried over magnesium sulfate. Evaporation of solvent afforded a solid. The crude solid was then purified via flash chromatography with step gradient of 2 to 4 to 6% methanol in chloroform on silica to obtain the desired compound as an oil.

### Step 3: Preparation of 6-ethoxy-indole-3-propionic acid methyl ester (25)

In a flask, the 6-ethoxy-indole-3-propionic acid 24 was treated with methanol (4 mol equiv), N,N′-Diisopropylcarbodiimide (2 mol equiv), a catalytic amount of dimethylaminopyridine in dichloromethane and stirred for 15 to 20 minutes at ambient conditions. The solvent was removed under reduced pressure and the mixture purified via flash chromatography on silica (chloroform) to obtain the desired compound as an off-white solid.

### Step 4: Preparation of 3-{6-ethoxy-1-[4-(4-trifluoromethyl-phenoxy)-benzesulfonyl]-1H-indol-3-yl}-propionic acid methyl ester (26)

Into a flask containing 6-ethoxy-indole-3-propionic acid methyl ester (25, 1 mmol) dissolved in 5 mL tetrahydrofuran, BEMP (1.1 mmol), and 4-(4-trifluoromethyl-phenoxy)-benzenesulfonyl chloride (20, 1.05 mmol) were combined and mixed at room temperature for 2 hours. The crude mixture was taken on to the next step.

### Step 5: Preparation of 3-{6-ethoxy-1-[4-(4-trifluoromethyl-phenoxy)-benzenesulfonyl]-1H-indol-3-yl}-propionic acid (P-0174)

Into a flask, the crude mixture from Step 4 was dissolved in 1M NaOH solution, and stirred for 4 hours at ambient temperature. The hydrolysis was monitored via LC-MS. Upon full transformation, the basic solution was neutralized with acetic acid, followed by removal of the solvent under reduced pressure to yield a crude solid. The crude material was then taken up in dimethylsulfoxide and purified via reverse phase HPLC with a 20-100% acetonitrile gradient (12 minute gradient). The purified material was then analyzed via HPLC to identify the pure fractions. The fractions were then combined and concentrated down to afford the desired compound as a solid. Calculated molecular weight of 533.53, MS (ESI) [M+H⁺]⁺=534.3.

Additional compounds were prepared following the protocol of Scheme 5, optionally replacing iodoethane with 2-iodopropane in Step 1, and/or optionally replacing 4-(4-trifluoromethyl-phenoxy)-benzenesulfonyl chloride 20 with an appropriate sulfonyl chloride in Step 4. The following compounds were prepared by this method, with the calculated molecular weight and measured mass (MS(ESI)) provided after the compound:
3-{6-Ethoxy-1-[4-(pyridin-3-yloxy)-benzenesulfonyl]-1H-indol-3-yl}-propionic acid (P0173), calculated MW 466.52, [M+H⁺]⁺=467.1,
3-{1-[3-(3,4-Dichloro-phenoxy)-benzenesulfonyl]-6-ethoxy-1H-indol-3-yl}-propionic acid (P-0175), calculated MW 534.42, [M−H⁺]⁻=533.9,
3-[6-Ethoxy-1-(6-morpholin-4-yl-pyridine-3-sulfonyl)-1H-indol-3-yl]-propionic acid (P-0176), calculated MW 459.53, [M+H⁺]⁺=460.3,
3-[6-Ethoxy-1-(6-phenoxy-pyridine-3-sulfonyl)-1H-indol-3-yl]-propionic acid (P-0177), calculated MW 466.52, [M+H⁺]⁺=467.1,
3-{6-Ethoxy-1-[3-(pyridine-2-carbonyl)-benzenesulfonyl]-1H-indol-3-yl}-propionic acid (P-0182), calculated MW 478.53, [M+H⁺]⁺=479.1,
3-{6-Ethoxy-1-[3-(pyridine-4-carbonyl)-benzenesulfonyl]-1H-indol-3-yl}-propionic acid (P-0183), calculated MW 478.53, [M+H⁺]⁺=479.1,
3-[1-(Biphenyl-2-sulfonyl)-6-ethoxy-1H-indol-3-yl]-propionic acid (P-0185), calculated MW 449.53, [M+H⁺]⁺=449.9,
3-[6-Ethoxy-1-(2-phenoxy-benzenesulfonyl)-1H-indol-3-yl]-propionic acid (P-0191), calculated MW 465.53, [M+H⁺]⁺=466.3,
3-{6-Ethoxy-1-[4-(4-methoxy-phenoxy)-benzenesulfonyl]-1H-indol-3-yl}-propionic acid (P-0195), calculated MW 495.56, [M+H⁺]⁺=496.3,
3-{1-[4-(3-Chloro-5-trifluoromethyl-pyridin-2-yloxy)-benzenesulfonyl]-6-ethoxy-1H-indol-3-yl}-propionic acid (P-0196), calculated MW 568.96, [M+H⁺]⁺=569.2,
3-[6-Ethoxy-1-(4′-methyl-biphenyl-2-sulfonyl)-1H-indol-3-yl]-propionic acid (P0201), calculated MW 463.56, [M+H⁺]⁺=464.3,
3-{6-Isopropoxy-1-[4-(4-methoxy-phenoxy)-benzenesulfonyl]-1H-indol-3-yl}-propionic acid (P-0206), calculated MW 509.58, [M+H⁺]⁺=510.3,
3-{6-Isopropoxy-1-[4-(4-trifluoromethyl-phenoxy)-benzenesulfonyl]-1H-indol-3-yl}-propionic acid (P-0207), calculated MW 547.55, [M+H⁺]⁺=548.3,
3-{6-Isopropoxy-1-[5-(1-methyl-5-trifluoromethyl-1H-pyrazol-3-yl)-thiophene-2-sulfonyl]-1H-indol-3-yl}-propionic acid (P-0208), calculated MW 541.57, [M+H⁺]⁺=543.1,
3-[6-Isopropoxy-1-(5-methyl-1-phenyl-1H-pyrazole-4-sulfonyl)-1H-indol-3-yl]-propionic acid (P-0232), calculated MW 467.55, [M+H⁺]⁺=468.3,
3-{1-[4-(3,4-Dichloro-phenoxy)-benzenesulfonyl]-6-isopropoxy-1H-indol-3-yl}-propionic acid (P-0233), calculated MW 548.45, [M+H⁺]⁺=550.3,
3-{1-[4-(3,5-Dichloro-phenoxy)-benzenesulfonyl]-6-isopropoxy-1H-indol-3-yl}-propionic acid (P-0234), calculated MW 548.45, [M−H⁺]⁻=547.9,
3-{1-[4-(3-Chloro-5-trifluoromethyl-pyridin-2-yloxy)-benzenesulfonyl]-6-isopropoxy-1H-3-yl}-propionic acid (P-0235), calculated MW 582.99, [M+H⁺]⁺=583.2,
3-{1-[3-(3,4-Dichloro-phenoxy)-benzenesulfonyl]-6-isopropoxy-1H-indol-3-yl}-propionic acid (P-0236), calculated MW 548.45, [M−H⁺]⁻=548.3,
3-[6-Isopropoxy-1-(4′-methoxy-biphenyl-4-sulfonyl)-1H-indol-3-yl]-propionic acid (P0237), calculated MW 493.58, [M+H⁺]⁺=494.3,
3-[6-Ethoxy-1-(5-methyl-1-phenyl-1H-pyrazole-4-sulfonyl)-1H-indol-3-yl]-propionic acid (P-0346), calculated MW 453.52, [M+H⁺]⁺=454.3,
3-{6-Ethoxy-1-[5-(2-methylsulfanyl-pyrimidin-4-yl)-thiophene-2-sulfonyl]-1H-indol-3-yl}-propionic acid (P-0347), calculated MW 503.62, [M+H⁺]⁺=504.3,
3-{6-Ethoxy-1-[4-(pyridin-2-yloxy)-benzenesulfonyl]-1H-indol-3-yl}-propionic acid (P0350), calculated MW 466.52, [M+H⁺]⁺=467.1,
3-{1-[4-(3,4-Dichloro-phenoxy)-benzenesulfonyl]-6-ethoxy-1H-indol-3-yl}-propionic acid (P-0351), calculated MW 534.42, [M−H⁺]⁻=533.9,
3-{1-[4-(3,5-Dichloro-phenoxy)-benzenesulfonyl]-6-ethoxy-1H-indol-3-yl}-propionic acid (P-0352), calculated MW 534.42, [M−H⁺]⁻=533.9,
3-[6-Ethoxy-1-(4′methoxy-biphenyl-4-sulfonyl)-1H-indol-3-yl]-propionic acid (P0353), calculated MW 479.56, [M+H⁺]⁺=479.9,
3-[6-Ethoxy-1-(5-pyridin-2-yl-thiophene-2-sulfonyl)-1H-indol-3-yl]-propionic acid (P0354), calculated MW 456.54, [M+H⁺]⁺=457.1,
3-(6-Ethoxy-1-{4-[(morpholine-4-carbonyl)-amino]-benzenesulfonyl}-1H-indol-3-yl)-propionic acid (P-0355), calculated MW 501.56, [M+H⁺]⁺=502.3, and
3-{6-Ethoxy-1-[5-(1-methyl-5-trifluoromethyl-1H-pyrazol-3-yl)-thiophene-2-sulfonyl]-1H-indol-3-yl}-propionic acid (P-0356), calculated MW 527.55, [M+H⁺]⁺=527.9

These compounds are shown in the following Table 6, indicating the iodoalkyl compound used in Step 1 (Column 2), and the sulfonyl chloride used in the Step 4 (Column 3), with the compound structure provided in Column 4 and the compound number provided in Column 1.

TABLE 6

| Cmpd. | | | |
|---|---|---|---|
| number | Iodo-alkyl | Sulfonyl chloride | Compound structure |
| P-0173 | 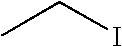 | 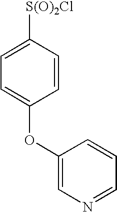 | 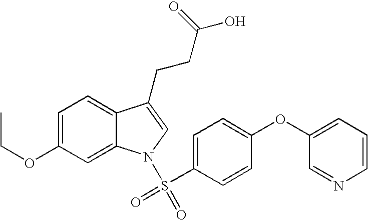 |
| P-0175 | 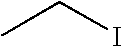 | 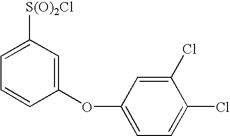 | 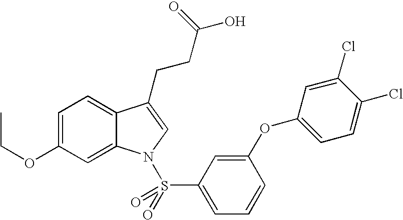 |
| P-0176 | 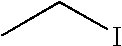 | 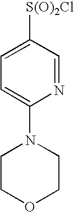 | 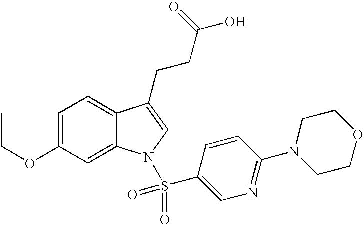 |
| P-0177 | 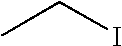 | 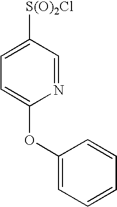 | 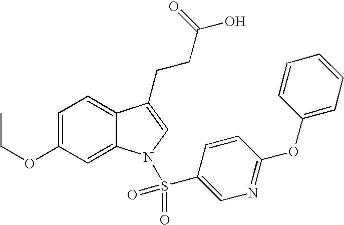 |
| P-0182 | 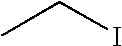 | 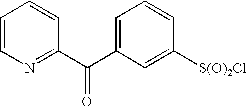 | 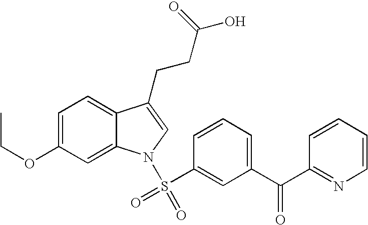 |
| P-0183 | 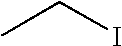 | 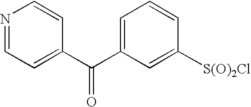 | 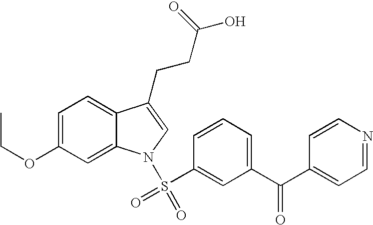 |
| P-0185 | 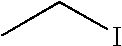 | 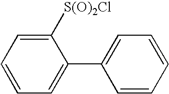 | 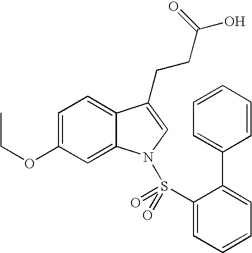 |
| P-0191 | 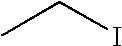 | 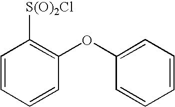 | 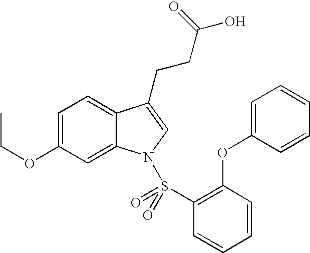 |
| P-0195 | 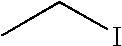 | 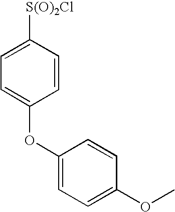 | 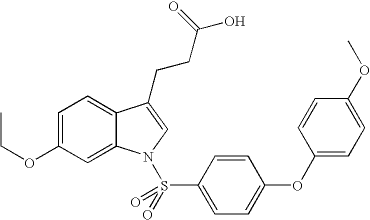 |
| P-0196 | 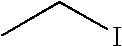 | 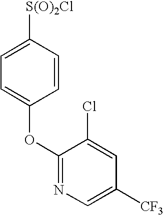 | 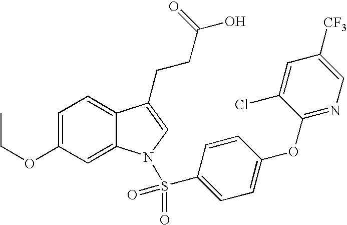 |
| P-0201 | 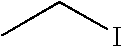 | 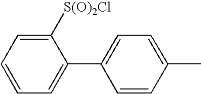 | 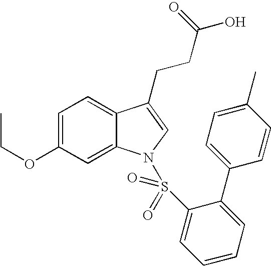 |
| P-0206 | 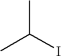 | 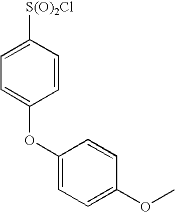 | 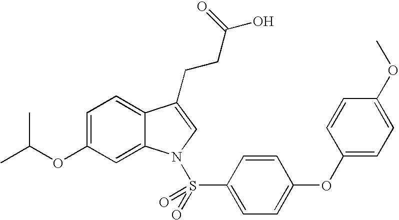 |
| P-0207 | 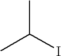 | 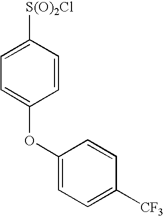 | 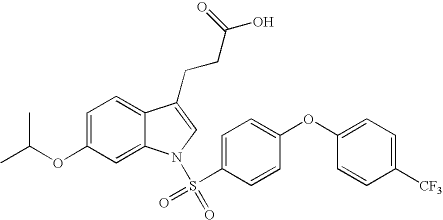 |
| P-0208 | 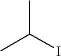 | 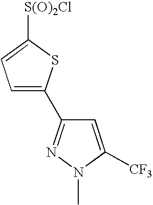 | 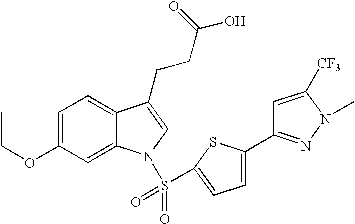 |
| P-0232 | 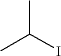 | 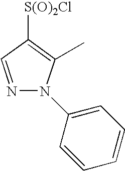 | 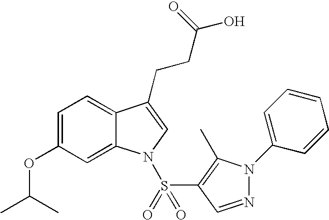 |
| P-0233 | 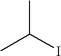 | 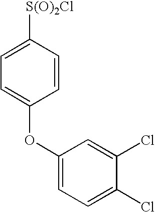 | 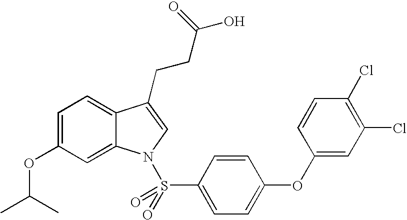 |
| P-0234 | 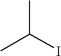 | 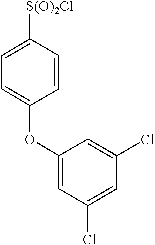 | 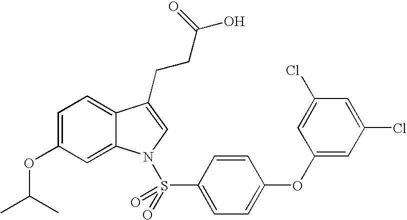 |
| P-0235 | 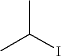 | 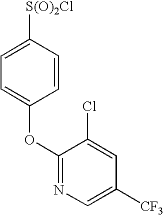 | 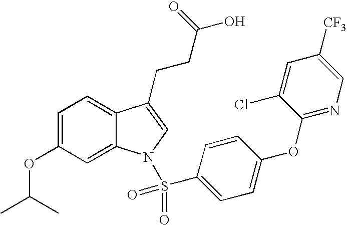 |
| P-0236 | 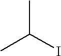 | 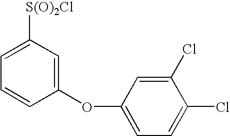 | 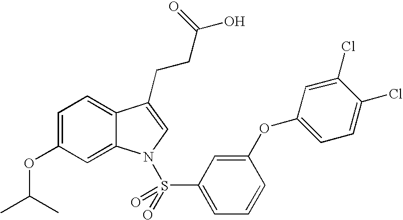 |
| P-0237 | 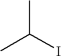 | 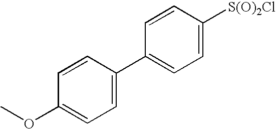 | 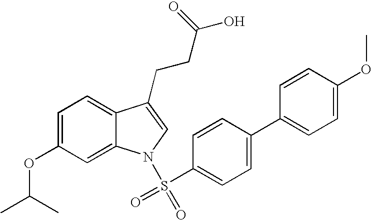 |
| P-0346 | 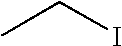 | 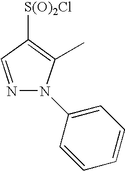 | 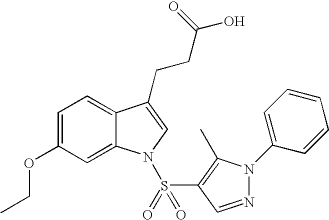 |
| P-0347 | 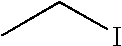 | 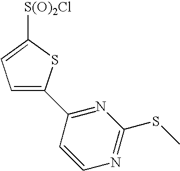 | 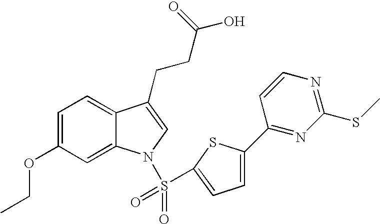 |
| P-0350 | 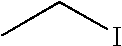 | 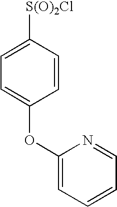 | 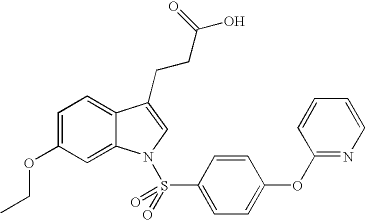 |
| P-0351 | 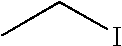 | 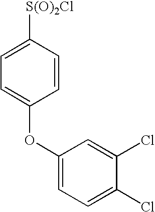 | 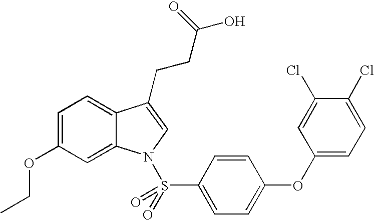 |
| P-0352 | 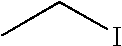 | 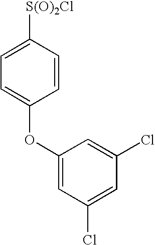 | 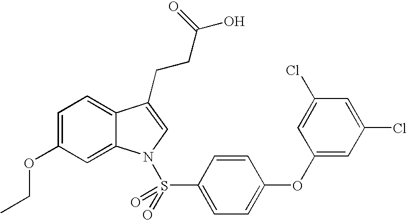 |
| P-0353 | 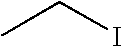 | 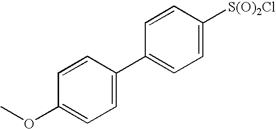 | 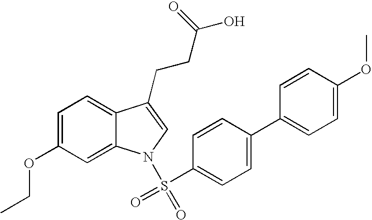 |
| P-0354 | 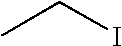 | 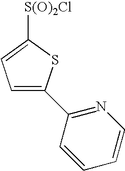 | 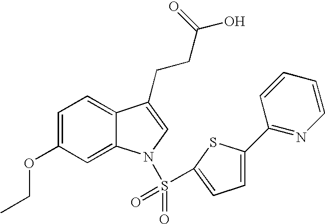 |
| P-0355 | 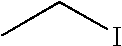 | 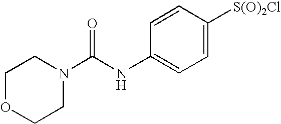 | 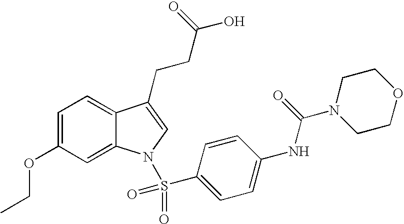 |
| P-0356 | 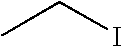 | 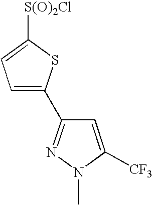 | 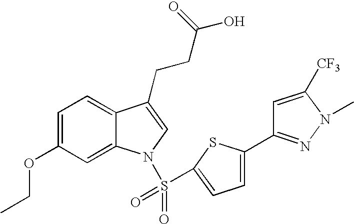 |

### Example 6

### Synthesis of 1-(4-butoxy-benzenesulfonyl)-5-methoxy-3-[2-(1H-tetrazol-5-yl)-ethyl]-1H-indole (P-0623).

Compound P-0623 was synthesized in four Steps as shown in Scheme 6.

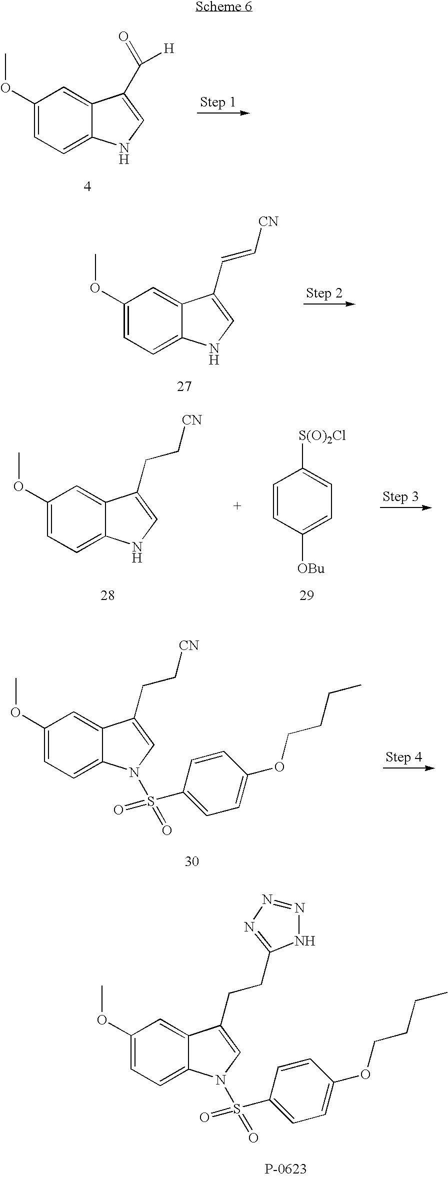

### Step 1: Preparation of (E)-3-(5-methoxy-1H-indol-3-yl)-acrylonitrile (27)

Into a 1-neck round-bottom flask, 5-methoxyindole-3-carboxaldehyde (4, 0.500 g, 0.00280 mol) was dissolved in tetrahydrofuran (18 mL, 0.23 mol). In a separate flask, diethyl cyanomethylphosphonate (0.909 mL, 0.00559 mol) was dissolved in 10 mL of tetrahydrofuran. The flask was cooled down and sodium hydride (224 mg, 0.00559 mol) was added to the flask under an atmosphere of argon. After the hydrogen gas evolution ceased, the solution was transferred into a syringe. The sodium phosphonoacetate tetrahydrofuran solution was added dropwise to the flask containing 5-methoxyindole-3-carboxyaldehyde at room temperature for 15 minutes. After the slow addition of the phosphonoacetate solution, the flask was heated at 55° C. overnight under an atmosphere of argon. The mixture was concentrated, then diluted with dichloromethane and washed with water (100 mL) three times. The combined organic layers were washed with brine one time, and dried over sodium sulfate. The dry organic layer was then filtered and the solvent removed by rotovap to afford a brown oil. The oil was purified with flash chromatography using 10-20% ethyl acetate in hexane. ¹H NMR is consistent with the compound structure set forth above.

### Step 2: Preparation of 3-(5-methoxy-1H-indol-3-yl)-propionitrile (28)

Into a flask, (E)-3-(5-methoxy-1H-indol-3-yl)-acrylonitrile (27, 190 mg, 0.00096 mol) was dissolved in tetrahydrofuran (30 mL, 0.4 mol). 5% Pd/C (5:95, palladium:carbon, 2.0E2 mg) was added and this mixture stirred overnight at ambient conditions under an atmosphere of hydrogen. The catalyst was filtered through celite, and the solvent was evaporated to afford a lightly colored oil. ¹H NMR is consistent with the compound structure set forth above.

### Step 3: Preparation of 3-[1-(4-butoxy-benzenesulfonyl)-5-methoxy-1H-indol-3-yl]propionitrile (30)

Into a round bottom flask, 3-(5-methoxy-1H-indol-3-yl)-propionitrile (28, 158 mg, 0.000789 mol) was suspended in toluene (2 mL, 0.02 mol). Potassium hydroxide (1 mL, 0.02 mol) and tetrabutylammonium hydrogen sulfate (7.5 mg, 0.000022 mol) were added. To this, 4-butoxy-benzenesulfonyl chloride (29, 156 μL, 0.000968 mol) was added and the reaction was stirred at ambient temperature for 5 hours. The reaction was diluted with ethyl acetate and water, the layers separated, and the aqueous layer extracted once with ethyl acetate. The combined organic layers were washed with water (3×), saturated sodium bicarbonate solution (1×), and brine (1×). The organic portion was dried over sodium sulfate and evaporated to dryness under reduced pressure. The product was purified using chromatography, eluting with ethyl acetate in hexanes. ¹H NMR is consistent with the compound structure set forth above.

### Step 4: Preparation of 1-(4-butoxy-benzenesulfonyl)-5-methoxy-3-[2-(1H-tetrazol-5-yl)-ethyl]-1H-indole (P-0623)

To a solution of 3-[1-(4-butoxy-benzenesulfonyl)-5-methoxy-1H-indol-3-yl]-propionitrile (30, 100 mg, 0.0002 mol) and azidotrimethylsilane (64.4 uL, 0.000485 mol) in tuluene (1 mL, 0.009 mol) was added dibutyloxostannane (6.0 mg, 0.000024 mol) and the mixture was heated at 110° C. overnight. The reaction mixture was concentrated in vacuo. The residue was dissolved in methanol and re-concentrated. The residue was partitioned between ethyl acetate and water. The organic phase was dried over sodium sulfate. The product was isolated with two successive prep TLC plate purification with 100% ethyl acetate with some acetic acid as solvent for the first run, followed by another TLC plate with 30% hexane 70% ethyl acetate with some formic acid. ¹H NMR is tent with the compound structure set forth above. Calculated molecular weight 455.54, MS (ESI) [M−H⁺]⁻=454.2.

### 1-(4-Butoxy-benzenesulfonyl)-5-methoxy-3-(1H-tetrazol-5-ylmethyl)-1H-indole P-624

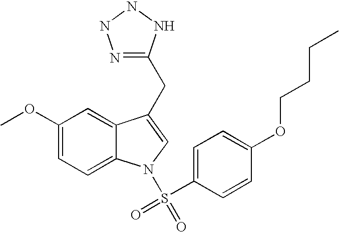 was prepared following Steps 3 and 4 of Scheme 6, replacing 3-(5-methoxy-1H-indol-3-yl)-propionitrile 28 with (5-methoxy-1H-indol-3-yl)-acetonitrile.

### Example 7

### Additional Compounds

Additional compounds of the invention were synthesized following the methods of the Examples above, or similar methods known to those of skill in the art, and are shown in the following Table 7, with the compound number in Column 1, compound structure in Column 2, compound name in Column 3, and calculated molecular weight and experimental mass spectrometry result in Columns 4 and 5.

TABLE 7

| | | | Molecular weight | |
|---|---|---|---|---|
| Comp. | | | | Measured |
| number | Structure | Name | Calc. | MS (ESI) |
| P-0005 | 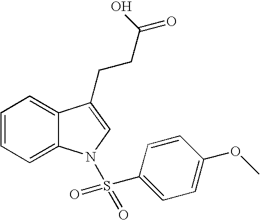 | 3-[1-(4-Methoxy- benzenesulfonyl)-1H- indol-3-yl]-propionic acid | 359.40 | [M − H⁺]⁻ =358.2 |
| P-0010 | 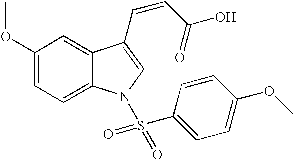 | (Z)-3-[5-Methoxy-1-(4- methoxy-benzenesulfonyl)- 1H-indol-3-yl]-acrylic acid | 387.41 | [M − H⁺]⁻ =386.1 |
| P-0011 | 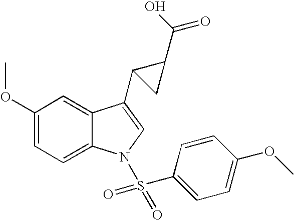 | 2-[5-Methoxy-1-(4-methoxy- benzenesulfonyl)-1H-indol- 3-yl]-cyclopropane carboxylic acid | 401.43 | [M − H⁺]− =400.2 |
| P-0013 | 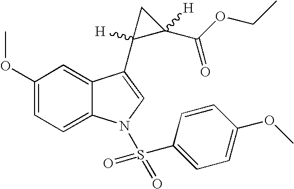 | 2-[5-Methoxy-1-(4-methoxy- benzenesulfonyl)-1H-indol- 3-yl]cyclopropane carboxylic acid ethyl ester | 429.49 | [M + H⁺]⁺ =430.4 |
| P-0021 | 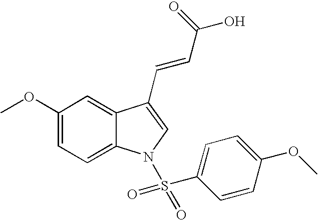 | (E)-3-[5-Methoxy-1-(4- methoxy-benzenesulfonyl)- 1H-indol-3-yl]-acrylic acid | 387.41 | [M + H⁺]⁺ =389.04 |
| P-0022 | 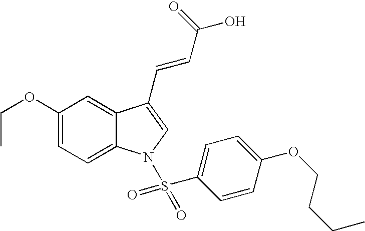 | (E)-3-[1-(4-Butoxy- benzenesulgonyl)-5-ethoxy- 1H-indol-3-yl]-acrylic acid | 443.52 | [M − H⁺]⁻ =441.90 |
| P-0023 | 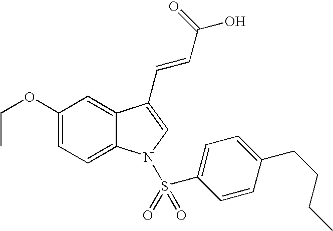 | (E)-3-[1-(4-Butyl- benzenesulfonyl)-5-ethoxy- 1H-indol-3-yl]-acrylic acid | 427.52 | [M − H⁺]⁻ =425.90 |
| P-0024 | 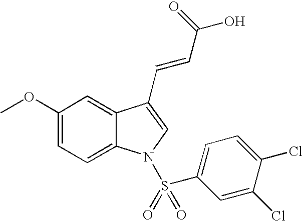 | (E)-3-[1-(3,4-Dichloro- benzenesulfonyl)-5- methoxy-1H-indol-3-yl]- acrylic acid | 373.42 | [M + H⁺]⁺ =374.2 |
| P-0025 | 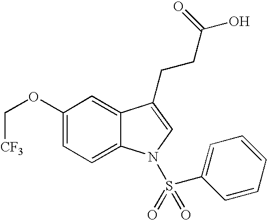 | 3-[1-Benzenesulfonyl-5- (2,2,2-trifluoro-ethoxy)-1H- indol-3-yl]-propionic acid | 444.47 | [M + H⁺]⁺ =445.3 |
| P-0027 | 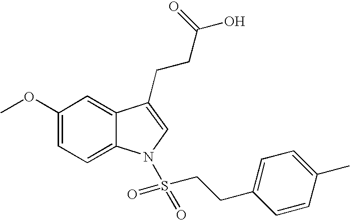 | 3-[5-Methoxy-1-(2-p-tolyl- ethanesulfonyl)-1H-indol-3- yl]-propionic acid | 401.48 | [M − H⁺]⁻ =400.1 |
| P-0028 | 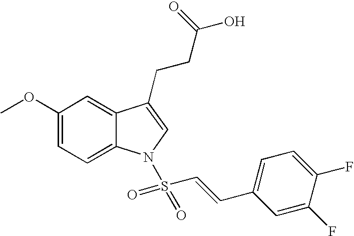 | 3-{1-[(E)-2-(3,4-Difluoro- phenyl)-ethenesulfonyl]-5- methoxy-1H-indol-3-yl}- propionic acid | 421.42 | [M − H⁺]⁻ =420.0 |
| P-0029 | 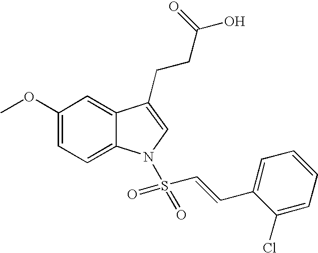 | 3-{1-[(E)-2-(2-Chloro- phenyl)-ethenesulfonyl]-5- methoxy-1H-indol-3-yl}- propionic acid | 419.88 | [M − H⁺]⁻ =418.0 |
| P-0030 | 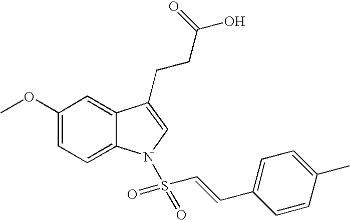 | 3-[5-Methoxy-1-((E)-2-p- tolyl-ethenesulfonyl)-1H- indol-3-yl]-propionic acid | 399.46 | [M − H⁺]⁻ =398.1 |
| P-0031 | 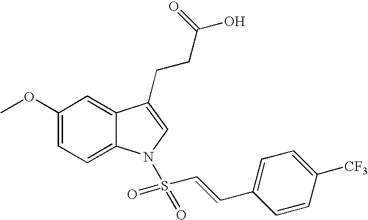 | 3-{5-Methoxy-1-[(E)-2-(4- trifluoromethyl-phenyl)- ethenesulfonyl]-1H-indol-3- yl}-propionic acid | 453.43 | [M − H⁺]⁻ =452.0 |
| P-0032 | 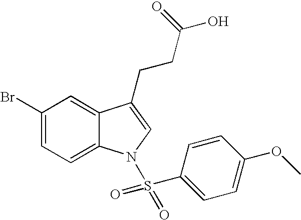 | 3-[5-Bromo-1-(4-methoxy- benzenesulfonyl)-1H-indol- 3-yl]-propionic acid | 436.99 | [M '1 H⁺]⁻ =436.11 |
| P-0039 | 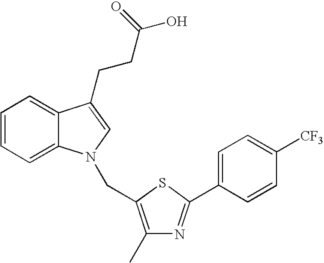 | 3-{1-[4-Methyl-2-(4- trifluoromethyl- phenyl)-thiazol-5-ylmethyl]- 1H-indol-3-yl}-propionic acid | 444.48 | [M + H⁺]⁺ =445.3 |
| P-0040 | 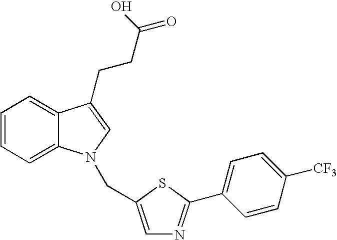 | 3-{1-[2-(4-Trifluoromethyl- phenyl)-thiazol-5-ylmethyl]- 1H-indol-3-yl}-propionic acid | 430.45 | [M + H⁺]⁺ =431.3 |
| P-0041 | 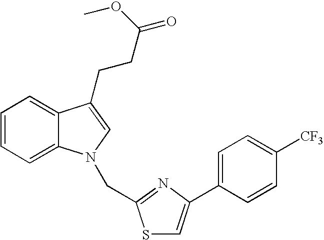 | 3-{1-[4-(4-Trifluoromethyl- phenyl)-thiazol-2-ylmethyl]- 1H-indol-3-yl}-propionic acid methyl ester | 444.48 | [M + H⁺]⁺ =445.3 |
| P-0042 | 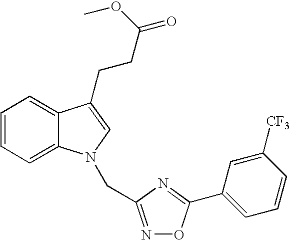 | 3-{1-[5-(3-Trifluoromethyl- phenyl)- [1,2,4]oxadiazol-3- ylmethyl]-1H-indol- 3-yl}-propionic acid methyl ester | 429.40 | [M + H⁺]⁺ =430.4 |
| P-0043 | 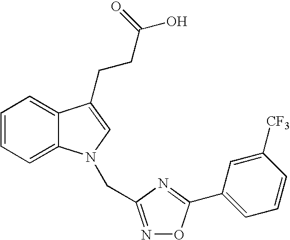 | 3-{1-[5-(3-Trifluoromethyl- phenyl)- [1,2,4]oxadiazol-3- ylmethyl]-1H-indol-3-yl}- propionic acid | 415.37 | [M + H⁺]⁺ =416.4 |
| P-0044 | 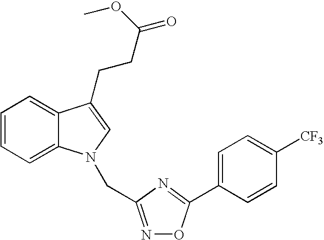 | 3-{1-[5-(4-Trifluoromethyl- phenyl)- [1,2,4]oxadiazol-3- ylmethyl]-1H-indol-3-yl}- propionic acid methyl ester | 429.40 | [M + H⁺]⁺ =430.3 |
| P-0045 | 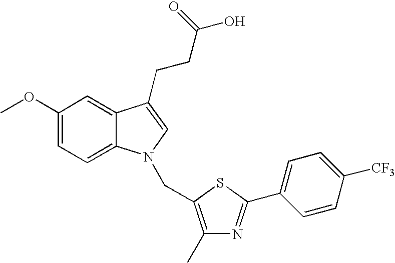 | 3-{5-Methoxy-1-[4-methyl- 2-(4-trifluoromethyl-phenyl)- thiazol-5-ylmethyl]-1H- indol-3-yl}-propionic acid | 474.50 | [M + H⁺]⁺ =475.2 |
| P-0046 | 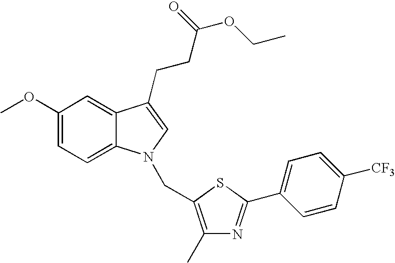 | 3-{5-Methoxy-1-[4-methyl- 2-(4-trifluoromethyl-phenyl)- thiazol-5-ylmethyl]-1H- indol-3-yl}-propionic acid ethyl ester | 502.56 | [M + H⁺]⁺ =503.3 |
| P-0054 | 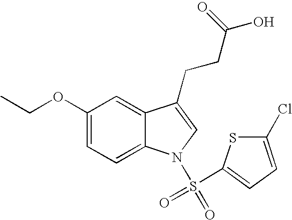 | 3-[1-(5-Chloro-thiophene-2- sulfonyl)-5-ethoxy-1H-indol- 3-yl]-propionic acid | 413.90 | [M + H⁺]⁺ =466.3 |
| P-0070 | 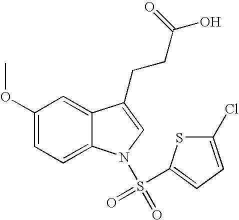 | 3-[1-(5-Chloro-thiophene-2- sulfonyl)-5-ethoxy-1H- indol-3-yl]propionic acid | 399.87 | [M + H⁺]⁺ =400.3 |
| P-0073 | 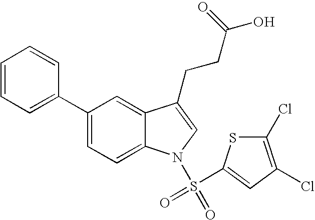 | 3-[1-(4,5-Dichloro- thiophene-2-sulfonyl)-5- phenyl-1H-indol-3-yl]- propionic acid | 480.39 | [M + H⁺]⁺ =481.1 |
| P-0074 | 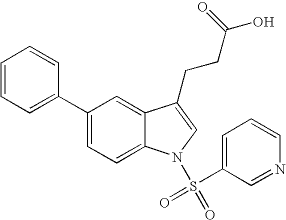 | 3-[5-Phenyl-1-(pyridine-3- sulfonyl)-1H-indol-3-yl]- propionic acid | 406.46 | [M + H⁺]⁺ =407.1 |
| P-0075 | 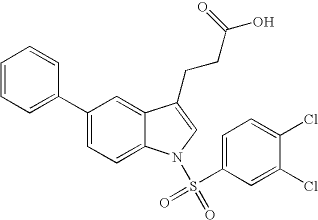 | 3-[1-(3,4-Dichloro- benzenesulfonyl)-5-phenyl- 1H-indol-3-yl]-propionic acid | 474.37 | |
| P-0090 | 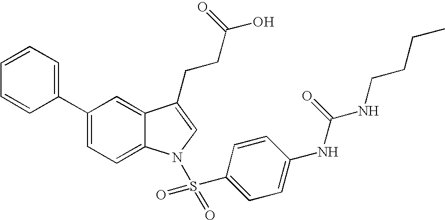 | 3-{1-[4-(3-Dichloro- benzenesulfonyl]-5-phenyl- 1H-indol-3-yl}-propionic acid | 519.62 | [M + H⁺]⁺ =520.3 |
| P-0092 | 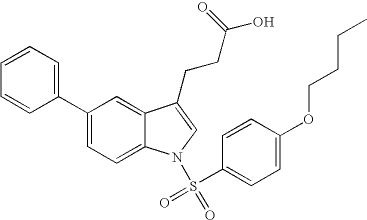 | 3-[1-(4-Butoxy- benzenesulfonyl)-5-phenyl- 1H-indol-3-yl]-propionic acid | 477.58 | [M − H⁺]⁻ =478.3 |
| P-0093 | 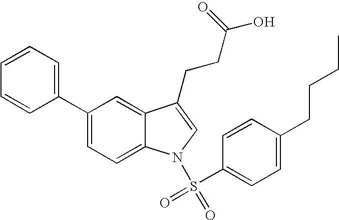 | 3-[1-(4-Butyl- benzenesulfonyl)-5-phenyl- 1H-indol-3-yl]-propionic acid | 461.58 | [M + H⁺]⁺ =462.3 |
| P-0094 | 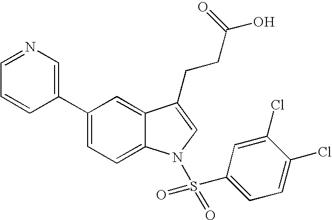 | 3-[1-(3,4-Dichloro- benzenesulfonyl)- 5-pyridin-3-yl-1H-indol-3- yl]-propionic acid | 475.35 | [M − H⁺]⁻ =471.5 |
| P-0096 | 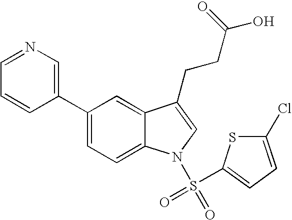 | 3-[1-(5-Chloro-thiophene-2- sulfonyl)-5-pyridin-3-yl-1H- indol-3-yl]-propionic acid | 446.93 | [M + H⁺]⁺ =447.1 |
| P-0108 | 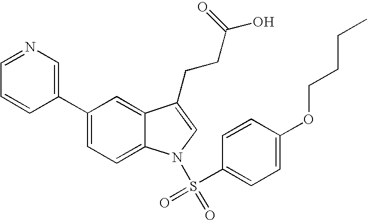 | 3-[1-(4-Butoxy- benzenesulfonyl)-5-pyridin- 3-yl-1H-indol-3-yl]- propionic acid | 478.57 | [M + H⁺]⁺ =479.1 |
| P-0109 | 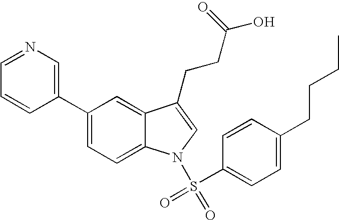 | 3-[1-(4-Butyl- benzenesulfonyl)-5-pyridin- 3-yl-1H-indol-3-yl]- propionic acid | 462.57 | [M + H⁺]⁺=463.1 |
| P-0110 | 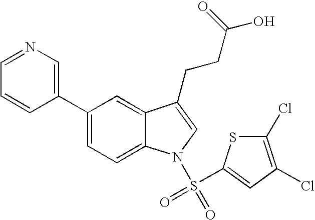 | 3-[1-(4,5-Dichloro- thiophene-2-sulfonyl)-5- pyridin-3-yl-1H-inddol-3-yl]- propionic acid | 481.38 | [M − H⁺]⁻ =481.1 |
| P-0111 | 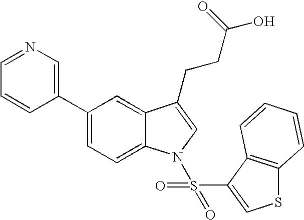 | 3-[1-(Benzo[b]thiophene-3- sulfonyl)-5-pyridin-3-yl-1H- indol-3-yl]propionic acid | 462.55 | [M + H⁺]⁺ =463.1 |
| P-0112 | 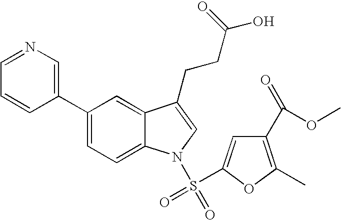 | 5-[3-(2-Carboxy-ethyl)-5- pyridin-3-yl-indole-1- sulfonyl]-2-methyl-furan-3- carboxylic acid methyl ester | 468.49 | [M + H⁺]⁺ =469.1 |
| P-0116 | 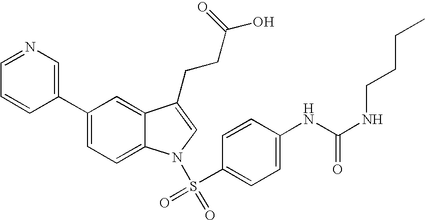 | 3-{1-[4-(3-Butyl-ureido)- benzenesulfonyl]-5-pyridin- 3-yl-1H-indol-3-yl}- propionic acid | 520.61 | [M + H⁺]⁺ =521.1 |
| P-0117 | 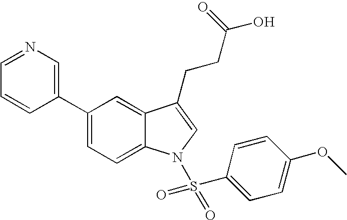 | 3-[1-(4-Methoxy- benzenesulfonyl)-5- pyridin-3-yl-1H-indol-3-yl]- propionic acid | 436.39 | [M + H⁺]⁺ =437.1 |
| P-0119 | 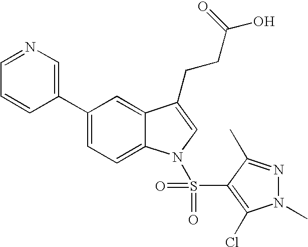 | 3-[1-(5-Chloro-1,3-ddimethyl- 1H-pyrazole-4-sulfonyl)-5- pyridin-3-yl-1H-indol-3-yl]- propionic acid | 458.93 | [M + H⁺]⁺ =459.1 |
| P-0121 | 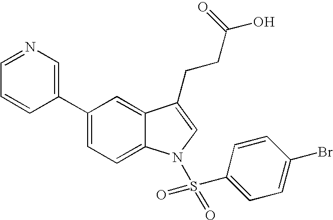 | 3-[1-(4-Bromo- benzenesulfonyl)-5-pyridin- 3-yl-1H-indol-3-yl]- propionic acid | 485.36 | [M + H⁺]⁺ =487.1 |
| P-0122 | 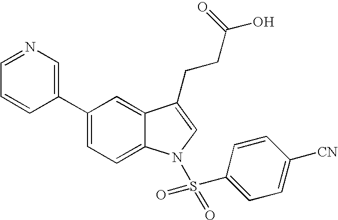 | 3-[1-(4-Cyano- benzenesulfonyl)-5-pyridin- 3-yl-1H-indol-3-yl]- propionic acid | 431.47 | [M + H⁺]⁺ =432.3 |
| P-0123 | 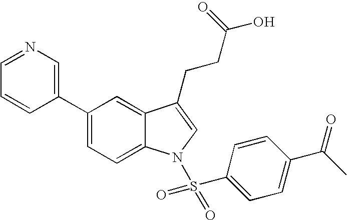 | 3-[1-(4-Acetyl- benzenesulfonyl)-5-pyridin- 3-yl-1H-indol-3-yl]- propionic acid | 448.50 | [M + H⁺]⁺ =449.1 |
| P-0124 | 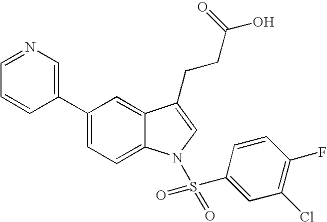 | 3-[1-(3-Chloro-4-fluoro- benzenesulfonyl)-5-pyridin- 3-yl-1H-indol-3-yl]- propionic acid | 458.90 | [M + H⁺]⁺ =459.1 |
| P-0126 | 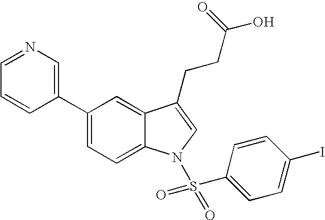 | 3-[1-(4-Iodo- benzenesulfonyl)-5-pyridin- 3-yl-1H-indol-3-yl]- propionic acid | 532.36 | [M + H⁺]⁺ =533.1 |
| P-0127 | 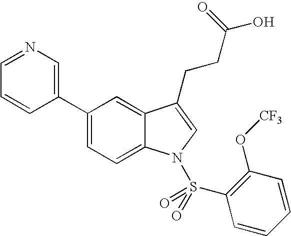 | 3-[5-Pyridin-3-yl-1-(2- trifluoromethoxy- benzenesulfonyl)-1H-indol- 3-yl]-propionic acid | 490.46 | [M + H⁺]⁺ =491.1 |
| P-0129 | 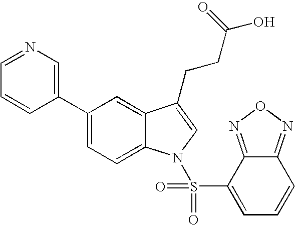 | 3-[1- (Benzo[1,2,5]oxadiazole-4- sulfonyl)-5-pyridin-3-yl-1H- indol-3-yyl]- propionic acid | 448.46 | [M + H⁺]⁺ =449.1 |
| P-0130 | 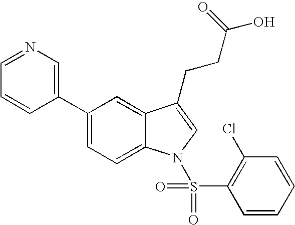 | 3-[1-(2-Chloro- benzenesulfonyl)-5-pyridin- 3-yl-1H-indol-3-yl]- propionic acid | 440.91 | [M + H⁺]⁺ =441.1 |
| P-0132 | 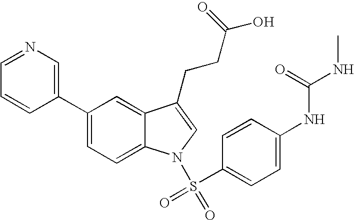 | 3-{1-[4-(3-methyl-ureido)- benzenessulfonyl]-5-pyridin- 3-yl-1H-indol-3-yl}- propionic acid | 478.53 | [M + H⁺]⁺ =479.1 |
| P-0133 | 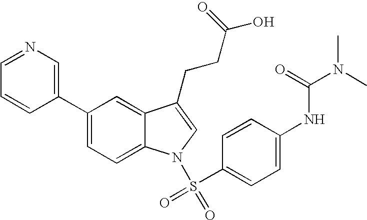 | 3-{1-[4-(3,3-Dimethyl- ureido)-benzenesulfonyl]-5- pyridin-3-yl-1H-indol-3-yl}- propionic acid | 492.56 | [M + H⁺]⁺ =493.1 |
| P-0134 | 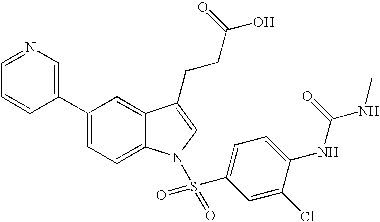 | 3-{1-[3-Chloro-4-(3-methyl- ureido)- benzenessulfonyl]-5-pyridin- 3-yl-1H-indol-3-yl}- propionic acid | 512.98 | [M + H⁺]⁺ =513.1 |
| P-0135 | 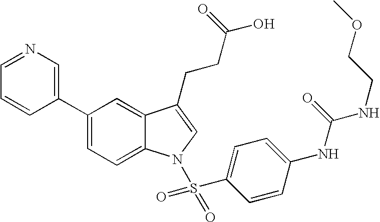 | 3-(1-{4-[3-(2-Methoxy- ethyl)-ureido]- benzenesulfonyl}-5-pyridin- 3-yl-1H-indol-3-yl)- propionic acid | 522.98 | [M + H⁺]⁺ =523.5 |
| P-0136 | 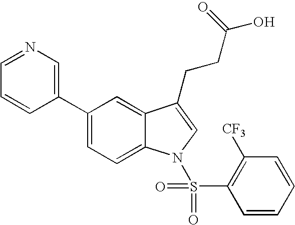 | 3-[5-Pyridin-3-yl-1-(2- trifluoromethyl- benzenesulfonyl)-1H-indol- 3-yl]-propionic acid | 474.46 | [M + H⁺]⁺ =475.1 |
| P-0138 | 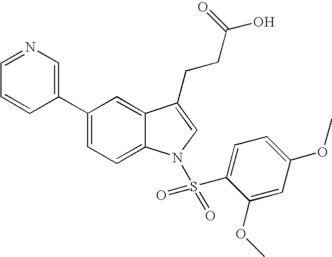 | 3-[1-(2,4-Dimethoxy- benzenesulfonyl)-5-pyridin- 3-yl-1H-indol-3-yl]- propionic acid | 466.52 | [M + H⁺]⁺ =467.1 |
| P-0139 | 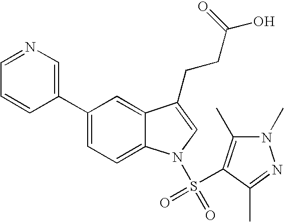 | 3-[5-Pyridin-3-yl-1-(1,3,5- trimethyl-1H-pyrazole-4- sulfonyl)-1H-indol-3-yl]- propionic acid | 438.51 | [M + H⁺]⁺ =439.5 |
| P-0140 | 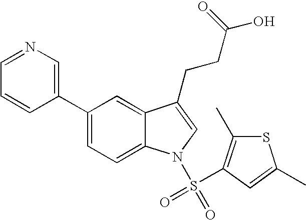 | 3-[1-(2,5-Dimethyl- thiophene-3-sulfonyl)-5- pyridin-3-yl-1H-indol-3-yl]- propionic acid | 440.54 | [M + H⁺]⁺ =441.1 |
| P-0141 | 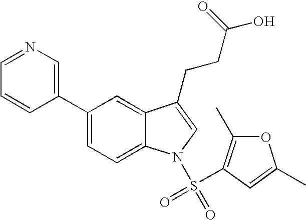 | 3-[1-(2,5-Dimethyl-furan-3- sulfonyl)-5-pyridin-3-yl-1H- indol-3-yl]-propionic acid | 424.48 | [M + H⁺]⁺ =425.1 |
| P-0142 | 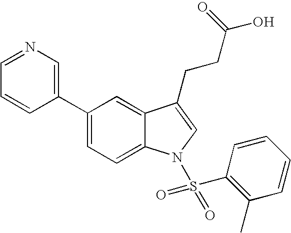 | 3-[5-Pyridin-3-yl-1-(toluene- 2-sulfonyl)-1H-indol-3-yl]- propionic acid | 420.49 | [M + H⁺]⁺ =521.1 |
| P-0143 | 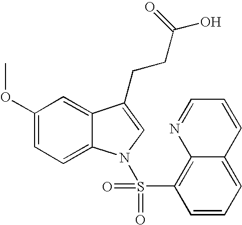 | 3-[5-Methoxy-1-(quinoline- 8-sulfonyl)-1H-indol-3-yl]- propionic acid | 410.45 | [M + H⁺]⁺ =411.1 |
| P-0144 | 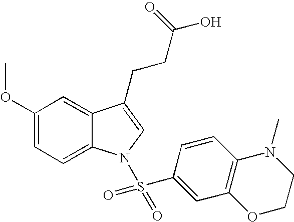 | 3-[5-Methoxy-1-(4-methyl- 3,4-dihydro-2H- benzo[1,4]oxazine-7- sulfonyl)-1H-indol-3-yl]- propionic acid | 430.48 | [M + H⁺]⁺ =431.5 |
| P-0145 | 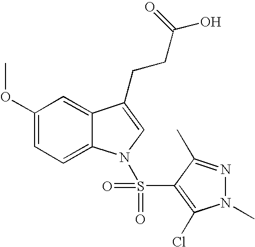 | 3-[1-(5-Chloro-1,3-dimethyl)- 1H-pyrazole-4-sulfonyl)-5- methoxy-1H-indol-3-yl]- propionic acid | 411.87 | [M + H⁺]⁺ =412.3 |
| P-0147 | 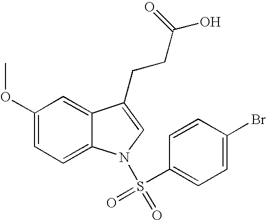 | 3-[1-(4-Bromo- benzenesulfonyl)-5- methoxy-1H-indol-3-yl]- propionic acid | 438.30 | [M + H⁺]⁺ =437.9 |
| P-0148 | 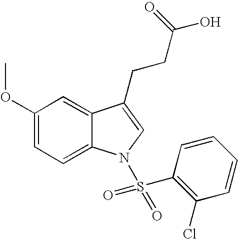 | 3-[1-(2-Chloro- benzenesulfonyl)-5- methoxy-1H-indol-3-yl]- propionic acid | 393.85 | [M + H⁺]⁺ =394.3 |
| P-0149 | 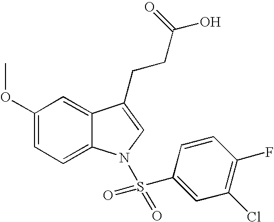 | 3-[1-(3-Chloro-4-fluoro- benzenesulfonyl)-5- methoxy-1H-indol-3-yl]- propionic acid | 411.84 | [M + H⁺]⁺ =411.9 |
| P-0153 | 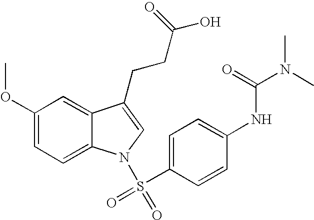 | 3-{1-[4-(3,3-Dimethyl- ureido)-benzenesulfonyl]-5- methoxy-1H-indol-3-yl}- propionic acid | 445.50 | [M + H⁺]⁺ =446.3 |
| P-0154 | 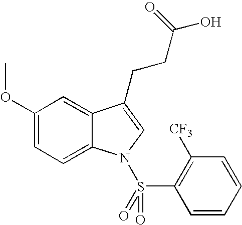 | 3-[5-Methoxy-1-(2- trifluoromethyl- benzenesulfonyl)-1H-indol- 3-yl]-propionic acid | 427.40 | ]M + H⁺]⁺ =427.9 |
| P-0156 | 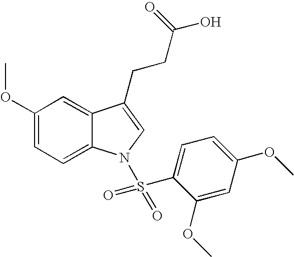 | 3-[1-(2,4-Dimethoxy- benzenesulfonyl)-5- methoxy-1H-indol-3-yl]- propionic acid | 419.46 | [M + H⁺]⁺ =429.9 |
| P-0157 | 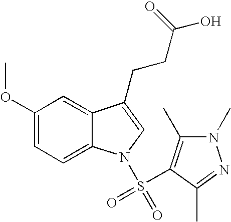 | 3-[5-methoxy-1-(1,3,5- trimethyl-1H-pyrazole-4- sulfonyl)-1H-indol-3-yl]- propionic acid | 391.45 | [M + H⁺]⁺ =392.3 |
| P-0158 | 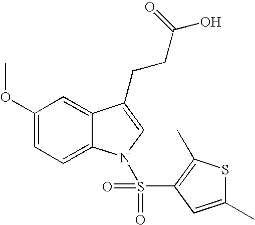 | 3-[1-(2,5-Dimethyl- thiophene-3-sulfonyl)-5- methoxy-1H-indol-3-yl]- propionic acid | 393.48 | [M + H⁺]⁺ =394.3 |
| P-0159 | 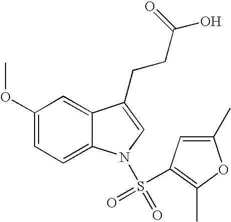 | 3-[1-(2,5-Dimthyyl-furan-3- sulfonyl)-5-methoxy-1H- indol-3-yl]-propionic acid | 377.42 | [M + H⁺]⁺ =378.3 |
| P-0160 | 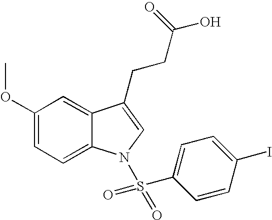 | 3-[1-(4-Iodo- benzenesulfonyl)-5- methoxy-1H-indol-3-yl]- propionic acid | 485.30 | [M + H⁺]⁺ =486.3 |
| P-0161 | 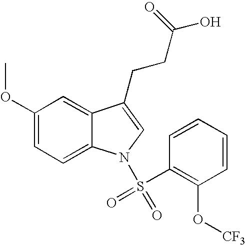 | 3-[5-Methoxy-1-(2- trifluoromethoxy- benzenesulfonyl)-1H-indol- 3-yl]-propionic acid | 443.40 | [M + H⁺]⁺ =444.3 |
| P-0163 | 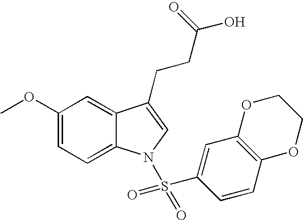 | 3-[1-(2,3-Dihydro- benzo[1,4]dioxine-6- sulfonyl)-5-methoxy-1H- indol-3-yl]-propionic acid | 417.44 | [M + H⁺]⁺ =418.3 |
| P-0164 | 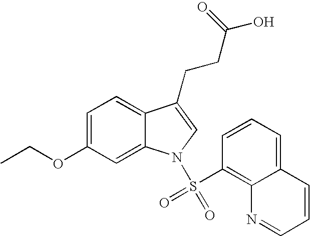 | 3-[6-Ethoxy-1-(quinoline-8- sulfonyl)-1H-indol-3-yl]- propionic acid | 424.48 | [M + H⁺]⁺ =425.1 |
| P-0165 | 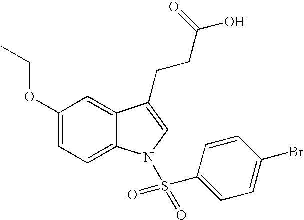 | 3-[1-(4-Bromo- benzenesulfonyl)-5-ethoxy- 1H-indole-3-yl]-prropionic acid | 452.33 | [M '1 H⁺]⁻ =451.9 |
| P-0166 | 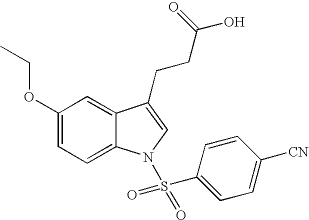 | 3-[1-(4-Cyano- benzenesulfonyl)-5-ethoxy- 1H-indol-3-yl]-propionic acid | 398.44 | [M + H⁺]⁺ =399.1 |
| P-0167 | 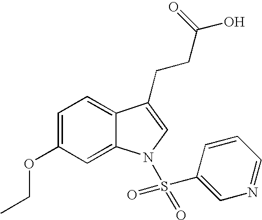 | 3-[5-Ethoxy-1-(pyridine-3- sulfonyl)-1H-indol-3-yl]- propionic acid | 374.42 | [M + H⁺]⁺ =375.1 |
| P-0169 | 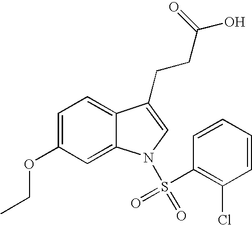 | 3-[6-Ethoxy-1-(4-methoxy- benzenesulfonyl)-1H-indol- 3-yl]-propionic acid | 403.46 | [M + H⁺]⁺ =404.3 |
| P-0170 | 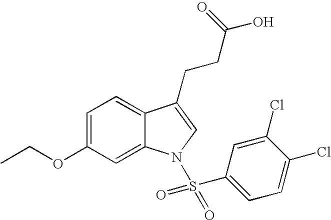 | 3-[1-(3,4-Dichloro- benzenesulfonyl)-6-ethoxy- 1H-indol-3-yl]-propionic acid | 442.32 | [M − H⁺]⁻ =442.3 |
| P-0171 | 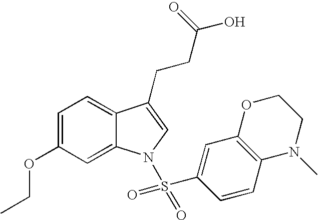 | 3-[6-Ethoxy-1-(4-methyl- 3,4-dihydro-2H- benzo[1,4]oxazine-7- sulfonyl)-1H-indol-3-yl]- propionic acid | 444.51 | [M + H⁺]⁺ =445.1 |
| P-0172 | 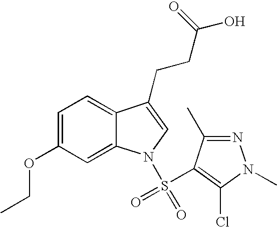 | 3-[1-(5-Chloro-1,3-dimethyl- 1H-pyrazole-4-sulfonyl)-6- ethoxy-1H-indol-3-yl]- propionic acid | 425.89 | [M + H⁺]⁺ =425.9 |
| P-0178 | 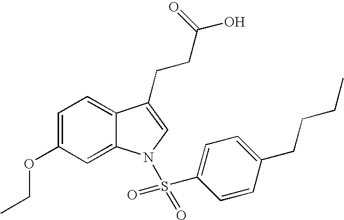 | 3-[1-(4-Butyl- benzenesulfonyl)-6-ethoxy- 1H-indol-3-yl]-propionic acid | 429.54 | [M + H⁺]⁺ =430.3 |
| P-0179 | 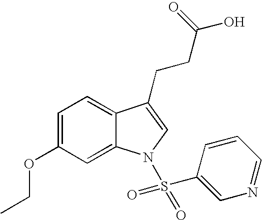 | 3-[6-Ethoxy-1-(pyridine-3- sulfonyl)-1-indol-3-yl]- propionic acid | 374.42 | [M + H⁺]⁺ =375.1 |
| P-0180 | 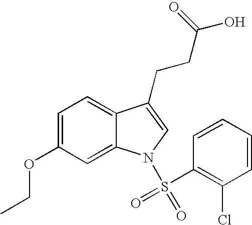 | 3-[1-(2-Chloro- benzenesulfonyl)-6-ethoxy- 1H-indol-3-yl]-propionic acid | 407.88 | [M + H⁺]⁺ =408.3 |
| P-0181 | 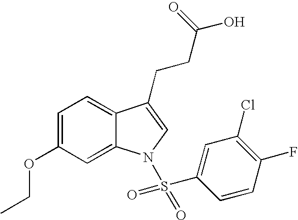 | 3-[1-(3-Chloro-4-fluoro- benzenesulfonyl)-6-ethoxy- 1H-indol-3-yl]-propionic acid | 425.87 | [M + H⁺]⁺ =426.3 |
| P-0184 | 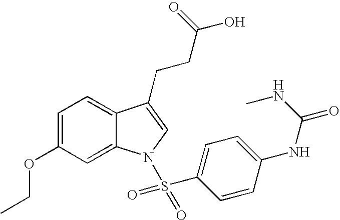 | 3-{6-Ethoxy-1-[4-(3-methyl- ureido)- benzenesulfonyl]-1H-indol- 3-yl}-propionic acid | 445.50 | [M + H⁺]⁺ =446.3 |
| P-0185 | 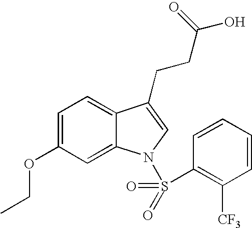 | 3-[6-Ethoxy-1-(2- trifluoromethyl- benzenesulfonyl)-1H-indol- 3-yl]-propionic acid | 441.43 | [M + H⁺]⁺ =442.3 |
| P-0187 | 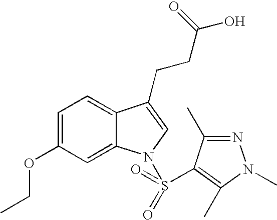 | 3-[6-Ethoxy-1-(1,3,5- trimethyl-1H-pyrazole-4- sulfonyl)-1H-indol-3-yl]- propionic acid | 405.48 | [M + H⁺]⁺ =406.3 |
| P-0188 | 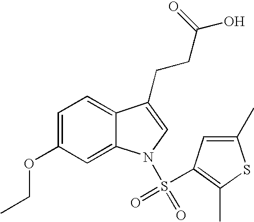 | 3-[1-(2,5-Dimethyl- thiophene-3-sulfonyl)-6- ethoxy-1H-indol-3-yl]- propionic acid | 407.51 | [M + H⁺]⁺ =408.3 |
| P-0189 | 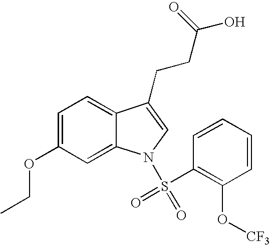 | 3-[6-Ethoxy-1-(2- trifluoromethoxy- benzenesulfonyl)-1H-indol- 3-yl]-propionic acid | 457.43 | [M + H⁺]⁺ =388.3 |
| P-0190 | 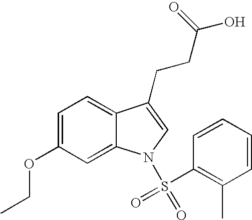 | 3-[6-Ethoxy-1-(toluene-2- sulfonyl)-1H-indol-3-yl]- propionic acid | 387.46 | [M + H⁺]⁺ =388.3 |
| P-0192 | 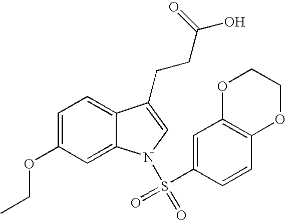 | 3-[1-(2,3-Dihydro- benzo[1,4]dioxine-6- sulfonyl)-6-ethoxy-1H-indol- 3-yl]-propionic acid | 431.67 | [M + H⁺]⁺ =432.3 |
| P-0193 | 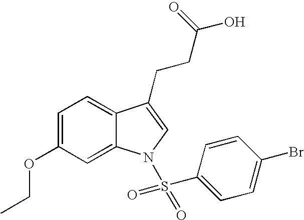 | 3-[1-(4-Bromo- benzenesulfonyl)-6-ethoxy- 1H-indol-3-yl]-propionic acid | 452.33 | [M − H⁺]⁻ =451.9 |
| P-0194 | 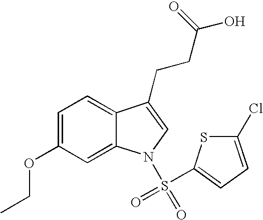 | 3-[1-(5-Chloro-thiophene-2- sulfonyl)-6-ethoxy-1H-indol- 3-yl]-propionic acid | 413.90 | [M − H⁺]⁻ =413.9 |
| P-0197 | 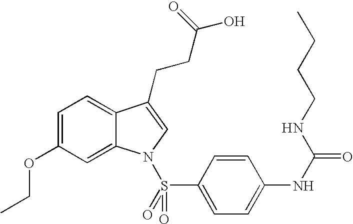 | 3-{1-[4-(3-Butyl-ureido- benzenesulfonyl]-6-ethoxy- 1H-indol-3-yl}-propionic acid | 487.58 | [M + H⁺]⁺ =488.3 |
| P-0198 | 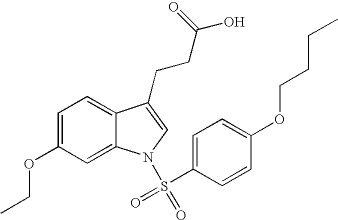 | 3-[1-(4-Butoxy- benzenesulfonyl)-6-ethoxy- 1H-indol-3-yl]-propionic acid | 445.54 | [M + H⁺]⁺ =446.3 |
| P-0199 | 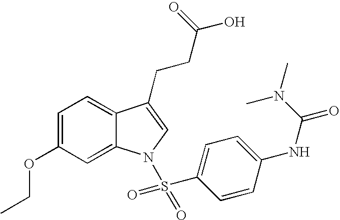 | 3-{1-[4-(3,3-Dimethyl- ureido)-benzenesulfonyl]-6- ethoxy-1H-indol-3-yl}- propionic acid | 459.53 | [M + H⁺]⁺ =499.9 |
| P-0200 | 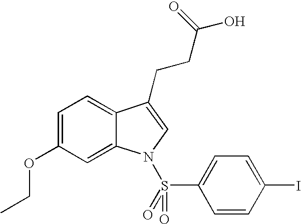 | 3-[6-Ethoxy-1-(4-iodo- benzenesulfonyl)-1H-indol- 3-yl]-propionic acid | 499.33 | [M + H⁺]⁺ =499.9 |
| P-0202 | 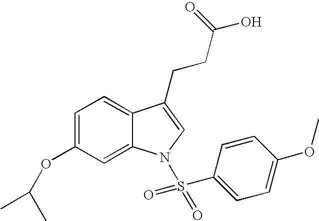 | 3-[6-Isopropoxy-1-(4- methoxy-benzenesulfonyl)- 1H-indol-3-yl]-propionic acid | 417.48 | [M + H⁺]⁺ =418.3 |
| P-0203 | 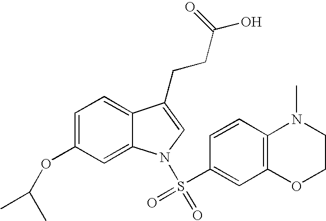 | 3-[6-Isopropoxy-1-(4- methyl-3,4-dihydro-2H- benzo[1,4]oxazine-7- sulfonyl)-1H-indol-3-yl]- propionic acid | 458.54 | [M + H⁺]⁺ =459.1 |
| P-0204 | 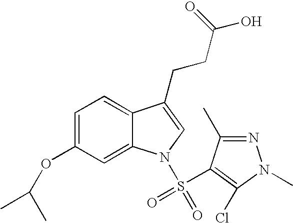 | 3-[1-(5-Chloro-1,3-dimethyl- 1H-pyrazole-4-sulfonyl)-6- isopropoxy-1H-indol-3-yl]- propionic acid | 439.62 | [M + H⁺]⁺ =440.3 |
| P-0205 | 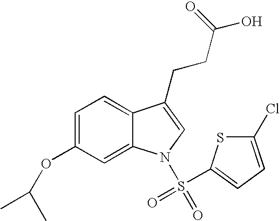 | 3-[1-(5-Chloro-thiophene-2- sulfonyl)-6-isopropoxy-1H- indol-3-yl]-propionic acid | 427.93 | [M + H⁺]⁺ =428.3 |
| P-0209 | 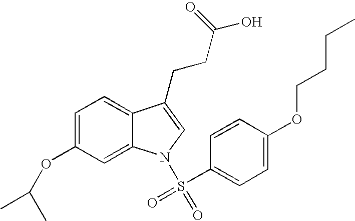 | 3-[1-(4-Butoxy- benzenesulfonyl)-6- isopropoxy-1H-indol-3-yl]- propionic acid | 459.57 | [M + H⁺]⁺ =460.3 |
| P-0210 | 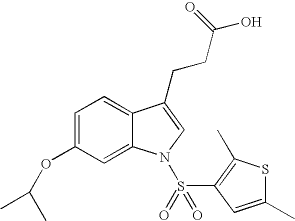 | 3-[1-(2,5-Dimethyl- thiophene-3-sulfonyl)-6- isopropoxy-1H-indol-3-yl]- propionic acid | 421.54 | [M + H⁺]⁺ =421.9 |
| P-0211 | 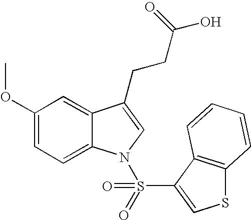 | 3-[1-(Benzo[b]thiophene-3- sulfonyl)-5-methoxy-1H- indol-3-yl]-propionic acid | 415.59 | [M − H⁺]⁻ =415.1 |
| P-0212 | 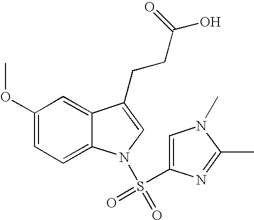 | 3-[1-(1,2-Dimethyl-1H- imidazole-4-sulfonyl)-5- methoxy-1H-indol-3-yl]- propionic acid | 377.42 | [M + H⁺]⁺ =378.3 |
| P-0213 | 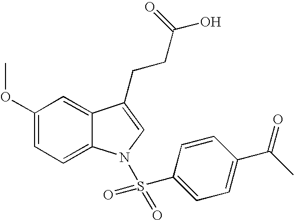 | 3-[1-(4-Acetyl- benzenesulfonyl)-5- methoxy-1H-indol-3-yl]- propionic acid | 401.44 | [M + H⁺]⁺ =401.9 |
| P-0217 | 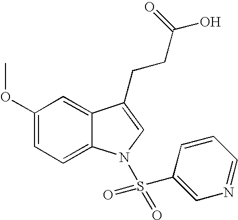 | 3-[5-Methoxy-1-(pyridin-3- sulfonyl)-1H-indol-3-yl]- propionic acid | 360.39 | [M + H⁺]⁺ =361.1 |
| P-0218 | 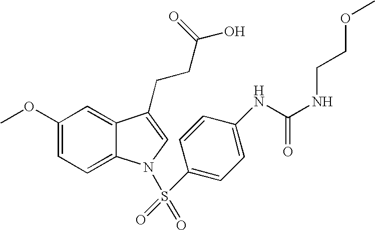 | 3-(5-Methoxy-1-{4-[3-(2- methoxy-ethyl)-ureido]- benzenesulfonyl}-1H-indol- 3-yl)-propionic acid | 475.52 | [M + H⁺]⁺ =475.9 |
| P-0219 | 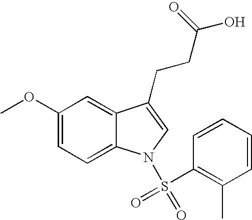 | 3-[5-Methoxy-1-(toluene-2- sulfonyl)-1H-indol-3-yl]- propionic acid | 373.43 | [M + H⁺]⁺ =374.3 |
| P-0220 | 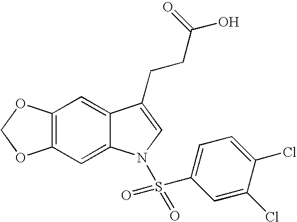 | 3-[5-(3,4-Dichloro- benzenesulfonyl)-5H- [1,3]dioxolo[4,5-f]indol-7- yl]-propionic acid | 442.48 | [M − H⁺]⁻ =442.3 |
| P-0221 | 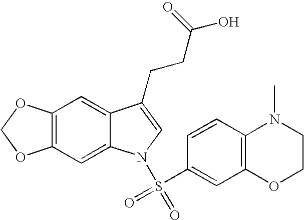 | 3-[5-(4-Methyl-3,4-dihydro- 2H-benzo[1,4]oxazine-7- sulfonyl)-5H- [1,3]dioxolo[4,5-f]indol-7- yl]-propionic acid | 444.47 | [M + H⁺]⁺ =445.1 |
| P-0222 | 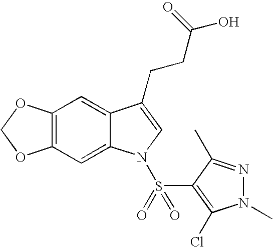 | 3-[5-(5-Chloro-1,3-dimethyl- 1H-pyrazole-4-sulfonyl)-5H- [1,3]dioxolo[4,5-f]indol-7- yl]-propionic acid | 425.85 | [M + H⁺]⁺ =425.9 |
| P-0223 | 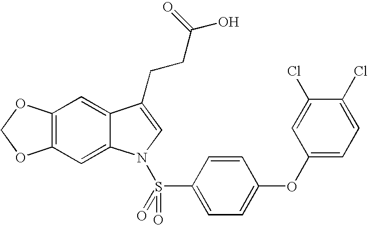 | 3-{5-[4-(3,4-Dicholoro- phenoxy)-benzenesulfonyl]- 5H-[1,3]dioxolo[4,5-f]indol-7-yl}-propionic acid | 534.38 | [M − H⁺]⁻ =533.9 |
| P-0224 | 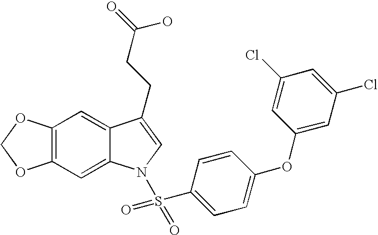 | 3-{5-[4-(3,5-Dichloro- phenoxy)-benzenesulfonyl]- 5H-[1,3]dioxolo[4,5-f]indol-7-yl}-propionic acid | 534.38 | [M − H⁺]⁻ =533.9 |
| P-0225 | 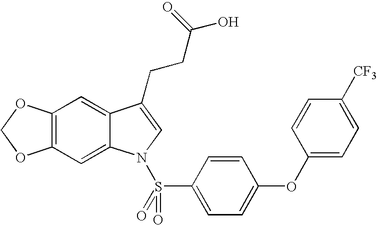 | 3-{5-[4-(4-Trifluoromethyl- phenoxy)-benzenesulfonyl]- 5H-[1,3]dioxolo[4,5-f]indol- 7-yl}-propionic acid | 533.48 | [M + H⁺]⁺ =533.9 |
| P-0226 | 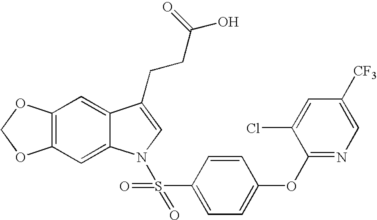 | 3-{5-[4-(3-Chloro-5- trifluoromethyl-pyridin-2- yloxy)-benzenesulfonyl]-5H- [1,3]dioxolo[4,5-f]indol-7- yl}-propionic acid | 568.92 | [M + H⁺]⁺ =569.2 |
| P-0227 | 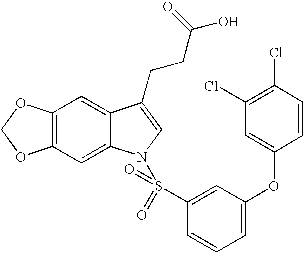 | 3-{5-[3-(3,4-Dichloro- phenoxy)-benzenesulfonyl]- 5H-[1,3]dioxolo[4,5-f]indol-7-yl}-propionic acid | 534.38 | [M − H⁺]⁻ =533.9 |
| P-0228 | 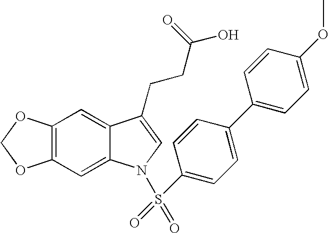 | 3-[5-(4′-Methoxy-biphenyl- 4-sulfonyl)-5H- [1,3]dioxolo[4,5f]indol-7- yl]-propionic acid | 479.51 | [M + H⁺]⁺ =479.9 |
| P-0229 | 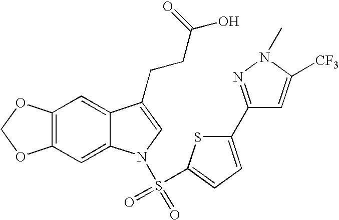 | 3-{5-[5-(1-Methyl-5- trifluoromethyl-1H-pyrazol- 3-yl)-thiophene-2-sulfonyl]- 5H-[1,3]dioxolo[4,5f]indol- 7-yl}-propionic acid | 527.50 | [M + H⁺]⁺ =527.9 |
| P-0230 | 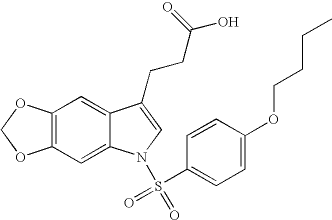 | 3-[5-(4-Butoxy- benzenesulfonyl)-5H- [1,3]dioxolo[4,5-f]indol-7- yl]-propionic acid | 445.50 | [M + H⁺]⁺ =446.3 |
| P-0231 | 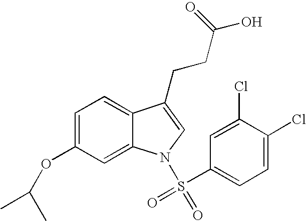 | 3-[1-(3,4-Dichloro- benzenesulfonyl)-6- isopropoxy-1H-indol-3-yl]- propionic acid | 456.35 | [M − H⁺]⁺ =456.3 |
| P-0238 | 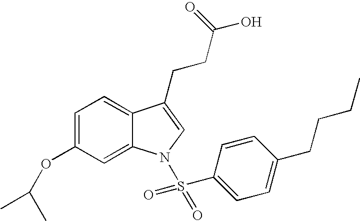 | 3-[1-(4-Butyl- benzenesulfonyl)-6- isoporpoxy-1H-indol-3-yl]- propionic acid | 443.57 | [M + H⁺]⁺ =444.3 |
| P-0239 | 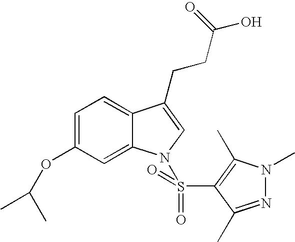 | 3-[6-Isopropoxy-1-(1,3,5- trimethyl-1H-pyrazole-4- sulfonyl)-1H-indol-3-yl]- propionic acid | 419.50 | [M + H⁺]⁺ =420.3 |
| P-0240 | 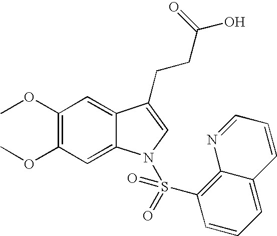 | 3-[5,6-Dimethoxy-1- (quinoline-8-sulfonyl)-1H- indol-3-yl]-propionic acid | 440.48 | [M + H⁺]⁺ =441.1 |
| P-0241 | 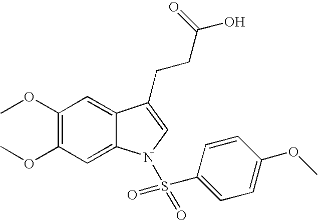 | 3-[5,6-Dimethoxy-1-(4- methoxy-benzenesulfonyl)- 1H-indol-3-yl]-propionic acid | 419.46 | [M + H⁺]⁺=419.9 |
| P-0242 | 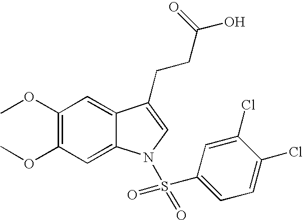 | 3-[1-(3,4-Dichloro- benzenesulfonyl)- 5,6-dimethoxy-1H-indol-3- yl]-propionic acid | 458.32 | [M − H⁺]⁻ =458.3 |
| P-0243 | 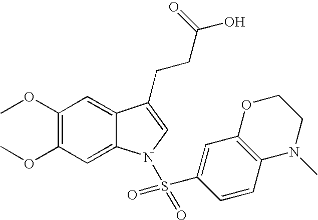 | 3-[5,6-Dimethoxy-1-(4- methyl-3,4-dihydro-2H- benzo[1,4]oxazine-7- sulfonyl)-1H-indol-3-yl]- propionic acid | 460.51 | [M + H⁺]⁺ =461.1 |
| P-0244 | 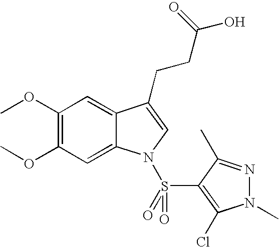 | 3-[1-(5-Chloro-1,3-dimethyl- 1H-pyrazole-4-sulfonyl)-5,6- dimethoxy-1H-indol-3-yl]- propionic acid | 441.89 | [M + H⁺]⁺ =442.3 |
| P-0245 | 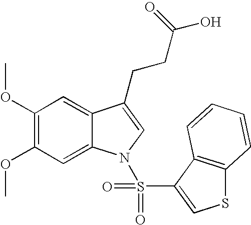 | 3-[1-(Benzo[b]thiophene-3- sulfonyl)-5,6-dimethoxy-1H- indol-3-yl]-propionic acid | 445.52 | [M + H⁺]⁺ =446.3 |
| P-0246 | 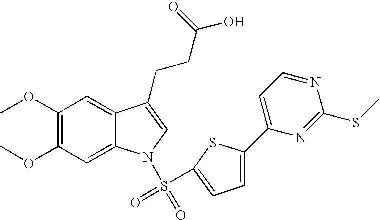 | 3-‡5,6-Dimethoxy-1-[5-(2- methylsulfanyl-pyrimidin-4- yl)-thiophene-2-sulfonyl]- 1H-indol-3-yl}-propionic acid | 519.62 | [M + H⁺]⁺ =520.3 |
| P-0247 | 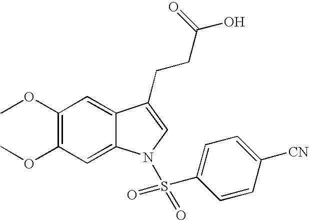 | 3-[1-(4-Cyano- benzenesulfonyl)-5,6- dimethoxy-1H-indol-3-yl]- propionic acid | 414.44 | [M + H⁺]⁺ =415.1 |
| P-0248 | 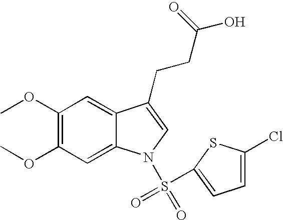 | 3-[1-(5-Chloro-thiophene-2- sulfonyl)-5,6-dimethoxy-1H- indol-3-yl]-propionic acid | 429.90 | [M + H⁺]⁺ =430.3 |
| P-0249 | 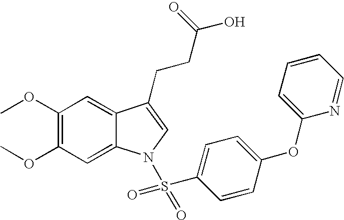 | 3-{5,6-Dimethoxy-1-[4- (pyridin-2-yloxy)- benzenesulfonyl]-1H-indol- 3-yl}-propionic acid | 482.52 | [M + H⁺]⁺ =483.1 |
| P-0250 | 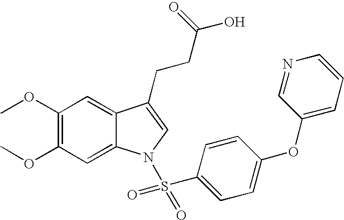 | 3-{5,6-Dimethoxy-1-[4- (pyridin-3-yloxy)- benzenesulfonyl]-1H-indol- 3-yl}-propionic acid | 482.52 | [M + H⁺]⁺ =483.1 |
| P-0251 | 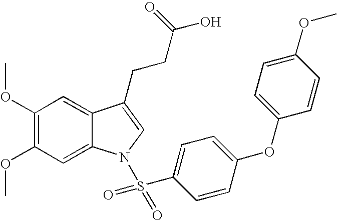 | 3-{5,6-Dimethoxy-1-[4-(4- methoxy-phenoxy)- benzenesulfonyl]-1H-indol- 3-yl}-propionic acid | 511.56 | [M + H⁺]⁺ =512.3 |
| P-0252 | 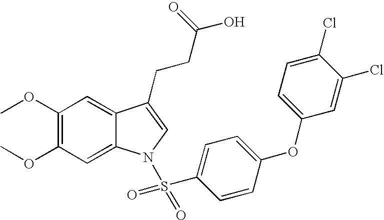 | 3-{1-[4-(3,4-Dichloro- phenoxy)-benzenesulfonyl]- 5,6-dimethoxy-1H-indol-3- yl}-propionic acid | 550.42 | [M − H⁺]⁻ =550.3 |
| P-0253 | 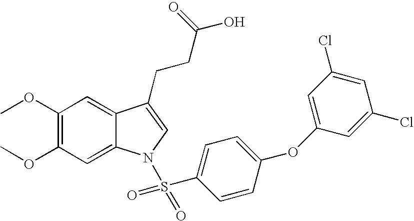 | 3-{1-[4-(3,5-Dichloro- phenoxy)-benzenesulfonyl]- 5,6-dimethoxy-1H-indol-3- yl}-propionic acid | 550.42 | [M − H⁺]⁻ =550.3 |
| P-0254 | 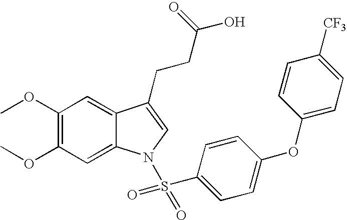 | 3-{5,6-Dimethoxy-1-[4-(4- trifluoromethyl-phenoxy)- benzenesulfonyl]-1H- indol-3-yl}-propionic acid | 549.53 | [M + H⁺]⁺ =550.3 |
| P-0255 | 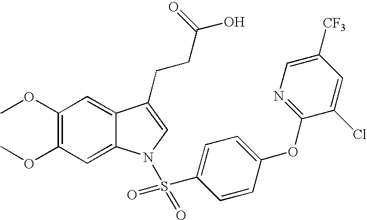 | 3-{1-[4-(3-Chloro-5- trifluoromethyl-pyridin-2- yloxy)-benzenesulfonyl]- 5,6-dimethoxy-1H-indol-3- yl}-propionic acid | 584.96 | [M + H⁺]⁺ =585.2 |
| P-0256 | 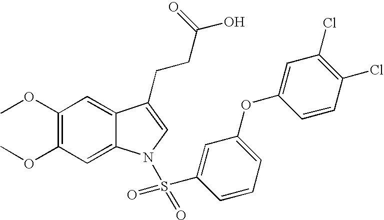 | 3-{1-[3-(3,4-Dichloro- phenoxy)-benzenesulfonyl]- 5,6-dimethoxy-1H-indol-3- yl}-propionic acid | 550.42 | [M − H⁺]⁻ =550.3 |
| P-0257 | 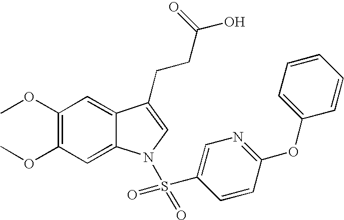 | 3-[5,6-Dimethoxy-1-1(6- phenoxy-pyridine-3- sulfonyl)-1H-indol-3-yl]- propionic acid | 482.52 | [M + H⁺]⁺ =483.1 |
| P-0258 | 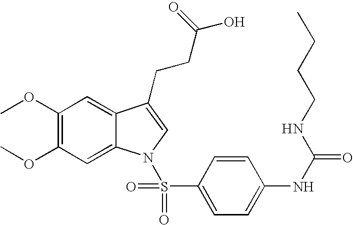 | 3-{1-[4-(3-Butyl-ureido)- benzenesulfonyl]-5,6- dimethoxy-1H-indol-3-yl}- propionic acid | 503.58 | [M + H⁺]⁺ =504.3 |
| P-0259 | 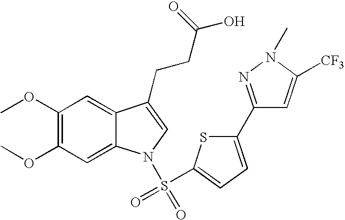 | 3-{5,6-Dimethoxy-1-[5-(1- methyl-5-trifluoromethyl- 1H-pyrazol-3-yl)-thiophene- 2-sulfonyl]-1H-indol-3-yl}- propionic acid | 543.54 | [M + H⁺]⁺ =544.3 |
| P-0260 | 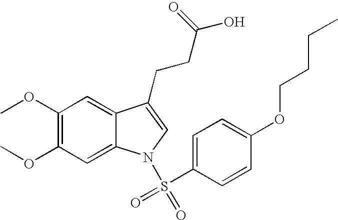 | 3-[1-(4-Butoxy- benzenesulfonyl)-5,6- dimthoxy-1H-indol-3-yl]- propionic acid | 461.54 | [M + H⁺]⁺ =462.3 |
| P-0261 | 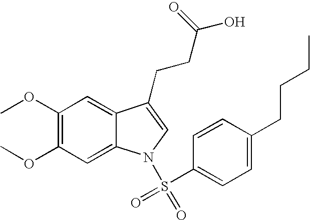 | 3-[1-(4-Butyl- benzenesulfonyl)-5,6- dimethoxy-1H-indol-3-yl]- propionic acid | 445.54 | [M + H⁺]⁺ =446.3 |
| P-0262 | 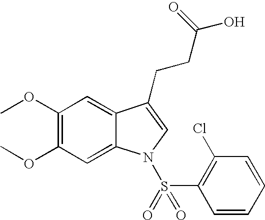 | 3-[1-(2-Chloro- benzenesulfonyl)-5,6- dimethoxy-1H-indol-3-yl]- propionic acid | 423.88 | [M + H⁺]⁺ =423.9 |
| P-0263 | 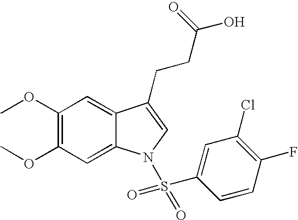 | 3-[1-(3-Chloro-4-fluoro- benzenesulfonyl)-5,6- dimethoxy-1H-indol-3-yl]- propionic acid | 441.87 | [M + H⁺]⁺ =442.3 |
| P-0264 | 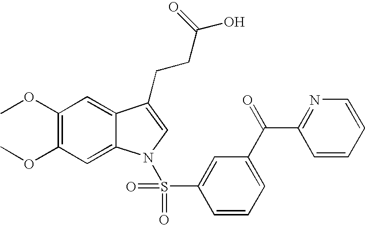 | 3-{5,6-Dimethoxy-1-[3- (pyridine-2-carbonyl)- benzenesulfonyl]-1H-indol- 3-yl}-propionic acid | 494.53 | [M + H⁺]⁺ =495.1 |
| P-0265 | 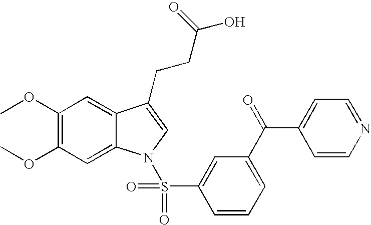 | 3-{5,6-Dimethoxy-1-[3- (pyridine-4- carbonyl)benzenesulfonyl]- 1H-indol-3-yl}-propionic acid | 494.53 | [M + H⁺]⁺ =495.1 |
| P-0266 | 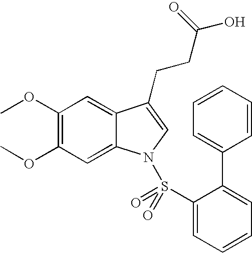 | 3-[1-(Biphenyl-2-sulfonyl)- 5,6-dimethoxy-1H-indol-3- yl]-propionic acid | 465.53 | [M + H⁺]⁺ =466.3 |
| P-0267 | 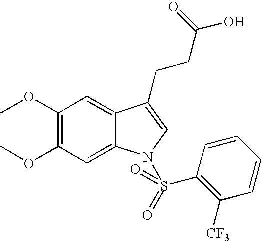 | 3-[5,6-Dimethoxy-1-(2- trifluoromethyl- benzenesulfonyl)-1H-indol- 3-yl]-propionic acid | 457.43 | [M + H⁺]⁺ =458.3 |
| P-0268 | 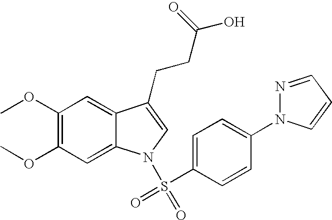 | 3-[5,6-Dimethoxy-1-(4- pyrazol-1-yl- benzenesulfonyl)-1H-indol- 3-yl]-propionic acid | 455.49 | [M + H⁺]⁺ =456.3 |
| P-0269 | 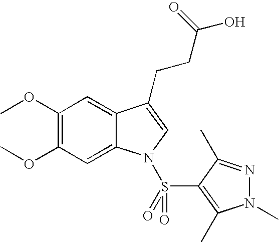 | 3-[5,6-Dimethoxy-1-(1,3,5- trimethyl-1H-pyrazole-4- sulfonyl)-1H-indol-3-yl]- propionic acid | 421.48 | [M + H⁺]⁺ =421.9 |
| P-0270 | 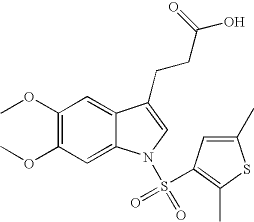 | 3-[1-(2,5-Dimethyl- thiophene-3-sulfonyl)-5,6- dimethoxy-1H-indol-3-yl]- propionic acid | 423.51 | [M + H⁺]⁺ =423.9 |
| P-0271 | 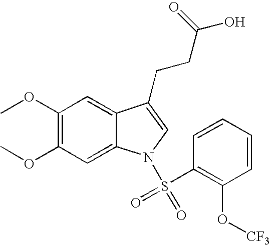 | 3-[5,6-Dimethoxy-1-(2- trifluoromethoxy- benzenesulfonyl)-1H-indol- 3-yl]-propionic acid | 473.43 | [M + H⁺]⁺ =473.9 |
| P-0272 | 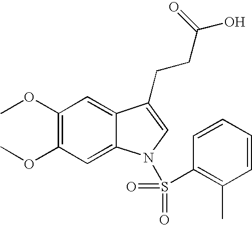 | 3-[5,6-Dimethoxy-1- (toluene-2-sulfonyl)-1H- indol-3-yl]-propionic acid | 403.46 | [M + H⁺]⁺ =404.3 |
| P-0273 | 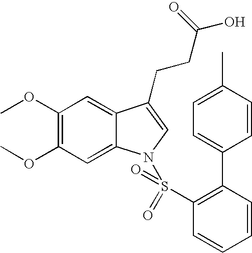 | 3-[5,6-Dimethoxy-1-(4′- methyl-biphenyl-2-sulfonyl)- 1H-indol-3-yl]-propionic acid | 479.56 | ]M + H⁺]⁺ =479.9 |
| P-0274 | 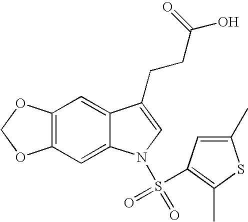 | 3-[5,6-Dimethoxy-1-(2- phenoxy-benzenesulfonyl)- 1H-indol-3-yl]-propionic acid | 4481.53 | [M + H⁺]⁺ =482.3 |
| P-0275 | 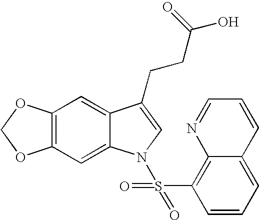 | 3-[5-(Quinoline-8-sulfonyl)- 5H-[1,3]dioxolo[4,5-f]indol- 7-yl]-propionic acid | 424.44 | [M + H⁺]⁺ =425.1 |
| P-0276 | 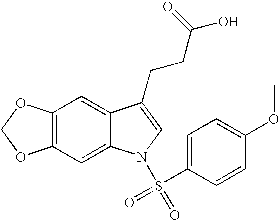 | 3-[5-(4-Methoxy- benzenesulfonyl)-5H- [1,3]dioxolo[4,5-f]indol-7- yl]-propionic acid | 403.41 | [M + H⁺]⁺ =404.3 |
| P-0277 | 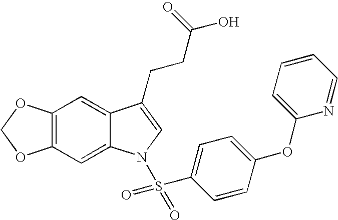 | 3-{5-[4-(Pyridin-2-yloxy)- benzenesulfonyl]- 5H[1,3]dioxolo[4,5-f]indol- 7-yl}-propionic acid | 466.47 | [M + H⁺]⁺ =467.1 |
| P-0278 | 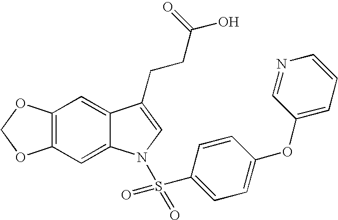 | 3-{5-]4- (Pyridin-3-yloxy)- benzenesulfonyl]-5H- [1,3]dioxolo[44,5-f]indol-7- yl}-propionic acid | 466.47 | [M + H⁺]⁺ =467.1 |
| P-0279 | 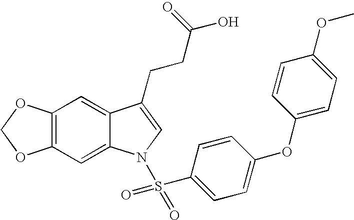 | 3-{5-[4-(4-Methoxy- phenoxy)-benzenesulfonyl]- 5H-[1,3]dioxolo[4,5-f]indol- 7-yl}-propionic acid | 495.51 | [M + H⁺]⁺ =495.9 |
| P-0280 | 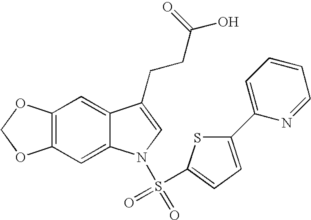 | 3-[5-(5-Pyridin-2-yl- thiophene-2-sulfonyl)-5H- [1,3]dioxolo[4,5-f]indol-7- yl]-propionic acid | 465.50 | [M + H⁺]⁺ =457.1 |
| P-0281 | 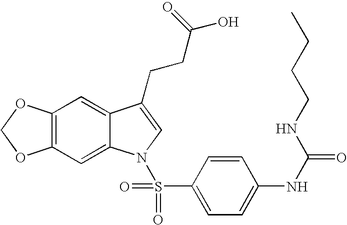 | 3-{5-[4-(3-Butyl-ureido)- benzenesulfonyl]-5H- [1,3]dioxolo[4,5-f]inddol-7- yl}-propionic acid | 487.54 | [M + H⁺]⁺ =488.3 |
| P-0282 | 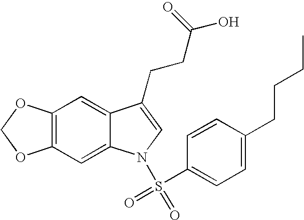 | 3-[5-(4-Butyl- benzenesulfonyl)-5H- [1,3]dioxolo[4,5-f]indol-7- yl]-propionic acid | 429.50 | [M + H⁺]⁺ =430.3 |
| P-0283 | 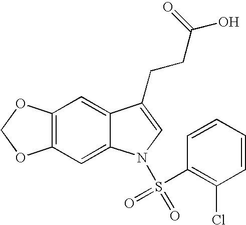 | 3-[5-(2-Chloro- benzenesulfonyl)-5H- [1,3]dioxolo[4,5-f]indol-7- yl]-propionic acid | 407.83 | [M + H⁺]⁺ =408.3 |
| P-0284 | 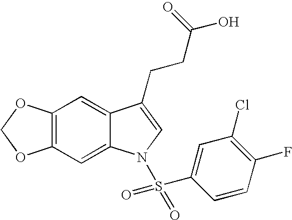 | 3-[5-(3-Chloro-4-fluoro- benzenesulfonyl)-5H- [1,3]dioxolo[4,5-f]indol-7- yl]-propionic acid | 425.82 | [M + H⁺]⁺ =426.3 |
| P-0285 | 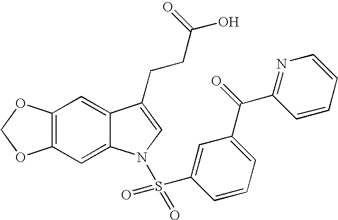 | 3-{5-[3-(Pyridine-2- carbonyl)-benzenesulfonyl]- 5H-[1,3]dioxolo[4,5-f]indol- 7-yl}-propionic acid | 478.48 | [M + H⁺]⁺ =497.1 |
| P-0286 | 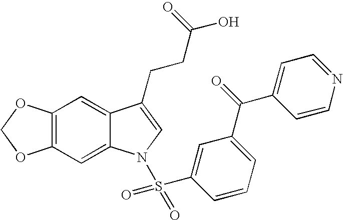 | 3-{5-[3-(Pyridine-4- carbonyl)-benzenesulfonyl]- 5H-[1,3]dioxolo[4,5-f]indol- 7-yl}-propionic acid | 478.48 | [M + H⁺]⁺ =479.1 |
| P-0287 | 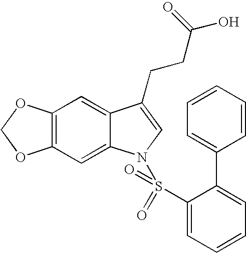 | 3-[5-(Biphenyl-2-sulfonyl)- 5H-[1,3]dioxolo[4,5-f]indol-7-yl]- propionic acid | 4449.49 | [M + H⁺]⁺ =450.3 |
| P-0288 | 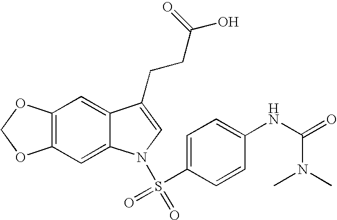 | 3-{5-[4-(3,3-Dimethyl- ureido)-benzenesulfonyl]- 5H-[1,3]dioxolo[4,5-f]indol- 7-yl}-propionic acid | 459.48 | [M + H⁺]⁺ =460.3 |
| P-0289 | 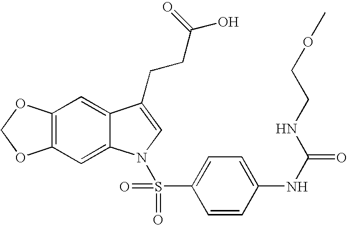 | 3-(5-{4-[3-(2-Methoxy- ethyl)-ureido]- benzenesulfonyl}-5H- [1,3]dioxolo[4,5-f]indol-7- yl)-propionic acid | 489.51 | [M + H⁺]⁺ =489.9 |
| P-0290 | 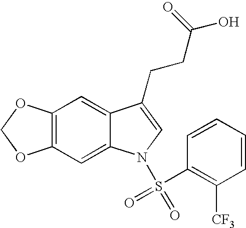 | 3-[5-(2-Trifluoromethyl- benzenesulfonyl)-5H- [1,3]dioxolo[4,5-f]indol-7- yl]-propionic acid | 441.39 | [M + H⁺]⁺ =442.3 |
| P-0291 | 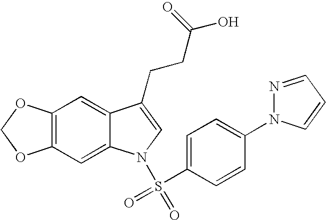 | 3-[5-(4-Pyrazol-1-yl- benzenesulfonyl)-5H- [1,3]dioxolo[4,5-f]indol-7- yl]-propionic acid | 439.45 | [M + H⁺]⁺ =440.3 |
| P-0292 | 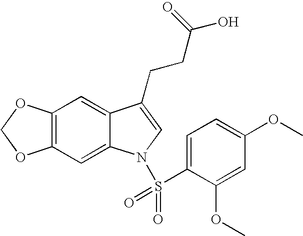 | 3-[5-(2,4-Dimethoxy- benzenesulfonyl)-5H- [1,3]dioxolo[4,5-f]indol-7- yl]-propionic acid | 433.44 | [M + H⁺]⁺ =434.3 |
| P-0293 | 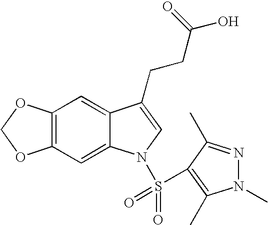 | 3-[5-(1,3,5-Trimethyl-1H- pyrazole-4-sulfonyl)-5H- [1,3]dioxolo[4,5-f]indol-7- yl]-propionic acid | 405.43 | [M + H⁺]⁺ =406.3 |
| P-0294 | 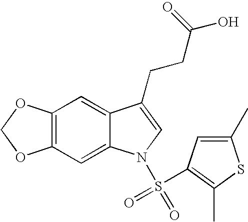 | 3-[5-(2,5-Dimethyl- thiophene-3-sulfonyl)-5H- [1,3]dioxolo[4,5-f]indol-7- yl]-propionic acid | 407.47 | [M + H⁺]⁺ =407.9 |
| P-0295 | 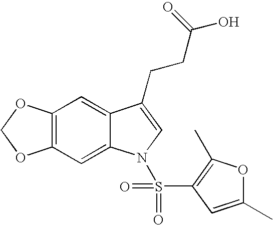 | 3-[5-(2,5-Dimethyl-furan-3- sulfonyl)-5H- [1,3]dioxolo[4,5-f]indol-7- yl]-propionic acid | 391.40 | [M + H⁺]⁺ =391.9 |
| P-0296 | 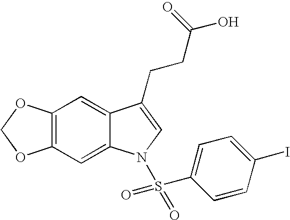 | 3-[5-(4-Iodo- benzenesulfonyl)-5H- [1,3]dioxolo[4,5-f]indol-7- yl]-propionic acid | 499.28 | [M + H⁺]⁺ =499.9 |
| P-0297 | 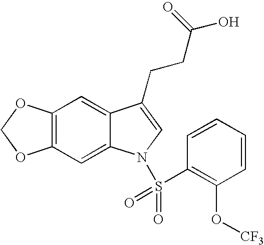 | 3-[5-(2-Trifluoromethoxy- benzenesulfonyl)-5H- [1,3]dioxolo[4,5-f]indol-7- yl]-propionic acid | 457.39 | [M + H⁺]⁺ =458.3 |
| P-0298 | 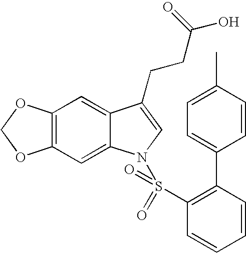 | 3-[5-(4′-methyl-biphenyl-2- sulfonyl)-5H- [1,3]dioxolo[4,5-f]inddol-7- yl]-propionic acid | 463.51 | [M + H⁺]⁺ =463.9 |
| P-0299 | 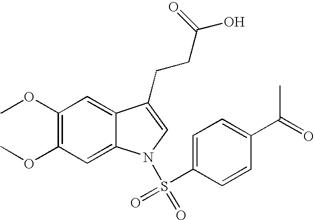 | 3-[1-(4-Acetyl- benzenesulfonyl)-5,6- dimethoxy-1H-indol-3-yl]- propionic acid | 431.47 | [M + H⁺]⁺ =432.3 |
| P-0300 | 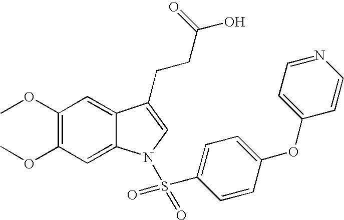 | 3-{5,6-Dimethoxy-1-[4- (pyridin-4-yloxy)- benzenesulfonyl]-1H-indol- 3-yl}-propionic acid | 482.52 | [M + H⁺]⁺ =483.1 |
| P-0301 | 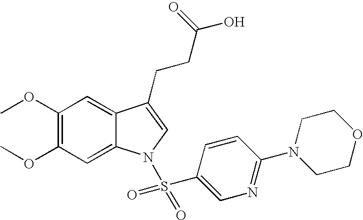 | 3-[5,6-Dimethoxy-1-(6- morpholin-4-yl-pyridine-3- sulfonyl)-1H-indol-3-yl]- propionic acid | 475.52 | |
| P-0302 | 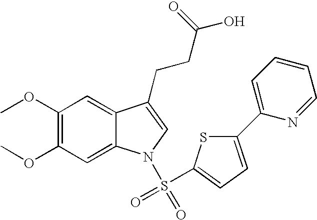 | 3-[5,6-Dimethoxy-1-(5- pyridin-2-yl-thiophene-2- sulfonyl)-1H-indol-3-yl]- propionic acid | 472.54 | [M + H⁺]⁺ =473.1 |
| P-0303 | 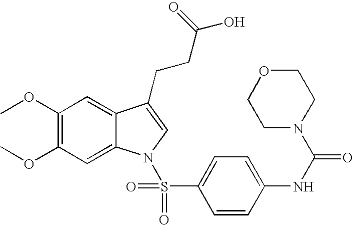 | 3-(5,6-Dimethoxy-1-{4- [(morpholine-4-carbonyl)- amino]-benzenesulfonyl}- 1H-indol-3-yl)-propionic acid | 517.56 | [M + H⁺]⁺ =518.3 |
| P-0304 | 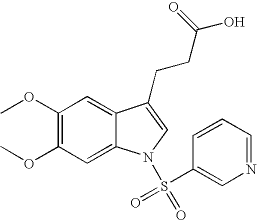 | 3-[5,6-Dimethoxy-1- (pyridine-3-sulfonyl)-1H- indol-3-yl]-propionic acid | 390.42 | [M + H⁺]⁺ =391.1 |
| P-0305 | 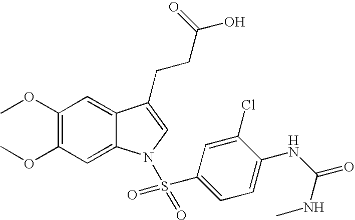 | 3-{1-[4-(Chloro-4-(3-methyl- ureido)- benzenesulfonyl]-5,6- dimethoxy-1H-indol-3-yl}- propionic acid | 495.94 | [M + H⁺]⁺ =496.3 |
| P-0306 | 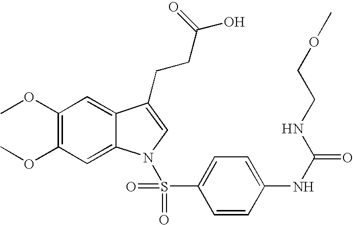 | 3-(5,6-Dimethoxy-1-{4-[3- (2-methoxy-ethyl)-ureido]- benzenesulfonyl}-1H-indol- 3-yl)-propionic acid | 505.55 | [M + H⁺]⁺ =505.9 |
| P-0307 | 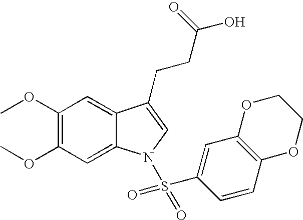 | 3-[1-(2,3-Dihydro- benzo[1,4]dioxine-6- sulfonyl)-5,6-dimethoxy-1H- indol-3-yl]-propanoic acid | 447.47 | [M + H⁺]⁺ =447.9 |
| P-0308 | 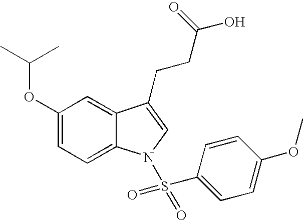 | 3-[5-Isopropoxy-1-(4- methoxy-benzenesulfonyl)- 1H-indol-3-yl]-propionic acid | 417.48 | [M + H⁺]⁺ =418.3 |
| P-0309 | 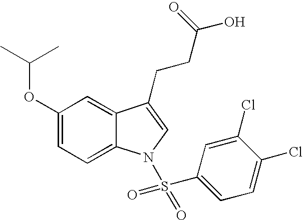 | 3-[1-(3,4-Dichloro- benzenesulfonyl)-5- issopropoxy-1H-indol-3-yl]- propionic acid | 456.35 | [M + H⁺]⁺ =456.3 |
| P-0310 | 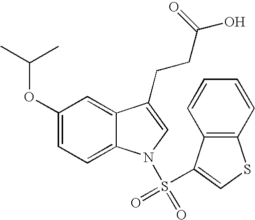 | 3-[1-(Benzo[b]thiophene-3- sulfonyl)-5-issopropoxy-1H- indol-3-yl]-propionic acid | 443.54 | [M + H⁺]⁺ =444.3 |
| P-0312 | 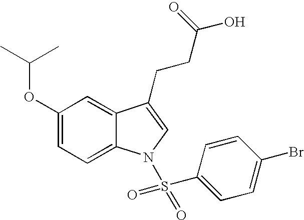 | 3-[1-(4-Bromo- benzenesulfonyl)-5- isopropoxy-1H-indol-3-yl]- propionic acid | 466.35 | [M + H⁺]⁺ =468.3 |
| P-0313 | 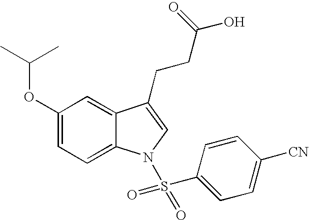 | 3-[1-(4-Cyano- benzenesulfonyl)-5- isopropoxy-1H-indol-3-yl]- propionic acid | 412.47 | [M + H⁺]⁺ =413.1 |
| P-0314 | 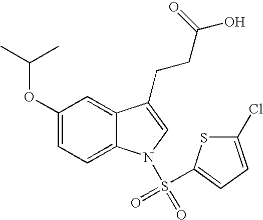 | 3-[1-(5-Chloro-thiophene-2- sulfonyl)-5-isopropoxy-1H- indol-3-yl]-propionic acid | 427.93 | [M − H⁺]⁻ =427.9 |
| P-0315 | 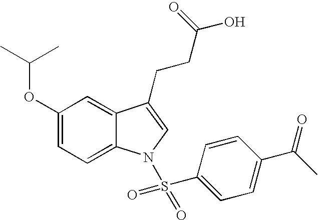 | 3-[1-(4-Acetyl- benzenesulfonyl)-5- isopropoxy-1H-indol-3-yl]- propionic acid | 429.50 | [M + H⁺]⁺ =430.3 |
| P-0325 | 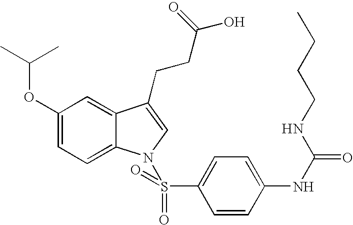 | 3-{1-[4-(3-Butyl-ureido)- benzenesulfonyl]-5- isopropoxy-1H-indol-3-yl}- propionic acid | 501.61 | [M + H⁺]⁺ =502.3 |
| P-0327 | 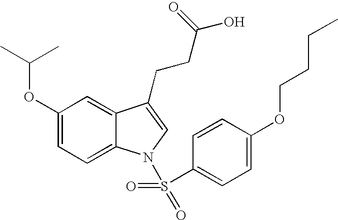 | 3-[1-(4-Butoxy- benzenesulfonyl)-5- isopropoxy-1H-indol-3-yl]- propionic acid | 459.57 | [M + H⁺]⁺ =460.3 |
| P-0328 | 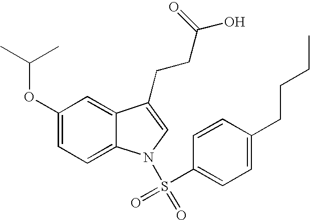 | 3-[1-(4-Butyl- benzenesulfonyl)-5- isopropoxy-1H-indol-3-yl]- propionic acid | 4443.57 | [M + H⁺]⁺ =444.3 |
| P-0329 | 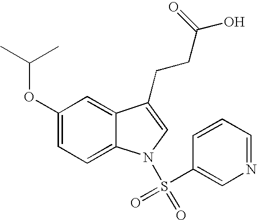 | 3-[5-Isopropoxy-1-(pyridine- 3-sulfonyl)-1H-indol-3-yl]- propionic acid | 388.45 | [M + H⁺]⁺ =389.1 |
| P-0330 | 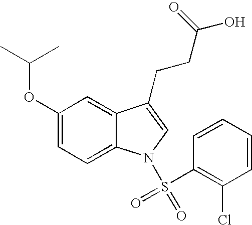 | 3-[1-(2-Chloro- benzenesulfonyl)-5- isopropoxy-1H-indol-3-yl]- propionic acid | 421.90 | [M + H⁺]⁺ =421.9 |
| P-0331 | 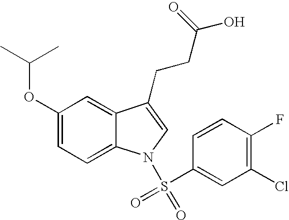 | 3-[1-(3-Chloro-4-fluoro- benzenesulfonyl)-5- isopropoxy-1H-indol-3-yl]- propionic acid | 439.59 | [M + H⁺]⁺ =440.3 |
| P-0333 | 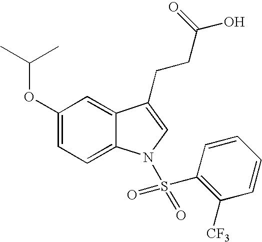 | 3-[5-Isopropoxy-1-(2- trifluoromethyl- benzenesulfonyl)-1H-indol- 3-yl]-propionic acid | 455.46 | [M + H⁺]⁺ =456.3 |
| P-0334 | 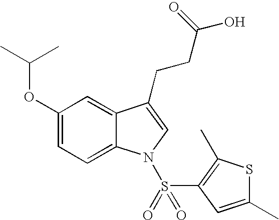 | 3-[1-(2,5-Dimethyl- thiophene-3-sulfonyl)-5- isopropoxy-1H-indol-3-yl]- propionic acid | 421.54 | [M + H⁺]⁺ =421.9 |
| P-0335 | 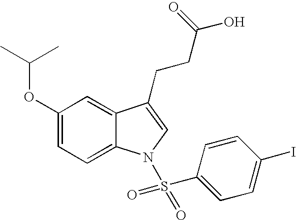 | 3-[1-(4-Iodo- benzenesulfonyl)-5- isopropoxy-1H-indol-3-yl]- propionic acid | 513.35 | [M + H⁺]⁺ =514.3 |
| P-0336 | 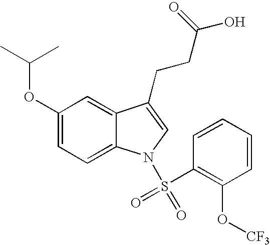 | 3-[5-Isopropoxy-1-(2- trifluoromethoxy- benzenesulfonyl)-1H-indol- 3-yl]-propionic acid | 471.46 | [M + H⁺]⁺ =472.3 |
| P-0337 | 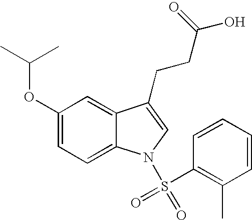 | 3-[5-Isopropoxy-1-(toluene- 2-sulfonyl)-1H-indol-3-yl]- propionic acid | 401.49 | [M + H⁺]⁺ =402.3 |
| P-0340 | 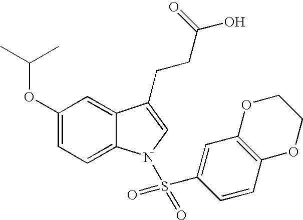 | 3-[1-(2,3-Dihydro- benzo[1,4]dioxine-6- sulfonyl)-5-isopropoxy-1H- indol-3-yl]-propionic acid | 445.50 | [M + H⁺]⁺ =446.3 |
| P-0341 | 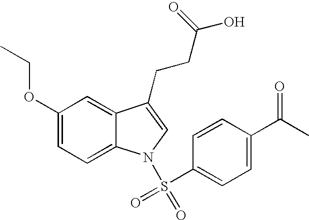 | 3-[1-(4-Acetyl- benzenesulfonyl)-5-ethoxy- 1H-indol-3-yl]-propionic acid | 415.47 | [M + H⁺]⁺ =416.3 |
| P-0343 | 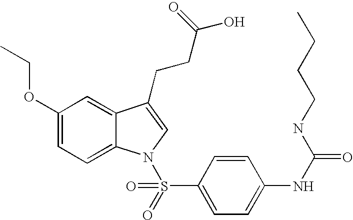 | 3-{1-[4-(3-Butyl-ureido)- benzenesulfonyl]-5-ethoxy- 1H-indol-3-yl}-propionic acid | 487.58 | [M + H⁺]⁺ =488.3 |
| P-0345 | 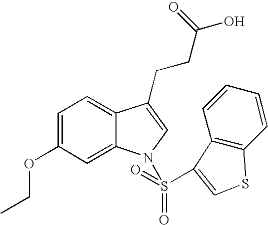 | 3-[1-(Benzo[b]thiophene-3- sulfonyl)-6-ethoxy-1H-indol- 3-yl]-propionic acid | 429.52 | [M + H⁺]⁺ =430.3 |
| P-0348 | 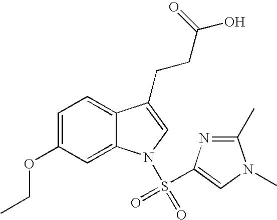 | 3-[1-(1,2-Dimethyl-1H- imidazole-4-sulfonyl)-6- ethoxy-1H-indol-3-yl]- propionic acid | 391.45 | [M + H⁺]⁺ =392.3 |
| P-0349 | 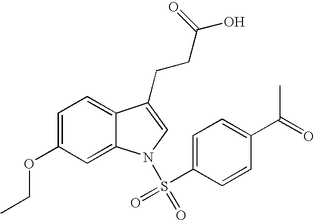 | 3-[1-(4-Acetyl- benzenesulfonyl)-6-ethoxy- 1H-indol-3-yl]-propionic acid | 415.47 | [M + H⁺]⁺ =416.3 |
| P-0350 | 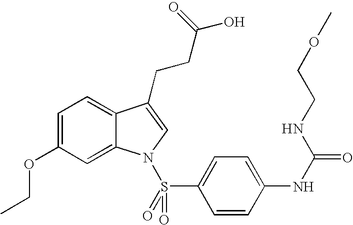 | 3-(6-Ethoxy-1-{4-[3-(2- methoxy-ethyl)-ureido]- benzenesulfonyl}-1H-indol- 3-yl)-propionic acid | 489.55 | [M + H⁺]⁺ =490.3 |
| P-0358 | 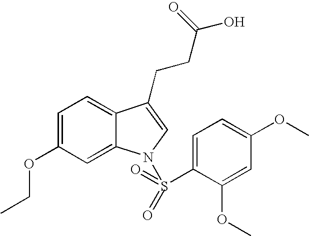 | 3-[1-(2,4-Dimethoxy- benzenesulfonyl)-6-ethoxy- 1H-indol-3-yl]-propionic acid | 433.48 | [M + H⁺]⁺ =434.3 |
| P-0359 | 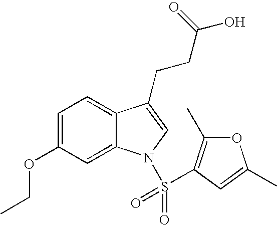 | 3-[1-(2,5-Dimethyl-furan-3- sulfonyl)-6-ethoxy-1H-indol- 3-yl]-propionic acid | 391.45 | [M + H⁺]⁺ =392.3 |
| P-0360 | 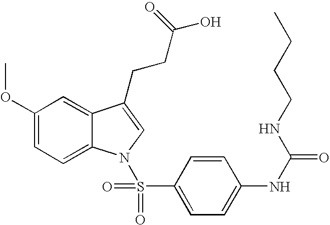 | 3-{1-[4-(3-Butyl-ureido)- benzenesulfonyl]-5- methoxy-1H-indol-3-yl}- propionic acid | 473.55 | [M + H⁺]⁺ =473.9 |
| P-0361 | 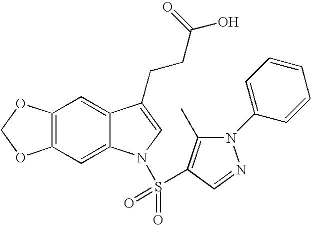 | 3-[5-(5-Methyl-1-phenyl-1H- pyrazole 4-sulfonyl)-5H- [1,3]dioxolo[4,5-f]indol-7-yl]propionic acid | 453.48 | [M + H⁺]⁺ =453.9 |
| P-0362 | 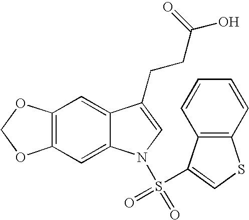 | 3-[5-(Benzo[b]thiophene-3- sulfonyl)-5H- [1,3]dioxolo[4,5-f]indol-7- yl]-propionic acid | 429.47 | [M + H⁺]⁺ =430.3 |
| P-0363 | 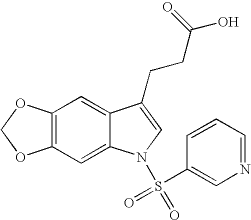 | 3-[5-(Pyridine-3-sulfonyl)- 5H-[1,3]-dioxolo[4,5-f]indol- 7-yl]-propionic acid | 374.37 | [M + H⁺]⁺ =375.1 |
| P-0364 | 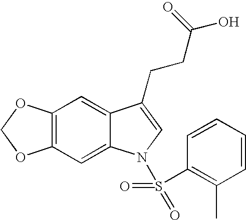 | 3-[5-(Toluene-2-sulfonyl)- 5H-[1,3]dioxolo[4,5-f]indol- 7-yl]-propionic acid | 387.41 | [M + H⁺]⁺ =387.9 |
| P-0365 | 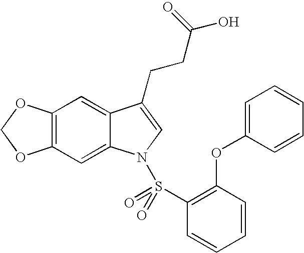 | 3-[5-(2-Phenoxy- benzenesulfonyl)-5H- [1,3]dioxolo[4,5-f]indol-7- yl]-propionic acid | 465.49 | [M + H⁺]⁺ =466.3 |
| P-0366 | 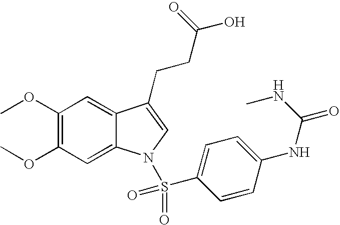 | 3-{5,6-Dimethoxy-1-[4-(3- methyl-ureido)- benzenesulfonyl]-1H-indol- 3-yl}-propionic acid | 461.50 | [M + H⁺]⁺ =462.3 |
| P-0367 | 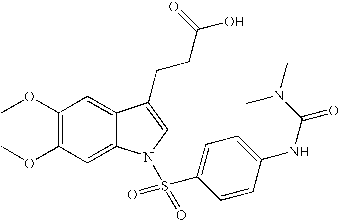 | 3-{1-[4-(3,3-Dimethyl- ureido)-benzenesulfonyl]- 5,6-dimethoxy-1H-indol-3- yl}-propionic acid | 475.52 | [M + H⁺]⁺ =475.9 |
| P-0368 | 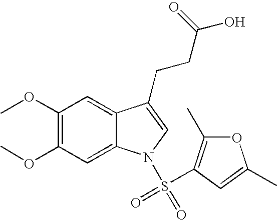 | 3-[1-(2,5-Dimethyl-furan-3- sulfonyl)-5,6-dimethoxy-1H- indol-3-yl]-propionic acid | 407.45 | [M + H⁺]⁺ =408.3 |
| P-0369 | 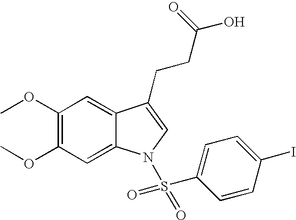 | 3-[1-(4-Iodo- benzenesulfonyl)-5,6- dimethoxy-1H-indol-3-yl]- propionic acid | 515.33 | [M + H⁺]⁺ =516.3 |
| P-0370 | 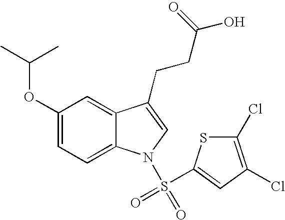 | 3-[1-(4,5-Dichloro- thiophene-2-sulfonyl)-5- isopropoxy-1H-indol-3-yl]- propionic acid | 462.37 | [M − H⁺]⁻ =461.9 |
| P-0372 | 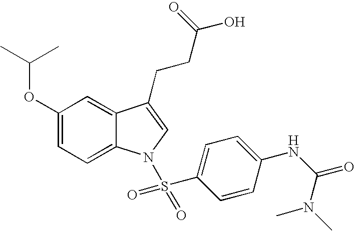 | 3-{1-(3,3-Dimethyl- ureido)-benzenesulfonyl]-5- isopropoxy-1H-indol-3-yl}- propionic acid | 473.55 | [M + H⁺]⁺ =474.3 |
| P-0373 | 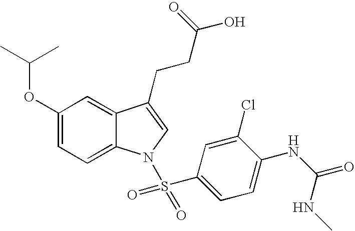 | 3-{1-[3-Chloro-4-(3-methyl- ureido)-benzenesulfonyl]-5- isopropoxy-1H-indol-3-yl}- propionic acid | 493.97 | [M + H⁺]⁺ =494.3 |
| P-0374 | 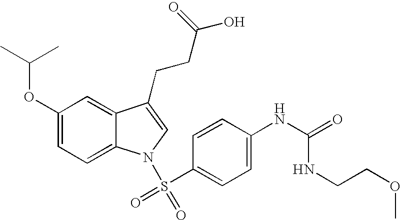 | 3-(5-Isopropoxy-1-{4-[3-(2- methoxy-ethyl)-ureido]- benzenesulfonyl}-1H- indol-3-yl)-propionic acid | 503.58 | [M + H⁺]⁺ =504.3 |
| P-0376 | 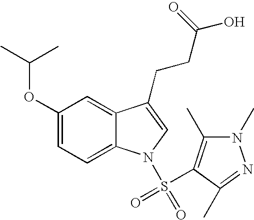 | 3-[5-Isopropoxy-1-(1,3,5- trimethyl-1H-pyrazole-4- sulfonyl)-1H-indol-3-yl]- propionic acid | 419.50 | [M + H⁺]⁺ =420.3 |
| P-0377 | 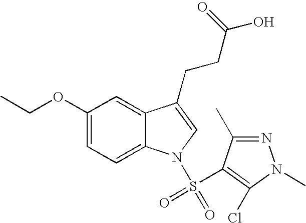 | 3-[1-(5-Chloro-1,3-dimethyl- 1H-pyrazole-4-sulfonyl)-5- ethoxy-1H-indol-3-yl]- propionic acid | 425.89 | [M + H⁺]⁺ =426.3 |
| P-0378 | 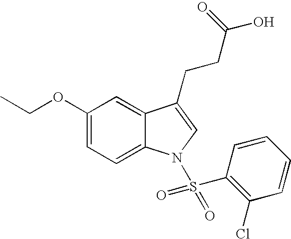 | 3-[1-(2-Chloro- benzenesulfonyl)-5-ethoxy- 1H-indol-3-yl]-propionic acid | 407.87 | [M + H⁺]⁺ =408.3 |
| P-0379 | 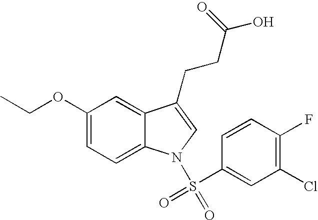 | 3-[1-(3-Chloro-4-fluoro- benzenesulfonyl)-5-ethoxy- 1H-indol-3-yl]-propionic acid | 425.86 | [M + H⁺⁺ =425.9 |
| P-0380 | 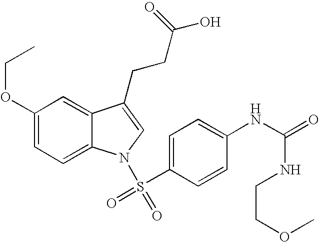 | 3-(5-Ethoxy-1-{4-[3-(2- methoxy-ethyl)-ureido]- benzenesulfonyl}-1H-indol- 3-yl)-propionic acid | 489.56 | [M + H⁺]⁺ =490.3 |
| P-0381 | 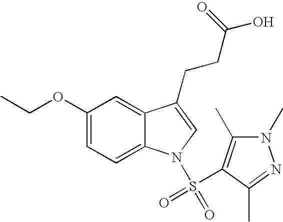 | 3-[5-Ethoxy-1-(1,3,5- trimethyl-1H-pyrazole-4- sulfonyl)-1H-indol-3-yl]- propionic acid | 405.47 | [M + H⁺]⁺ =406.3 |
| P-0382 | 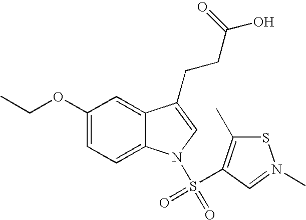 | 3-[1-(2,5-Dimethyl- thiophene-3-sulfonyl)-5- ethoxy-1H-indol-3-yl]- propionic acid | 407.51 | [M + H⁺]⁺ =408.3 |
| P-0383 | 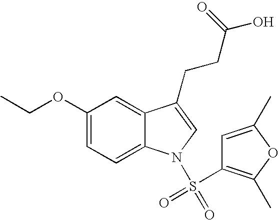 | 3-[1-(2,5-Dimethyl-furan-3- sulfonyl)-5-ethoxy-1H-indol- 3-yl]-propionic acid | 391.44 | [M + H⁺]⁺ =392.3 |
| P-0384 | 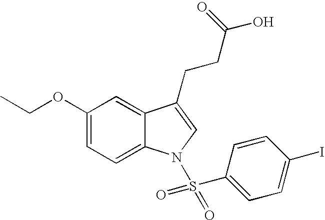 | 3-[5-Ethoxy-1-(4-iodo- benzenesulfonyl)-1H-indol- 3-yl]-propionic acid | 499.32 | [M + H⁺]⁺ =500 |
| P-0385 | 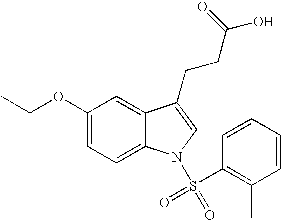 | 3-[5-Ethoxy-1-(toluene-2- sulfonyl)-1H-indol-3-yl]- propionic acid | 387.45 | [M + H⁺]⁺ =388.3 |
| P-0387 | 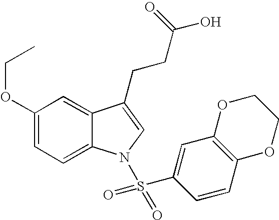 | 3-[1-(2,3-Dihydro- benzo[1.4]dioxine-6- sulfonyl)-5-ethoxy-1H-indol- 3-yl]-propionic acid | 431.46 | [M + H⁺]⁺ =432.3 |
| P-0389 | 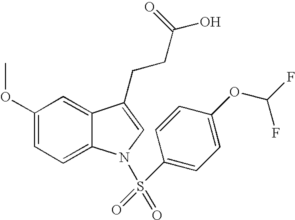 | 3-[1-(4-Difluoromethoxy- benzenesulfonyl)-5- methoxy-1H-indol-3-yl]- propionic acid | 425.1 | [M − H⁺]⁻ =442 |
| P-0390 | 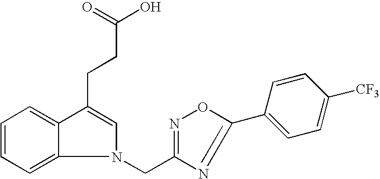 | 3-{1-[5-(4-Trifluoromethyl- phenyl)-[1,2,4]oxadiazol-3- ylmethyl]-1H-indol-3-yl}- propionic acid | 415.37 | [M + H⁺]⁺ =416.4 |
| P-0559 | 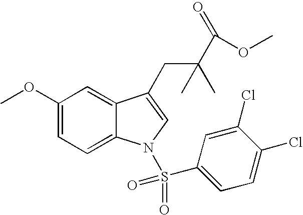 | 3-[1-(23,4-Dichloro- benzenesulfonyl)-5- methoxy-1H-indol-3-yl]-2,2- dimethyl-propionic acid methyl ester | 470.37 | [M + H⁺]⁺ =470.1; 472.1 |
| P-0569 | 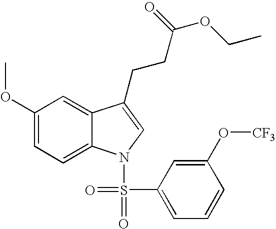 | 3-[5-Methoxy-1-(3- trifluoromethoxy- benzenesulfonyl)-1H-indol- 3-yl]-propionic acid ethyl ester | 471.45 | |
| P-0571 | 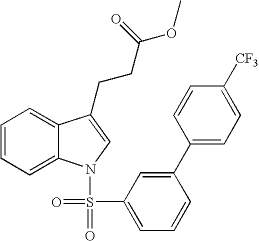 | 3-[1-(4′-Trifluoromethyl- biphenyl-3-sulfonyl)-1H- indol-3-yl]-propionic acid methyl ester | 487.50 | |
| P-0576 | 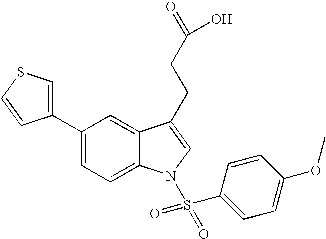 | 3-[1-(4-methoxy- benzenesulfonyl)-5- thiophen-3-yl-1H-indol-3- yl]-propionic acid | 441.53 | |
| P-0577 | 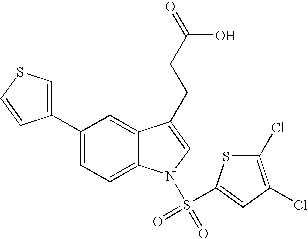 | 3-[1-(4,5-Dichloro- thiophene-2-sulfonyl)-5- thiophen-3-yl-1H-indol-3- yl]-propionic acid | 486.42 | |
| P-0578 | 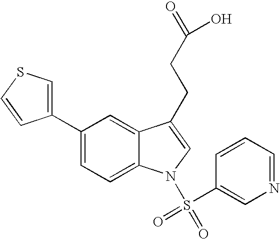 | 3-[1-(Pyridine-3-sulfonyl)-5- thiophen-3-yl-1H-indol-3- yl]-propionic acid | 412.49 | |
| P-0579 | 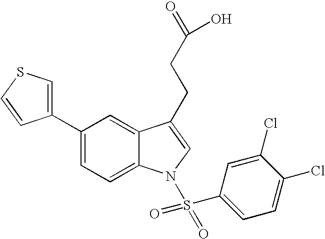 | 3-[1-(3,4-Dichloro- benzenesulfonyl)-5- thiophen-3-yl-1H-indol-3- yl]-propionic acid | 480.39 | |
| P-0580 | 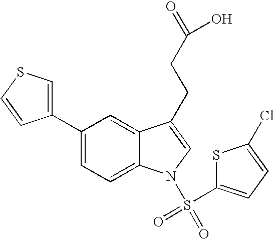 | 3-[1-(5-Chloro-thiophene-2- sulfonyl)-5-thiophen-3-yl- 1H-indol-3-yl]-propionic acid | 451.97 | |
| P-0591 | 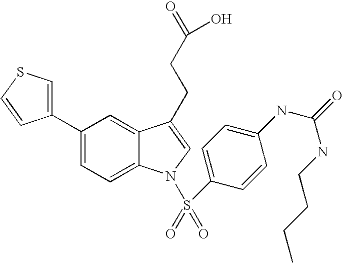 | 3-{1-[4-(3-Butyl-ureido)- benzenesulfonyl]-5- thiophen-3-yl-1H-indol-3- yl}-propionic acid | 525.65 | |
| P-0593 | 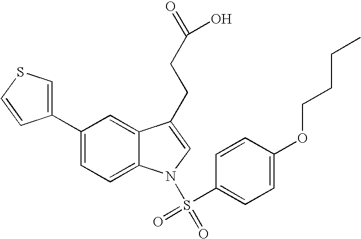 | 3-[1-(4-Butoxy- benzenesulfonyl)-5- thiophen-3-yl-1H-indol-3- yl]-propionic acid | 483.61 | |
| P-0594 | 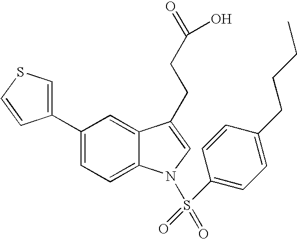 | 3-[1-(4-Butyl- benzenesulfonyl)-5- thiophen-3-yl-1H-indol-3- yl]-propionic acid | 467.61 | |
| P-0595 | 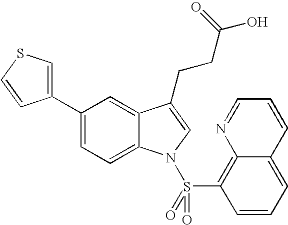 | 3-[1-(Quinoline-8-sulfonyl)- 5-thiophen-3-yl-1H-indol-3- yl]-propionic acid | 462.55 | |
| P-0596 | 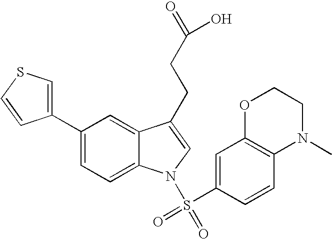 | 3-[1-(4-Methyl-3,4-dihydro- 2H-benzo[1,4]oxazine-7- sulfonyl)-5-thiophen-3-yl- 1H-indol-3-yl]-propionic acid | 482.58 | |
| P-0597 | 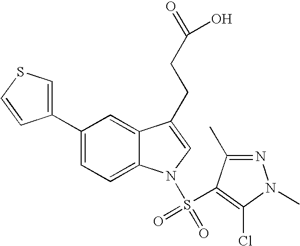 | 3-[1-(5-Chloro-1,3-dimethyl- 1H-pyrazole-4-sulfonyl)-5- thiophen-3-yl-1H-indol-3- yl]-propionic acid | 463.96 | |
| P-0598 | 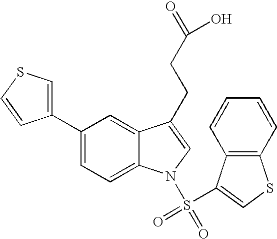 | 3-[1-(Benzo[b]thiophene-3- sulfonyl)-5-thiophen-3-yl- 1H-indol-3-yl]-propionic acid | 467.59 | |
| P-0600 | 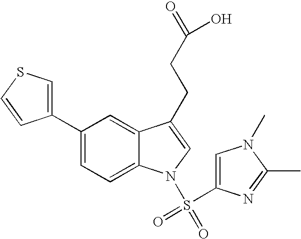 | 3-[1-(1,2-Dimethyl-1H- imidazole-4-sulfonyl)-5- thiophen-3-yl-1H-indol-3- yl]-propionic acid | 429.52 | |
| P-0602 | 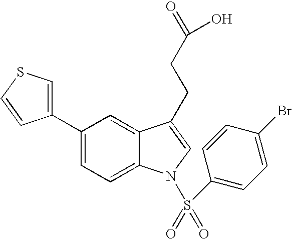 | 3-[1-(4-Bromo- benzenesulfonyl)-5- thiophen-3-yl-1H-indol-3- yl]-propionic acid | 490.40 | |
| P-0603 | 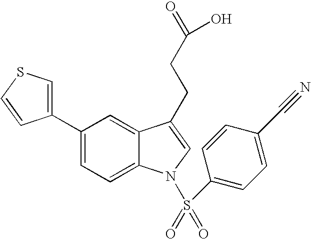 | 3-[1-(4-Cyano- benzenesulfonyl)-5- thiophen-3-yl-1H-indol-3- yl]-propionic acid | 436.51 | |
| P-0604 | 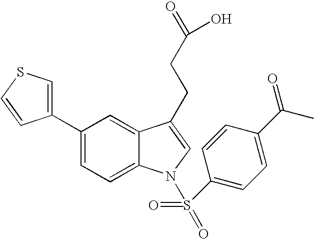 | 3-[1-(4-Acetyl- benzenesulfonyl)-5- thiophen-3-yl-1H-indol-3- yl]-propionic acid | 453.54 | |
| P-0605 | 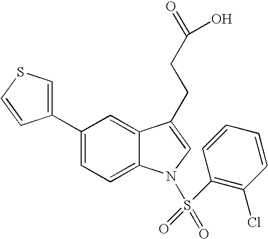 | 3-[1-(2-Chloro- benzenesulfonyl)-5- thiophen-3-yl-1H-indol-3- yl]-propionic acid | 445.95 | |
| P-0606 | 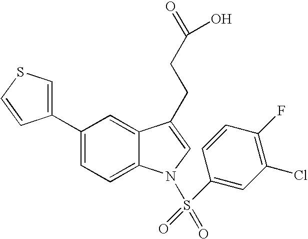 | 3-[1-(3-Chloro-4-fluoro- benzenesulfonyl)-5- thiophen-3-yl-1H-indol-3- yl]-propionic acid | 463.94 | |
| P-0609 | 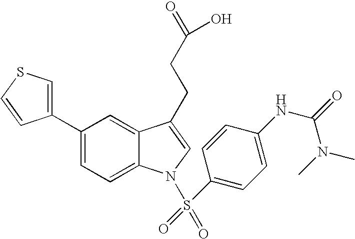 | 3-{1-[4-(3,3-Dimethyl- ureido)-benzenesulfonyl]-5- thiophen-3-yl-1H-indol-3- yl}-propionic acid | 497.59 | |
| P-0610 | 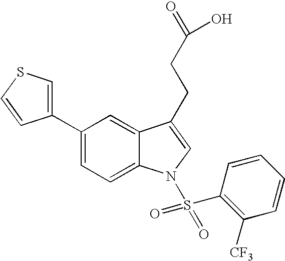 | 3-[5-Thiophen-3-yl-1-(2- trifluoromethyl- benzenesulfonyl)-1H-indol- 3-yl]-propionic acid | 479.50 | |
| P-0611 | 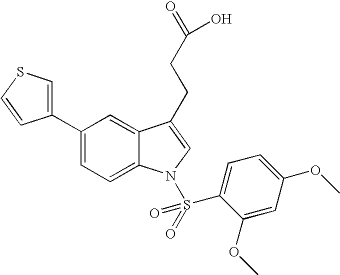 | 3-[1-(2,4-Dimethoxy- benzenesulfonyl)-5- thiophen-3-yl-1H-indol-3- yl]-propionic acid | 471.55 | |
| P-0612 | 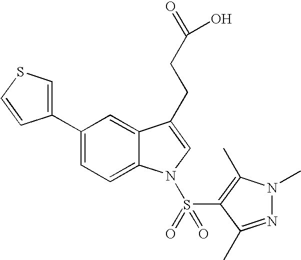 | 3-[5-Thiophen-3-yl-1-(1,3,5- trimethyl-1H-pyrazole-4- sulfonyl)-1H-indol-3-yl]- propionic acid | 443.55 | |
| P-0613 | 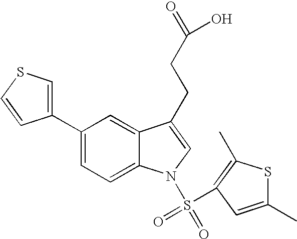 | 3-[1-(2,5-Dimethyl- thiophene-3-sulfonyl)-5- thiophgen-3-yl-1H-indol-3- yl]-propionic acid | 445.58 | |
| P-0614 | 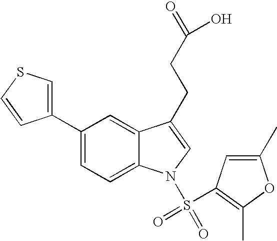 | 3-[1-(2,5-Dimethyl-furan-3- sulfonyl)-5-thiophen-3-yl- 1H-indol-3-yl]-propionic acid | 429.52 | |
| P-0615 | 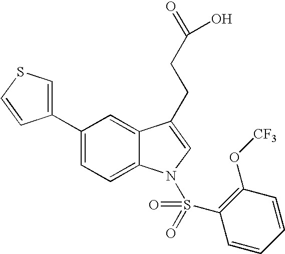 | 3-[5-Thiophen-3-yl-1-(2- trifluoromethoxy- benzenesulfonyl)-1H-indol- 3-yl]-propionic acid | 495.50 | |
| P-0616 | 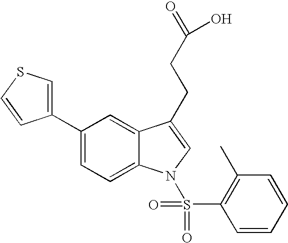 | 3-[5-Thiophen-3-yl-1- (toluene-2-sulfonyl)-1H- indol-3-yl]-propionic acid | 425.53 | |
| P-0619 | 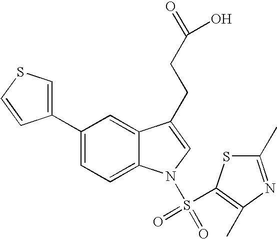 | 3-[1-(2,4-Dimethyl-thiazole- 5-sulfonyl)-5-thiophen-3-yl- 1H-indol-3-yl]-propionic acid | 446.57 | |
| P-0620 | 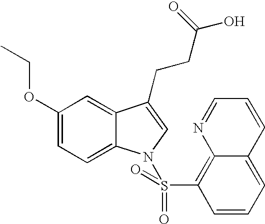 | 3-[5-Ethoxy-1-(quinoline-8- sulfonyl)-1H-indol-3-yl]- propionic acid | 424.48 | |
| P-0621 | 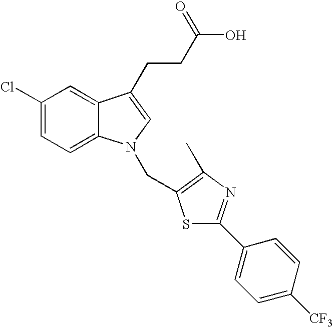 | 3-{5-Chloro-1-[4-methyl-2- (4-trifluoromethyl-phenyl)- thiazol-5-ylmethyl]-1H- indol-3-yl}-propionic acid | 478.92 | |
| P-0622 | 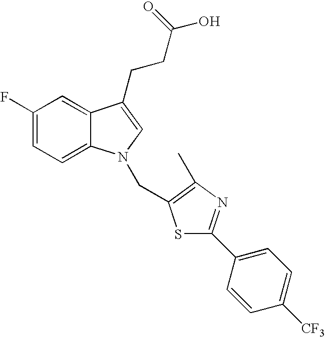 | 3-{5-Fluoro-1-[4-methyl-2- (4-trifluoromethyl-phenyl)- thiazol-5-ylmethyl]-1H- indol-3-yl}-propionic acid | 462.47 | |

### Example 8

### Expression and Purification of PPARs for use in Biochemical and Cell Assays

Genetic Engineering:

Plasmids encoding the Ligand-binding domains (LBDs) of PPARα, PPARYγ, and PPARδ were engineered using common polymerase chain reaction (PCR) methods (pGal4-PPARα-LBD, pGal4-PPAR-γLBD, pGal4-PPARδ-LBD). The relevant DNA sequences and encoded protein sequences used in the assay are shown for each (see below). Complementary DNA cloned from various human tissues were purchased from Invitrogen, and these were used as substrates in the PCR reactions. Specific custom synthetic oligonucleotide primers (Invitrogen, see below) were designed to initiate the PCR product, and also to provide the appropriate restriction enzyme cleavage sites for ligation with the plasmids.

The plasmids used for ligation with the receptor-encoding inserts were either pET28 (Novagen) or a derivative of pET28, pET-BAM6, for expression using E. coli. In each of these cases the receptor LBD was engineered to include a Histidine tag for purification using metal affinity chromatography.

Protein Expression and Purification of PPAR's:

For protein expression, plasmids containing genes of interest were transformed into E. coli strain BL21(DE3)RIL (Invitrogen) and transformants selected for growth on LB agar plates containing appropriate antibiotics. Single colonies were grown for 4 hrs at 37° C. in 200 ml LB media. For PPARα and PPARγ all protein expression was performed by large scale fermentation using a 30 L bioreactor. 400 ml of starter culture was added to 30 L TB culture and allowed to grow at 37° C. until an OD600 nm of 2-5 was obtained. The culture was cooled to 20° C. and 0.5 mM IPTG added, the culture was allowed to grow for a further 18 hrs.

For PPARδ protein expression, single colonies were grown for 4 hrs at 37° C. in 200 ml LB media. 16×1 L of fresh TB media in 2.8 L flasks were inoculated with 10 ml of starter culture and grown with constant shaking at 37° C. Once cultures reached an absorbance of 1.0 at 600 nm, an additive to improve the solubility of the PPARδ was added to the culture and 30 min later, 0.5 mM IPTG was added and cultures allowed to grow for a further 12 to 18 hrs at 20° C. Cells were harvested by centrifugation and pellets frozen at −80° C. until ready for lysis/purification.

For protein purification; all operations were carried out at 4° C. Frozen E. coli cell pellets were resuspended in lysis buffer and lysed using standard mechanical methods. Soluble proteins were purified via poly-Histidine tags using immobilized metal affinity purification (IMAC). For each of the PPAR's described all have been purified using a 3 step purification process utilizing IMAC, size exclusion chromatography and ion exchange chromatography. For PPARα the poly-Histidine tag was optionally removed using Thrombin (Calbiochem). In the case of PPARδ, during protein purification the solubility improving additive was present in order to maintain protein stability. During the final step of purification solubility improving additives were desalted away before concentration.

Plasmid Sequence and PCR Primer Information:

PPARα: (Nucleic acid SEQ ID NO:_) (Protein SEQ ID NO:_)
P332. pET28 PPARA E199-Y468-X
taatacgactcactataggggaattgt
gagcggataacaattcccctctagaaataattttgtttaactttaagaaggagatatacc
atgggcagcagccatcatcatcatcatcacagcagcggcctggtgccgcgcggcaggcat
M G S S H H H H H H S S G L V P R G S H
atggaaactgcagatctcaaatctctggccaagagaatctacgaggcctacttgaagaac
M E T A D L K S L A K R I Y E A Y L K N
ttcaacatgaacaaggtcaaagcccgggtcatcctctcaggaaaggccagtaacaatcca
F N M N K V K A R V I L S G K A S N N P
ccttttgtcatacatgatatggagacactgtgtatggctgagaagacgctggtgggcaag
P F V I H D M E T L C M A E K T L V A K
ctggtggccaatggcatccagaacaaggaggcggaggtccgcatctttcactgctgccag
L V A N G I Q N K E A E V R I F H C C Q
tgcacgtcagtggagaccgtcacggagctcacggaattcgccaaggccatcccaggcttc
C T S V E T V T E L T E F A K A I P G F
gcaaacttggacctgaacgatcaagtgacattgctaaaatacggagtttatgaggccata
A N L D L N D Q V T L L K Y G V Y E A I
ttcgccatgctgtcttctgtgatgaacaaagacgggatgctggtagcgtatggaaatggg
F A M L S S V M N K D G M L V A Y G N G
tttataactcgtgaattcctaaaaagcctaaggaaaccgttctgtgatatcatggaaccc
F I T R E F L K S L R K P F C D I M E P
aagtttgattttgccatgaagttcaatgcactggaactggatgacagtgatatctcgctt
K F D F A M K F N A L E L D D S D I S L
tttgtggctgctatcatttgctgtggagatcgtcctggccttctaaacgtaggacacatt
F V A A I I C C G D R P G L L N V G H I
gaaaaaatgcaggagggtattgtacatgtgctcagactccacctgcagagcaaccacccg
E K M Q E G I V H V L R L H L Q S N H P
gacgatatctttctcttcccaaaacttcttcaaaaaatggcagacctccggcagctggtg
D D I F L F P K L L Q K M A D L R Q L V
acggagcatgcgcagctggtgcagatcatcaagaagacggagtcggatgctgcgctgcac
T E H A Q L V Q I I K K T E S D A A L H
ccgctactgcaggagatctacagggacatgtactgagtcgacaagcttgcggccgcactc
P L L Q E I Y R D M Y -
gagcaccaccaccaccaccactgagat

PCR Primers:

| | | | |
|---|---|---|---|
| PPARA | PPARA-S | GCTGACACATATGGAAACTGCAGATCTCAAATC | (SEQ ID NO:_) |
| | PPARA-A | GTGACTGTCGACTCAGTACATGTCCCTGTAGA | (SEQ ID NO:_) |

PPARγ: (Nucleic acid SEQ ID NO:_) (Protein SEQ ID NO:_)
P333. pET28 PPARG E205-Y475-X
taatacgactcactataggggaattgt
gagcggataacaattcccctctagaaataattttgtttaactttaagaaggagatatacc
atgggcagcagccatcatcatcatcatcacagcagcggcctggtgccgcgcggcagccat
M G S S H H H H H H S S G L V P R G S H
atggagtccgctgacctccgggccctggcaaaacatttgtatgactcatacataaagtcc
M E S A D L R A L A K H L Y D S Y I K S
ttcccgctgaccaaagcaaaggcgagggcgatcttgacaggaaagacaacagacaaatca
F P L T K A K A R A I L T G K T T D K S
ccattcgttatctatgacatgaattccttaatgatgggagaagataaaatcaagttcaaa
P F V I Y D M N S L M M G E D K I K F K
cacatcacccccctgcaggagcagagcaaagaggtggccatccgcatctttcagggctgc
H I T P L Q E Q S K E V A I R I F Q G C
cagtttcgctccgtggaggctgtgcaggagatcacagagtatgccaaaagcattcctggt
Q F R S V E A V Q E I T E Y A K S I P G
tttgtaaatcttgacttgaacgaccaagtaactctcctcaaatatggagtccacgagatc
F V N L D L N D Q V T L L K Y G V H E I
atttacacaatgctggcctccttgatgaataaagatggggttctcatatccgagggccaa
I Y T M L A S L M N K D G V L I S E G Q
ggcttcatgacaagggagtttctaaagagcctgcgaaagccttttggtgactttatggag
G F M T R E F L K S L R K P F G D F M E
cccaagtttgagtttgctgtgaagttcaatgcactggaattagatgacagcgacttggca
P K F E F A V K F N A L E L D D S D L A
atatttattgctgtcattattctcagtggagaccgcccaggtttgctgaatgtgaagccc
I F I A V I I L S G D R P G L L N V K P
attgaagacattcaagacaacctgctacaagccctggagctccagctgaagctgaaccac
I E D I Q D N L L Q A L E L Q L K L N H
cctgagtcctcacagctgtttgccaagctgctccagaaaatgacagacctcagacagatt
P E S S Q L F A K L L Q K M T D L R Q I
gtcacggaacatgtgcagctactgcaggtgatcaagaagacggagacagacatgagtctt
V T E H V Q L L Q V I K K T E T D M S L
cacccgctcctgcaggagatctacaaggacttgtactaggtcgacaagcttgcggccgca
H P L L Q E I Y K D L Y -
ctcgagcaccaccaccaccaccactgagat

PCR Primers:

| | | | |
|---|---|---|---|
| PPARG | PPARG-S | GCTCAGACATATGGAGTCCGCTGACCTCCGGGC | (SEQ ID NO:_) |
| | PPARG-A | GTGACTGTCGACCTAGTACAAGTCCTTGTAGA | (SEQ ID NO:_) |

PPARδ: (Nucleic acid SEQ ID NO:_) (Protein SEQ ID NO:_)
P1057. pET BAM6 PPARD G165-Y441-X
taatacgactcactataggggaattgt
gagcggataacaattcccctctagaaataattttgtttaactttaagaaggagatatacc
atgaaaaaaggtcaccaccatcaccatcacggatcccagtacaacccacaggtggccgac
M K K G H H H H H H G S Q Y N P Q V A D
ctgaaggccttctccaagcacatctacaatgcctacctgaaaaacttcaacatgaccaaa
L K A F S K H I Y N A Y L K N F N M T K
aagaaggcccgcagcatcctcaccggcaaagccagccacacggcgccctttgtgatccac
K K A R S I L T G K A S H T A P F V I H
gacatcgagacattgtggcaggcagagaaggggctggtgtggaagcagttggtgaatggc
D I E T L W Q A E K G L V W K Q L V N G
ctgcctccctacaaggagatcagcgtgcacgtcttctaccgctgccagtgcaccacagtg
L P P Y K E I S V H V F Y R C Q C T T V
gagaccgtgcgggagctcactgagttcgccaagagcatccgcagcttcagcaggctcttc
E T V R E L T E F A K S I P S F S S L F
ctcaacgaccaggttacccttctcaagtatggcgtgcacgaggccatcttcggcatgctg
L N D Q V T L L K Y G V H E A I F A M L
gcctctatcgtcaacaaggacgggctgctggtagccaacggcagtggctttgtcacccgt
A S I V N K D G L L V A N G S G F V T R
gagttcctgcgcagcctccgcaaacccttcagtgatatcattgagcctaagtttgaattt
E F L R S L R K P F S D I I E P K F E F
gctgtcaagttcaacgccctggaacttgatgacagtgacctgggcCtattcattgcgggc
A V K F N A L E L D D S D L A L F I A A
atcattctgtgtggagaccggccaggcctcatzgaacgttccacgggtggaggctatccag
I I L C G D R P G L M N V P R V E A I Q
gacaccatcctgcgtgccctcgaattccacctgcaggccaaccaccctgatgcccagtac
D T I L R A L E F H L Q A N H P D A Q Y
ctcttccccaagctgctgcagaagatggctgacctgcggcaactggtcaccgagcacggc
L F P K L L Q K M A D L R Q L V T E H A
cagatgatgcagcggatcaagaagaccgaaaccgagacctcgctgcaccctctgctccag
Q M M Q R I K K T E T E T S L H P L L Q
gagatctacaaggacatgtactaagtcgaccaccaccaccaccaccactgagatccggct
E I Y K D M Y -
ggccctactggccgaaaggaattcgaggccagcagggccaccgctgagcaataactagca
taaccccttggggcctctaaacgggtcttgaggggttttttg

PCR Primers:

PPARD

(SEQ ID NO:_)

| | |
|---|---|
| PPARD-G165 | GTTGGATCCCAGTACAACCCACAGGTGGC |

(SEQ ID NO:_)

| | |
|---|---|
| PPARD-A | GTGACTGTCGACTTAGTACATGTCCTTGTAGA |

### Example 9

### Bio-Chemical Screening

The homogenous Alpha screen assay was used in the agonist mode to determine the ligand dependent interaction of the PPARs (α,δ,γ) with the coactivator Biotin-PGC-1 peptide (biotin-AHX-DGTPPPQEAEEPSLLKKLLLAPANT-CONH₂ (SEQ ID NO:______), supplied by Wyeth). All compounds tested were serially diluted 1:3 into DMSO for a total of 8 concentration points. Samples were prepared with His-tagged PPAR-LBD prepared per Example 8. Ni-chelate acceptor beads were added that bind to the his-tagged PPAR-LBD and streptavidin donor beads were added that bind to the biotin of the coactivator (Perkin-Elmer #6760619M) such that agonist activity correlates to signal from the donor and acceptor beads in close proximity. Each sample was prepared by mixing 1 μl of compound and 15 μl of 1.33× receptor/peptide mix, incubating for 15 minutes at room temperature, then adding 4 μl of 4× beads in assay buffer. The assay buffer was 50 mM HEPES, pH 7.5, 50 mM KCl, 1 mM DTT and 0.8% BSA. Final concentrations for each sample were 25 nM biotin-PGC-1 peptide, 20 nM PPARγ or 10 nM PPARα or δ, and each bead at 5 μg/ml, with compound added to the desired concentration resulting in final DMSO of 5%. WY-14643(PPARα), farglitazar (PPARγ) and bezafibrate (PPARδ) were assayed as control samples. The samples were incubated for 1hour in the dark at room temperature before taking the reading in the Fusion alpha or Alpha Quest reader. The signal vs. compound concentration was used to determine the EC₅₀. The data was expressed in μMol/L. The data points from the Fusion alpha instrument were transferred to Assay Explorer® (MDL) to generate a curve and calculate the inflection point of the curve as EC₅₀.

### Example 10

### Co-Transfection Assay

This assay serves to confirm the observed biochemical activity (Example 9) on the modulation of intended target molecule(s) at the cellular level. 293T cells (ATCC) were seeded at 1-2×10⁶ cells per well of a 6 well plate (Corning 3516) in 3 ml of growth medium (Dulbecco's eagle medium, Mediatech, with 10% FBS). These were incubated to 80-90% confluent and the medium was removed by aspirating. These cells were transfected with PPAR LBD and luciferase such that agonist results in activation of the luciferase. Measurement of luciferase activity of transfected cells treated with compounds directly correlates with agonist activity. To 100 μl of serum free growth medium was added 1 μg of pFR-Luc (Stratagene catalog number 219050), 6 μl Metafectene (Biontex, Inc.) and 1 mg of the pGal4-PPAR-LBD(α; γ or 67 from Example 8). This was mixed by inverting, then incubated for 15-20 minutes at room temperature, and diluted with 900 μl of serum free growth medium. This was overlayed onto the 293T cells and incubated for 4-5 hours at 37° C. in CO₂ incubator. The transfection medium was removed by aspirating and growth medium was added and the cells incubated for 24 hours. The cells were then suspended in 5 ml of growth medium and diluted with an additional 15 ml of growth medium. For each test sample, 95 μl of the transfected cells were transferred per well of a 96 well culture plate. Compounds tested were diluted in DMSO to 200× the desired final concentration. This was diluted 10× with growth medium and 5 μl was added to the 95 μl of transfected cells. The plate was incubated for 24 hours 37° C. in CO₂ incubator. Luciferase reaction mixture was prepared by mixing 1 ml of lysis buffer, 1 ml of substrate in lysis buffer, and 3 ml of reaction buffer (Roche Diagnostics Luciferase assay kit #1814036). For each sample well, the growth medium was replaced with 50 ml of reaction mixture and the plate shaken for 15-20 minutes, and the luminescence was measured on a Victor2 V plate reader (Perkin Elmer). The signal vs. compound concentration was used to determine the EC₅₀.

This assay serves to confirm the observed biochemical activity (Example 9) on the modulation of intended target molecule(s) at the cellular level. Compounds having EC₅₀ of less than or equal to 1 μM in either of the biochemical assay of Example 9 or this cell based assay for at least one of the PPARs are shown in Table 8. Additional compounds disclosed in PCT publication WO 2005/009958 demonstrated EC₅₀ of less than or equal to 1 μM for at least one of PPARs. These were compounds 1, 22, 29, 41, 43, 45, 47, 51, 53, 55, 59, 63, 65, 67, 69, 77, 79, 82, 83, 90, 92, 94, 101, 102, 107, 108, 109, 110, 111, 112, 113, 115, and 116 from Table 1 beginning on page 184 of the published application and compound 119 (Example 81), compound 121 (Example 99) and Compound 126 (Example 103) from the application.

TABLE 8
Compounds having EC₅₀ of less than or equal to 1 μM in at least one of
PPARα, PPARγ and PPARδ activity assays.

P-0001, P-0002, P-0003, P-0004, P-0007, P-0008, P-0010, P-0015, P-0016, P-0017,
P-0018, P-0019, P-0020, P-0022, P-0026, P-0031, P-0032, P-0033, P-0034, P-0035,
P-0037, P-0039, P-0046, P-0048, P-0049, P-0050, P-0051, P-0052, P-0053, P-0054,
P-0055, P-0056, P-0057, P-0058, P-0060, P-0063, P-0064, P-0066, P-0067, P-0068,
P-0069, P-0070, P-0071, P-0072, P-0080, P-0082, P-0092, P-0099, P-0100, P-0108,
P-0144, P-0147, P-0149, P-0150, P-0151, P-0155, P-0158, P-0159, P-0160, P-0163,
P-0165, P-0166, P-0167, P-0174, P-0175, P-0188, P-0203, P-0207, P-0208, P-0209,
P-0210, P-0214, P-0215, P-0219, P-0220, P-0221, P-0222, P-0223, P-0224, P-0225,
P-0226, P-0227, P-0228, P-0229, P-0230, P-0231, P-0236, P-0270, P-0276, P-0277,
P-0278, P-0279, P-0280, P-0282, P-0283, P-0284, P-0285, P-0286, P-0289, P-0290,
P-0293, P-0294, P-0295, P-0296, P-0297, P-0298, P-0308, P-0309, P-0310, P-0311,
P-0315, P-0316, P-0317, P-0318, P-0319, P-0320, P-0322, P-0323, P-0324, P-0326,
P-0327, P-0328, P-0329, P-0330, P-0331, P-0334, P-0335, P-0337, P-0340, P-0341,
P-0343, P-0344, P-0347, P-0351, P-0356, P-0359, P-0360, P-0361, P-0362, P-0363,
P-0364, P-0371, P-0373, P-0376, P-0377, P-0378, P-0379, P-0380, P-0381, P-0382,
P-0383, P-0384, P-0385, P-0386, P-0387, P-0388, P-0389, P-0395, P-0396, P-0398,
P-0399, P-0400, P-0401, P-0402, P-0404, P-0405, P-0408, P-0409, P-0411, P-0412,
P-0413, P-0415, P-0419, P-0420, P-0422, P-0423, P-0424, P-0427, P-0430, P-0431,
P-0434, P-0435, P-0436, P-0437, P-0438, P-0439, P-0440, P-0446, P-0447, P-0448,
P-0449, P-0450, P-0451, P-0452, P-0454, P-0455, P-0456, P-0458, P-0462, P-0463,
P-0464, P-0465, P-0466, P-0467, P-0468, P-0470, P-0471, P-0472, P-0473, P-0474,
P-0475, P-0476, P-0477, P-0478, P-0479, P-0481, P-0482, P-0483, P-0484, P-0485,
P-0486, P-0487, P-0488, P-0489, P-0490, P-0491, P-0492, P-0493, P-0494, P-0495,
P-0496, P-0497, P-0498, P-0499, P-0501, P-0502, P-0503, P-0504, P-0505, P-0506,
P-0508, P-0509, P-0510, P-0511, P-0512, P-0513, P-0514, P-0515, P-0516, P-0517,
P-0518, P-0519, P-0520, P-0521, P-0523, P-0524, P-0529, P-0530, P-0533, P-0535,
P-0537, P-0538, P-0539, P-0540, P-0541, P-0549, P-0552, P-0553, P-0555, P-0560,
P-0561, P-0564, P-0566, P-0567, P-0568, P-0570, P-0572

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. For example, variations can be made to exemplary compounds of I, Ia, Ib, II, or III to provide additional active compounds. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values are provided for embodiments, additional embodiments are described by taking any 2 different values as the endpoints of a range. Such ranges are also within the scope of the described invention.

Thus, additional embodiments are within the scope of the invention and within the following claims.

1. A compound having the chemical structure 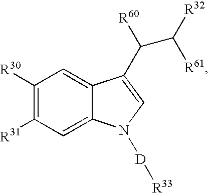
all salts, prodrugs, tautomers and isomers thereof,
wherein:
R³⁰ and R³¹ are independently selected from the group consisting of hydrogen, halogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted heteroaryl, —OH, —OR³⁴, —SR³⁵, —NR³⁶R³⁷, —C(Z)NR³⁸R³⁹, —C(Z)R⁴⁰, —S(O)₂NR³⁸R³⁹, and —S(O)nR⁴¹; or
R³⁰ and R³¹ combine to form a fused ring, wherein the combined R³⁰ and R³¹ are of the formula 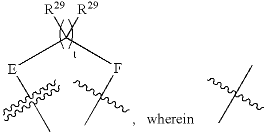
indicates the point of attachment of R³⁰ to the indole ring and 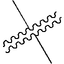
indicates the point of attachment of R³¹ to the indole ring;
E and F are independently selected from the group consisting of CR²⁹R²⁹, O, S(O)₂ and NR⁴⁴;
R²⁹ at each occurrence is independently selected from the group consisting of hydrogen, fluoro, optionally fluoro substituted lower alkyl, optionally fluoro substituted lower alkoxy, and optionally fluoro substituted lower alkylthio;
R⁴⁴ is hydrogen or lower alkyl;
t is 1 or 2;
R³² is selected from the group consisting of —C(O)OR²⁶, —C(O)NR²⁷R²⁸, and a carboxylic acid isostere;
R³³ is L-R⁴² or heteroaryl optionally substituted with one or more substituents selected from the group consisting of halogen, optionally substituted lower alkyl, optionally substituted lower alkenyl, optionally substituted lower alkynyl, optionally substituted cycloalkyl, optionally substituted aryl, optionally substituted heterocycloalkyl, optionally substituted heteroaryl, —OH, —NO₂, —CN, —OR³⁴, —SR³⁵, —NR³⁶R³⁷, —C(Z)NR³⁸R³⁹, —C(Z)R⁴⁰, —S(O)₂NR³⁸R³⁹, and —S(O)ₙR⁴¹;
L is —(CR⁵¹R⁵²)ₘ— or —CR⁵⁵═CR⁵⁶—;
D is —CR⁵¹R⁵²— or —S(O)₂—;
R³⁴ is selected from the group consisting of optionally substituted lower alkyl, optionally substituted C₃₋₆ alkenyl, provided, however, that when R³⁴ is optionally substituted C₃₋₆ alkenyl, no alkene carbon thereof is bound to the O of —OR³⁴, optionally substituted C₃₋₆ alkynyl, provided, however, that when R³⁴ is optionally substituted C₃₋₆ alkynyl, no alkyne carbon thereof is bound to the O of —OR³⁴, optionally substituted cycloalkyl, optionally substituted heterocycloalkyl, optionally substituted aryl, optionally substituted heteroaryl, —C(Z)R⁴⁰, and —C(Z)NR³⁸R³⁹;
R³⁵ is selected from the group consisting of optionally substituted lower alkyl, optionally substituted C₃₋₆ alkenyl, provided, however, that when R³⁵ is optionally substituted C₃₋₆ alkenyl, no alkene carbon thereof is bound to the S of —SR³⁵ or the O of —OR³⁵, optionally substituted C₃₋₆ alkynyl, provided, however, that when R³⁵ is optionally substituted C₃₋₆ alkynyl, no alkyne carbon thereof is bound to the S of —SR³⁵ or the O of —OR³⁵, optionally substituted cycloalkyl, optionally substituted heterocycloalkyl, optionally substituted aryl, and optionally substituted heteroaryl;
R36 and R37 are independently selected from the group consisting of hydrogen, optionally substituted lower alkyl, optionally substituted C₃₋₆ alkenyl, provided, however, that when R³⁶ and/or R³⁷ are optionally substituted C₃₋₆ alkenyl, no alkene carbon thereof is bound to the N of —NR³⁶R³⁷, optionally substituted C₃₋₆ alkynyl, provided, however, that when R³⁶ and/or R³⁷ are optionally substituted C₃₋₆ alkynyl, no alkyne carbon thereof is bound to the N of —NR³⁶R³⁷, optionally substituted cycloalkyl, optionally substituted heterocycloalkyl, optionally substituted aryl, optionally substituted heteroaryl, —C(Z)R⁴⁰, —C(Z)NR³⁸R³⁹, —S(O)₂R⁴¹, and —S(O)₂NR³⁸R³⁹;
R³⁸ and R³⁹ are independently selected from the group consisting of hydrogen, optionally substituted lower alkyl, optionally substituted C₃₋₆ alkenyl, provided, however, that when R³⁸ and/or R³⁹ are optionally substituted C₃₋₆ alkenyl, no alkene carbon thereof is bound to the N of NR³⁸R³⁹, optionally substituted C₃₋₆ alkynyl, provided, however, that when R³⁸ and/or R³⁹ are optionally substituted C₃₋₆ alkynyl, no alkyne carbon thereof is bound to the N of NR³⁸R³⁹, optionally substituted cycloalkyl, optionally substituted heterocycloalkyl, optionally substituted aryl, and optionally substituted heteroaryl;
R⁴⁰ is selected from the group consisting of optionally substituted lower alkyl, optionally substituted C₃₋₆ alkenyl, provided, however, that when R⁴⁰ is optionally substituted C₃₋₆ alkenyl, no alkene carbon thereof is bound to —C(Z)—, optionally substituted C₃₋₆ alkynyl, provided, however, that when R⁴⁰ is optionally substituted C₃₋₆ alkynyl, no alkyne carbon thereof is bound to —C(Z)—, optionally substituted cycloalkyl, optionally substituted heterocycloalkyl, optionally substituted aryl, optionally substituted heteroaryl, —OH, and —OR³⁵;
R⁴¹ is selected from the group consisting of optionally substituted lower alkyl, optionally substituted C₃₋₆ alkenyl, provided, however, that when R⁴¹ is optionally substituted C₃₋₆ alkenyl, no alkene carbon thereof is bound to —S(O)n—, optionally substituted C₃₋₆ alkynyl, provided, however, that when R⁴¹ is optionally substituted C₃₋₆ alkynyl, no alkyne carbon thereof is bound to —S(O)ₙ—, optionally substituted cycloalkyl, optionally substituted heterocycloalkyl, optionally optionally substituted aryl, and optionally substituted heteroaryl;
R⁴² is aryl or heteroaryl, wherein aryl or heteroaryl are optionally substituted with one or more substituents selected from the group consisting of halogen, optionally substituted lower alkyl, optionally substituted lower alkenyl, optionally substituted lower alkynyl, optionally substituted cycloalkyl, optionally substituted aryl, optionally substituted heterocycloalkyl, optionally substituted heteroaryl, —OH, —NO₂, —CN, —OR³⁴, —SR³⁵, —NR³⁶R³⁷, —C(Z)NR³⁸R³⁹, —C(Z)R⁴⁰, —S(O)₂NR³⁸R³⁹, and —S(O)ₙR⁴¹;
R⁵¹ and R⁵² are independently selected from the group consisting of hydrogen, fluoro, optionally substituted lower alkyl, optionally substituted cycloalkyl, optionally substituted heterocycloalkyl, optionally substituted aryl, and optionally substituted heteroaryl;
or any two of R⁵¹ and R⁵² on the same carbon or on adjacent carbons may be combined to form an optionally substituted 3-7 membered monocyclic cycloalkyl or optionally substituted 5-7 membered monocyclic heterocycloalkyl;
R⁵⁵ and R⁵⁶ are independently selected from the group consisting of hydrogen, optionally substituted lower alkyl, optionally substituted cycloalkyl, optionally substituted heterocycloalkyl, optionally substituted aryl, and optionally substituted heteroaryl; or
R⁵⁵ and R⁵⁶ combine to form an optionally substituted 5-7 membered monocyclic cycloalkyl or optionally substituted 5-7 membered monocyclic heterocycloalkyl;
R60 and R61 are each hydrogen, or R⁶⁰ and R⁶¹ combine to form optionally substituted 3-7 membered monocyclic cycloalkyl;
R²⁶ is selected from the group consisting of hydrogen, lower alkyl, phenyl, 5-7 membered monocyclic heteroaryl, 3-7 membered monocyclic cycloalkyl, and 5-7 membered monocyclic heterocycloalkyl, wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, and wherein lower alkyl is optionally substituted with one or more substituents selected from the group consisting of fluoro, —OH, —NH₂, lower alkoxy, fluoro substituted lower alkoxy, lower alkylthio and fluoro substituted lower alkylthio, provided, however, that when R²⁶ is lower alkyl, any substitution on the lower alkyl carbon bound to the O of OR²⁶ is fluoro;
R²⁷ and R²⁸ are independently selected from the group consisting of hydrogen, lower alkyl, phenyl, 5-7 membered monocyclic heteroaryl, 3-7 membered monocyclic cycloalkyl, and 5-7 membered monocyclic heterocycloalkyl, wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, and wherein lower alkyl is optionally substituted with one or more substituents selected from the group consisting of fluoro, —OH, —NH₂, lower alkoxy, fluoro substituted lower alkoxy, lower alkylthio and fluoro substituted lower alkylthio, provided, however, that when R²⁷ and/or R²⁸ is lower alkyl, any substitution on the lower alkyl carbon bound to the N of NR²⁷R²⁸ is fluoro; or
R²⁷ and R²⁸ together with the nitrogen to which they are attached form a 5-7 membered monocyclic heterocycloalkyl or a 5 or 7 membered nitrogen containing monocyclic heteroaryl, wherein the monocyclic heterocycloalkyl or monocyclic nitrogen containing heteroaryl is optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
n is 1, or 2;
m is 1, 2, or 3; and
Z is O or S.
provided, however, that when D is —S(O)₂—, R³⁰ is OCH₃, R³¹ is H, and R³² is COOH or COOCH₃, then R³³ is not unsubstituted thiophenyl.

2. The compound according to claim 1, wherein D is —CR⁵¹R⁵²—.

3. The compound according to claim 1, wherein D is —S(O)₂—.

4. The compound according to claim 1, wherein R³³ is substituted heteroaryl.

5. The compound according to claim 4, wherein:
R³³ is heteroaryl substituted with one or more substituents selected from the group consisting of lower alkyl, wherein lower alkyl is substituted with optionally substituted cycloalkyl, optionally substituted heterocycloalkyl, optionally substituted aryl or optionally substituted heteroaryl, optionally substituted cycloalkyl, optionally substituted aryl, optionally substituted heterocycloalkyl, optionally substituted heteroaryl, —OR³⁴, —SR³⁰, —NR³⁶R³⁷, —C(Z)NR³⁸R³⁹, —C(Z)R⁴⁰, —S(O)₂NR³⁸R³⁹, and —S(O)ₙR⁴¹;
wherein wherein one of R³⁶ and R³⁷ is selected from the group consisting of lower alkyl, wherein lower alkyl is substituted with optionally substituted aryl or optionally substituted heteroaryl, optionally substituted cycloalkyl, optionally substituted heterocycloalkyl, optionally substituted aryl, optionally substituted heteroaryl, —C()R⁴⁰, —C(Z)NR³⁸R³⁹, —S(O)₂R⁴¹, and —S(O)₂NR³⁸R³⁹, and the other of R³⁶ and R³⁷ is hydrogen or lower alkyl;
wherein one of R³⁸ and R³⁹ is selected from the group consisting of lower alkyl, wherein lower alkyl is substituted with optionally substituted aryl or optionally substituted heteroaryl, optionally substituted cycloalkyl, optionally substituted heterocycloalkyl, optionally substituted aryl, and optionally substituted heteroaryl, and the other of R³⁸ and R³⁹ is hydrogen or lower alkyl; and
wherein R³⁴, R³⁵, R⁴⁰, and R⁴¹ are independently selected from the group consisting of lower alkyl, wherein lower alkyl is substituted with optionally substituted aryl or optionally substituted heteroaryl, optionally substituted cycloalkyl, optionally substituted heterocycloalkyl, optionally substituted aryl, and optionally substituted heteroaryl.

6. The compound according to claim 5,
wherein
R³⁰ and R³¹ are independently selected from the group consisting of hydrogen, halogen, optionally substituted lower alkyl, optionally substituted lower alkoxy, optionally substituted aryloxy, optionally substituted heteroaryloxy, optionally substituted cycloalkyl, optionally substituted heterocycloalkyl, optionally substituted aryl and optionally substituted heteroaryl, or
R³⁰ and R³¹ combine to form a fused ring wherein E and F are O, t is 1 or 2, and each R²⁹ is hydrogen.

7. The compound according to claim 6, wherein R³¹ is hydrogen.

8. The compound according to claim 6, wherein R³⁰ and R³¹ are independently optionally substituted lower alkoxy, or R³⁰ and R³¹ combine to form a fused ring wherein E and F are O, t is 1 or 2, and each R²⁹ is hydrogen.

9. The compound according to claim 6, wherein D is —S(O)₂—.

10. The compound according to claim 6, wherein D is —CH₂—.

11. A compound having the chemical structure 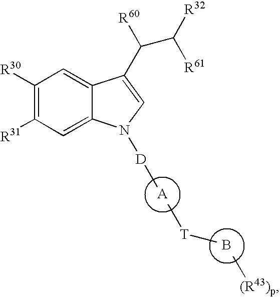
all salts, prodrugs, tautomers and isomers thereof,
wherein:
D is —CR⁵¹R⁵²— or —S(O)₂—;
R³⁰ and R³¹ are independently selected from the group consisting of hydrogen, halogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted heteroaryl, —OH, —OR³⁴, —SR³⁵, —NR³⁶R³⁷, —C(Z)NR³⁸R³⁹, —C(Z)R⁴⁰, —S(O)₂NR³⁸R³⁹, and —S(O)ₙR⁴¹; or
R³⁰ and R³¹ combine to form a fused ring, wherein the combined R³⁰ and R³¹ are of the formula 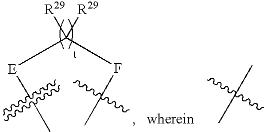
indicates the point of attachment of R³⁰ to the indole ring and 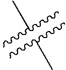
indicates the point of attachment of R³¹ to the indole ring;
E and F are independently selected from the group consisting of CR²⁹R²⁹, O, S(O)₂ and NR⁴⁴;
R²⁹ at each occurrence is independently selected from the group consisting of hydrogen, fluoro, optionally fluoro substituted lower alkyl, optionally fluoro substituted lower alkoxy, and optionally fluoro substituted lower alkylthio;
R⁴⁴ is hydrogen or lower alkyl;
t is 1 or 2;
R³² is selected from the group consisting of —C(O)OR²⁶, —C(O)NR²⁷R²⁸, and a carboxylic acid isostere;
R⁶⁰ and R⁶¹ are each hydrogen, or R⁶⁰ and R⁶¹ combine to form optionally substituted 3-7 membered monocyclic cycloalkyl;
A is arylene or heteroarylene, wherein arylene or heteroarylene are optionally substituted with one or more substituents selected from the group consisting of halogen, —OH, lower alkyl, lower alkoxy, and lower alkylthio, wherein lower alkyl and the lower alkyl chains of lower alkoxy and lower alkylthio are optionally substituted with one or more substituents selected from the group consisting of fluoro, —OH, lower alkoxy, and lower alkylthio, provided, however, that any substitution of the carbon bound to the lower alkoxy O or lower alkylthio S is fluoro;
T is a covalent bond or is selected from the group consisting of —(CR⁵¹R⁵²)ₘ—, —(CR⁵¹R⁵²)_{q}O(CR⁵¹R⁵²)ᵣ—, —(CR⁵¹R⁵²)_{q}S(CR⁵¹R⁵²) ᵣ—, —(CR⁵¹R⁵²)_{q}NR⁵³(CR⁵¹ R⁵²)ᵣ—, —(CR⁵¹R⁵²)_{q}C(Z)(CR⁵¹R⁵²)ᵣ—, —(CR⁵¹R⁵²)_{q}S(O) ₙ(CR⁵¹R⁵²)ᵣ—, —(CR⁵¹R⁵²)_{q}C(Z)NR⁵⁴(CR⁵¹R⁵²)ᵣ—, —(CR⁵¹R⁵²) _{q}NR⁵⁴C(Z)(CR⁵¹R⁵²)ᵣ—, —(CR⁵¹R⁵²)_{q}NR⁵⁴C(Z)NR⁵⁴(CR⁵¹R⁵²)ᵣ—, —(CR⁵¹R⁵²) _{q}NR⁵⁴S(O)₂(CR⁵¹R⁵²)ᵣ—, —(CR⁵¹R⁵²)_{q}S(O)₂NR⁵⁴(CR⁵¹R⁵²)ᵣ—, and —(CR⁵¹R⁵²) _{q}NR⁵⁴S(O)₂NR⁵⁴(CR⁵¹R⁵²)ᵣ—;
R⁵¹ and R⁵² are independently selected from the group consisting of hydrogen, fluoro, optionally substituted lower alkyl, optionally substituted cycloalkyl, optionally substituted heterocycloalkyl, optionally substituted aryl, and optionally substituted heteroaryl;
or any two of R⁵¹ and R⁵² on the same carbon or on adjacent carbons may be combined to form an optionally substituted 3-7 membered monocyclic cycloalkyl or optionally substituted 5-7 membered monocyclic heterocycloalkyl;
m is 1, 2, or 3;
q and r are independently 0, 1, or 2;
B is selected from the group consisting of cycloalkyl, heterocycloalkyl, aryl, and heteroaryl;
R⁴³ at each occurence is independently selected from the group consisting of halogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted heteroaryl, —OH, —OR³⁴, —SR³⁵, —NR^{36 R}³⁷, —C(Z)NR³⁸R³⁹, —C(Z)R⁴⁰, —S(O)₂NR³⁸R³⁹, and —S(O)ₙR⁴¹;
R⁵³ is selected from the group consisting of hydrogen, optionally substituted lower alkyl, optionally substituted C₃₋₆ alkenyl, provided, however, that when R⁵³ is optionally substituted C₃₋₆ alkenyl, no alkene carbon thereof is bound to the N of —NR⁵³—, optionally substituted C₃₋₆ alkynyl, provided, however, that when R⁵³ is optionally substituted C₃₋₆ alkynyl, no alkyne carbon thereof is bound to the N of —NR⁵³—, optionally substituted cycloalkyl, optionally substituted heterocycloalkyl, optionally substituted aryl, optionally substituted heteroaryl, —C(Z)NR³⁸R³⁹, —C(Z)R⁴⁰, —S(O)₂NR³⁸R³⁹, and —S(O)₂R⁴¹;
R⁵⁴ at each occurrence is independently selected from the group consisting of hydrogen, optionally substituted lower alkyl, optionally substituted C₃₋₆ alkenyl, provided, however, that when R⁵⁴ is optionally substituted C₃₋₆ alkenyl, no alkene carbon thereof is bound to the N of —NR⁵⁴—, optionally substituted C₃₋₆ alkynyl, provided, however, that when R⁵⁴ is optionally substituted C₃₋₆ alkynyl, no alkyne carbon thereof is bound to the N of —NR⁵⁴—, optionally substituted cycloalkyl, optionally substituted heterocycloalkyl, optionally substituted aryl, and optionally substituted heteroaryl;
p is 0, 1, 2 or 3;
n is 1, or 2;
Z is O or S;
R³⁴ is selected from the group consisting of optionally substituted lower alkyl, optionally substituted C₃₋₆ alkenyl, provided, however, that when R³⁴ is optionally substituted C₃₋₆ alkenyl, no alkene carbon thereof is bound to the O of —OR³⁴, optionally substituted C₃₋₆ alkynyl, provided, however, that when R³⁴ is optionally substituted C₃₋₆ alkynyl, no alkyne carbon thereof is bound to the O of —OR³⁴, optionally substituted cycloalkyl, optionally substituted heterocycloalkyl, optionally substituted aryl, optionally substituted heteroaryl, —C(Z)R⁴⁰, and —C(Z)NR³⁸R³⁹;
R³⁵ is selected from the group consisting of optionally substituted lower alkyl, optionally substituted C₃₋₆ alkenyl, provided, however, that when R³⁵ is optionally substituted C₃₋₆ alkenyl, no alkene carbon thereof is bound to the S of —SR³⁵ or the O of —OR³⁵, optionally substituted C₃₋₆ alkynyl, provided, however, that when R³⁵ is optionally substituted C₃₋₆ alkynyl, no alkyne carbon thereof is bound to the S of —SR³⁵ or the O of —OR³⁵, optionally substituted cycloalkyl, optionally substituted heterocycloalkyl, optionally substituted aryl, and optionally substituted heteroaryl;
R³⁶ and R³⁷ are independently selected from the group consisting of hydrogen, optionally substituted lower alkyl, optionally substituted C₃₋₆ alkenyl, provided, however, that when R³⁶ and/or R³⁷ are optionally substituted C₃₋₆ alkenyl, no alkene carbon thereof is bound to the N of —NR³⁶R³⁷, optionally substituted C₃₋₆ alkynyl, provided, however, that when R³⁶ and/or R³⁷ are optionally substituted C₃₋₆ alkynyl, no alkyne carbon thereof is bound to the N of —NR³⁶R³⁷, optionally substituted cycloalkyl, optionally substituted heterocycloalkyl, optionally substituted aryl, optionally substituted heteroaryl, —C(Z)R⁴⁰, —C(Z)NR³⁸R³⁹, —S(O)₂R⁴¹, and —S(O)₂NR³⁸R³⁹;
R³⁸ and R³⁹ are independently selected from the group consisting of hydrogen, optionally substituted lower alkyl, optionally substituted C₃₋₆ alkenyl, provided, however, that when R³⁸ and/or R³⁹ are optionally substituted C₃₋₆ alkenyl, no alkene carbon thereof is bound to the N of NR³⁸R³⁹, optionally substituted C₃₋₆ alkynyl, provided, however, that when R³⁸ and/or R³⁹ are optionally substituted C₃₋₆ alkynyl, no alkyne carbon thereof is bound to the N of NR³⁸R³⁹, optionally substituted cycloalkyl, optionally substituted heterocycloalkyl, optionally substituted aryl, and optionally substituted heteroaryl;
R⁴⁰ is selected from the group consisting of optionally substituted lower alkyl, optionally substituted C₃₋₆ alkenyl, provided, however, that when R⁴⁰ is optionally substituted C₃₋₆ alkenyl, no alkene carbon thereof is bound to —C(Z)—, optionally substituted C₃₋₆ alkynyl, provided, however, that when R⁴⁰ is optionally substituted C₃₋₆ alkynyl, no alkyne carbon thereof is bound to —C(Z)—, optionally substituted cycloalkyl, optionally substituted heterocycloalkyl, optionally substituted aryl, optionally substituted heteroaryl, —OH, and —OR³⁵;
R⁴¹ is selected from the group consisting of optionally substituted lower alkyl, optionally substituted C₃₋₆ alkenyl, provided, however, that when R⁴¹ is optionally substituted C₃₋₆ alkenyl, no alkene carbon thereof is bound to —S(O)ₙ—, optionally substituted C₃₋₆ alkynyl, provided, however, that when R⁴¹ is optionally substituted C₃₋₆ alkynyl, no alkyne carbon thereof is bound to —S(O)ₙ—, optionally substituted cycloalkyl, optionally substituted heterocycloalkyl, optionally optionally substituted aryl, and optionally substituted heteroaryl; provided, however, said compound is not 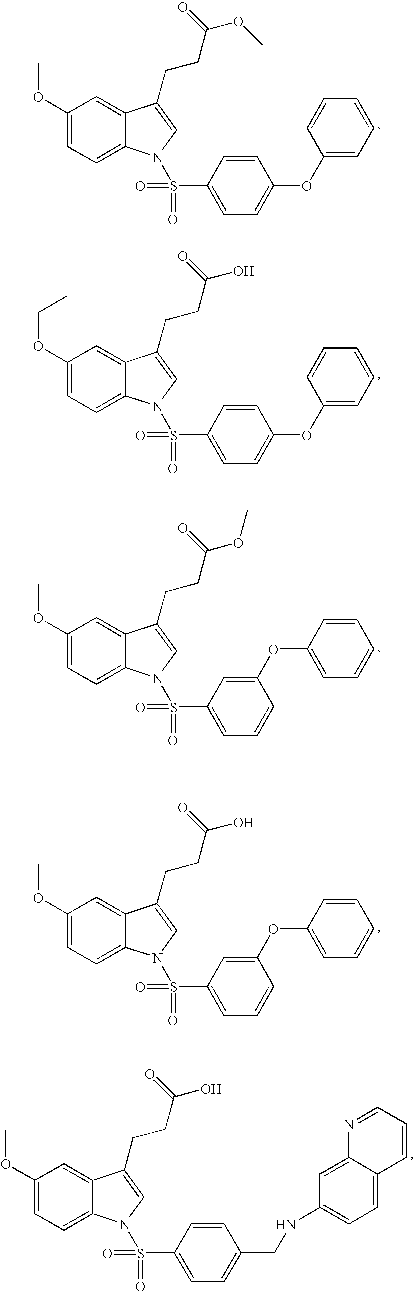 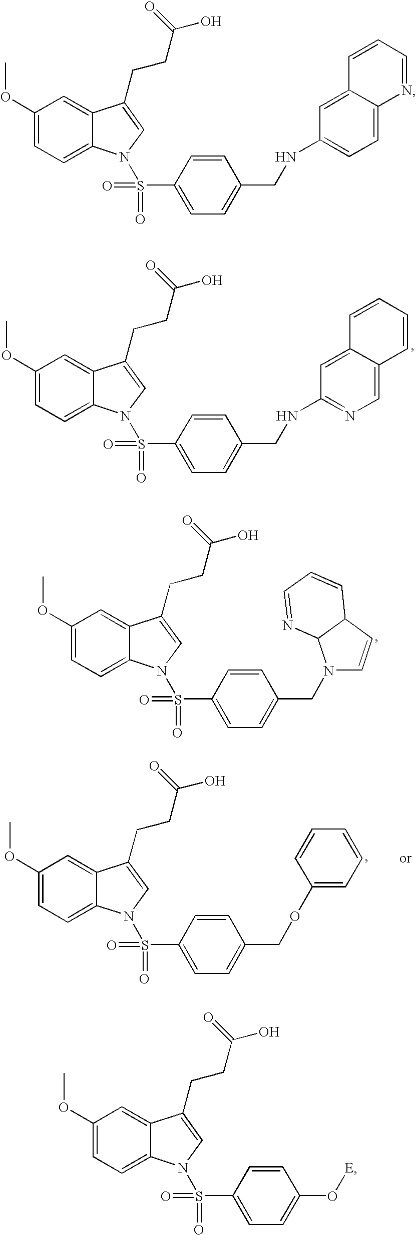
wherein E is 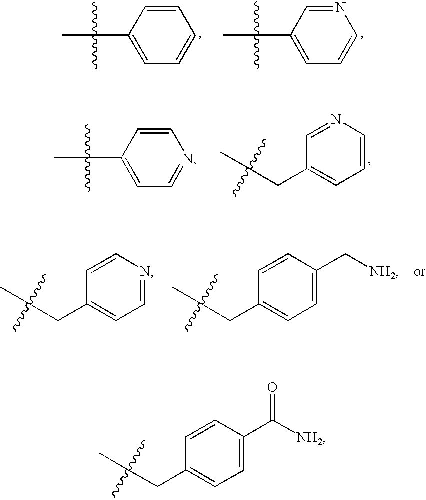
wherein 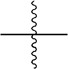
indicates the point of attachment of E to O.

12. The compound according to claim 11, wherein A is phenyl and T-B is ortho to D.

13. The compound according to claim 12, wherein D is —S(O)₂—.

14. The compound according to claim 12, wherein D is —CR⁵¹R⁵²—.

15. The compound according to claim 11, wherein R⁴³ is selected from the group consisting of halogen, —OH, optionally substituted lower alkyl, optionally substituted lower alkenyl, optionally substituted lower alkynyl, —OR³⁴, —SR³⁵, —NR³⁶R³⁷, —C(Z)NR³⁸R³⁹, —C(Z)R⁴⁰, —S(O)₂NR³⁸R³⁹, and —S(O)ₙR⁴¹, wherein R³⁴, R³⁵, R³⁶, R³⁷, R³⁸, R³⁹, R⁴⁰ and R⁴¹ are other than a member selected from the group consisting of optionally substituted cycloalkyl, optionally substituted heterocycloalkyl, optionally substituted aryl, and optionally substituted heteroaryl, or lower alkyl substituted with optionally substituted cycloalkyl, optionally substituted heterocycloalkyl, optionally substituted aryl, or optionally substituted heteroaryl.

16. The compound according to claim 15, wherein R³⁰ and R³¹ are independently selected from the group consisting of hydrogen, halogen, optionally substituted lower alkyl, optionally substituted lower alkoxy, optionally substituted aryloxy, optionally substituted heteroaryloxy, optionally substituted cycloalkyl, optionally substituted heterocycloalkyl, optionally substituted aryl, and optionally substituted heteroaryl, or R³⁰ and R³¹ combine to form a fused ring wherein E and F are O, t is 1 or 2, and each R²⁹ is hydrogen.

17. The compound according to claim 16, wherein R³¹ is hydrogen.

18. The compound according to claim 16, wherein R³⁰ and R³¹ are independently optionally substituted lower alkoxy, or R³⁰ and R³¹ combine to form a fused ring wherein E and F are O, t is 1 or 2, and each R²⁹ is hydrogen.

19. The compound according to claim 11, wherein D is —S(O)₂—.

20. The compound according to claim 11, wherein D is —CH₂—.

21. The compound of claim 11, wherein the compound is selected from the group consisting of 3-{1-[5-(2,4-Dimethoxy-pyrimidin-5-yl)-thiophene-2-sulfonyl]-5-methoxy- 1H-indol-3-yl}-propionic acid,
3-{5-Chloro-1-[5-(2,4-dimethoxy-pyrimidin-5-yl)-thiophene-2-sulfonyl]-1H-indol-3-yl}-propionic acid,
3-{1-[5-(6-Benzyloxy-pyridin-3-yl)-thiophene-2-sulfonyl]-5-methoxy-1H-indol-3-yl}-propionic acid,
3-{1 -[5-(2,6-Dimethoxy-pyridin-3-yl)-thiophene-2-sulfonyl]-5-methoxy-1H-indol-3-yl}-propionic acid,
3-{1-[5-(4-Benzyloxy-phenyl)-thiophene-2-sulfonyl]-5-ethoxy-1H-indol-3-yl}-propionic acid,
3-{5-Ethoxy-1-[5-(6-methoxy-pyridin-3-yl)-thiophene-2-sulfonyl]-1H-indol-3-yl}-propionic acid,
3-{1-[5-(3-Chloro-4-fluoro-phenyl)-thiophene-2-sulfonyl]-5-methoxy-1H-indol-3-yl}-propionic acid,
3-{1-[5-(3-Fluoro-4-methoxy-phenyl)-thiophene-2-sulfonyl]-5-methoxy-1H-indol-3-}-propionic acid,
3-{5-Methoxy-1-[5-(6-methoxy-pyridin-3-yl)-thiophene-2-sulfonyl]-1H-indol-3-yl}-propionic acid,
3-{5-Methoxy-1-[5-(4-trifluoromethoxy-phenyl)-thiophene-2-sulfonyl]-1H-indol-3-yl}-propionic acid,
3-{1-[5-(4-Ethoxy-phenyl)-thiophene-2-sulfonyl]-5-methoxy-1H-indol-3-yl}-propionic acid,
3-{5-Methoxy-1-[5-(4-trifluoromethyl-phenyl)-thiophene-2-sulfonyl]-1H-indol-3-yl}-propionic acid,
3-[5-Ethoxy-1-(4′-propyl-biphenyl-2-sulfonyl)-1H-indol-3-yl]-propionic acid,
3-[1-(3′,4′-Dimethyl-biphenyl-2-sulfonyl)-5-ethoxy-1H-indol-3-yl]-propionic acid,
3-[5-Ethoxy-1-(5-methyl-3-p-tolyl-thiophene-2-sulfonyl)-1H-indol-3-yl]-propionic acid,
3-[1-(4′-Trifluoromethyl-biphenyl-3-sulfonyl)-1H-indol-3-yl]-propionic acid, and
3-[5-Methoxy-1-(4′-trifluoromethyl-biphenyl-3-sulfonyl)-1H-indol-3-yl]-propionic acid.

22. A method for treating a subject suffering from or at risk of a disease or condition for which PPAR modulation provides a therapeutic benefit, comprising
administering to said subject a therapeutically effective amount of a compound according to claim 1.

23. A method for treating a subject suffering from or at risk of a disease or condition for which PPAR modulation provides a therapeutic benefit, comprising
administering to said subject a therapeutically effective amount of a compound according to claim 11.

24. The method according to claim 22 or 23, wherein said compound is approved for administration to a human.

25. The method according to claim 22 or 23, wherein said disease or condition is a PPAR-mediated disease or condition.

26. The method according to claim 22 or 23, wherein said disease or condition is selected from the group consisting of obesity, overweight condition, bulimia, anorexia nervosa, hyperlipidemia, dyslipidemia, hypoalphalipoproteinemia, hypertriglyceridemia, hypercholesterolemia, low HDL, Metabolic Syndrome, Type II diabetes mellitus, Type I diabetes, hyperinsulinemia, impaired glucose tolerance, insulin resistance, a diabetic complication of neuropathy, nephropathy, retinopathy, diabetic foot ulcer or cataracts, hypertension, coronary heart disease, heart failure, congestive heart failure, atherosclerosis, arteriosclerosis, stroke, cerebrovascular disease, myocardial infarction, peripheral vascular disease, vitiligo, uveitis, pemphigus foliaceus, inclusion body myositis, polymyositis, dermatomyositis, scleroderma, Grave's disease, Hashimoto's disease, chronic graft versus host disease, rheumatoid arthritis, inflammatory bowel syndrome, Crohn's disease, systemic lupus erythematosis, Sjogren's Syndrome, multiple sclerosis, asthma, chronic obstructive pulmonary disease, polycystic kidney disease, polycystic ovary syndrome, pancreatitis, nephritis, hepatitis, eczema, psoriasis, dermatitis, impaired wound healing, Alzheimer's disease, Parkinson's disease, amyotrophic lateral sclerosis, spinal cord injury, acute disseminated encephalomyelitis, Guillain-Barre syndrome, thrombosis, infarction of the large or small intestine, renal insufficiency, erectile dysfunction, urinary incontinence, neurogenic bladder, ophthalmic inflammation, macular degeneration, pathologic neovascularization, HCV infection, HIV infection, Helicobacter pylori infection, neuropathic or inflammatory pain, infertility, and cancer.

27. A composition comprising:
a pharmaceutically acceptable carrier; and
a compound according to claim 1.

28. A composition comprising:
a pharmaceutically acceptable carrier; and
a compound according to claim 11.

29. A kit comprising a compound according to claim 1.

30. A kit comprising a compound according to claim 11.

31. A kit comprising a composition according to claim 27.

32. A kit comprising a composition according to claim 28.

33. A method for treating a subject suffering from or at risk of a disease or condition for which PPAR modulation provides a therapeutic benefit, comprising:
administering to said subject a therapeutically effective amount of a PPAR modulator
having the chemical structure of 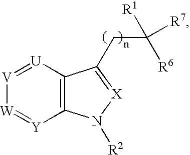
all salts, prodrugs, tautomers and isomers thereof,xxxxx
wherein:
U, V, W, X, and Y are independently N or CR⁸, wherein at most two of U, V, W, and Y are N;
R¹ is selected from the group consisting of C(O)OR¹⁶ and a carboxylic acid isostere;
R² is selected from the group consisting of hydrogen, optionally substituted lower alkyl —CH₂—CR¹²═CR¹³R¹⁴, —CH₂—C≡CR¹⁵, optionally substituted cycloalkyl, optionally substituted heterocycloalkyl, optionally substituted aryl, optionally substituted heteroaryl, —C(Z)NR¹⁰R¹¹, —C(Z)R²⁰, —S(O)₂NR¹⁰R¹¹ and —S(O)₂R²¹;
R⁶ and R⁷ are independently selected from the group consisting of hydrogen, optionally substituted lower alkyl, optionally substituted cycloalkyl, optionally substituted heterocycloalkyl, optionally substituted aryl, and optionally substituted heteroaryl; or
R⁶ and R⁷ combine to form a 3-7 membered monocyclic cycloalkyl or 5-7 membered monocyclic heterocycloalkyl;
R⁸ is selected from the group consisting of hydrogen, halogen, optionally substituted lower alkyl, —CH₂—CR¹²═CR¹³R¹⁴, —CH₂—≡CR¹⁵, optionally substituted cycloalkyl, optionally substituted heterocycloalkyl, optionally substituted aryl, optionally substituted heteroaryl, —OR⁹, —SR⁹, —NR¹⁰R¹¹, —C(Z)NR¹⁰R¹¹, —C(X)R²⁰, —S(O)₂NR¹⁰R¹¹, and —S(O)₂R²¹;
R⁹ is selected from the group consisting of optionally substituted lower alkyl, optionally substituted cycloalkyl, optionally substituted heterocycloalkyl, optionally substituted aryl, and optionally substituted heteroaryl;
R¹⁰ and R¹¹ are independently selected from the group consisting of hydrogen, optionally substituted lower alkyl, optionally substituted cycloalkyl, optionally substituted heterocycloalkyl, optionally substituted aryl, and optionally substituted heteroaryl; or
R¹⁰ and R¹¹ together with the nitrogen to which they are attached form a 5-7 membered monocyclic heterocycloalkyl or a 5 or 7 membered monocyclic nitrogen containing heteroaryl;
R¹⁶ is selected from the group consisting of hydrogen, optionally substituted lower alkyl, optionally substituted cycloalkyl, optionally substituted heterocycloalkyl, optionally substituted aryl, and optionally substituted heteroaryl;
R²⁰ is selected from the group consisting of —CH₂—CR¹²═CR¹³R¹⁴, —CH₂—C≡CR¹⁵, optionally substituted lower alkyl, optionally substituted cycloalkyl, optionally substituted heterocycloalkyl, optionally substituted aryl, and optionally substituted heteroaryl;
R²¹ is selected from the group consisting of —OR¹⁷, —CH₂—CR¹²═CR¹³R^{14=l , —CH}₂—C≡CR¹⁵, optionally substituted cycloalkyl, optionally substituted heterocycloalkyl, optionally substituted aryl, and optionally substituted heteroaryl;
R¹², R¹³, R¹⁴, and R¹⁵ are independently selected from the group consisting of optionally substituted lower alkyl, optionally substituted cycloalkyl, optionally substituted heterocycloalkyl, optionally substituted aryl, and optionally substituted heteroaryl;
R¹⁷ is selected from the group consisting of optionally substituted alkyl, optionally substituted cycloalkyl, optionally substituted heterocycloalkyl, optionally substituted aryl, optionally substituted heteroaryl, and —C(O)R¹⁸;
R¹⁸ is selected from the group consisting of hydrogen, optionally substituted lower alkyl, optionally substituted cycloalkyl, optionally substituted heterocycloalkyl, optionally substituted aryl, and optionally substituted heteroaryl;
Z is O or S; and
n=0, 1, or 2;
wherein said disease or condition is selected from the group consisting of vitiligo, uveitis, pemphigus foliaceus, inclusion body myositis, polymyositis, dermatomyositis, scleroderma, Grave's disease, Hashimoto's disease, chronic graft versus host disease, rheumatoid arthritis, inflammatory bowel syndrome, Crohn's disease, systemic lupus erythematosis, Sjogren's Syndrome, multiple sclerosis, asthma, chronic obstructive pulmonary disease, polycystic kidney disease, polycystic ovary syndrome, pancreatitis, nephritis, and hepatitis), dermatitis, impaired wound healing, Alzheimer's disease, Parkinson's disease, amyotrophic lateral sclerosis, spinal cord injury, acute disseminated encephalomyelitis, Guillain-Barre syndrome, infarction of the large or small intestine, renal insufficiency, erectile dysfunction, urinary incontinence, neurogenic bladder, ophthalmic inflammation, macular degeneration, pathologic neovascularization, HCV infection, HIV infection, Helicobacter pylori infection, neuropathic pain, inflammatory pain, and infertility.

34. The method according to claim 33, wherein said PPAR modulator has the chemical structure of 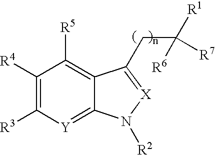
wherein:
U is CR⁸, wherein R⁸ is R⁵;
V is CR⁸, wherein R⁸ is R⁴;
W is CR⁸, wherein R⁸ is R³;
R³, R⁴, and R⁵ are independently selected from the group consisting of hydrogen, halogen, optionally substituted lower alkyl, —CH₂—CR¹²═CR¹³R¹⁴, —CH₂—C≡CR¹⁵, optionally substituted cycloalkyl, optionally substituted heterocycloalkyl, optionally substituted aryl, optionally substituted heteroaryl, —OR⁹, —SR⁹, —NR¹⁰R¹¹, —C(Z)NR¹⁰R¹¹, —C(Z)R²⁰, —S(O)₂NR¹⁰R¹¹, and —S(O)₂R²¹.

35. The method according to claim 33, wherein said PPAR modulator has the chemical structure of 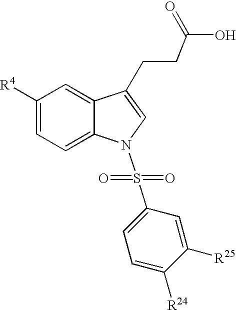
wherein:
U is CR⁸, wherein R⁸ is H;
V is CR⁸, wherein R⁸ is R⁴;
W is CR⁸, wherein R⁸ is H;
X is CR⁸, wherein R⁸ is H;
Y is CR⁸, wherein R⁸ is H;
n is 1;
R¹ is —COOH;
R⁶ and R⁷ are hydrogen;
R² is —S(O)₂R²¹, wherein R²¹ is 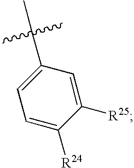
R⁴ is selected from the group consisting of hydrogen, halogen, optionally substituted lower alkyl, optionally substituted cycloalkyl, optionally substituted heterocycloalkyl, optionally substituted aryl, optionally substituted heteroaryl, —OR⁹, —SR⁹, —NR¹⁰R¹¹, —C(Z)NR¹⁰R¹¹, —C(Z)R²⁰, —S(O)₂NR¹⁰R¹¹, and —S(O)₂R²¹;
R²⁴ is selected from the group consisting of hydrogen, halogen, optionally substituted lower alkyl, —OR¹⁹, and —O(CH₂)ₚO-aryl;
p is 1, 2, 3,or 4;
R²⁵ is selected from the group consisting of hydrogen, halogen, optionally substituted lower alkyl, and —OR¹⁹; or
R²⁴ and R²⁵ combine to form cycloalkyl, heterocycloalkyl, aryl or heteroaryl fused with the phenyl ring; and
R¹⁹ is selected from the group consisting of optionally substituted lower alkyl and optionally substituted aryl.

36. The method according to claim 33, 34, or 35, wherein the disease or condition is selected from the group consisting of Alzheimer's disease, Parkinson's disease, amyotrophic lateral sclerosis, rheumatoid arthritis, inflammatory bowel syndrome, Crohn's disease, multiple sclerosis, infertility, asthma, chronic obstructive pulmonary disease, and macular degeneration.

37. The method according to claim 22 or 23, wherein said disease or condition is selected from the group consisting of Alzheimer's disease, Parkinson's disease, amyotrophic lateral sclerosis, rheumatoid arthritis, inflammatory bowel syndrome, Crohn's disease, multiple sclerosis, infertility, asthma, chronic obstructive pulmonary disease, and macular degeneration.


SEQUENCE LISTING (19 sequences)

SEQ ID NO: 1 - DNA, 10049 bp, Homo sapiens
gcgccgcctccttcggcgttcgccccacggaccggcaggcggcggaccgcggcccaggctgaagctcagggccctgtctgctctgtggactcaacagtttgtggcaagacaagctcagaactgagaagctgtcaccacagttctggaggctgggaagttcaagatcaaagtgccagcagattcagtgtcatgtgaggacgtgcttcctgcttcatagataagagtagcttggagctcggcggcacaaccagcaccatctggtcgcgatggtggacacggaaagcccactctgccccctctccccactcgaggccggcgatctagagagcccgttatctgaagagttcctgcaagaaatgggaaacatccaagagatttcgcaatccatcggcgaggatagttctggaagctttggctttacggaataccagtatttaggaagctgtcctggctcagatggctcggtcatcacggacacgctttcaccagcttcgagcccctcctcggtgacttatcctgtggtccccggcagcgtggacgagtctcccagtggagcattgaacatcgaatgtagaatctgcggggacaaggcctcaggctatcattacggagtccacgcgtgtgaaggctgcaagggcttctttcggcgaacgattcgactcaagctggtgtatgacaagtgcgaccgcagctgcaagatccagaaaaagaacagaaacaaatgccagtattgtcgatttcacaagtgcctttctgtcgggatgtcacacaacgcgattcgttttggacgaatgccaagatctgagaaagcaaaactgaaagcagaaattcttacctgtgaacatgacatagaagattctgaaactgcagatctcaaatctctggccaagagaatctacgaggcctacttgaagaacttcaacatgaacaaggtcaaagcccgggtcatcctctcaggaaaggccagtaacaatccaccttttgtcatacatgatatggagacactgtgtatggctgagaagacgctggtggccaagctggtggccaatggcatccagaacaaggaggcggaggtccgcatctttcactgctgccagtgcacgtcagtggagaccgtcacggagctcacggaattcgccaaggccatcccaggcttcgcaaacttggacctgaacgatcaagtgacattgctaaaatacggagtttatgaggccatattcgccatgctgtcttctgtgatgaacaaagacgggatgctggtagcgtatggaaatgggtttataactcgtgaattcctaaaaagcctaaggaaaccgttctgtgatatcatggaacccaagtttgattttgccatgaagttcaatgcactggaactggatgacagtgatatctccctttttgtggctgctatcatttgctgtggagatcgtcctggccttctaaacgtaggacacattgaaaaaatgcaggagggtattgtacatgtgctcagactccacctgcagagcaaccacccggacgatatctttctcttcccaaaacttcttcaaaaaatggcagacctccggcagctggtgacggagcatgcgcagctggtgcagatcatcaagaagacggagtcggatgctgcgctgcacccgctactgcaggagatctacagggacatgtactgagttccttcagatcagccacaccttttccaggagttctgaagctgacagcactacaaaggagacgggggagcagcacgattttgcacaaatatccaccactttaaccttagagcttggacagtctgagctgtaggtaaccggcatattattccatatctttgttttaaccagtacttctaagagcatagaactcaaatgctgggggtaggtggctaatctcaggactgggaagattacggcgaattatgctcaatggtctgattttaactcacccgatgttaatcaatgcacattgctttagatcacattcgtgatttaccatttaattaactggtaacctcaaaattcgtggcctgtcttcccattcaccccgcttttgactattgtgctcctttataattctgaaaactaatcagcactttttaacaatgtttataatcctataagtctagatgtatccaaaggtgaagtatgtaaaaagcagcaaaatatttatttcaaagacttcacttctgtttcctgaatctaaagaaagacaacatgctgctttttaatcataggatggagaattttaaagaactgtttgggccaggcacagtcgctcatacttgtaatcccagcactttgggaggccgaggcgggtggatcacaaggtcagcagatcgagaccatcctggccaacatggtgaaaccctgtctctactaaaaatacaaaaattagccgggtgtggtggcacatgcctgtaatcccagctactcgggaagctgaggcaggagaattgcttgaaccagggagttggaggttgcagtgagctaagactgcaccactgcactccagcctggtgacagaacgagactctgtcttaaaaacaaacaaacaaaaaaaaaatctgttagataagctatcaaaatgcagctgttgttttgtttttggctcactgttttcgtggttgtaactaatatgtggaaaggcccatttccaggtttgcgtagaagagcccagaaaacagagtctcaagacccccgctctggactgtcataagctagcacccgtggtaagcgggacgagacaagctcccgaagcccgccagcttcctgctccactcagctccgtccagtcaacctgaacccacccagtccagctgtctgtgggaatggtggtgttcttagggacagactgacaccttacttgtcagtgttcctccgggccccatttggcagctcccgtatcttttgttatgttgcttttaaagatatgatgttttattgttttaactcttggtgacagtagatgctctctggagcgcagacgaggcacatgtgtcttcatagcctgggctgggtgggagccagtcaccctgcggatcgagagagggggtagagtcttcttcaaatggcagttttacttcaaatggcagatttcacaagagttggttattttttacaatggtttaggttgttaagtctcctttgtatgtaaggtagttttttcaacatctaaaatttttgttttagccttcaaaaccaacttaccaacctcagtccagctgggaaggcagcgttgattatggtagtttgtcaagaatatatggacctggaaacactttctctctctgtccacctggtagataaattgtcctgttgagaatttttagatctggactggaactgccaggaccaccgcctccagggagtcgctgggcacctggaggtatcgtcgatgcctctcccccatctttagaaaatttggctcttctgaggtcattattattttaagaatgattaggattgataagggtcccatgaccagcattatgaaaatgcgagagtgggaaggacacagtgtgagacttccactagaaaaaagtgaaagttagggttaggacatccttttttaaaaattacaaatttagtccgttttggtttttgtaatcaggctaggcacagtggctcacacatggaatcccagcactttgggaggccgaggtgggaggatcacttgagcccaggagttcgagaccagcctaggcaacatagcaagaccctgtctgtacacaaaatttaaaaattagttcatcggggtggcacacatcagtagtcccagctactctgcaggctgaggtgggaggattgcttgaacccaggaggtcgaggctgcagtgagctgtgatctcaccactgcattccagcctgggtgacagagttagattccaccctctcccaccccggcaaaaaaaaaaaaaaaagatgcaatcaaaggggctgttggccagcaatggcagcagcagcggcgggcagtctgcccaagtgtcttaggaaccaaaagcaaataaaagtgtttccatatatgccaccagccaagtggccatcctaattcagaaagaagctagcctttgagtgtctgtcatggtgcatccgtttcagtattatttcctaaaatgagaagcccctgtgtcaacaagatccaggggctggagcccaatgccaagcctgtgttgtccccagcgaccctgcagctgctcgctctgatgtaccctgtgccattcaaggagatgtggtccaggaaagtgagcctcatggttttcagagaagtcattgttctgtttacattttcataaaacctgtttaaaatagctccccgtctcaggctttcagcagtaacagtgagctgactggcaagttcgatgttagctcccgggacactcagcagcgatggtgagcattttggtttccttaaggcccagcaagacttccagggacatctctggtgaagccagaatggagacacccgtgacctcaggctgaaagtcactcgacattggtctcttgtgttgatagggaaggaaatcaggcattcctatttctttaaataacaaaaccactaattgccactcaatgctggaatattttgggtcacctaatcatagatttctcagggcatcaatactcaaatataggctgattatgccccagttcaaatgggaactattaacagagtgcatttcttgcttgctgggtttcaacagacatcagccaaaagaacaaaagagatgtcaggacagattccaggagtgtcggagcacatgtgtggcacccgctccctctggcagcgaatgtaggaagtcgccaaatttacccactcttcaacaagtcattgtttaaacacggtttttcattttctcaacttttaatagcaaaaagtgccaaagtcctcagagacctaacagccttggtctaccgtgctgaccagggtgaaggcacggcgagggactcctcccagacgtgcctcttgtgtgccagctggctgtggctcgggagcagacgcaggcctctccattgtccaggggagcctggcggcgcatccctcctctcccacctcctggcacttccagctgggtgtcccacatgttggattccgtccccaccacacttccagagaccggagaactgtgcagggcctaaggccgtttggatgaattgtcaaaacaagatgcttccagttacagcggcaggagcgggactgggagcacgggctgacggctgctggtgcctttcttcccacctcgcttgcctgtttccgcttgacccttcctccagctccgatgagaagagtataaagcatcttcctaacgggtgtgtttgctatacgaacataatggacgtgaagtggggcagaaacccagaactcagcattcaaggatgcccaggagagctgtccctgttttaaagagctgtgttttgttttgtttcgcatttagagagcagacaaggcacccttctgctgcgctgatacgtttcttacactgggccattttagacccccagggaaacagccttcctggagcgttgtctggaggttccagggacagggcagcctcccagagccgagcaagagctcaaggtacaaatgagagatttgctataccgtgagaagtcaacaacttagccaccacttccccgcaatggaccatgtaacaaatacctcagcaggccctgcaaaaggccatgctagagctgaggcgcacagcctgtggcctctgtagttagggcaggtgggatggagactccttgagtgcacacacctgagcctgcccacacacaggggagcagcatctcgtatgacgtctggaaggaacttcggttgtgtaaagggagccttgaagatacgtgcaaaaggtgctaccccaatttggtgaaactgacattgggcacgtcttgggcttaggagaagcggccgatggtcccggcctgcagtgacaaacccccctccccgcaccgcccccagcaccccctctcctcttcacctcttcctgctggccacgaggaagccacttcctcagagagaccctaccagatgcggatggaaacagatgcaccaaagcaagccctgatgaaaccgcgacttcctaaggtctgtctcctctgaacttgcacctgggcctctctgtgtttggttccaagcacttcccacctcaaactcccattttcaaaccactgtatctctgcgcacatctgctacttaccagccgcatacatgatggagggttttttggtcctgatccagtggccacacctgtctttgaaatgtctcactgaactccagttttaaaatagattcattgcttcaacacagcaagcccaatgcacccagctaagactggcttgaccgacagcctggcctttggtggggggcttcctggggcctggggaaagctggccaccttcaacagctggtacctcttcaacagtgtggcctttcaaaatgcagatgccaccaggagaacatgcccacagctcaccacctatggatgccatggctctgggcagctttcaaagcaggttcctgtggtctcctcagctgtttgagggggtaacagcaaatcagcctccattttaaaatgaaaacaccagcctccagatgtagggcctgctgggtgttgctagccgctggtccccaggcacggtgcactttctccacctcctgcagcctccctgttgtttctagactcttgcacctggtgagtgcaaggataggtgacccaggggcctgcagccttgtcctcagctcccatctcctggactgccagcctcaccctctgcagttagcatggttggcctgatgcagggatcccgagggattactttttagaccttctttcacattcagaaaagtagtatagattcaggagaggcaagaaaattatgctgtccatagaagtcacccatgaagactgatgccaccacctgaaggctcatgattgttaaaaatgtccacgggaacctctcgtccacaggaggtttgtctcaacacttcccatttttacggcattggcattgccaagcatggggaagtatctgctcttctcatgttaaaagtggcccagcttttcttaactcagtccaagctgacttgtttagctgcactggaatttcttaccaaccaaatatttgcatcgagcaaagggggctgtgtgcacctccctaatggcagcgatgatggctgctgtcattcaagcccatcttcagacgtcacagtctggaagtgaaatgtccacaaacatctgtggcagaaaaggctatacggaccacccagttgtgctgcagctttacagagcaaggaagggttgtggcaaataaatgattaacctgcctcgactgtgctgagggcaacaaaggccatctcaccaaaggattattcgatgccattaaatcatcccgtgaccttcctgcttccgagtccatggcctttgcccagggcatgtactcccctgagaggccttctgcctagaaagatctatgactgggttccaaagttgaggcctaggtttttgctgggatttagatattttcaggcaccattttgacagcattcaggaaaacggttattgaccccatagactagggtaagaataaaggcaataaatttggtctgactcagaatataggagatccatatatttctctggaaaccacagtgtacactaaaatgtgaaattgaaggttttgttaaaaagaaaaagataatgagcttcatgctttgtttaattacataatgatttccattacgctatttctgtgaaatgcagcaggttcttaaacgttatttcagtggcatgggctggaagcttatcacaaaaagccatgtgtgtggccttatcagaacagaaagagacaggctggtgcccaaggctgctgcctgctccaccttttgccagctctggacatctgaggacgtcccggcagatctggaatggggccctcaactgaccatttgcttctcagaatttcagtttgagacatgagaggtataatcagttacttttctccccccagagaaacccttttgtgaggggagaggagctatggtatgtggttcagctgaaacacatacaactgcatccttttggagtcctttgccaacaaaaacagaccaacagaccagatggtgtccatgttcaatatcatgtcttgatggacgcagctgatgacctcaaatacttgagtggtctcatggctgttagatggattatttgaaaaaaaaaaaaaaaaaagagagaaaaaataattgatttttacatcagagatagcaaactaagacctggggaggggggtcagcttttattttattttattttttttaagtttgctagttgggtcaaatgtgaggaggagggagtctacctgccacctcttctcttgcccctcttctgcccacacatccagcatccaaaatccattcatttaatgaattgataaagtgccgtgcaaactggtgcacaaacaggcccccagtccacgcagcctggctcctaggaaaagtggtgaccgggcgtgggggggcatgccgcagccctgggacacagtcgggcaccttccccggacccccaggccttggctgtgcctcaagtcagagagggtcagccttcaggccccggagacgagtgactggccgatcatttcacaataaaatcactcacttttggcaacttcactttttttaaggcacagtcagttccttttctcatgtacctcacaaaagatgaagaccatgtagtactctttttggtaaagttacagtgttcatgttaaatatcacttttttctacattgtgtggtaaaaagaactacgttaatagctatatcttaaatactgtgatttgactttttgaaaaatatcctaatacaaatattttactaacttacaatcactcatttaataagaaacatttggattcttttgaaatcagtgttaattgactcatattcttaaaagcctggctcttgaccctattggaaacacaaaggaagctgaaatcaaacatctaaaatacactgcgtacacgtgtgcgtgcacacacacacacacacacacacacacacagctcttcatttctcctgagccatgcagaatttactttcaatgtggaaatctgttccctttaccacactgtatatgcacagagcacaagagaggctatctctagtcacttccaccagcgaggccttagactccgtattagaggccaccgatttcatacaacagtgtttcgctaaagacccttcactattcttgtttagtaaatagctgtctgctcttcagggaactgttacctatgggttattaccaaagaacgctggcaattggaaatgtcctgatggaaattctttgcacgtgccggttctctggcatcctccaggtggcccaacccaaagcagaaagcagaaaccacagaccccgtgagtctccccataccttgtttccaataacttggcaaaacttcttggtgcatattggttacaccctctgggattcataatgccattaggctaaaaccctaagagagagggttgacagaaacacacgcgagaatgaggcagatcccagagcaaggactgggcccagactctccacatgtgctctactagtgagtgccttatactctcagtattttggggcttacagcttcttatttgtgctaaaaaggtgcagttccaaagtaggaactgccacacaggccccagcatcctctctccaacttcatacctctctcctggtggggggagcgggcatccaggacctccggaatcaaggatgtgcagagaagagcgaaagtaatttttctagtcacatgaactgattggttccaggcaattagaaaatggctataaaataaccttaattttaaaaaaaaatcttgggtcttcgttttcctattaggagactgaactgaccacatgtattgatttatatcctgaatatatgggaacttctgtgtttgggatgtcctactgtaagactgatgaatgtacagagttaatttcagggtacagttttgccttaatggttttaaaaaataaactattttttaaaatttt

SEQ ID NO: 2 - protein, 468 aa, Homo sapiens
MVDTESPLCPLSPLEAGDLESPLSEEFLQEMGNIQEISQSIGEDSSGSFGFTEYQYLGSCPGSDGSVITDTLSPASSPSSVTYPVVPGSVDESPSGALNIECRICGDKASGYHYGVHACEGCKGFFRRTIRLKLVYDKCDRSCKIQKKNRNKCQYCRFHKCLSVGMSHNAIRFGRMPRSEKAKLKAEILTCEHDIEDSETADLKSLAKRIYEAYLKNFNMNKVKARVILSGKASNNPPFVIHDMETLCMAEKTLVAKLVANGIQNKEAEVRIFHCCQCTSVETVTELTEFAKAIPGFANLDLNDQVTLLKYGVYEAIFAMLSSVMNKDGMLVAYGNGFITREFLKSLRKPFCDIMEPKFDFAMKFNALELDDSDISLFVAAIICCGDRPGLLNVGHIEKMQEGIVHVLRLHLQSNHPDDIFLFPKLLQKMADLRQLVTEHAQLVQIIKKTESDAALHPLLQEIYRDMY

SEQ ID NO: 3 - DNA, 1863 bp, Homo sapiens
actgatgtcttgactcatgggtgtattcacaaattctgttacttcaagtctttttcttttaacggattgatcttttgctagatagagacaaaatatcagtgtgaattacagcaaacccctattccatgctgttatgggtgaaactctgggagattctcctattgacccagaaagcgattccttcactgatacactgtctgcaaacatatcacaagaaatgaccatggttgacacagagatgccattctggcccaccaactttgggatcagctccgtggatctctccgtaatggaagaccactcccactcctttgatatcaagcccttcactactgttgacttctccagcatttctactccacattacgaagacattccattcacaagaacagatccagtggttgcagattacaagtatgacctgaaacttcaagagtaccaaagtgcaatcaaagtggagcctgcatctccaccttattattctgagaagactcagctctacaataagcctcatgaagagccttccaactccctcatggcaattgaatgtcgtgtctgtggagataaagcttctggatttcactatggagttcatgcttgtgaaggatgcaagggtttcttccggagaacaatcagattgaagcttatctatgacagatgtgatcttaactgtcggatccacaaaaaaagtagaaataaatgtcagtactgtcggtttcagaaatgccttgcagtggggatgtctcataatgccatcaggtttgggcggatgccacaggccgagaaggagaagctgttggcggagatctccagtgatatcgaccagctgaatccagagtccgctgacctccgggccctggcaaaacatttgtatgactcatacataaagtccttcccgctgaccaaagcaaaggcgagggcgatcttgacaggaaagacaacagacaaatcaccattcgttatctatgacatgaattccttaatgatgggagaagataaaatcaagttcaaacacatcacccccctgcaggagcagagcaaagaggtggccatccgcatctttcagggctgccagtttcgctccgtggaggctgtgcaggagatcacagagtatgccaaaagcattcctggttttgtaaatcttgacttgaacgaccaagtaactctcctcaaatatggagtccacgagatcatttacacaatgctggcctccttgatgaataaagatggggttctcatatccgagggccaaggcttcatgacaagggagtttctaaagagcctgcgaaagccttttggtgactttatggagcccaagtttgagtttgctgtgaagttcaatgcactggaattagatgacagcgacttggcaatatttattgctgtcattattctcagtggagaccgcccaggtttgctgaatgtgaagcccattgaagacattcaagacaacctgctacaagccctggagctccagctgaagctgaaccaccctgagtcctcacagctgtttgccaagctgctccagaaaatgacagacctcagacagattgtcacggaacacgtgcagctactgcaggtgatcaagaagacggagacagacatgagtcttcacccgctcctgcaggagatctacaaggacttgtactagcagagagtcctgagccactgccaacatttcccttcttccagttgcactattctgagggaaaatctgacacctaagaaatttactgtgaaaaagcattttaaaaagaaaaggttttagaatatgatctattttatgcatattgtttataaagacacatttacaatttacttttaatattaaaaattaccatattatgaaaaaaaaaaaaaaaa

SEQ ID NO: 4 - protein, 505 aa, Homo sapiens
MGETLGDSPIDPESDSFTDTLSANISQEMTMVDTEMPFWPTNFGISSVDLSVMEDHSHSFDIKPFTTVDFSSISTPHYEDIPFTRTDPVVADYKYDLKLQEYQSAIKVEPASPPYYSEKTQLYNKPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 5 - DNA, 3328 bp, Homo sapiens
gttttggcaggagcgggagaattctgcggagcctgcgggacggcggcggtggcgccgtaggcagccgggacagtgttgtacagtgttttgggcatgcacgtgatactcacacagtggcttctgctcaccaacagatgaagacagatgcaccaacgagggtctggaatggtctggagtggtctggaaagcagggtcagatacccctggaaaactgaagcccgtggagcagtgatctctacaggactgcttcaaggctgatgggaaccaccctgtagaggtccatctgcgttcagacccagacgatgccagagctatgactgggcctgcaggtgtggcgccgaggggagatcagccatggagcagccacaggaggaagcccctgaggtccgggaagaggaggagaaagaggaagtggcagaggcagaaggagccccagagctcaatgggggaccacagcatgcacttccttccagcagctacacagacctctcccggagctcctcgccaccctcactgctggaccaactgcagatgggctgtgacggggcctcatgcggcagcctcaacatggagtgccgggtgtgcggggacaaggcatcgggcttccactacggtgttcatgcatgtgaggggtgcaagggcttcttccgtcgtacgatccgcatgaagctggagtacgagaagtgtgagcgcagctgcaagattcagaagaagaaccgcaacaagtgccagtactgccgcttccagaagtgcctggcactgggcatgtcacacaacgctatccgttttggtcggatgccggaggctgagaagaggaagctggtggcagggctgactgcaaacgaggggagccagtacaacccacaggtggccgacctgaaggccttctccaagcacatctacaatgcctacctgaaaaacttcaacatgaccaaaaagaaggcccgcagcatcctcaccggcaaagccagccacacggcgccctttgtgatccacgacatcgagacattgtggcaggcagagaaggggctggtgtggaagcagttggtgaatggcctgcctccctacaaggagatcagcgtgcacgtcttctaccgctgccagtgcaccacagtggagaccgtgcgggagctcactgagttcgccaagagcatccccagcttcagcagcctcttcctcaacgaccaggttacccttctcaagtatggcgtgcacgaggccatcttcgccatgctggcctctatcgtcaacaaggacgggctgctggtagccaacggcagtggctttgtcacccgtgagttcctgcgcagcctccgcaaacccttcagtgatatcattgagcctaagtttgaatttgctgtcaagttcaacgccctggaacttgatgacagtgacctggccctattcattgcggccatcattctgtgtggagaccggccaggcctcatgaacgttccacgggtggaggctatccaggacaccatcctgcgtgccctcgaattccacctgcaggccaaccaccctgatgcccagtacctcttccccaagctgctgcagaagatggctgacctgcggcaactggtcaccgagcacgcccagatgatgcagcggatcaagaagaccgaaaccgagacctcgctgcaccctctgctccaggagatctacaaggacatgtactaacggcggcacccaggcctccctgcagactccaatggggccagcactggaggggcccacccacatgacttttccattgaccagctctcttcctgtctttgttgtctccctctttctcagttcctctttcttttctaattcctgttgctctgtttcttcctttctgtaggtttctctcttcccttctcccttgccctccctttctctctccaccccccacgtctgtcctcctttcttattctgtgagatgttttgtattatttcaccagcagcatagaacaggacctctgcttttgcacaccttttccccaggagcagaagagagtggggcctgccctctgccccatcattgcacctgcaggcttaggtcctcacttctgtctcctgtcttcagagcaaaagacttgagccatccaaagaaacactaagctctctgggcctgggttccagggaaggctaagcatggcctggactgactgcagccccctatagtcatggggtccctgctgcaaaggacagtgggcaggaggccccaggctgagagccagatgcctccccaagactgtcattgcccctccgatgctgaggccacccactgacccaactgatcctgctccagcagcacacctcagccccactgacacccagtgtccttccatcttcacactggtttgccaggccaatgttgctgatggcccctccagcacacacacataagcactgaaatcactttacctgcaggctccatgcacctcccttccctccctgaggcaggtgagaacccagagagaggggcctgcaggtgagcaggcagggctgggccaggtctccggggaggcaggggtcctgcaggtcctggtgggtcagcccagcacctgctcccagtgggagcttcccgggataaactgagcctgttcattctgatgtccatttgtcccaatagctctactgccctccccttcccctttactcagcccagctggccacctagaagtctccctgcacagcctctagtgtccggggaccttgtgggaccagtcccacaccgctggtccctgccctcccctgctcccaggttgaggtgcgctcacctcagagcagggccaaagcacagctgggcatgccatgtctgagcggcgcagagccctccaggcctgcaggggcaaggggctggctggagtctcagagcacagaggtaggagaactggggttcaagcccaggcttcctgggtcctgcctggtcctccctcccaaggagccattctgtgtgtgactctgggtggaagtgcccagcccctgcccctacgggcgctgcagcctcccttccatgccccaggatcactctctgctggcaggattcttcccgctccccacctacccagctgatgggggttggggtgcttcctttcaggccaaggctatgaagggacagctgctgggacccacctccccctccccggccacatgccgcgtccctgccccgacccgggtctggtgctgaggatacagctcttctcagtgtctgaacaatctccaaaattgaaatgtatatttttgctaggagccccagcttcctgtgtttttaatataaatagtgtacacagactgacgaaactttaaataaatgggaattaaatatttaaaaaaaaaa

SEQ ID NO: 6 - protein, 441 aa, Homo sapiens
MEQPQEEAPEVREEEEKEEVAEAEGAPELNGGPQHALPSSSYTDLSRSSSPPSLLDQLQMGCDGASCGSLNMECRVCGDKASGFHYGVHACEGCKGFFRRTIRMKLEYEKCERSCKIQKKNRNKCQYCRFQKCLALGMSHNAIRFGRMPEAEKRKLVAGLTANEGSQYNPQVADLKAFSKHIYNAYLKNFNMTKKKARSILTGKASHTAPFVIHDIETLWQAEKGLVWKQLVNGLPPYKEISVHVFYRCQCTTVETVRELTEFAKSIPSFSSLFLNDQVTLLKYGVHEAIFAMLASIVNKDGLLVANGSGFVTREFLRSLRKPFSDIIEPKFEFAVKFNALELDDSDLALFIAAIILCGDRPGLMNVPRVEAIQDTILRALEFHLQANHPDAQYLFPKLLQKMADLRQLVTEHAQMMQRIKKTETETSLHPLLQEIYKDMY

SEQ ID NO: 7 - DNA, 1014 bp, Artificial Sequence
  Description of Artificial Sequence Synthetic construct
  CDS (88)..(960)
taatacgactcactataggggaattgtgagcggataacaattcccctctagaaataattttgtttaactttaagaaggagatataccatgggcagcagccatcatcatcatcatcacagcagcggcctggtgccgcgcggcagccatatggaaactgcagatctcaaatctctggccaagagaatctacgaggcctacttgaagaacttcaacatgaacaaggtcaaagcccgggtcatcctctcaggaaaggccagtaacaatccaccttttgtcatacatgatatggagacactgtgtatggctgagaagacgctggtggccaagctggtggccaatggcatccagaacaaggaggcggaggtccgcatctttcactgctgccagtgcacgtcagtggagaccgtcacggagctcacggaattcgccaaggccatcccaggcttcgcaaacttggacctgaacgatcaagtgacattgctaaaatacggagtttatgaggccatattcgccatgctgtcttctgtgatgaacaaagacgggatgctggtagcgtatggaaatgggtttataactcgtgaattcctaaaaagcctaaggaaaccgttctgtgatatcatggaacccaagtttgattttgccatgaagttcaatgcactggaactggatgacagtgatatctccctttttgtggctgctatcatttgctgtggagatcgtcctggccttctaaacgtaggacacattgaaaaaatgcaggagggtattgtacatgtgctcagactccacctgcagagcaaccacccggacgatatctttctcttcccaaaacttcttcaaaaaatggcagacctccggcagctggtgacggagcatgcgcagctggtgcagatcatcaagaagacggagtcggatgctgcgctgcacccgctactgcaggagatctacagggacatgtactgagtcgacaagcttgcggccgcactcgagcaccaccaccaccaccactgagat

SEQ ID NO: 8 - protein, 291 aa, Artificial Sequence
  Description of Artificial Sequence Synthetic construct
MGSSHHHHHHSSGLVPRGSHMETADLKSLAKRIYEAYLKNFNMNKVKARVILSGKASNNPPFVIHDMETLCMAEKTLVAKLVANGIQNKEAEVRIFHCCQCTSVETVTELTEFAKAIPGFANLDLNDQVTLLKYGVYEAIFAMLSSVMNKDGMLVAYGNGFITREFLKSLRKPFCDIMEPKFDFAMKFNALELDDSDISLFVAAIICCGDRPGLLNVGHIEKMQEGIVHVLRLHLQSNHPDDIFLFPKLLQKMADLRQLVTEHAQLVQIIKKTESDAALHPLLQEIYRDMY

SEQ ID NO: 9 - DNA, 33 bp, Artificial Sequence
  Description of Artificial Sequence Synthetic primer
gctgacacatatggaaactgcagatctcaaatc

SEQ ID NO: 10 - DNA, 32 bp, Artificial Sequence
  Description of Artificial Sequence Synthetic primer
gtgactgtcgactcagtacatgtccctgtaga

SEQ ID NO: 11 - DNA, 1017 bp, Artificial Sequence
  Description of Artificial Sequence Synthetic constuct
  CDS (88)..(963)
taatacgactcactataggggaattgtgagcggataacaattcccctctagaaataattttgtttaactttaagaaggagatataccatgggcagcagccatcatcatcatcatcacagcagcggcctggtgccgcgcggcagccatatggagtccgctgacctccgggccctggcaaaacatttgtatgactcatacataaagtccttcccgctgaccaaagcaaaggcgagggcgatcttgacaggaaagacaacagacaaatcaccattcgttatctatgacatgaattccttaatgatgggagaagataaaatcaagttcaaacacatcacccccctgcaggagcagagcaaagaggtggccatccgcatctttcagggctgccagtttcgctccgtggaggctgtgcaggagatcacagagtatgccaaaagcattcctggttttgtaaatcttgacttgaacgaccaagtaactctcctcaaatatggagtccacgagatcatttacacaatgctggcctccttgatgaataaagatggggttctcatatccgagggccaaggcttcatgacaagggagtttctaaagagcctgcgaaagccttttggtgactttatggagcccaagtttgagtttgctgtgaagttcaatgcactggaattagatgacagcgacttggcaatatttattgctgtcattattctcagtggagaccgcccaggtttgctgaatgtgaagcccattgaagacattcaagacaacctgctacaagccctggagctccagctgaagctgaaccaccctgagtcctcacagctgtttgccaagctgctccagaaaatgacagacctcagacagattgtcacggaacatgtgcagctactgcaggtgatcaagaagacggagacagacatgagtcttcacccgctcctgcaggagatctacaaggacttgtactaggtcgacaagcttgcggccgcactcgagcaccaccaccaccaccactgagat

SEQ ID NO: 12 - protein, 292 aa, Artificial Sequence
  Description of Artificial Sequence Synthetic constuct
MGSSHHHHHHSSGLVPRGSHMESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 13 - DNA, 33 bp, Artificial Sequence
  Description of Artificial Sequence Synthetic primer
gctcagacatatggagtccgctgacctccgggc

SEQ ID NO: 14 - DNA, 32 bp, Artificial Sequence
  Description of Artificial Sequence Synthetic primer
gtgactgtcgacctagtacaagtccttgtaga

SEQ ID NO: 15 - DNA, 1089 bp, Artificial Sequence
  Description of Artificial Sequence Synthetic construct
  CDS (88)..(948)
taatacgactcactataggggaattgtgagcggataacaattcccctctagaaataattttgtttaactttaagaaggagatataccatgaaaaaaggtcaccaccatcaccatcacggatcccagtacaacccacaggtggccgacctgaaggccttctccaagcacatctacaatgcctacctgaaaaacttcaacatgaccaaaaagaaggcccgcagcatcctcaccggcaaagccagccacacggcgccctttgtgatccacgacatcgagacattgtggcaggcagagaaggggctggtgtggaagcagttggtgaatggcctgcctccctacaaggagatcagcgtgcacgtcttctaccgctgccagtgcaccacagtggagaccgtgcgggagctcactgagttcgccaagagcatccccagcttcagcagcctcttcctcaacgaccaggttacccttctcaagtatggcgtgcacgaggccatcttcgccatgctggcctctatcgtcaacaaggacgggctgctggtagccaacggcagtggctttgtcacccgtgagttcctgcgcagcctccgcaaacccttcagtgatatcattgagcctaagtttgaatttgctgtcaagttcaacgccctggaacttgatgacagtgacctggccctattcattgcggccatcattctgtgtggagaccggccaggcctcatgaacgttccacgggtggaggctatccaggacaccatcctgcgtgccctcgaattccacctgcaggccaaccaccctgatgcccagtacctcttccccaagctgctgcagaagatggctgacctgcggcaactggtcaccgagcacgcccagatgatgcagcggatcaagaagaccgaaaccgagacctcgctgcaccctctgctccaggagatctacaaggacatgtactaagtcgaccaccaccaccaccaccactgagatccggctggccctactggccgaaaggaattcgaggccagcagggccaccgctgagcaataactagcataaccccttggggcctctaaacgggtcttgaggggttttttg

SEQ ID NO: 16 - protein, 287 aa, Artificial Sequence
  Description of Artificial Sequence Synthetic construct
MKKGHHHHHHGSQYNPQVADLKAFSKHIYNAYLKNFNMTKKKARSILTGKASHTAPFVIHDIETLWQAEKGLVWKQLVNGLPPYKEISVHVFYRCQCTTVETVRELTEFAKSIPSFSSLFLNDQVTLLKYGVHEAIFAMLASIVNKDGLLVANGSGFVTREFLRSLRKPFSDIIEPKFEFAVKFNALELDDSDLALFIAAIILCGDRPGLMNVPRVEAIQDTILRALEFHLQANHPDAQYLFPKLLQKMADLRQLVTEHAQMMQRIKKTETETSLHPLLQEIYKDMY

SEQ ID NO: 17 - DNA, 29 bp, Artificial Sequence
  Description of Artificial Sequence Synthetic primer
gttggatcccagtacaacccacaggtggc

SEQ ID NO: 18 - DNA, 32 bp, Artificial Sequence
  Description of Artificial Sequence Synthetic primer
gtgactgtcgacttagtacatgtccttgtaga

SEQ ID NO: 19 - protein, 25 aa, Artificial Sequence
  Description of Artificial Sequence Synthetic peptide
DGTPPPQEAEEPSLLKKLLLAPANT